(19) United States
(12) Patent Application Publication
(10) Pub. No.: US 2024/0391931 A1
(43) Pub. Date: Nov. 28, 2024

(54) TRICYCLIC FUSED PYRIMIDINE COMPOUNDS FOR USE AS HER2 INHIBITORS

(71) Applicant: Bristol-Myers Squibb Company, Princeton, NJ (US)

(72) Inventors: Laura Akullian D'Agostino, Sudbury, MA (US); Xinchao Chen, Schenectady, NY (US); Claudio Emundo Chuaqui, Arlington, MA (US); Hormoz Mazdiyasni, Marlborough, MA (US); Guobin Miao, Lexington, MA (US); Deqiang Niu, Lexington, MA (US)

(21) Appl. No.: 18/294,280

(22) PCT Filed: Aug. 4, 2022
(86) PCT No.: PCT/US2022/074518

§ 371 Date: Feb. 1, 2024

Related U.S. Application Data
(60) Provisional application No. 63/229,804, filed on Aug. 5, 2021.

(51) Int. Cl. C07D 495/14; A61K 31/519; A61K 31/5377; A61K 31/55; A61K 31/69; A61P 35/00; C07D 471/14; C07D 519/00; C07F 5/02

(52) U.S. Cl. CPC C07D 495/14; A61K 31/519; A61K 31/5377; A61K 31/55; A61K 31/69; A61P 35/00; C07D 471/14; C07D 519/00; C07F 5/025

(57) ABSTRACT

Provided herein are tricyclic fused pyrimidine compounds, and pharmaceutically acceptable salts thereof. Methods of use, and pharmaceutical compositions of these compounds are disclosed.

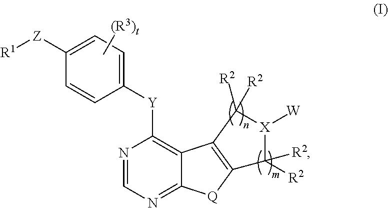

### FIELD

Provided herein are tricyclic fused pyrimidine compounds, compositions comprising the compounds, and methods for treating, preventing, and managing various disorders.

### BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (3rd ed., Mosby, St. Louis, Mo., 1993).

There is an enormous variety of cancers which are described in detail in the medical literature. Examples include cancer of the lung, colon, rectum, prostate, breast, brain, and intestine. The incidence of cancer continues to climb as the general population ages, as new cancers develop, and as susceptible populations (e.g., people infected with AIDS or excessively exposed to sunlight) grow. A tremendous demand therefore exists for new methods and compositions that can be used to treat patients with cancer.

Epidermal growth factor receptors (EGFRs) comprise a family consisting of four known tyrosine kinase receptors, HER1 (EGFR, ErbB1), HER2 (neu, ErbB2), HER3 (ErbB3), and HER4 (ErbB4). These receptors are activated by a number of ligands including EGF, TGFα, epiregulin, amphiregulin, and heregulins (neuregulins). The HER family receptors generate cell signaling cascades that transduce extracellular stimulation into intracellular events that control various cellular functions including proliferation, differentiation, and apoptosis. These receptors are elevated and abnormally activated in a large number of epithelial tumors, and this increase has been associated with the disruption of normal cellular control resulting in more aggressive tumors and a poor disease prognosis. No irreversible HER2 inhibitors with selectivity over EGFR have been reported yet.

Amplification or overexpression of HER2 occurs in approximately 15-30% of breast cancers and 10-30% of gastric/gastroesophageal cancers and serves as a prognostic and predictive biomarker. HER2 overexpression has also been seen in other cancers like stomach cancer, ovarian cancer, endometrial cancer, uterine serous endometrial carcinoma, uterine cervix cancer, bladder cancer, lung cancer, colon cancer, head and neck cancer, and esophageal cancer. Iqbal et al., Molecular Biology International, 2014, Article ID 852748. Breast cancer primarily metastasizes to the bone, lungs, regional lymph nodes, liver and brain. Metastatic HER2 positive breast cancer that has reached the CNS creates additional challenge for the treatment as the drug needs to penetrate blood-brain barrier.

HER2 aberrations (gene amplification, gene mutations, and protein overexpression) are reported in diverse malignancies. About 1-37% of tumors of the following types harbor HER2 aberrations: bladder cancer, cervix cancer, colorectal cancer, endometrial cancer, germ cell cancer, glioblastoma, head and neck cancer, liver cancer, lung cancer, ovarian cancer, pancreatic cancer, and salivary duct carcinoma. Yan et al., Cancer Treatment Reviews, 40:770-780 (2014).

In lung carcinogenesis, HER2 mutations are thought to be more clinically relevant than overexpression or gene amplification. Non-small cell lung cancer (NSCLC) accounts for 80-85% of cases of lung cancer, and HER2 mutations in NSCLC are present in approximately 4% of this subset of lung cancer patients, suggesting that thousands of patients per year may possibly benefit from therapy targeting HER2 mutations. Garrido-Castro et al., Translational Lung Cancer Research, 2(2):122-127 (2013).

It has been reported that about 92% of HER2 mutations are in-frame insertions in exon 20 which ranged from 3 to 12 bp, all nested in the most proximal region of the exon, between codons 775 and 881. The 12 bp insertion is the most common mutation (about 83%) showing a duplication/insertion of 4 amino acids (YVMA) at codon 775 (referred here as HER2YVMA). The 3 bp insertion is the second most common (about 8%) and is characterized as a complex insertion-substitution G776delinsVC (referred here as HER2VC). Two point mutations are also reported, L755S and G776C, corresponding to about 8% of HER2 mutations. HER2 V777_G778insCG mutation is also identified. Arcila et al., Clin. Cancer Res., 18(18), 17 pages (2012).

There remains a high unmet medical need for therapeutical agents that target overexpression or amplification of HER2 and/or HER2 mutations. It is also desirable that the therapeutical agents are selective over EGFR, and/or have potential for CNS penetration.

### SUMMARY

Provided herein are tricyclic fused pyrimidine compounds, and pharmaceutically acceptable salts, solvates (e.g., hydrate), prodrugs, tautomers, stereoisomers, enantiomers, or isotopologues thereof, or a mixture thereof. In one embodiment, provided herein are tetrahydropyridothienopyrimidine compounds, and pharmaceutically acceptable salts, solvates (e.g., hydrate), prodrugs, tautomers, stereoisomers, enantiomers, or isotopologues thereof, or a mixture thereof.

In one embodiment, provided herein is a compound of Formula (I):

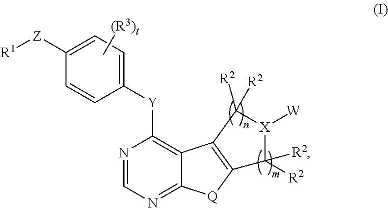

or a pharmaceutically acceptable salt, solvate (e.g., hydrate), prodrug, tautomer, stereoisomer, enantiomer, or isotopologue thereof, or a mixture thereof, wherein X, Y, Z, Q, n, m, t, R¹, R², R³, and W are defined herein or elsewhere.

Also provided herein are methods of treating and managing various diseases or disorders. The methods comprise administering to a patient in need of such treatment or management a therapeutically effective amount of a compound provided herein, or a pharmaceutically acceptable salt, solvate (e.g., hydrate), prodrug, tautomer, stereoisomer, enantiomer, or isotopologue thereof.

Also provided herein are methods of preventing various diseases and disorders, which comprise administering to a patient in need of such prevention a prophylactically effective amount of a compound provided herein, or a pharmaceutically acceptable salt, solvate (e.g., hydrate), prodrug, tautomer, stereoisomer, enantiomer, or isotopologue thereof.

Also provided herein are pharmaceutical compositions, single unit dosage forms, dosing regimens and kits which comprise a compound provided herein, or a pharmaceutically acceptable salt, solvate (e.g., hydrate), prodrug, tautomer, stereoisomer, enantiomer, or isotopologue thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. In case of conflict, the present application, including any definitions herein, will control.

### DETAILED DESCRIPTION

### Definitions

To facilitate understanding of the disclosure set forth herein, a number of terms are defined below. Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

As used herein, and unless otherwise specified, the term “alkyl” refers to a linear or branched saturated monovalent hydrocarbon radical. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 12 (C₁₋₁₂), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms. The alkyl can be unsubstituted or substituted with one or more substituents.

As used herein, and unless otherwise specified, the term “alkenyl” refers to a linear or branched monovalent hydrocarbon radical, which contains one or more, in one embodiment, one to five, carbon-carbon double bonds. The term “alkenyl” also embraces radicals having “cis” and “trans” configurations, or alternatively, “E” and “Z” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 12 (C₂₋₁₂), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl. The alkenyl can be unsubstituted or substituted with one or more substituents.

As used herein, and unless otherwise specified, the term “alkynyl” refers to a linear or branched monovalent hydrocarbon radical, which contains one or more, in one embodiment, one to five, carbon-carbon triple bonds. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 12 (C₂₋₁₂), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 12 (C₃₋₁₂), 3 to 10 (C₃₋₁₀), or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms. The alkynyl can be unsubstituted or substituted with one or more substituents.

As used herein, and unless otherwise specified, the term “cycloalkyl” refers to a cyclic saturated or partially saturated monovalent hydrocarbon radical. The term “cycloalkyl” also encompasses fused cycloalkyl, bridged cycloalkyl, and spiro cycloalkyl. In certain embodiments, the cycloalkyl has from 3 to 20 (C₃₋₂₀), from 3 to 15 (C₃₋₁₅), from 3 to 12 (C₃₋₁₂), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, cycloheptadienyl, cycloheptatrienyl, decalinyl, and adamantyl. The cycloalkyl can be unsubstituted or substituted with one or more substituents.

As used herein, and unless otherwise specified, the term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. The term “aryl” also refers to bicyclic, tricyclic, or other multicyclic hydrocarbon rings, where at least one of the rings is aromatic and the others of which may be saturated, partially unsaturated, or aromatic, for example, dihydronaphthyl, indenyl, indanyl, or tetrahydronaphthyl (tetralinyl). The aryl can be unsubstituted or substituted with one or more substituents.

As used herein, and unless otherwise specified, the term “heteroalkyl” refers to an alkyl radical that has one or more skeletal chain atoms selected from an atom other than carbon, e.g., oxygen, nitrogen, sulfur, and phosphorus, or combinations thereof. A numerical range can be given to refer to the chain length in total. For example, a —CH₂OCH₂CH₃ radical is referred to as a “C4” heteroalkyl. Connection to the parent molecular structure can be through either a heteroatom or a carbon in the heteroalkyl chain. One or more heteroatom(s) in the heteroalkyl radical can be optionally oxidized. One or more nitrogen atoms, if present, can also be optionally quaternized. The heteroalkyl can be unsubstituted or substituted with one or more substituents.

As used herein, and unless otherwise specified, the term “heteroaryl” refers to a monocyclic aromatic group and/or multicyclic aromatic group that contain at least one aromatic ring, wherein at least one aromatic ring contains one or more heteroatoms independently selected from O, S, and N. Each ring of a heteroaryl group can contain one or two O atoms, one or two S atoms, and/or one to four N atoms, provided that the total number of heteroatoms in each ring is four or less and each ring contains at least one carbon atom. The heteroaryl may be attached to the main structure at any heteroatom or carbon atom which results in the creation of a stable compound. In certain embodiments, the heteroaryl has from 5 to 20, from 5 to 15, or from 5 to 10 ring atoms. The term “heteroaryl” also refers to bicyclic, tricyclic, or other multicyclic rings, where at least one of the rings is aromatic and the others of which may be saturated, partially unsaturated, or aromatic, wherein at least one aromatic ring contains one or more heteroatoms independently selected from O, S, and N. Examples of monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to, carbazolyl, benzindolyl, phenanthrollinyl, acridinyl, phenanthridinyl, and xanthenyl. The heteroaryl can be unsubstituted or substituted with one or more substituents.

As used herein, and unless otherwise specified, the term “heterocyclyl” (or “heterocyclic”) refers to a monocyclic non-aromatic ring system and/or multicyclic ring system that contains at least one non-aromatic ring, wherein one or more of the non-aromatic ring atoms are heteroatoms independently selected from O, S, or N; and the remaining ring atoms are carbon atoms. The term “heterocyclyl” also encompasses fused heterocyclyl, bridged heterocyclyl, and spiro heterocyclyl. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the nitrogen or sulfur ring atoms may be optionally oxidized, and the nitrogen ring atoms may be optionally quaternized. The term “heterocyclyl” also refers to bicyclic, tricyclic, or other multicyclic rings, where at least one of the rings is non-aromatic and the others of which may be saturated, partially unsaturated, or aromatic, wherein at least one non-aromatic ring contains one or more heteroatoms independently selected from O, S, and N. The heterocyclyl may be attached to the main structure at any heteroatom or carbon atom which results in the creation of a stable compound. Examples of such heterocyclic radicals include, but are not limited to, acridinyl, azepinyl, benzimidazolyl, benzindolyl, benzoisoxazolyl, benzisoxazinyl, benzodioxanyl, benzodioxolyl, benzofuranonyl, benzofuranyl, benzonaphthofuranyl, benzopyranonyl, benzopyranyl, benzotetrahydrofuranyl, benzotetrahydrothienyl, benzothiadiazolyl, benzothiazolyl, benzothiophenyl, benzotriazolyl, benzothiopyranyl, benzoxazinyl, benzoxazolyl, benzothiazolyl, β-carbolinyl, carbazolyl, chromanyl, chromonyl, cinnolinyl, coumarinyl, decahydroisoquinolinyl, dibenzofuranyl, dihydrobenzisothiazinyl, dihydrobenzisoxazinyl, dihydrofuryl, dihydropyranyl, dioxolanyl, dihydropyrazinyl, dihydropyridinyl, dihydropyrazolyl, dihydropyrimidinyl, dihydropyrrolyl, dioxolanyl, 1,4-dithianyl, furanonyl, furanyl, imidazolidinyl, imidazolinyl, imidazolyl, imidazopyridinyl, imidazothiazolyl, indazolyl, indolinyl, indolizinyl, indolyl, isobenzotetrahydrofuranyl, isobenzotetrahydrothienyl, isobenzothienyl, isochromanyl, isocoumarinyl, isoindolinyl, isoindolyl, isoquinolinyl, isothiazolidinyl, isothiazolyl, isoxazolidinyl, isoxazolyl, morpholinyl, naphthyridinyl, octahydroindolyl, octahydroisoindolyl, oxadiazolyl, oxazolidinonyl, oxazolidinyl, oxazolopyridinyl, oxazolyl, oxiranyl, perimidinyl, phenanthridinyl, phenathrolinyl, phenarsazinyl, phenazinyl, phenothiazinyl, phenoxazinyl, phthalazinyl, piperazinyl, piperidinyl, 4-piperidonyl, pteridinyl, purinyl, pyrazinyl, pyrazolidinyl, pyrazolyl, pyridazinyl, pyridinyl, pyridopyridinyl, pyrimidinyl, pyrrolidinyl, pyrrolinyl, pyrrolyl, quinazolinyl, quinolinyl, quinoxalinyl, quinuclidinyl, tetrahydrofuryl, tetrahydrofuranyl, tetrahydroisoquinolinyl, tetrahydropyranyl, tetrahydrothienyl, tetrazolyl, thiadiazolopyrimidinyl, thiadiazolyl, thiamorpholinyl, thiazolidinyl, thiazolyl, thienyl, triazinyl, triazolyl, and 1,3,5-trithianyl. The heterocyclyl or heterocyclic group can be unsubstituted or substituted with one or more substituents.

As used herein, and unless otherwise specified, the term “aralkyl” or “arylalkyl” refers to a monovalent alkyl group substituted with aryl, wherein alkyl and aryl are defined above. In certain embodiments, both alkyl and aryl may independently be unsubstituted or substituted with one or more substituents. Examples of such aralkyl groups include, but are not limited to, benzyl and phenethyl groups and fused (cycloalkylaryl)alkyl groups such as 4-ethyl-indanyl.

As used herein, and unless otherwise specified, the term “heteroarylalkyl” refers to a monovalent alkyl group substituted with heteroaryl, wherein alkyl and heteroaryl are defined above. In certain embodiments, both alkyl and heteroaryl may independently be unsubstituted or substituted with one or more substituents. Examples of such heteroarylalkyl groups include, but are not limited to, furan-2-yl methyl, furan-3-yl methyl, pyridin-3-yl methyl, and indol-2-yl propyl.

As used herein, and unless otherwise specified, the term “heterocyclylalkyl” refers to a monovalent alkyl group substituted with heterocyclyl, wherein alkyl and heterocyclyl are defined above. In certain embodiments, both alkyl and heterocyclyl may independently be unsubstituted or substituted with one or more substituents. Examples of such heterocyclylalkyl groups include, but are not limited to, 4-ethyl-morpholinyl, 4-propylmorpholinyl, and tetrahydrofuran-2-yl ethyl.

As used herein, and unless otherwise specified, the term “cycloalkylalkyl” refers to a monovalent alkyl group substituted with cycloalkyl, wherein alkyl and cycloalkyl are defined above. In certain embodiments, both alkyl and cycloalkyl may independently be unsubstituted or substituted with one or more substituents. Examples of such cycloalkylalkyl groups include, but are not limited to, methylcyclopropyl, methylcyclobutyl, methylcyclopentyl, methylcyclohexyl, ethylcyclopropyl, ethylcyclobutyl, ethylcyclopentyl, ethylcyclohexyl, propylcyclopentyl, propylcyclohexyl and the like.

As used herein, and unless otherwise specified, the term “halogen”, “halide” or “halo” refers to fluorine, chlorine, bromine, and/or iodine.

As used herein, and unless otherwise specified, the terms “haloalkyl,” “haloalkenyl,” “haloalkynyl,” and “haloalkoxy” refer to alkyl, alkenyl, alkynyl, and alkoxy structures that are substituted with one or more halo groups or with combinations thereof.

As used herein, and unless otherwise specified, the term “alkoxy” refers to —O-(alkyl), wherein alkyl is defined above.

As used herein, and unless otherwise specified, the term “aryloxy” refers to —O-(aryl), wherein aryl is defined above.

As used herein, and unless otherwise specified, the term “alkyl sulfonyl” refers to —SO₂-alkyl, wherein alkyl is defined above. Examples of such alkyl sulfonyl groups include, but are not limited to, —SO₂—CH₃, —SO₂—CH₂CH₃, —SO₂—(CH₂)₂CH₃, —SO₂—(CH₂)₃CH₃, —SO₂—(CH₂)₄CH₃, —SO₂—(CH₂)₅CH₃, and the like.

As used herein, and unless otherwise specified, the term “carboxyl” and “carboxy” refers to

### —COOH.

As used herein, and unless otherwise specified, the term “alkoxycarbonyl” refers to —C(═O)O-(alkyl), wherein alkyl is defined above. Examples of such alkoxycarbonyl groups include, but are not limited to, —C(═O)O—CH₃, —C(═O)O—CH₂CH₃, —C(═O)O—(CH₂)₂CH₃, —C(═O)O—(CH₂)₃CH₃, —C(═O)O—(CH₂)₄CH₃, —C(═O)O—(CH₂)₅CH₃, and the like.

As used herein, and unless otherwise specified, the term “alkoxyalkyl” refers to -(alkyl)-O-(alkyl), wherein each alkyl is independently an alkyl group as defined above. Examples of such alkoxyalkyl groups include, but are not limited to, —CH₂OCH₃, —CH₂OCH₂CH₃, —(CH₂)₂OCH₂CH₃, —(CH₂)₂O(CH₂)₂CH₃, and the like.

As used herein, and unless otherwise specified, the term “arylalkyloxy” refers to —O-(alkyl)-(aryl), wherein alkyl and aryl are defined above. Examples of such arylalkyloxy groups include, but are not limited to, —O—(CH₂)₂phenyl, —O—(CH₂)₃phenyl, —O—CH(phenyl)₂, —O—CH(phenyl)₃, —O—(CH₂)tolyl, —O—(CH₂)anthracenyl, —O—(CH₂)fluorenyl, —O—(CH₂)indenyl, —O—(CH₂)azulenyl, —O—(CH₂)naphthyl, and the like.

As used herein, and unless otherwise specified, the term “cycloalkyloxy” refers to —O-(cycloalkyl), wherein cycloalkyl is defined above.

As used herein, and unless otherwise specified, the term “cycloalkylalkyloxy” refers to —O-(alkyl)-(cycloalkyl), wherein cycloalkyl and alkyl are defined above.

As used herein, and unless otherwise specified, the term “acyl” refers to —C(O)—R^{a}, wherein R^{a} can be, but is not limited to, hydrogen, alkyl, heteroalkyl, alkenyl, alkynyl, aryl, cycloalkyl, heteroaryl, heterocyclyl, each of which is defined above. In certain embodiments, R^{a} may be unsubstituted or substituted with one or more substituents.

As used herein, and unless otherwise specified, the term “acyloxy” refers to —O—C(O)—R^{a}, wherein R^{a} can be, but is not limited to, hydrogen, alkyl, heteroalkyl, alkenyl, alkynyl, aryl, cycloalkyl, heteroaryl, heterocyclyl, each of which is defined above. In certain embodiments, R^{a} may be unsubstituted or substituted with one or more substituents.

As used herein, and unless otherwise specified, the term “amino” refers to —N(R^{b})(R^{b}), wherein each R^{b} independently can be, but is not limited to, hydrogen, alkyl, heteroalkyl, alkenyl, alkynyl, aryl, cycloalkyl, heteroaryl, heterocyclyl, each of which is defined above. When a —N(R^{b})(R^{b}) group has two R^{b} other than hydrogen, they can be combined with the nitrogen atom to form a ring. In one embodiment, the ring is a 3-, 4-, 5-, 6-, 7-, or 8-membered ring. In one embodiment, one or more ring atoms are heteroatoms independently selected from 0, S, or N. The term “amino” also includes N-oxide —N⁺(R^{b})(R^{b})O⁻. In certain embodiments, each R^{b} or the ring formed by —N(R^{b})(R^{b}) independently may be unsubstituted or substituted with one or more substituents.

As used herein, and unless otherwise specified, the term “amide” or “amido” refers to —C(O)N(R)₂ or —NRC(O)R^{b}, wherein each R^{b} independently can be, but is not limited to, hydrogen, alkyl, heteroalkyl, alkenyl, alkynyl, aryl, cycloalkyl, heteroaryl, heterocyclyl, each of which is defined above. When a —C(O)N(R)₂ group has two R^{b} other than hydrogen, they can be combined with the nitrogen atom to form a ring. In one embodiment, the ring is a 3-, 4-, 5-, 6-, 7-, or 8-membered ring. In one embodiment, one or more ring atoms are heteroatoms independently selected from O, S, or N. In certain embodiments, each R^{b} or the ring formed by —N(R^{b})(R^{b}) independently may be unsubstituted or substituted with one or more substituents.

As used herein, and unless otherwise specified, the term “aminoalkyl” refers to -(alkyl)-(amino), wherein alkyl and amino are defined above.

As used herein, and unless otherwise specified, the term “aminoalkoxy” refers to —O-(alkyl)-(amino), wherein alkyl and amino are defined above.

As used herein, and unless otherwise specified, the term “alkylamino” refers to —NH(alkyl) or —N(alkyl)(alkyl), wherein alkyl is defined above. Examples of such alkylamino groups include, but are not limited to, —NHCH₃, —NHCH₂CH₃, —NH(CH₂)₂CH₃, —NH(CH₂)₃CH₃, —NH(CH₂)₄CH₃, —NH(CH₂)₅CH₃, —N(CH₃)₂, —N(CH₂CH₃)₂, —N((CH₂)₂CH₃)₂, —N(CH₃)(CH₂CH₃), and the like.

As used herein, and unless otherwise specified, the term “arylamino” refers to —NH(aryl) or —N(aryl)(aryl), wherein aryl is defined above. Examples of such arylamino groups include, but are not limited to, —NH(phenyl), —NH(tolyl), —NH(anthracenyl), —NH(fluorenyl), —NH(indenyl), —NH(azulenyl), —NH(pyridinyl), —NH(naphthyl), and the like.

As used herein, and unless otherwise specified, the term “arylalkylamino” refers to —NH-(alkyl)-(aryl), wherein alkyl and aryl are defined above. Examples of such arylalkylamino groups include, but are not limited to, —NH—CH₂-(phenyl), —NH—CH₂-(tolyl), —NH—CH₂-(anthracenyl), —NH—CH₂-(fluorenyl), —NH—CH₂-(indenyl), —NH—CH₂-(azulenyl), —NH—CH₂-(pyridinyl), —NH—CH₂-(naphthyl), —NH—(CH₂)₂-(phenyl) and the like.

As used herein, and unless otherwise specified, the term “cycloalkylamino” refers to —NH-(cycloalkyl), wherein cycloalkyl is defined above. Examples of such cycloalkylamino groups include, but are not limited to, —NH-cyclopropyl, —NH-cyclobutyl, —NH-cyclopentyl, —NH— cyclohexyl, —NH-cycloheptyl, and the like.

As used herein, and unless otherwise specified, the term “alkylaminoalkyl” refers to -(alkyl)-NH(alkyl) or -(alkyl)-N(alkyl)(alkyl), wherein each “alkyl” is independently an alkyl group defined above. Examples of such alkylaminoalkyl groups include, but are not limited to, —CH₂—NH—CH₃, —CH₂—NHCH₂CH₃, —CH₂—NH(CH₂)₂CH₃, —CH₂—NH(CH₂)₃CH₃, —CH₂—NH(CH₂)₄CH₃, —CH₂—NH(CH₂)₅CH₃, —(CH₂)₂—NH—CH₃, —CH₂—N(CH₃)₂, —CH₂—N(CH₂CH₃)₂, —CH₂—N((CH₂)₂CH₃)₂, —CH₂—N(CH₃)(CH₂CH₃), —(CH₂)₂—N(CH₃)₂, and the like.

As used herein, and unless otherwise specified, the term “hydroxyalkyl” refers to -(alkyl)-OH, wherein alkyl is defined above.

As used herein, and unless otherwise specified, the term “sulfanyl”, “sulfide”, or “thio” refers to —S—R^{a}, wherein R^{a} can be, but is not limited to, alkyl, heteroalkyl, alkenyl, alkynyl, aryl, cycloalkyl, heteroaryl, heterocyclyl, each of which is defined above. In certain embodiments, R^{a} may be unsubstituted or substituted with one or more substituents.

As used herein, and unless otherwise specified, the term “sulfoxide” refers to —S(O)—R^{a}, wherein R^{a} can be, but is not limited to, alkyl, heteroalkyl, alkenyl, alkynyl, aryl, cycloalkyl, heteroaryl, heterocyclyl, each of which is defined above. In certain embodiments, R^{a} may be unsubstituted or substituted with one or more substituents.

As used herein, and unless otherwise specified, the term “sulfonyl” or “sulfone” refers to —S(O)₂—R^{a}, wherein R^{a} can be, but is not limited to, alkyl, heteroalkyl, alkenyl, alkynyl, aryl, cycloalkyl, heteroaryl, heterocyclyl, each of which is defined above. In certain embodiments, R^{a} may be unsubstituted or substituted with one or more substituents.

As used herein, and unless otherwise specified, the term “sulfonamido” or “sulfonamide” refers to —S(═O)₂—N(R^{b})₂ or —N(R^{b})—S(═O)₂—R^{b}, wherein each R^{b} independently can be, but is not limited to, hydrogen, alkyl, heteroalkyl, alkenyl, alkynyl, aryl, cycloalkyl, heteroaryl, heterocyclyl, each of which is defined above. When a —C(O)N(R)₂ group has two R^{b} other than hydrogen, they can be combined with the nitrogen atom to form a ring. In one embodiment, the ring is a 3-, 4-, 5-, 6-, 7-, or 8-membered ring. In one embodiment, one or more ring atoms are heteroatoms independently selected from O, S, or N. In certain embodiments, each R^{b} or the ring formed by —N(R^{b})(R^{b}) independently may be unsubstituted or substituted with one or more substituents.

As used herein, and unless otherwise specified, the term “boronic acid” refers to a —B(OH)₂ radical or a chemical compound comprising a —B(OH)₂ moiety.

“Azide” refers to a —N₃ radical.

“Cyano” refers to a —CN radical.

“Nitro” refers to the —NO₂ radical.

“Oxa” refers to the —O— radical.

“Oxo” refers to the ═O radical.

When the groups described herein are said to be “substituted,” they may be substituted with any appropriate substituent or substituents. Illustrative examples of substituents include, but are not limited to, those found in the exemplary compounds and embodiments disclosed herein, as well as halogen (chloro, iodo, bromo, or fluoro); alkyl; alkenyl; alkynyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aryloxyamine, aralkoxyamine; N-oxide; hydrazine; hydrazide; hydrazone; azide; isocyanate; isothiocyanate; cyanate; thiocyanate; oxo (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, and unless otherwise specified, the term “isomer” refers to different compounds that have the same molecular formula. “Stereoisomers” are isomers that differ only in the way the atoms are arranged in space. “Atropisomers” are stereoisomers from hindered rotation about single bonds. “Enantiomers” are a pair of stereoisomers that are non-superimposable mirror images of each other. A mixture of a pair of enantiomers in any proportion can be known as a “racemic” mixture. “Diastereoisomers” are stereoisomers that have at least two asymmetric atoms, but which are not mirror-images of each other. The absolute stereochemistry can be specified according to the Cahn-Ingold-Prelog R—S system. When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. However, the sign of optical rotation, (+) and (−), is not related to the absolute configuration of the molecule, R and S. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. Optically active (R)- and (S)-isomers can be prepared, for example, using chiral synthons or chiral reagents, or resolved using conventional techniques.

As used herein, and unless otherwise specified, the term “stereomerically enriched” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, greater than about 99% by weight of one stereoisomer of the compound and less than about 1% by weight of the other stereoisomers of the compound. All such isomeric forms are included within the embodiments disclosed herein, including mixtures thereof.

As used herein, and unless otherwise specified, the terms “optically active,” “enantiomerically active,” “enantiomerically enriched,” or “enantiomerically pure” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%.

As used herein, and unless otherwise specified, the term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

As used herein, and unless otherwise specified, the terms “treat,” “treating,” and “treatment” refer to the eradication or amelioration of a disease or disorder, or of one or more symptoms associated with the disease or disorder. In general, treatment occurs after the onset of the disease or disorder. In certain embodiments, the terms refer to minimizing the spread or worsening of the disease or disorder resulting from the administration of one or more prophylactic or therapeutic agents to a subject with such a disease or disorder.

As used herein, and unless otherwise specified, the terms “prevent,” “preventing,” and “prevention” refer to the prevention of the onset, recurrence or spread of a disease or disorder, or of one or more symptoms thereof. In general, prevention occurs prior to the onset of the disease or disorder.

As used herein, and unless otherwise specified, the terms “manage,” “managing,” and “management” refer to preventing or slowing the progression, spread or worsening of a disease or disorder, or of one or more symptoms thereof. Sometimes, the beneficial effects that a subject derives from a prophylactic or therapeutic agent do not result in a cure of the disease or disorder.

As used herein, and unless otherwise specified, the term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

As used herein, and unless otherwise specified, the term “IC₅₀” refers an amount, concentration, or dosage of a compound that is required for 50% inhibition of a maximal response in an assay that measures such response.

As used herein, and unless otherwise specified, the term “selective inhibition” or “selectively inhibit” as applied to a biologically active agent refers to the agent's ability to selectively reduce the target signaling activity as compared to off-target signaling activity, via direct or indirect interaction with the target. In one embodiment, the ratio of selectivity can be greater than a factor of about 1, greater than a factor of about 2, greater than a factor of about 3, greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, greater than a factor of about 10,000, or greater than a factor of about 20,000, where selectivity can be measured by ratio of IC₅₀ values, which in turn can be measured by, e.g., in vitro or in vivo assays such as those described in Examples described herein.

As used herein, and unless otherwise specified, the term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, FL, 2004.

As used herein, and unless otherwise specified, the term “pharmaceutically acceptable form” of a compound includes, but is not limited to, pharmaceutically acceptable salts, hydrates, solvates, isomers, prodrugs, and isotopically labeled derivatives of the compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, naphthalene-m,n-bissulfonates, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, naphthalene-m,n-bissulfonic acids and the like.

Pharmaceutically acceptable salts derived from appropriate bases include alkali metal, alkaline earth metal, ammonium and N*(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

As used herein, and unless otherwise specified, the term “solvate” refers to a compound provided herein or a salt thereof, which further includes a stoichiometric or non-stoichiometric amount of solvent bound by non-covalent intermolecular forces. Where the solvent is water, the solvate is a hydrate.

As used herein, and unless otherwise specified, the term “tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution.

In certain embodiments, the pharmaceutically acceptable form is a prodrug. As used herein, and unless otherwise specified, the term “prodrug” of a compound refers to compounds that are transformed in vivo to yield the compound or a pharmaceutically acceptable form of the compound. A prodrug can be inactive when administered to a subject, but is converted in vivo to an active compound, for example, by hydrolysis (e.g., hydrolysis in blood). The term “prodrug” is also meant to include any covalently bonded carriers, which release the active compound in vivo when such prodrug is administered to a subject. Prodrugs of an active compound, as described herein, can be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. Other examples of prodrugs include compounds that comprise —NO, —NO₂, —ONO, or —ONO₂ moieties. Prodrugs can typically be prepared using well-known methods, such as those described in Burger's Medicinal Chemistry and Drug Discovery, 172-178, 949-982 (Manfred E. Wolff ed., 5th ed., 1995), and Design of Prodrugs (H. Bundgaard ed., Elsevier, New York, 1985).

Unless otherwise stated, structures depicted herein are also meant to include compounds which differ only in the presence of one or more isotopically enriched atoms. Examples of isotopes that can be incorporated into disclosed compounds include isotopes of hydrogen, carbon, nitrogen, oxygen, phosphorus, sulfur, fluorine, and chlorine, such as, e.g., ²H, ³H, ¹³C, ¹⁴C, ¹⁵N, ¹⁸O, ¹⁷O, ³¹P, ³²P ³⁵S, ¹⁸F, and ³⁶Cl, respectively. For example, compounds having the present structures except for the replacement or enrichment of a hydrogen by deuterium or tritium at one or more atoms in the molecule, or the replacement or enrichment of a carbon by ¹³C or 14C at one or more atoms in the molecule, are within the scope of this disclosure. In one embodiment, provided herein are isotopically labeled compounds having one or more hydrogen atoms replaced by or enriched by deuterium. In one embodiment, provided herein are isotopically labeled compounds having one or more hydrogen atoms replaced by or enriched by tritium. In one embodiment, provided herein are isotopically labeled compounds having one or more carbon atoms replaced or enriched by ¹³C. In one embodiment, provided herein are isotopically labeled compounds having one or more carbon atoms replaced or enriched by ¹⁴C.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, and unless otherwise specified, the terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer of a compound described herein.

As used herein, and unless otherwise specified, the terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

### Compounds

Provided herein are tricyclic fused pyrimidine compounds, and pharmaceutically acceptable salts, solvates (e.g., hydrates), prodrugs, tautomers, stereoisomers, enantiomers, or isotopologues thereof, or a mixture thereof.

In one embodiment, provided herein is a compound of Formula (I):

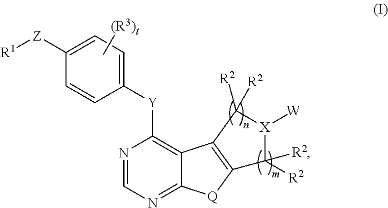

or a pharmaceutically acceptable salt, solvate (e.g., hydrate), prodrug, tautomer, stereoisomer, enantiomer, or isotopologue thereof, or a mixture thereof, wherein:
X is CR⁵ or N;
Y is NR⁶, CR⁷R⁸, or O;
Z is NR⁶ or O;
Q is S, NR⁶, or CR⁷R⁸;
n is 1, 2, 3, or 4;
m is 1, 2, 3, or 4;
t is 0, 1, 2, 3, or 4;
R¹ is aryl, heteroaryl, cycloalkyl, or heterocyclyl;
each instance of R² is independently hydrogen, alkyl, alkenyl, alkynyl, haloalkyl, hydroxyalkyl, heteroalkyl, halogen, cyano, nitro, amido, cycloalkyl, heterocyclyl, aryl, heteroaryl, —OR′, or —NR′R″, wherein R′ and R″ are independently hydrogen, alkyl, heteroalkyl, aryl, or heteroaryl, or R′ and R″ are taken together with nitrogen to form a cyclic moiety;
or two R² are taken together to form a C₁-C₃ alkylene;
each instance of R³ is independently alkyl, alkenyl, alkynyl, haloalkyl, hydroxyalkyl, heteroalkyl, halogen, hydroxyl, alkoxy, cyano, nitro, amino, amido, cycloalkyl, heterocyclyl, aryl, or heteroaryl;
each instance of R⁵, R⁶, R⁷, and R⁸ is independently hydrogen or alkyl;
W is
   -L-(NR ⁶)ₛ—C(═O)—(CR¹⁰=CR¹⁰)—R¹¹,
   -L-(NR⁶)ₛ—C(═O)—C(═CR¹⁰R¹⁰)—R¹¹
   -L-(CR¹⁰=CR¹⁰)—C(═O)—(NR ⁶)ₛ—R¹¹,
   -L-C(═CR¹⁰R¹⁰)—C(═O)—(NR ⁶)ₛ—R¹¹,
   -L-(NR ⁶)ₛ—S(═O)₂—(CR¹⁰=CR¹⁰)—R¹¹,
   -L-(NR ⁶)ₛ—S(═O)₂—C(═CR¹⁰R¹⁰)—R¹¹,
   -L-(CR¹⁰=CR¹⁰)—S(═O)₂—(NR⁶)ₛ—R¹¹; or
   -L-C(═CR¹⁰R¹⁰)—S(═O)₂—(NR ⁶)ₛ—R¹¹;
L is a bond or C₁-C₃ alkylene;
s is 0 or 1;
each instance of R¹⁰ is independently hydrogen, alkyl, haloalkyl, hydroxyalkyl, heteroalkyl, alkoxy, cycloalkyl, heterocyclyl, aryl, heteroaryl, or halo;
R¹¹ is hydrogen, —OR¹², —(C₁-C₃ alkylene)-OR¹², —NR²R¹³, —(C₁-C₃ alkylene)-NR¹²R¹³, cycloalkyl, —(C₁-C₃ alkylene)-cycloalkyl, heterocyclyl, —(C₁-C₃ alkylene)-heterocyclyl, aryl, —(C₁-C₃ alkylene)-aryl, heteroaryl, or —(C₁-C₃ alkylene)-heteroaryl; and R¹² and R¹³ are independently hydrogen, alkyl, haloalkyl, hydroxyalkyl, heteroalkyl, alkoxy, cycloalkyl, heterocyclyl, aryl, heteroaryl, cycloalkylalkyl, heterocyclylalkyl, arylalkyl, or heteroarylalkyl, or R¹² and R¹³ are taken together with nitrogen to form a cyclic moiety.

In one embodiment, n is 1. In another embodiment, n is 2. In yet another embodiment, n is 3. In yet another embodiment, n is 4.

In one embodiment, m is 1. In another embodiment, m is 2. In yet another embodiment, m is 3. In yet another embodiment, m is 4.

In one embodiment, n is 1, and m is 1 or 2. In another embodiment, n is 1 or 2, and m is 1.

In one embodiment, n is 2, and m is 1. In one embodiment, provided herein is a compound of Formula (II-1):

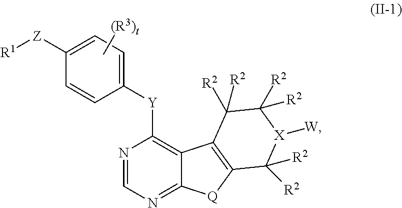

or a pharmaceutically acceptable salt, solvate (e.g., hydrate), prodrug, tautomer, stereoisomer, enantiomer, or isotopologue thereof, or a mixture thereof.

In one embodiment, n is 1, and m is 2. In one embodiment, provided herein is a compound of Formula (II-2):

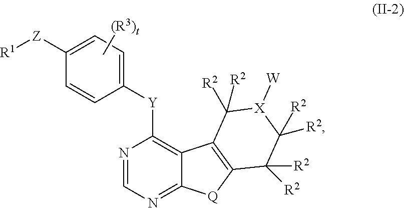

or a pharmaceutically acceptable salt, solvate (e.g., hydrate), prodrug, tautomer, stereoisomer, enantiomer, or isotopologue thereof, or a mixture thereof.

In one embodiment, n is 1, and m is 1. In one embodiment, provided herein is a compound of Formula (II-3):

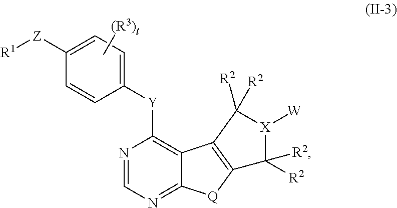

or a pharmaceutically acceptable salt, solvate (e.g., hydrate), prodrug, tautomer, stereoisomer, enantiomer, or isotopologue thereof, or a mixture thereof.

In one embodiment, n is 2, and m is 2. In one embodiment, provided herein is a compound of Formula (II-4):

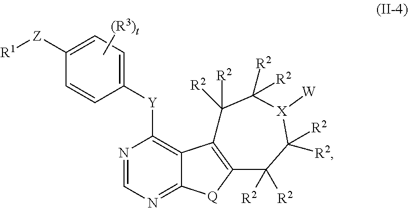

or a pharmaceutically acceptable salt, solvate (e.g., hydrate), prodrug, tautomer, stereoisomer, enantiomer, or isotopologue thereof, or a mixture thereof.

In one embodiment, n is 3, and m is 1. In one embodiment, provided herein is a compound of Formula (II-5):

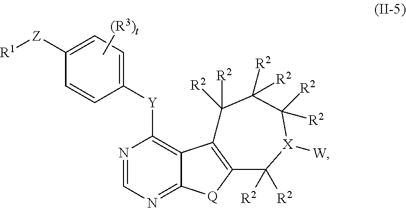

or a pharmaceutically acceptable salt, solvate (e.g., hydrate), prodrug, tautomer, stereoisomer, enantiomer, or isotopologue thereof, or a mixture thereof.

In one embodiment, n is 1, and m is 3. In another embodiment, n is 1, and m is 4. In yet another embodiment, n is 2, and m is 3. In yet another embodiment, n is 2, and m is 4. In yet another embodiment, n is 3, and m is 2. In yet another embodiment, n is 3, and m is 3. In yet another embodiment, n is 3, and m is 4. In yet another embodiment, n is 4, and m is 1. In yet another embodiment, n is 4, and m is 2. In yet another embodiment, n is 4, and m is 3. In yet another embodiment, n is 4, and m is 4.

In one embodiment, t is 0. In another embodiment, t is 1. In yet another embodiment, t is 2. In yet another embodiment, t is 3. In yet another embodiment, t is 4.

In one embodiment, when t is 1, the moiety is

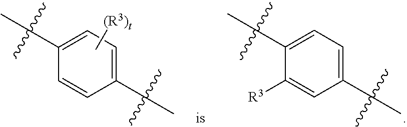

In another embodiment, when t is 1, the moiety

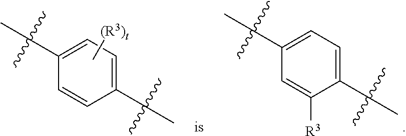

In one embodiment, provided herein is a compound of Formula (III-1), (III-2), (III-3), (III-4) or (III-5):

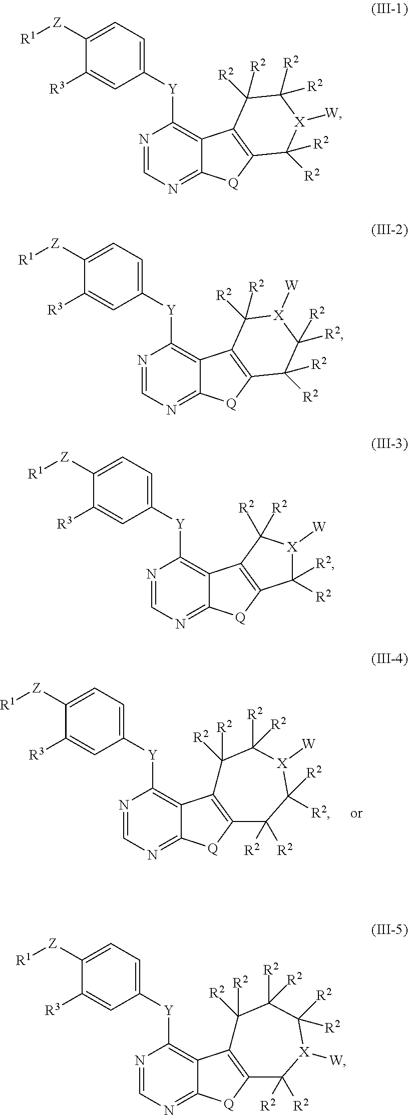

or a pharmaceutically acceptable salt, solvate (e.g., hydrate), prodrug, tautomer, stereoisomer, enantiomer, or isotopologue thereof, or a mixture thereof.

In one embodiment, when t is 2, the moiety

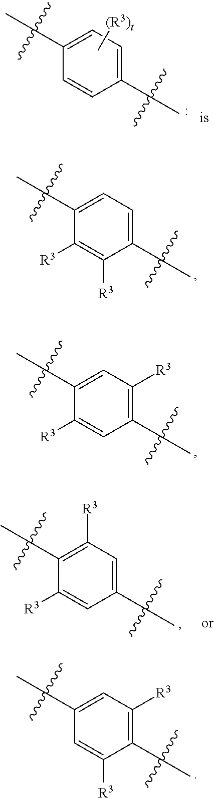

In one embodiment, when t is 3, the moiety

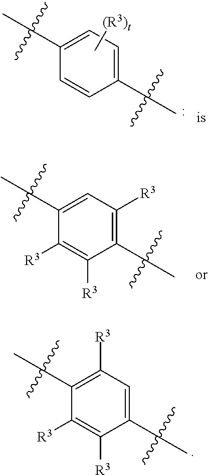

In one embodiment, when t is 4, the moiety

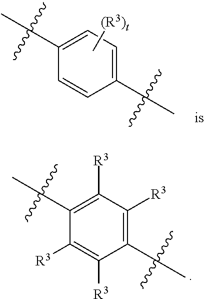

In one embodiment, Y is NR⁶. In one embodiment, Y is NH. In one embodiment, Y is NR⁶, wherein R⁶ is alkyl. In one embodiment, Y is NR⁶, wherein R⁶ is C₁₋₄ alkyl. In one embodiment, Y is NR⁶, wherein R⁶ is methyl.

In one embodiment, Y is CR⁷R⁸. In one embodiment, Y is CH₂. In one embodiment, Y is CR⁷R⁸, wherein one of R⁷ and R⁸ is H and the other is alkyl. In one embodiment, Y is CR⁷R⁸, wherein one of R⁷ and R⁸ is H and the other is C₁₋₄ alkyl. In one embodiment, Y is CR⁷R⁸, wherein one of R⁷ and R⁸ is H and the other is methyl. In one embodiment, Y is CR⁷R⁸, wherein R⁷ and R⁸ are independently alkyl. In one embodiment, Y is CR⁷R⁸, wherein R⁷ and R⁸ are independently C₁₋₄ alkyl. In one embodiment, Y is CR⁷R⁸, wherein R⁷ and R⁸ are both methyl.

In one embodiment, Y is O.

In one embodiment, Z is NR⁶. In one embodiment, Z is NH. In one embodiment, Z is NR⁶, wherein R⁶ is alkyl. In one embodiment, Z is NR⁶, wherein R⁶ is C₁₋₄ alkyl. In one embodiment, Z is NR⁶, wherein R⁶ is methyl.

In one embodiment, Z is O.

In one embodiment, Y is NH, and Z is O. In another embodiment, Y is NH, and Z is NH. In another embodiment, Y is O, and Z is O. In another embodiment, Y is O, and Z is NH.

In one embodiment, Q is S.

In one embodiment, Q is NR⁶. In one embodiment, Q is NH. In one embodiment, Q is NR⁶, wherein R⁶ is alkyl. In one embodiment, Q is NR⁶, wherein R⁶ is C₁₋₄ alkyl. In one embodiment, Q is NR⁶, wherein R⁶ is methyl.

In one embodiment, Q is CR⁷R⁸. In one embodiment, Q is CH₂. In one embodiment, Q is CR⁷R⁸, wherein one of R⁷ and R⁸ is H and the other is alkyl. In one embodiment, Q is CR⁷R⁸, wherein one of R⁷ and BY is H and the other is C₁₋₄ alkyl. In one embodiment, Q is CR⁷R⁸, wherein one of R⁷ and BY is H and the other is methyl. In one embodiment, Q is CR⁷R⁸, wherein R⁷ and R⁸ are independently alkyl. In one embodiment, Q is CR⁷R⁸, wherein R⁷ and R⁸ are independently C₁₋₄ alkyl. In one embodiment, Q is CR⁷R⁸, wherein R⁷ and R⁸ are both methyl.

In one embodiment, provided herein is a compound of Formula (IV-1), (IV-2), (IV-3), (IV-4), (IV-5), IV-6), or (IV-7):

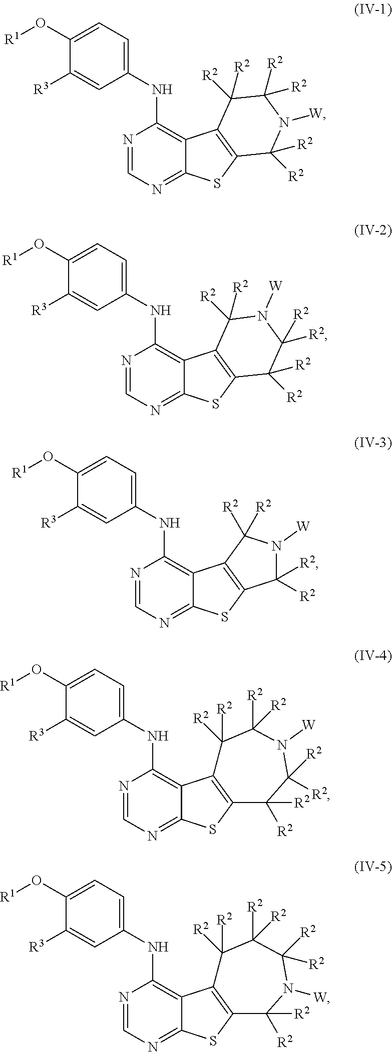

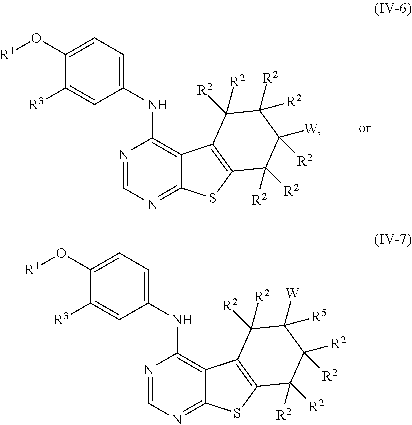

or a pharmaceutically acceptable salt, solvate (e.g., hydrate), prodrug, tautomer, stereoisomer, enantiomer, or isotopologue thereof, or a mixture thereof.

In one embodiment, X is CR⁵. In one embodiment, X is CH. In one embodiment, X is CR⁵, wherein R⁵ is alkyl. In one embodiment, X is CR⁵, wherein R⁵ is C₁₋₄ alkyl. In one embodiment, X is CR⁵, wherein R⁵ is methyl.

In one embodiment, X is N.

In one embodiment, W is -L-(NR⁶)ₛ—C(═O)—(CR¹⁰=CR¹⁰)—R¹¹. In one embodiment, W is —NR⁶—C(═O)—(CR¹⁰=CR¹⁰)—R¹¹. In one embodiment, W is —NH—C(═O)—(CR¹⁰=CR¹⁰)—R¹¹. In one embodiment, W is -L-C(═O)—(CR¹⁰=CR¹⁰)—R¹¹. In one embodiment, W is —CH₂—C(═O)—(CR¹⁰=CR¹⁰)—R¹¹. In one embodiment, W is —C(═O)—(CR¹⁰=CR¹⁰)—R¹¹.

In one embodiment, W is -L-(NR⁶)ₛ—C(═O)—C(═CR¹⁰R¹⁰)—R¹¹. In one embodiment, W is —NR⁶—C(═O)—C(═CR¹⁰R¹⁰)—R¹¹. In one embodiment, W is —NH—C(═O)—C(═CR¹⁰R¹⁰)—R¹¹. In one embodiment, W is -L-C(═O)—C(═CR¹⁰R¹⁰)—R¹¹. In one embodiment, W is —CH₂—C(═O)—C(═CR¹⁰R¹⁰)—R¹¹. In one embodiment, W is —C(═O)—C(═CR¹⁰R¹⁰)—R¹¹.

In one embodiment, W is -L-(CR¹⁰=CR¹⁰)—C(═O)—(NR⁶)ₛ—R¹¹. In one embodiment, W is —(CR¹⁰=CR¹⁰)—C(═O)—NR⁶—R¹¹. In one embodiment, W is —(CR¹⁰=CR¹⁰)—C(═O)—NH—R¹¹. In one embodiment, W is -L-(CR¹⁰=CR¹⁰)—C(═O)—R¹¹. In one embodiment, W is —CH₂—(CR¹⁰=CR¹⁰)—C(═O)—R¹¹. In one embodiment, W is —(CR¹⁰=CR¹⁰)—C(═O)—R¹¹.

In one embodiment, W is -L-C(═CR¹⁰R¹⁰)—C(═O)—(NR⁶)ₛ—R¹¹. In one embodiment, W is —C(═CR¹⁰R¹⁰)—C(═O)—NR⁶—R¹¹. In one embodiment, W is —C(═CR¹⁰R¹⁰)—C(═O)—NH—R¹¹. In one embodiment, W is -L-C(═CR¹⁰R¹⁰)—C(═O)—R¹¹. In one embodiment, W is —CH₂—C(═CR¹⁰R¹⁰)—C(═O)—R¹¹. In one embodiment, W is —C(═CR¹⁰R¹⁰)—C(═O)—R¹¹.

In one embodiment, W is -L-(NR⁶)ₛ—S(═O)₂—(CR¹⁰=CR¹⁰)—R¹¹. In one embodiment, W is —NR⁶—S(═O)₂—(CR¹⁰=CR¹⁰)—R¹¹. In one embodiment, W is —NH—S(═O)₂—(CR¹⁰=CR¹⁰)—R¹¹. In one embodiment, W is -L-S(═O)₂—(CR¹⁰=CR¹⁰)—R¹¹. In one embodiment, W is —CH₂—S(═O)₂—(CR¹⁰=CR¹⁰)—R¹¹. In one embodiment, W is —S(═O)₂—(CR¹⁰=CR¹⁰)—R¹¹.

In one embodiment, W is -L-(NR⁶)ₛ—S(═O)₂—C(═CR¹⁰R¹⁰)—R¹¹. In one embodiment, W is —NR⁶—S(═O)₂—C(═CR¹⁰R¹⁰)—R¹¹. In one embodiment, W is —NH—S(═O)₂—C(═CR¹⁰R¹⁰)—R¹¹. In one embodiment, W is -L-S(═O)₂—C(═CR¹⁰R¹⁰)—R¹¹. In one embodiment, W is —CH₂—S(═O)₂—C(═CR¹⁰R¹⁰)—R¹¹. In one embodiment, W is —S(═O)₂—C(═CR¹⁰R¹⁰)—R¹¹.

In one embodiment, W is -L-(CR¹⁰=CR¹⁰)—S(═O)₂—(NR⁶)ₛ—R¹¹. In one embodiment, W is —(CR¹⁰=CR¹⁰)—S(═O)₂—NR⁶—R¹¹. In one embodiment, W is —(CR¹⁰=CR¹⁰)—S(═O)₂—NH—R¹¹. In one embodiment, W is -L-(CR¹⁰=CR¹⁰)—S(═O)₂—R¹¹. In one embodiment, W is —CH₂—(CR¹⁰=CR¹⁰)—S(═O)₂—R¹¹. In one embodiment, W is —(CR¹⁰=CR¹⁰)—S(═O)₂—R¹¹.

In one embodiment, W is -L-C(═CR¹⁰R¹⁰)—S(═O)₂—(NR⁶)ₛ—R¹¹. In one embodiment, W is —C(═CR¹⁰R¹⁰)—S(═O)₂—NR⁶—R¹¹. In one embodiment, W is —C(═CR¹⁰R¹⁰)—S(═O)₂—NH—R¹¹. In one embodiment, W is -L-C(═CR¹⁰R¹⁰)—S(═O)₂—R¹¹. In one embodiment, W is —CH₂—C(═CR¹⁰R¹⁰)—S(═O)₂—R¹¹. In one embodiment, W is —C(═CR¹⁰R¹⁰)—S(═O)₂—R¹¹.

In one embodiment, W is:

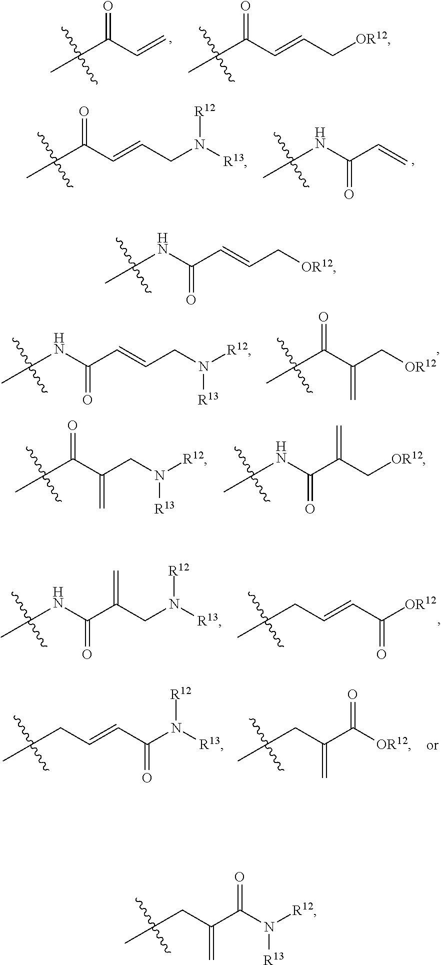

In one embodiment, W is:

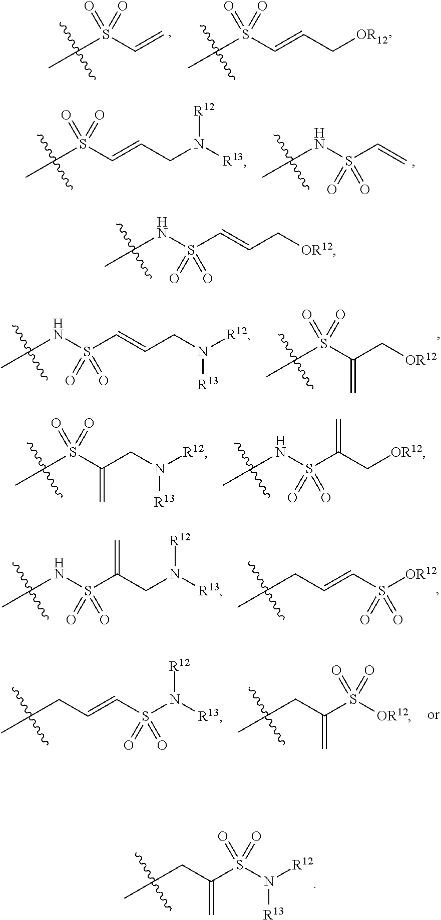

In one embodiment, X is N, and W is —C(═O)—(CR¹⁰=CR¹⁰)—R¹¹. In one embodiment, X is N, and W is —C(═O)—C(═CR¹⁰R¹⁰)—R¹¹. In one embodiment, X is N, and W is —S(═O)₂—(CR¹⁰=CR¹⁰)—R¹¹. In one embodiment, X is N, and W is —S(═O)₂—C(═CR⁰R¹⁰)—R¹¹.

In one embodiment, X is CR⁵, and W is —(NR⁶)ₛ—C(═O)—(CR¹⁰=CR¹⁰)—R¹¹. In one embodiment, X is CR⁵, and W is —(NR⁶)ₛ—C(═O)—C(═CR¹⁰R¹⁰)—R¹¹. In one embodiment, X is CR⁵, and W is —(NR⁶)ₛ—S(═O)₂—(CR¹⁰=CR¹⁰)—R¹¹. In one embodiment, X is CR⁵, and W is —(NR⁶)ₛ—S(═O)₂—C(═CR¹OR¹⁰)—R¹¹.

In one embodiment, the carbon-carbon double bond (CR¹⁰=CR¹⁰) in W has a E-configuration. In one embodiment, the carbon-carbon double bond (CR¹⁰=CR¹⁰) in W has a Z-configuration. In one embodiment, the carbon-carbon double bond C(═CR¹⁰R¹⁰) in W has a E-configuration. In one embodiment, the carbon-carbon double bond C(═CR¹⁰R¹⁰) in W has a Z-configuration.

In one embodiment, L is a bond. In one embodiment, L is a C₁-C₃ alkylene. In one embodiment, L is a methylene. In one embodiment, L is CH₂. In one embodiment, the alkylene is unsubstituted. In another embodiment, the alkylene is substituted with one or more of substituents. In one embodiment, the alkylene is substituted with one or more of alkyl, cycloalkyl, aryl, heteroaryl, heterocyclyl, or halo.

In one embodiment, s is 0. In another embodiment, s is 1.

In one embodiment, each instance of R¹⁰ is independently hydrogen, alkyl, cycloalkyl, heterocyclyl, aryl, heteroaryl, or halo. In one embodiment, each instance of R¹⁰ is independently hydrogen or alkyl. In one embodiment, each instance of R¹⁰ is independently hydrogen or C₁₋₄ alkyl. In one embodiment, each instance of R¹⁰ is independently hydrogen or methyl. In one embodiment, both of R¹⁰ are hydrogen. In one embodiment, one of R¹⁰ is hydrogen and the other is methyl. In one embodiment, both of R¹⁰ are methyl.

In one embodiment, provided herein is a compound of Formula (V-1), (V-2), (V-3), (V-4), (V-5), (V-6), (V-7), (V-8), (V-9), (V-10), (V-11), (V-12), (V-13), or (V-14):

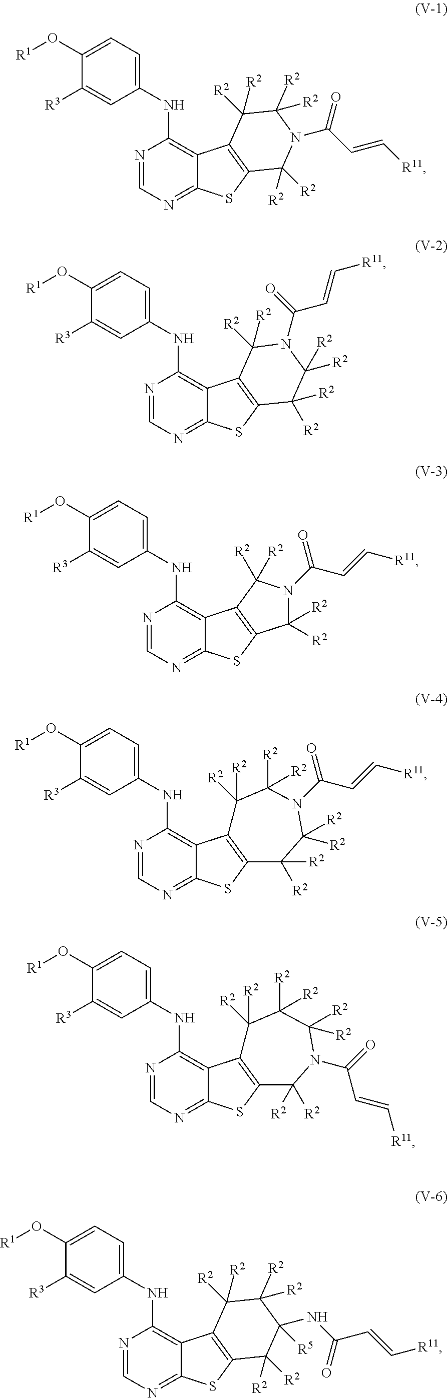

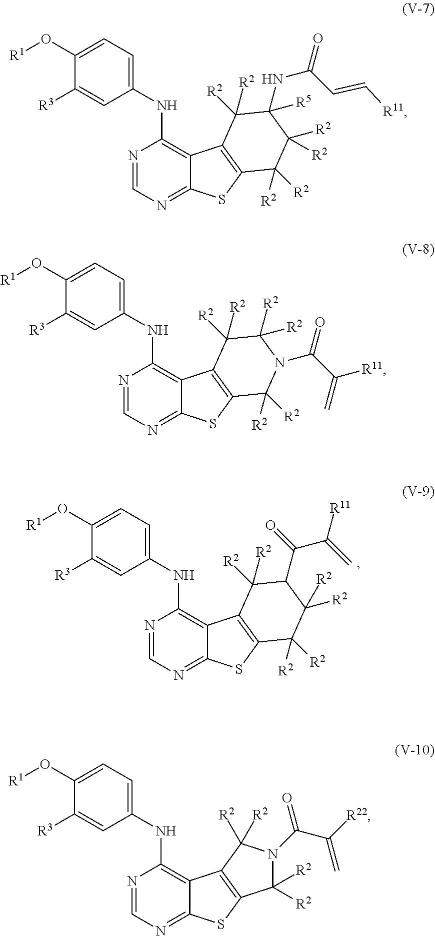

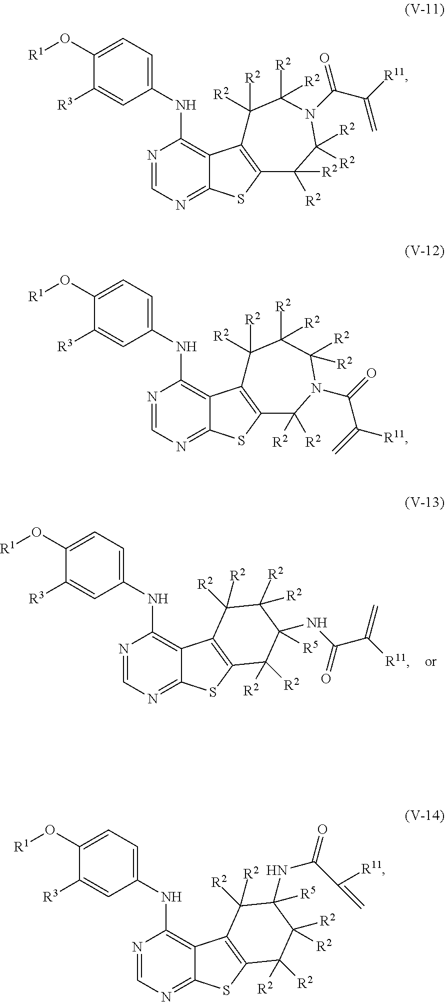

or a pharmaceutically acceptable salt, solvate (e.g., hydrate), prodrug, tautomer, stereoisomer, enantiomer, or isotopologue thereof, or a mixture thereof.

In one embodiment, R¹¹ is hydrogen.

In one embodiment, R¹¹ is cycloalkyl. In one embodiment, R¹¹ is —(C₁-C₃ alkylene)-cycloalkyl. In one embodiment, R¹¹ is —CH₂-cycloalkyl.

In one embodiment, R¹¹ is heterocyclyl. In one embodiment, R¹¹ is —(C₁-C₃ alkylene)-heterocyclyl. In one embodiment, R ¹¹ is —CH₂-heterocyclyl.

In one embodiment, R ¹¹ is aryl. In one embodiment, R ¹¹ is —(C₁-C₃ alkylene)-aryl. In one embodiment, R¹¹ is —CH₂-aryl.

In one embodiment, R¹¹ is heteroaryl. In one embodiment, R¹¹ is —(C₁-C₃ alkylene)-heteroaryl. In one embodiment, R ¹¹ is —CH₂-heteroaryl.

In one embodiment, R¹¹ is —OR¹². In one embodiment, R¹¹ is —(C₁-C₃ alkylene)-OR¹². In one embodiment, R¹¹ is —CH₂—OR¹². In one embodiment, R¹¹ is —CH₂—OEt. In one embodiment, R¹² is hydrogen. In one embodiment, R¹² is alkyl. In one embodiment, R¹² is heteroalkyl. In one embodiment, R¹² is cycloalkyl, heterocyclyl, aryl, or heteroaryl. In one embodiment, R¹² is cycloalkylalkyl, heterocyclylalkyl, arylalkyl, or heteroarylalkyl.

In one embodiment, R¹¹ is —NR¹²R¹³. In one embodiment, R¹¹ is —(C₁-C₃ alkylene)-NR¹²R¹³. In one embodiment, R¹¹ is —CH₂—NR²R¹³.

In one embodiment, R¹² and R¹³ are independently hydrogen or alkyl. In one embodiment, R¹² and R¹³ are independently hydrogen or C₁₋₄ alkyl. In one embodiment, R¹² and R¹³ are independently hydrogen or methyl. In one embodiment, R¹² and R¹³ are both hydrogen. In one embodiment, one of R¹² and R¹³ is hydrogen and the other is methyl. In one embodiment, R¹² and R¹³ are both methyl. In one embodiment, R¹¹ is —CH₂—NMe₂. In one embodiment, one of R¹² and R¹³ is hydrogen or alkyl, and the other is heteroalkyl. In one embodiment, one of R¹² and R¹³ is hydrogen or alkyl, and the other is cycloalkyl, heterocyclyl, aryl, or heteroaryl. In one embodiment, one of R¹² and R¹³ is hydrogen or alkyl, and the other is cycloalkylalkyl, heterocyclylalkyl, arylalkyl, or heteroarylalkyl.

In one embodiment, R¹² and R¹³ are taken together with nitrogen to form a cyclic moiety. In one embodiment, R¹¹ is —CH₂—NR¹²R¹³, wherein R¹² and R¹³ are taken together with nitrogen to form a cyclic moiety.

In one embodiment, the cycloalkyl (including cycloalkyl moiety in cycloalkylalkyl) in R¹¹, R¹², and R¹³ is independently a C₃₋₁₀ cycloalkyl. Exemplary cycloalkyl groups include, but are not limited to, cyclopropyl, cyclobutyl, cyclopentyl, cyclohexyl, cycloheptyl, decalinyl, and adamantyl. In one embodiment, the cycloalkyl is cyclopropyl. In one embodiment, the cycloalkyl is cyclobutyl. In one embodiment, the cycloalkyl is cyclopentyl. In one embodiment, the cycloalkyl is cyclohexyl.

In one embodiment, the aryl (including aryl moiety in arylalkyl) in R¹¹, R¹², and R¹³ is independently a C₆₋₁₄ aryl. Exemplary aryl groups include, but are not limited to, phenyl, naphthyl, fluorenyl, azulenyl, anthryl, phenanthryl, and pyrenyl. In one embodiment, the aryl is phenyl. In one embodiment, the aryl is naphthyl.

In one embodiment, the heterocyclyl (including heterocyclyl moiety in heterocyclylalkyl) in R¹¹, R¹², and R¹³ is independently a 3-14 membered heterocyclyl.

In one embodiment, the heterocyclyl is a 3-8 membered monocyclic heterocyclyl. In one embodiment, the heterocyclyl is a 5-membered monocyclic heterocyclyl. In one embodiment, the heterocyclyl is a 6-membered monocyclic heterocyclyl. Exemplary monocyclic heterocyclyl groups include, but are not limited to, aziridinyl, oxiranyl, thiorenyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, dioxolanyl, oxathiolanyl and dithiolanyl, piperidinyl, tetrahydropyranyl, dihydropyridinyl, thianyl, piperazinyl, morpholinyl, dithianyl, dioxanyl, azepanyl, oxepanyl, thiepanyl, oxazepanyl, diazepanyl, azocanyl, oxazocanyl, diazocanyl, oxecanyl, and thiocanyl. In one embodiment, the heterocyclyl is azetidinyl. In one embodiment, the heterocyclyl is pyrrolidinyl. In one embodiment, the heterocyclyl is piperidinyl. In one embodiment, the heterocyclyl is piperazinyl. In one embodiment, the heterocyclyl is morpholinyl.

In one embodiment, the heterocyclyl is a bicyclic heterocyclyl. In one embodiment, the heterocyclyl is a fused heterocyclyl. In one embodiment, the heterocyclyl is a 5,6-fused heterocyclyl. In one embodiment, the heterocyclyl is a 6,6-fused heterocyclyl. In one embodiment, the heterocyclyl is a bridged heterocyclyl. In one embodiment, the heterocyclyl is a spiro heterocyclyl. In one embodiment, the heterocyclyl is a bicyclic heterocyclyl, wherein a first ring selected from the group consisting of aziridinyl, oxiranyl, thiorenyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, dioxolanyl, oxathiolanyl, dithiolanyl, piperidinyl, tetrahydropyranyl, dihydropyridinyl, thianyl, piperazinyl, morpholinyl, dithianyl, dioxanyl, azepanyl, oxepanyl, thiepanyl, oxazepanyl, diazepanyl, azocanyl, oxazocanyl, diazocanyl, oxecanyl, and thiocanyl is fused, bridged, or spiroed with a second ring. In one embodiment, the first ring is azetidinyl. In one embodiment, the first ring is pyrrolidinyl. In one embodiment, the first ring is piperidinyl. In one embodiment, the first ring is piperazinyl. In one embodiment, the first ring is morpholinyl.

In one embodiment, the heteroaryl (including heteroaryl moiety in heteroarylalkyl) in R¹¹, R¹², and R¹³ is independently a 5-14 membered heteroaryl.

In one embodiment, the heteroaryl is a monocyclic heteroaryl. In one embodiment, the heteroaryl is a 5-10 membered monocyclic heteroaryl. In one embodiment, the heteroaryl is a 5-membered monocyclic heteroaryl. In one embodiment, the heteroaryl is a 6-membered monocyclic heteroaryl. Exemplary 5-membered monocyclic heteroaryl groups include, but are not limited to, pyrrolyl, furanyl, thiophenyl, imidazolyl, pyrazolyl, oxazolyl, isoxazolyl, thiazolyl, isothiazolyl, triazolyl, oxadiazolyl, thiadiazolyl, and tetrazolyl. Exemplary 6-membered monocyclic heteroaryl groups include, but are not limited to, pyridinyl, pyridazinyl, pyrimidinyl, pyrazinyl, triazinyl and tetrazinyl.

In one embodiment, the heteroaryl is a bicyclic heteroaryl. In one embodiment, the heteroaryl is a 5,6-bicyclic heteroaryl. In one embodiment, the heteroaryl is a 6,6-bicyclic heteroaryl. Exemplary 5,6-bicyclic heteroaryl groups include, but are not limited to, indolyl, isoindolyl, indazolyl, benztriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, but are not limited to, naphthyridinyl, pteridinyl, quinolinyl, isoquinolinyl, cinnolinyl, quinoxalinyl, phthalazinyl and quinazolinyl.

In one embodiment, the cyclic moiety (formed by NR¹²R¹³) is a heterocyclyl. In one embodiment, the cyclic moiety is a 3-14 membered heterocyclyl.

In one embodiment, the cyclic moiety is a monocyclic heterocyclyl. In one embodiment, the cyclic moiety is a 3-8 membered monocyclic heterocyclyl. In one embodiment, the cyclic moiety is a 5-membered monocyclic heterocyclyl. In one embodiment, the cyclic moiety is a 6-membered monocyclic heterocyclyl. Exemplary monocyclic heterocyclyl groups include, but are not limited to, aziridinyl, azetidinyl, pyrrolidinyl, dihydropyrrolyl, piperidinyl, dihydropyridinyl, piperazinyl, morpholinyl, azepanyl, oxazepanyl, diazepanyl, and azocanyl. In one embodiment, the cyclic moiety is azetidinyl. In one embodiment, the cyclic moiety is pyrrolidinyl. In one embodiment, the cyclic moiety is piperidinyl. In one embodiment, the cyclic moiety is piperazinyl. In one embodiment, the cyclic moiety is morpholinyl.

In one embodiment, the cyclic moiety is a bicyclic heterocyclyl. In one embodiment, the cyclic moiety is a fused heterocyclyl. In one embodiment, the cyclic moiety is a 5,6-fused heterocyclyl. In one embodiment, the cyclic moiety is a 6,6-fused heterocyclyl. In one embodiment, the cyclic moiety is a bridged heterocyclyl. In one embodiment, the cyclic moiety is a spiro heterocyclyl. In one embodiment, the cyclic moiety is a bicyclic heterocyclyl, wherein a first ring selected from the group consisting of aziridinyl, azetidinyl, pyrrolidinyl, dihydropyrrolyl, piperidinyl, dihydropyridinyl, piperazinyl, morpholinyl, azepanyl, oxazepanyl, diazepanyl, and azocanyl is fused, bridged, or spiroed with a second ring. In one embodiment, the first ring is azetidinyl. In one embodiment, the first ring is pyrrolidinyl. In one embodiment, the first ring is piperidinyl. In one embodiment, the first ring is piperazinyl. In one embodiment, the first ring is morpholinyl.

In one embodiment, the cyclic moiety (formed by NR¹²R¹³) is a heteroaryl. In one embodiment, the cyclic moiety is a 5-14 membered heteroaryl.

In one embodiment, the cyclic moiety is a monocyclic heteroaryl. In one embodiment, the cyclic moiety is a 5-10 membered monocyclic heteroaryl. In one embodiment, the cyclic moiety is a 5-membered monocyclic heteroaryl. In one embodiment, the cyclic moiety is a 6-membered monocyclic heteroaryl. Exemplary 5-membered monocyclic heteroaryl groups include, but are not limited to, pyrrolyl, imidazolyl, pyrazolyl, oxazolyl, isoxazolyl, thiazolyl, isothiazolyl, triazolyl, oxadiazolyl, thiadiazolyl and tetrazolyl. Exemplary 6-membered monocyclic heteroaryl groups include, but are not limited to, pyridinyl, pyridazinyl, pyrimidinyl, pyrazinyl, triazinyl and tetrazinyl.

In one embodiment, the cyclic moiety is a bicyclic heteroaryl. In one embodiment, the cyclic moiety is a 5,6-bicyclic heteroaryl. In one embodiment, the cyclic moiety is a 6,6-bicyclic heteroaryl. Exemplary 5,6-bicyclic heteroaryl groups include, but are not limited to, indolyl, isoindolyl, indazolyl, benztriazolyl, benzimidazolyl, benzoxazolyl, benzisoxazolyl, benzoxadiazolyl, benzthiazolyl, benzisothiazolyl, benzthiadiazolyl, and purinyl. Exemplary 6,6-bicyclic heteroaryl groups include, but are not limited to, naphthyridinyl, pteridinyl, quinolinyl, isoquinolinyl, cinnolinyl, quinoxalinyl, phthalazinyl, and quinazolinyl.

In one embodiment, the cyclic moiety (formed by NR¹²R¹³), cycloalkyl, heterocyclyl, aryl, or heteroaryl groups in R¹¹, R¹², and R¹³ is a monocyclic group. In one embodiment, the cyclic moiety, cycloalkyl, heterocyclyl, aryl, or heteroaryl groups in R¹¹, R¹², and R¹³ is a multicyclic group. In one embodiment, the multicyclic group is a fused ring group, a bridged ring group, or a spiro ring group.

In one embodiment, R¹¹ is:

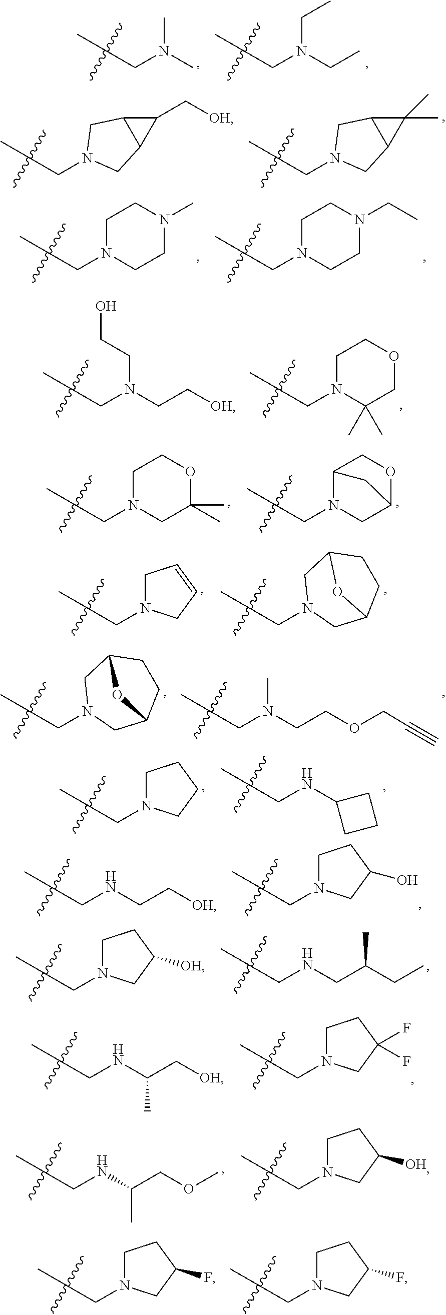

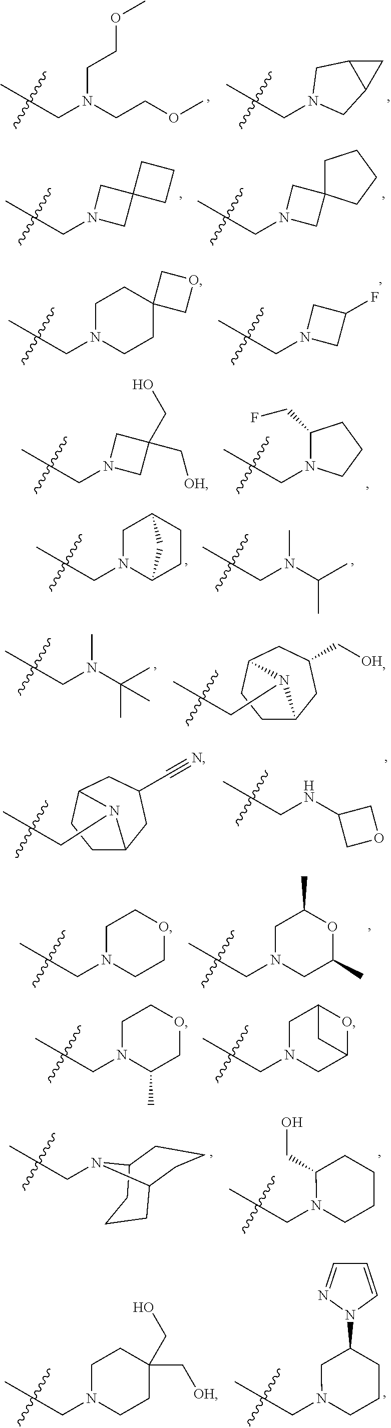

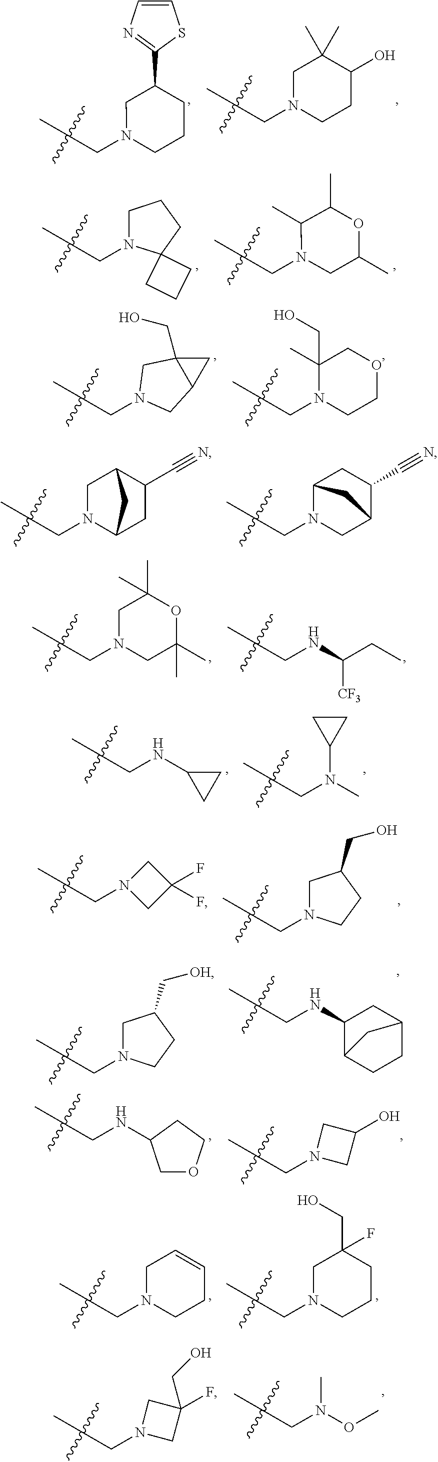

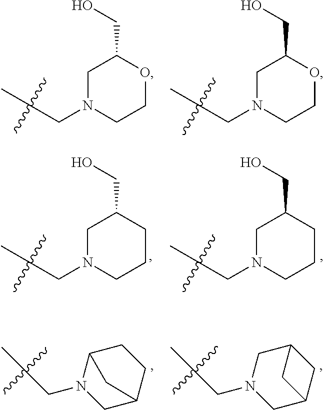 [SMILES: *CN1CCO[C@@H](CO)C1 ; *CN1CCO[C@H](CO)C1 ; *CN1CCC[C@@H](CO)C1 ; *CN1CCC[C@H](CO)C1 ; *CN1CC2CCC1C2 ; *CN1CC2CC(C2)C1]

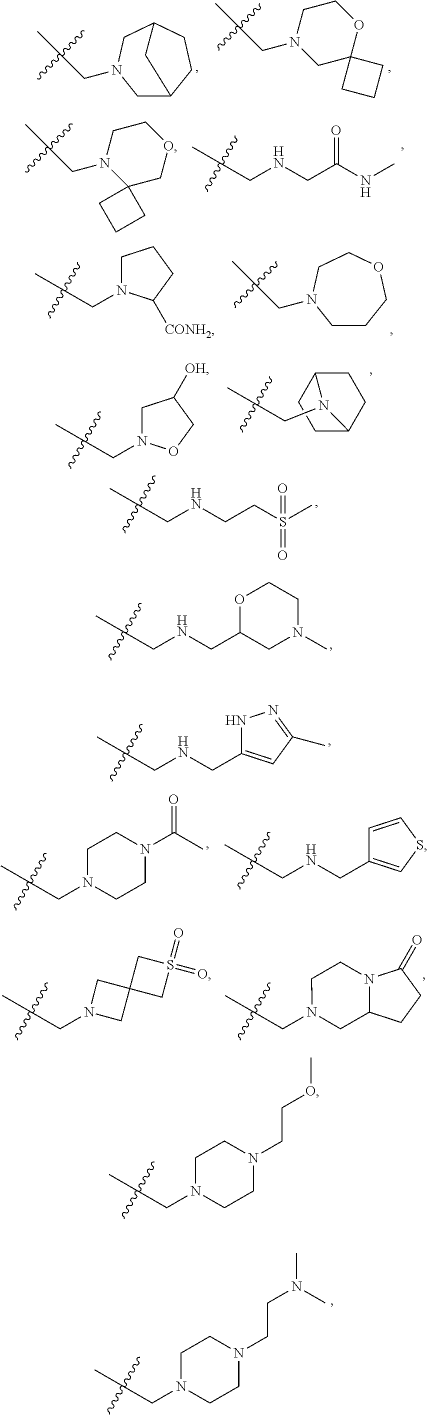

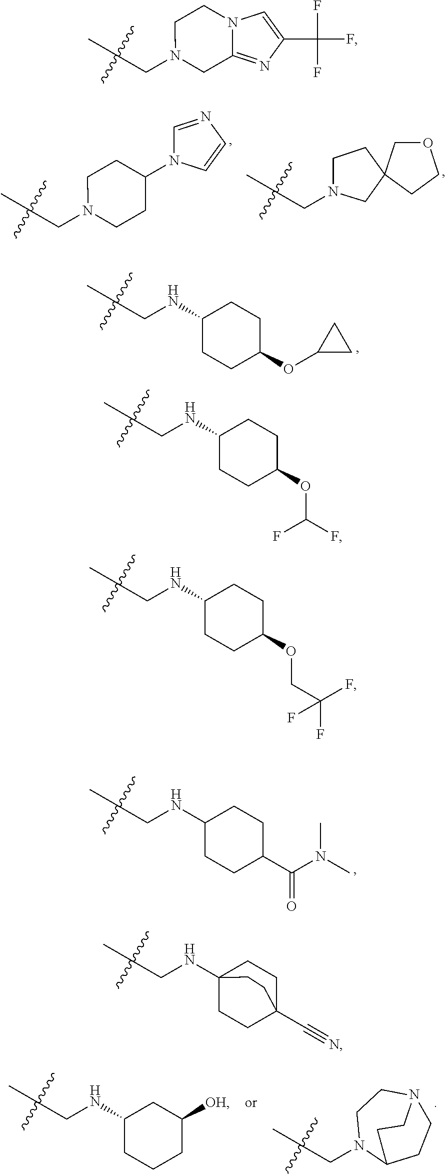

In one embodiment, the OR¹², NR²R¹³, the cyclic moiety (formed by NR¹²R¹³) cycloalkyl, heterocyclyl, aryl, or heteroaryl groups in R¹¹, R¹², and R³ is any one of the R¹¹ groups provided above without the —CH₂— linker.

In one embodiment, R¹¹, R¹², and R ¹³ are independently optionally substituted with 1, 2, 3, 4, 5, or 6 of R¹⁴. In one embodiment, the cycloalkyl, heterocyclyl, aryl, or heteroaryl group in R¹¹, R¹², and R¹³ or the cyclic moiety (formed by NR¹²R¹³) is optionally substituted with 1, 2, 3, 4, 5, or 6 of R¹⁴. In one embodiment, each instance of R¹⁴ is independently selected from the group consisting of alkyl, alkenyl, alkynyl, haloalkyl, heteroalkyl, halogen, hydroxyl, alkoxy, hydroxyalkyl, cyano, nitro, amino, amido, cycloalkyl, heterocyclyl, aryl, heteroaryl, acyl, N-acyl, carbonyl, oxo, sulfonyl, sulfonamide, and boronic acid.

In one embodiment, each instance of R¹⁴ is independently selected from the group consisting of methyl, ethyl, hydroxyl, and hydroxylmethyl.

In one embodiment, each instance of R¹⁴ independently is optionally substituted with one or more groups selected from alkyl, alkenyl, alkynyl, haloalkyl, heteroalkyl, halogen, hydroxyl, alkoxy, hydroxyalkyl, cyano, nitro, amino, amido, cycloalkyl, heterocyclyl, aryl, heteroaryl, acyl, N-acyl, carbonyl, oxo, sulfonyl, sulfonamide, and boronic acid.

In one embodiment, R¹ is aryl. In one embodiment, R¹ is C₆-C₁₀ aryl. In one embodiment, R¹ is phenyl. In one embodiment, R¹ is naphthyl. In one embodiment, R¹ is 1-naphthyl. In one embodiment, R¹ is 2-naphthyl. In another embodiment, R¹ is fluorenyl, azulenyl, anthryl, phenanthryl, or pyrenyl.

In one embodiment, R¹ is heteroaryl. In one embodiment, R¹ is 5- to 18-membered heteroaryl. In one embodiment, the heteroaryl comprises 1, 2, or 3 of heteroatoms independently selected from the group consisting of N, S, and O.

In one embodiment, R¹ is monocyclic heteroaryl. In one embodiment, R¹ is 5- to 10-membered monocyclic heteroaryl. In one embodiment, R¹ is a 5-membered monocyclic heteroaryl. In one embodiment, R¹ is a 6-membered monocyclic heteroaryl. In one embodiment, R¹ is pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, or triazinyl. In one embodiment, R¹ is pyridyl, pyridazinyl, oxazolyl, thiazolyl, oxadizolyl, piperidinyl, pyrazolyl, or pyrrolyl. In one embodiment, wherein R¹ is pyridyl. In one embodiment, wherein R¹ is 2-pyridyl. In one embodiment, wherein R¹ is 3-pyridyl. In one embodiment, wherein R¹ is 4-pyridyl.

In one embodiment, R¹ is bicyclic heteroaryl. In one embodiment, R¹ is a 5,6-bicyclic heteroaryl. In one embodiment, R¹ is a 6,6-bicyclic heteroaryl. In one embodiment, R¹ is indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, or tetrahydroquinolinyl.

In one embodiment, R¹ is tricyclic heteroaryl. In one embodiment, R¹ is carbazolyl, benzindolyl, phenanthrollinyl, acridinyl, phenanthridinyl, or xanthenyl.

In one embodiment, R¹ is cycloalkyl. In one embodiment, R¹ is cyclopropyl, cyclobutyl, cyclopentyl, cyclohexyl, cycloheptyl, decalinyl, or adamantyl.

In one embodiment, R¹ is heterocyclyl. In one embodiment, R¹ is tetrahydrofuranyl, 2,3-dihydro-4H-pyranyl, pyrrolinyl, pyrrolidinyl, 1,3-thiazolidinyl, morpholinyl, piperidinyl, piperazinyl, dihydropyridinyl, dihydropyrimidinyl, or azepanyl.

In one embodiment, R¹ is:

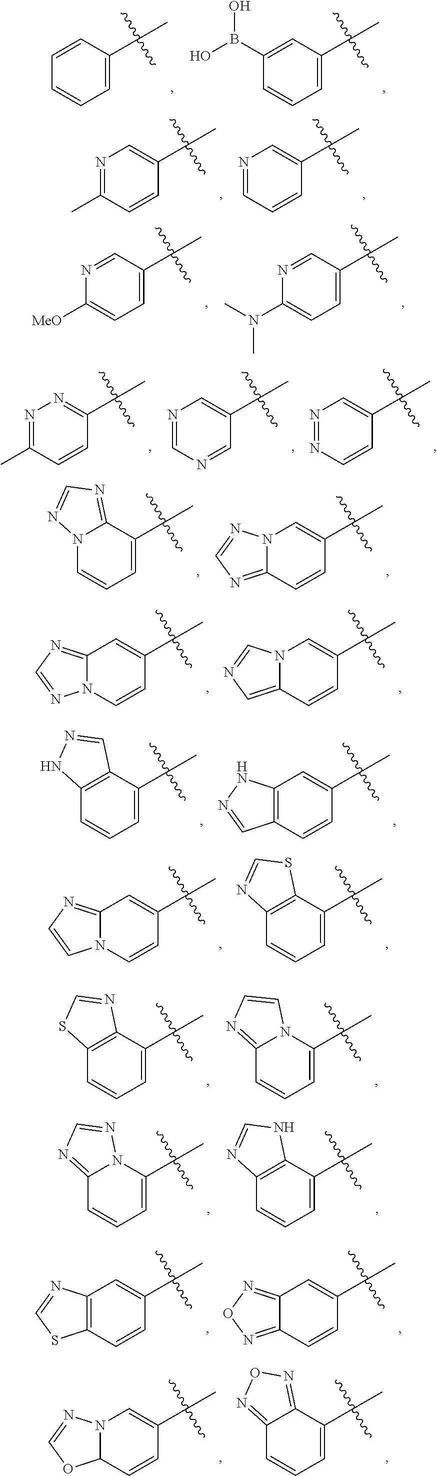

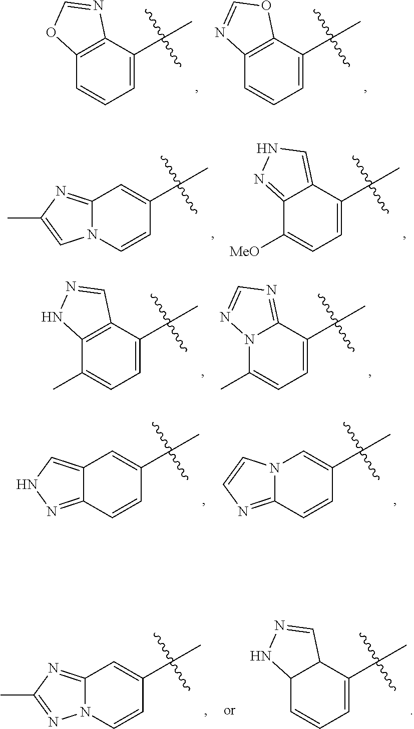

In one embodiment, R¹ is unsubstituted. In one embodiment, R¹ is substituted with 1 of R⁹. In one embodiment, R¹ is substituted with 2 of R⁹. In one embodiment, R¹ is substituted with 3 of R⁹. In one embodiment, R¹ is substituted with 4 of R⁹. In one embodiment, R¹ is substituted with 5 of R⁹. In one embodiment, R¹ is substituted with 6 of R⁹.

In one embodiment, R¹ is unsubstituted pyridyl. In one embodiment, R¹ is pyridyl substituted with 1 of R⁹. In one embodiment, R¹ is pyridyl substituted with 2 of R⁹. In one embodiment, R¹ is pyridyl substituted with 3 of R⁹. In one embodiment, R¹ is pyridyl substituted with 4 of R⁹.

In one embodiment, R¹ is unsubstituted 3-pyridyl. In one embodiment, R¹ is 3-pyridyl substituted with 1 of R⁹. In one embodiment, R¹ is 3-pyridyl substituted with 2 of R⁹. In one embodiment, R¹ is 3-pyridyl substituted with 3 of R⁹. In one embodiment, R¹ is 3-pyridyl substituted with 4 of R⁹.

In one embodiment, R¹ is

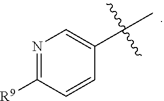

In one embodiment, R¹ is

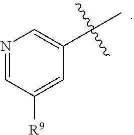

In one embodiment, R¹ is

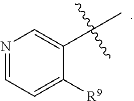

In one embodiment, R¹ is

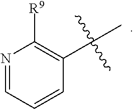

In one embodiment, provided herein is a compound of the Formula (VI-1):

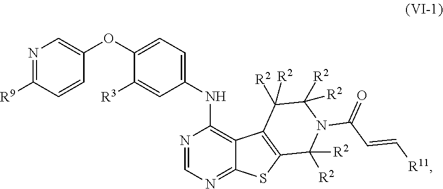

or a pharmaceutically acceptable salt, solvate (e.g., hydrate), prodrug, tautomer, stereoisomer, enantiomer, or isotopologue thereof, or a mixture thereof.

In one embodiment, each instance of R⁹ is independently selected from the group consisting of hydrogen, alkyl, alkenyl, alkynyl, haloalkyl, heteroalkyl, halogen, hydroxyl, alkoxy, hydroxyalkyl, cyano, nitro, amino, amido, cycloalkyl, heterocyclyl, aryl, heteroaryl, acyl, N-acyl, carbonyl, oxo, sulfonyl, sulfonamide, and boronic acid. In one embodiment, each instance of R⁹ is independently hydrogen, alkyl, alkenyl, alkynyl, haloalkyl, heteroalkyl, halogen, hydroxyl, alkoxy, cyano, nitro, amino, amido, cycloalkyl, heterocyclyl, aryl, heteroaryl, N-acyl, carbonyl, sulfonyl, sulfonamide, or boronic acid. In one embodiment, each instance of R⁹ is independently hydrogen, alkyl, alkoxy, or boronic acid. In one embodiment, R⁹ is methyl or methoxy. In one embodiment, R⁹ is methyl. In one embodiment, R⁹ is methoxy.

In one embodiment, each instance of R² is independently hydrogen, alkyl, alkenyl, alkynyl, haloalkyl, heteroalkyl, halogen, cyano, nitro, amido, cycloalkyl, heterocyclyl, aryl, heteroaryl, —OR′, or —NR′R″, wherein R′ and R¹¹ are independently hydrogen, alkyl, heteroalkyl, aryl, or heteroaryl, or R′ and R¹¹ are taken together with nitrogen to form a cyclic moiety. In one embodiment, each instance of R² is independently hydrogen, alkyl, or alkoxy. In one embodiment, all of R² are hydrogen. In one embodiment, at least one of R² is not hydrogen.

In one embodiment, the cyclic moiety (formed by NR′R″) is a heterocyclyl. In one embodiment, the cyclic moiety is a 3-14 membered heterocyclyl.

In one embodiment, the cyclic moiety is a monocyclic heterocyclyl. In one embodiment, the cyclic moiety is a 3-8 membered monocyclic heterocyclyl. In one embodiment, the cyclic moiety is a 5-membered monocyclic heterocyclyl. In one embodiment, the cyclic moiety is a 6-membered monocyclic heterocyclyl. Exemplary monocyclic heterocyclyl groups include, but are not limited to, aziridinyl, azetidinyl, pyrrolidinyl, dihydropyrrolyl, piperidinyl, dihydropyridinyl, piperazinyl, morpholinyl, azepanyl, oxazepanyl, diazepanyl, and azocanyl. In one embodiment, the cyclic moiety is azetidinyl. In one embodiment, the cyclic moiety is pyrrolidinyl. In one embodiment, the cyclic moiety is piperidinyl. In one embodiment, the cyclic moiety is piperazinyl. In one embodiment, the cyclic moiety is morpholinyl.

In one embodiment, the cyclic moiety is a bicyclic heterocyclyl. In one embodiment, the cyclic moiety is a fused heterocyclyl. In one embodiment, the cyclic moiety is a 5,6-fused heterocyclyl. In one embodiment, the cyclic moiety is a 6,6-fused heterocyclyl. In one embodiment, the cyclic moiety is a bridged heterocyclyl. In one embodiment, the cyclic moiety is a spiro heterocyclyl. In one embodiment, the cyclic moiety is a bicyclic heterocyclyl, wherein a first ring selected from the group consisting of aziridinyl, azetidinyl, pyrrolidinyl, dihydropyrrolyl, piperidinyl, dihydropyridinyl, piperazinyl, morpholinyl, azepanyl, oxazepanyl, diazepanyl, and azocanyl is fused, bridged, or spiroed with a second ring. In one embodiment, the first ring is azetidinyl. In one embodiment, the first ring is pyrrolidinyl. In one embodiment, the first ring is piperidinyl. In one embodiment, the first ring is piperazinyl. In one embodiment, the first ring is morpholinyl.

In one embodiment, the cyclic moiety (formed by NR′R″) is a heteroaryl. In one embodiment, the cyclic moiety is a 5-14 membered heteroaryl.

In one embodiment, the cyclic moiety is a monocyclic heteroaryl. In one embodiment, the cyclic moiety is a 5-10 membered monocyclic heteroaryl. In one embodiment, the cyclic moiety is a 5-membered monocyclic heteroaryl. In one embodiment, the cyclic moiety is a 6-membered monocyclic heteroaryl. Exemplary 5-membered monocyclic heteroaryl groups include, but are not limited to, pyrrolyl, imidazolyl, pyrazolyl, oxazolyl, isoxazolyl, thiazolyl, isothiazolyl, triazolyl, oxadiazolyl, thiadiazolyl and tetrazolyl. Exemplary 6-membered monocyclic heteroaryl groups include, but are not limited to, pyridinyl, pyridazinyl, pyrimidinyl, pyrazinyl, triazinyl and tetrazinyl.

In one embodiment, the cyclic moiety is a bicyclic heteroaryl. In one embodiment, the cyclic moiety is a 5,6-bicyclic heteroaryl. In one embodiment, the cyclic moiety is a 6,6-bicyclic heteroaryl. Exemplary 5,6-bicyclic heteroaryl groups include, but are not limited to, indolyl, isoindolyl, indazolyl, benztriazolyl, benzimidazolyl, benzoxazolyl, benzisoxazolyl, benzoxadiazolyl, benzthiazolyl, benzisothiazolyl, benzthiadiazolyl, and purinyl. Exemplary 6,6-bicyclic heteroaryl groups include, but are not limited to, naphthyridinyl, pteridinyl, quinolinyl, isoquinolinyl, cinnolinyl, quinoxalinyl, phthalazinyl, and quinazolinyl.

In one embodiment, the

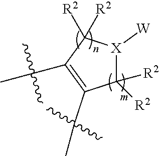

moiety is

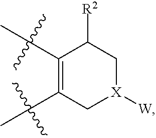

or an enantiomer thereof, or a mixture thereof. In one embodiment, the

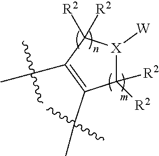

moiety is

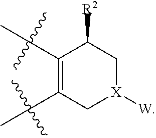

In one embodiment, the

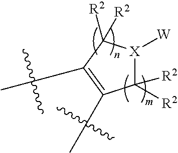

moiety is

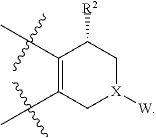

In one embodiment, the

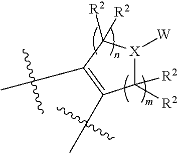

moiety is

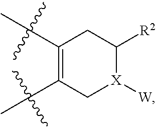

or an enantiomer thereof, or a mixture thereof. In one embodiment, the

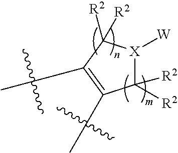

moiety is

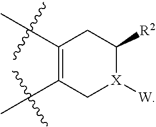

In one embodiment, the

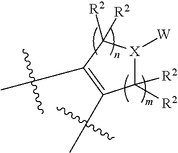

moiety is

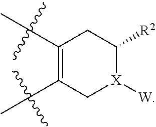

In one embodiment, the

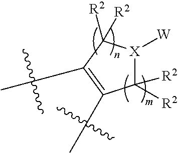

moiety is

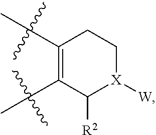

or an enantiomer thereof, or a mixture thereof. In one embodiment, the

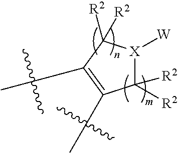

moiety is

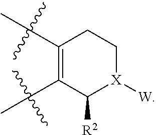

In one embodiment, the

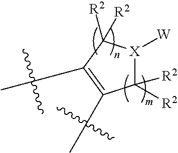

moiety is

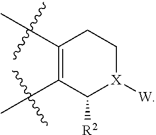

In one embodiment, the

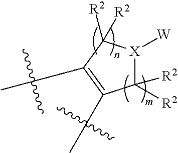

moiety is

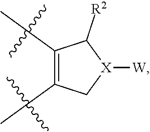

or an enantiomer thereof, or a mixture thereof. In one embodiment the

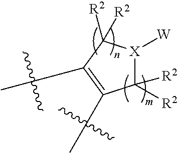

moiety is

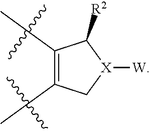

In one embodiment, the

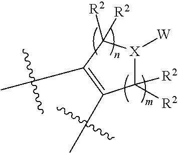

moiety is

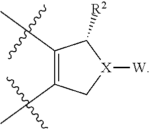

In one embodiment, the

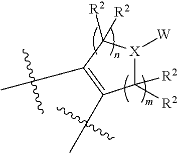

moiety is

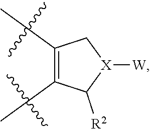

or an enantiomer thereof, or a mixture thereof. In one embodiment, the

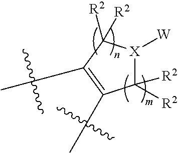

moiety is

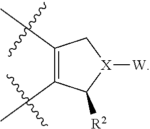

In one embodiment, the

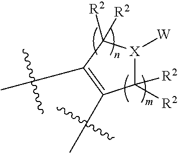

moiety is

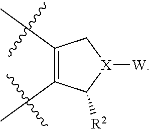

In one embodiment, two R² are taken together to form a C₁-C₃ alkylene. In one embodiment, two R² are taken together to form a methylene. In one embodiment, two R² are taken together to form a —CH₂—. In one embodiment, two R² are taken together to form a C₂ alkylene. In one embodiment, two R² are taken together to form a —CH₂CH₂—. In one embodiment, two R² are taken together to form a C₃ alkylene. In one embodiment, two R² are taken together to form a —CH₂CH₂CH₂—. In one embodiment, the alkylene is unsubstituted. In one embodiment, the alkylenen is substituted with one or more of alkyl or halo. In one embodiment, the alkylenen is substituted with one or more of methyl.

In one embodiment, the

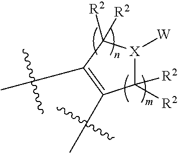

moiety is

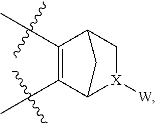

or an enantiomer thereof, or a mixture thereof. In one embodiment, the

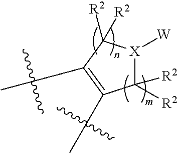

moiety is

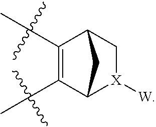

In one embodiment, the

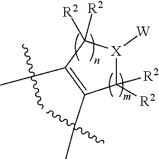

moiety is

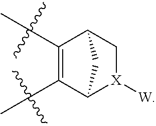

In one embodiment, each instance of R³ is independently alkyl, haloalkyl, alkoxy, or halogen. In one embodiment, each instance of R³ is independently alkyl. In one embodiment, each instance of R³ is independently C₁₋₄ alkyl. In one embodiment, the alkyl is unsubstituted. In one embodiment, the alkyl is substituted with one or more of halo. In one embodiment, the alkyl is substituted with one or more of fluoro.

In one embodiment, each instance of R³ is independently methyl. In one embodiment, each instance of R³ is independently halo. In one embodiment, each instance of R³ is independently chloro.

In one embodiment, each instance of R³ is independently methyl, —CF₃, methoxy, fluoro, or chloro.

In one embodiment, provided herein is a compound listed in Table 1, or a pharmaceutically acceptable salt, solvate (e.g., hydrate), prodrug, tautomer, stereoisomer, enantiomer, or isotopologue thereof, or a mixture thereof.

TABLE 1

| Compound | Structure |
|---|---|
| 2 | 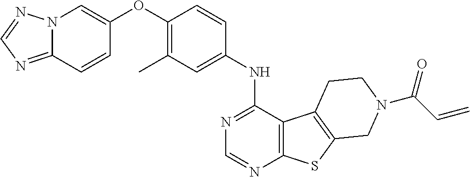 [SMILES: C=CC(=O)N1CCc2c(sc3ncnc(Nc4ccc(Oc5ccc6ncnn6c5)c(C)c4)c23)C1] |
| 3 | 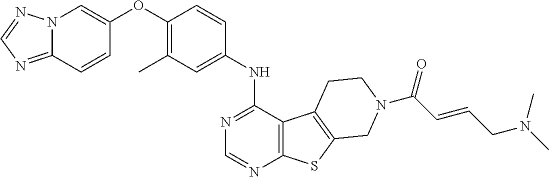 [SMILES: Cc1cc(Nc2ncnc3sc4c(c23)CCN(C(=O)/C=C/CN(C)C)C4)ccc1Oc1ccc2ncnn2c1] |
| 4 | 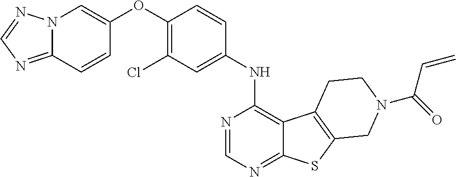 [SMILES: C=CC(=O)N1CCc2c(sc3ncnc(Nc4ccc(Oc5ccc6ncnn6c5)c(Cl)c4)c23)C1] |
| 5 | 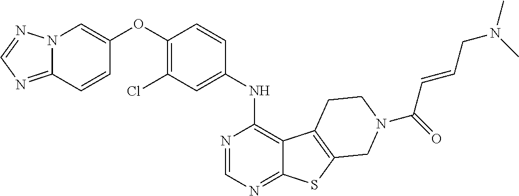 [SMILES: CN(C)C/C=C/C(=O)N1CCc2c(sc3ncnc(Nc4ccc(Oc5ccc6ncnn6c5)c(Cl)c4)c23)C1] |
| 6 | 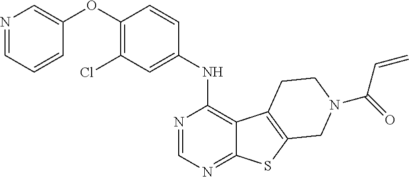 [SMILES: C=CC(=O)N1CCc2c(sc3ncnc(Nc4ccc(Oc5cccnc5)c(Cl)c4)c23)C1] |
| 7 | 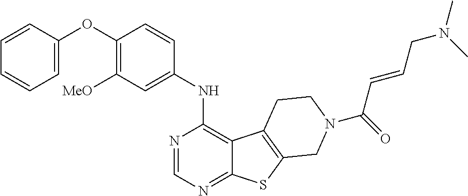 [SMILES: COc1cc(Nc2ncnc3sc4c(c23)CCN(C(=O)/C=C/CN(C)C)C4)ccc1Oc1ccccc1] |
| 8 | 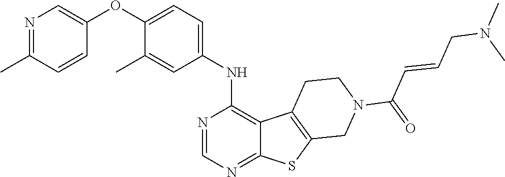 |
| 11 | 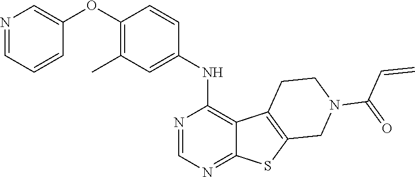 [SMILES: C=CC(=O)N1CCc2c(sc3ncnc(Nc4ccc(Oc5cccnc5)c(C)c4)c23)C1] |
| 12 | 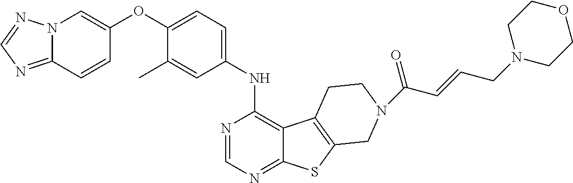 [SMILES: Cc1cc(Nc2ncnc3sc4c(c23)CCN(C(=O)/C=C/CN2CCOCC2)C4)ccc1Oc1ccc2ncnn2c1] |
| 13 | 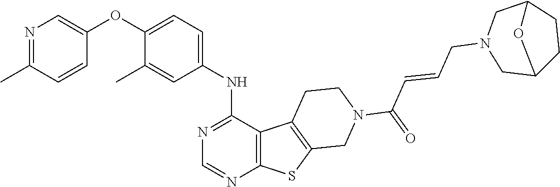 [SMILES: Cc1ccc(Oc2ccc(Nc3ncnc4sc5c(c34)CCN(C(=O)/C=C/CN3CC4CCC(C3)O4)C5)cc2C)cn1] |
| 14 | 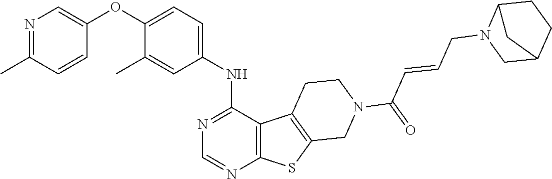 [SMILES: Cc1ccc(Oc2ccc(Nc3ncnc4sc5c(c34)CCN(C(=O)/C=C/CN3CC4CCC3C4)C5)cc2C)cn1] |
| 15 | 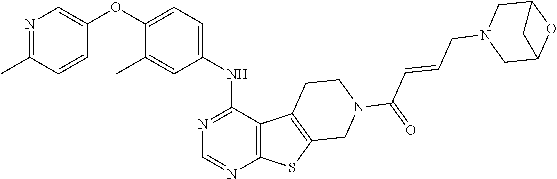 [SMILES: Cc1ccc(Oc2ccc(Nc3ncnc4sc5c(c34)CCN(C(=O)/C=C/CN3CC4CC(C3)O4)C5)cc2C)cn1] |
| 16 | 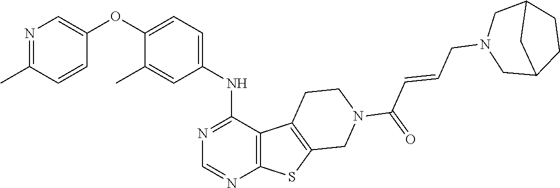 [SMILES: Cc1ccc(Oc2ccc(Nc3ncnc4sc5c(c34)CCN(C(=O)/C=C/CN3CC4CCC(C4)C3)C5)cc2C)cn1] |
| 17 | 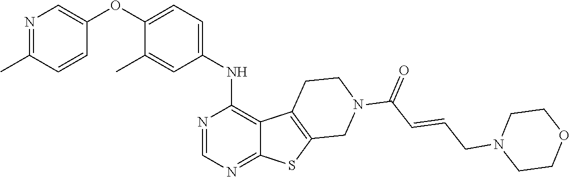 [SMILES: Cc1ccc(Oc2ccc(Nc3ncnc4sc5c(c34)CCN(C(=O)/C=C/CN3CCOCC3)C5)cc2C)cn1] |
| 18 | 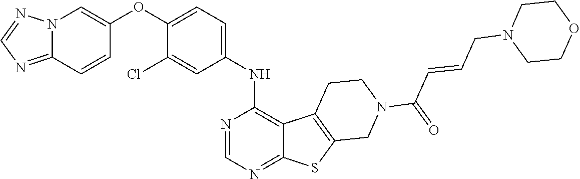 [SMILES: O=C(/C=C/CN1CCOCC1)N1CCc2c(sc3ncnc(Nc4ccc(Oc5ccc6ncnn6c5)c(Cl)c4)c23)C1] |
| 20 | 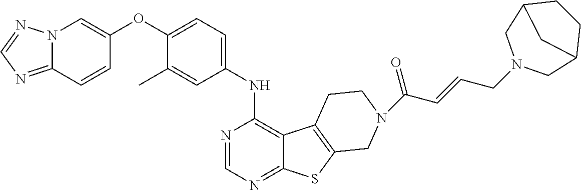 [SMILES: Cc1cc(Nc2ncnc3sc4c(c23)CCN(C(=O)/C=C/CN2CC3CCC(C3)C2)C4)ccc1Oc1ccc2ncnn2c1] |
| 21 | 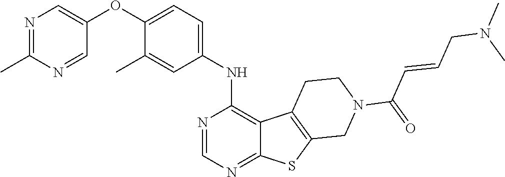 |
| 22 | 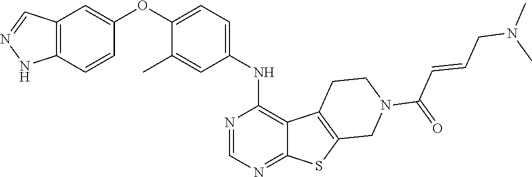 [SMILES: Cc1cc(Nc2ncnc3sc4c(c23)CCN(C(=O)/C=C/CN(C)C)C4)ccc1Oc1ccc2[nH]ncc2c1] |
| 23 | 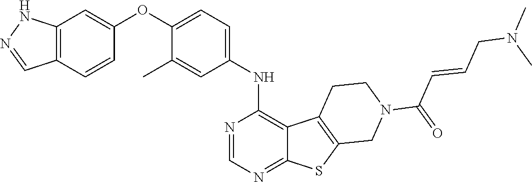 [SMILES: Cc1cc(Nc2ncnc3sc4c(c23)CCN(C(=O)/C=C/CN(C)C)C4)ccc1Oc1ccc2cn[nH]c2c1] |
| 26 | 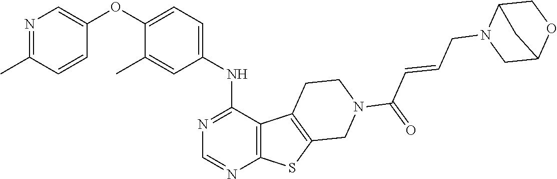 [SMILES: Cc1ccc(Oc2ccc(Nc3ncnc4sc5c(c34)CCN(C(=O)/C=C/CN3CC4CC3CO4)C5)cc2C)cn1] |
| 27 | 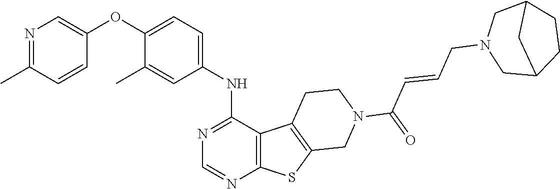 [SMILES: Cc1ccc(Oc2ccc(Nc3ncnc4sc5c(c34)CCN(C(=O)/C=C/CN3CC4CCC(C4)C3)C5)cc2C)cn1] |
| 28 | 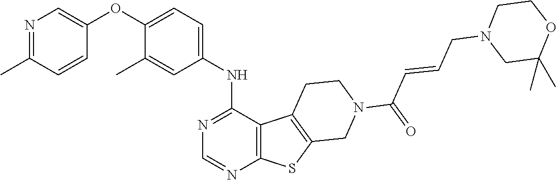 |
| 29 | 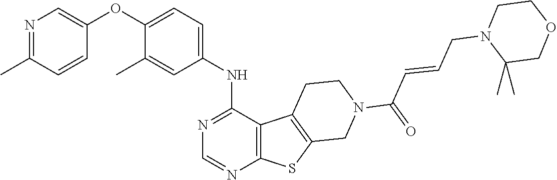 [SMILES: Cc1ccc(Oc2ccc(Nc3ncnc4sc5c(c34)CCN(C(=O)/C=C/CN3CCOCC3(C)C)C5)cc2C)cn1] |
| 30 | 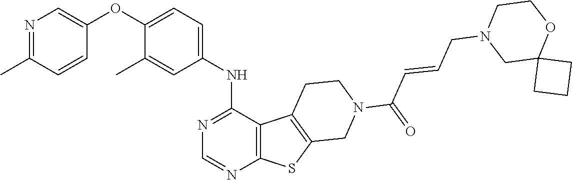 [SMILES: Cc1ccc(Oc2ccc(Nc3ncnc4sc5c(c34)CCN(C(=O)/C=C/CN3CCOC4(CCC4)C3)C5)cc2C)cn1] |
| 31 | 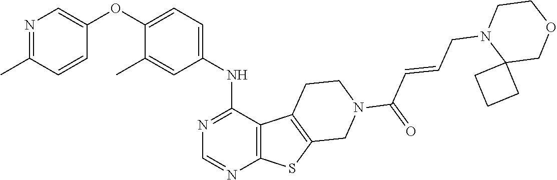 [SMILES: Cc1ccc(Oc2ccc(Nc3ncnc4sc5c(c34)CCN(C(=O)/C=C/CN3CCOCC34CCC4)C5)cc2C)cn1] |
| 32 | 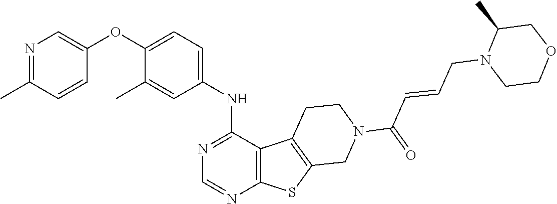 [SMILES: Cc1ccc(Oc2ccc(Nc3ncnc4sc5c(c34)CCN(C(=O)/C=C/CN3CCOC[C@@H]3C)C5)cc2C)cn1] |
| 33 | 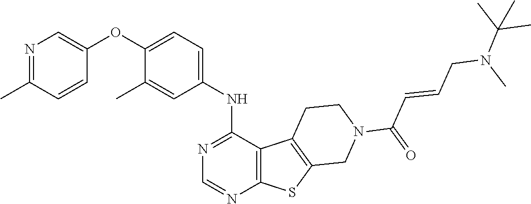 [SMILES: Cc1ccc(Oc2ccc(Nc3ncnc4sc5c(c34)CCN(C(=O)/C=C/CN(C)C(C)(C)C)C5)cc2C)cn1] |
| 34 | 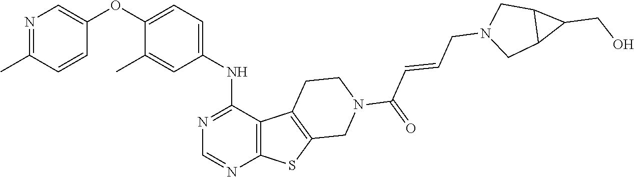 [SMILES: Cc1ccc(Oc2ccc(Nc3ncnc4sc5c(c34)CCN(C(=O)/C=C/CN3CC4C(CO)C4C3)C5)cc2C)cn1] |
| 36 | 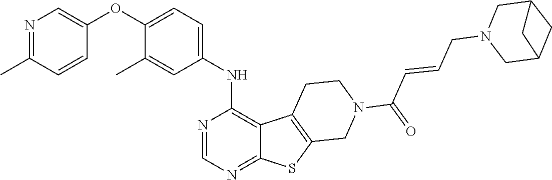 [SMILES: Cc1ccc(Oc2ccc(Nc3ncnc4sc5c(c34)CCN(C(=O)/C=C/CN3CC4CC(C4)C3)C5)cc2C)cn1] |
| 37 | 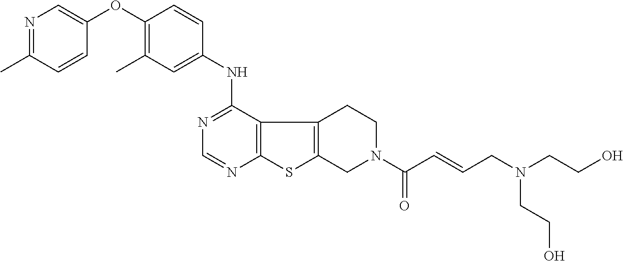 [SMILES: Cc1ccc(Oc2ccc(Nc3ncnc4sc5c(c34)CCN(C(=O)/C=C/CN(CCO)CCO)C5)cc2C)cn1] |
| 38 | 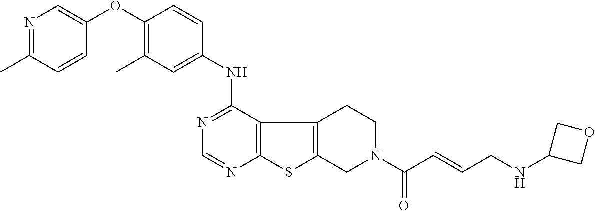 [SMILES: Cc1ccc(Oc2ccc(Nc3ncnc4sc5c(c34)CCN(C(=O)/C=C/CNC3COC3)C5)cc2C)cn1] |
| 39 | 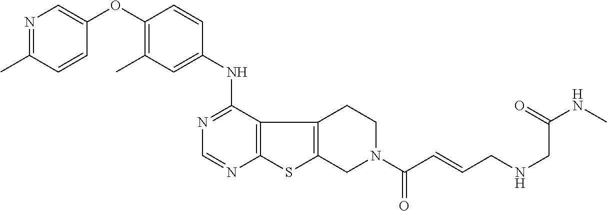 |
| 40 | 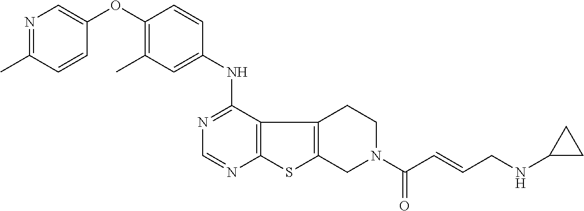 [SMILES: Cc1ccc(Oc2ccc(Nc3ncnc4sc5c(c34)CCN(C(=O)/C=C/CNC3CC3)C5)cc2C)cn1] |
| 41 | 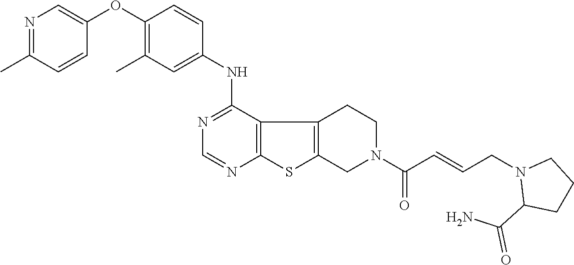 [SMILES: Cc1ccc(Oc2ccc(Nc3ncnc4sc5c(c34)CCN(C(=O)/C=C/CN3CCCC3C(N)=O)C5)cc2C)cn1] |
| 42 | 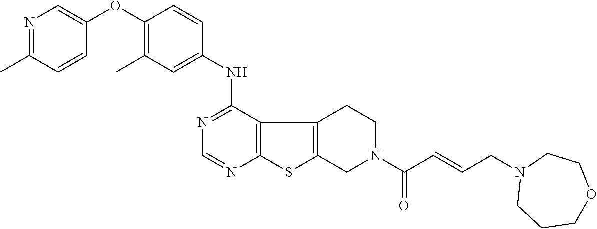 [SMILES: Cc1ccc(Oc2ccc(Nc3ncnc4sc5c(c34)CCN(C(=O)/C=C/CN3CCCOCC3)C5)cc2C)cn1] |
| 43 | 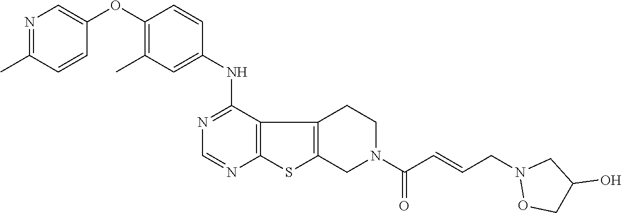 [SMILES: Cc1ccc(Oc2ccc(Nc3ncnc4sc5c(c34)CCN(C(=O)/C=C/CN3CC(O)CO3)C5)cc2C)cn1] |
| 44 | 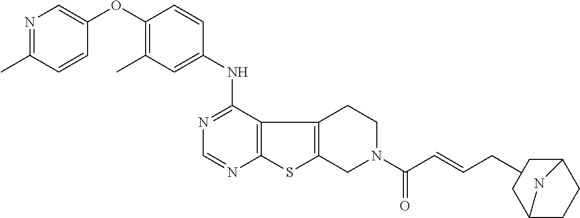 |
| 45 | 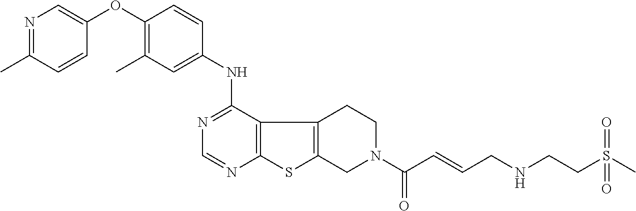 |
| 46 | 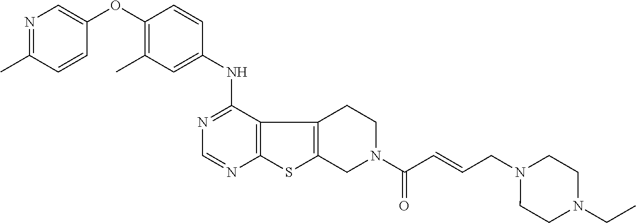 [SMILES: CCN1CCN(C/C=C/C(=O)N2CCc3c(sc4ncnc(Nc5ccc(Oc6ccc(C)nc6)c(C)c5)c34)C2)CC1] |
| 47 | 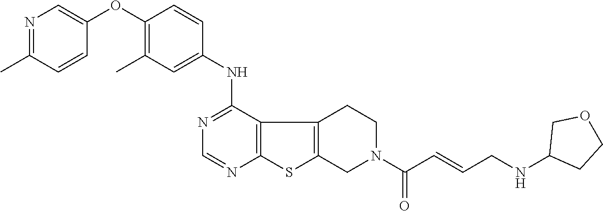 [SMILES: Cc1ccc(Oc2ccc(Nc3ncnc4sc5c(c34)CCN(C(=O)/C=C/CNC3CCOC3)C5)cc2C)cn1] |
| 48 | 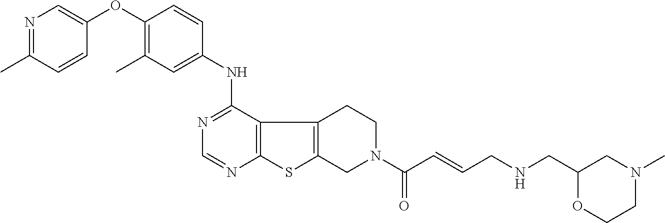 |
| 49 | 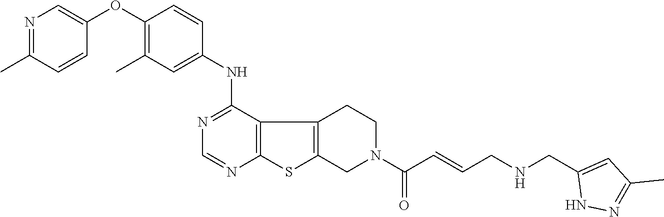 |
| 51 | 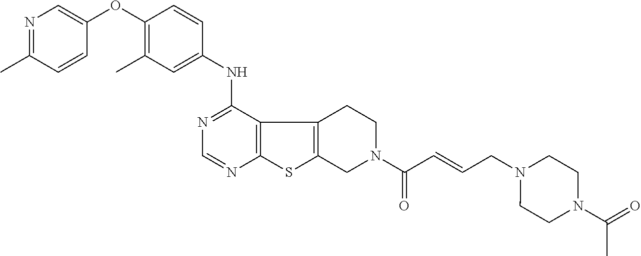 [SMILES: CC(=O)N1CCN(C/C=C/C(=O)N2CCc3c(sc4ncnc(Nc5ccc(Oc6ccc(C)nc6)c(C)c5)c34)C2)CC1] |
| 52 | 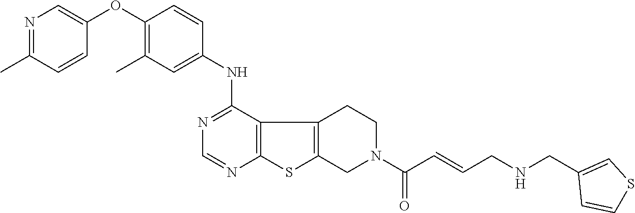 [SMILES: Cc1ccc(Oc2ccc(Nc3ncnc4sc5c(c34)CCN(C(=O)/C=C/CNCc3ccsc3)C5)cc2C)cn1] |
| 53 | 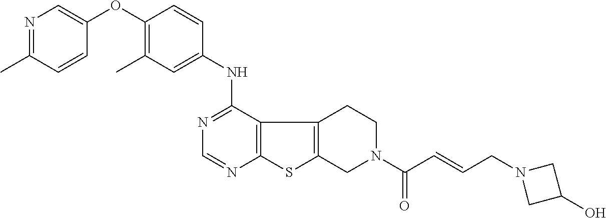 [SMILES: Cc1ccc(Oc2ccc(Nc3ncnc4sc5c(c34)CCN(C(=O)/C=C/CN3CC(O)C3)C5)cc2C)cn1] |
| 54 | 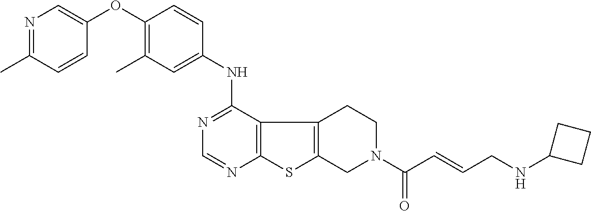 [SMILES: Cc1ccc(Oc2ccc(Nc3ncnc4sc5c(c34)CCN(C(=O)/C=C/CNC3CCC3)C5)cc2C)cn1] |
| 55 | 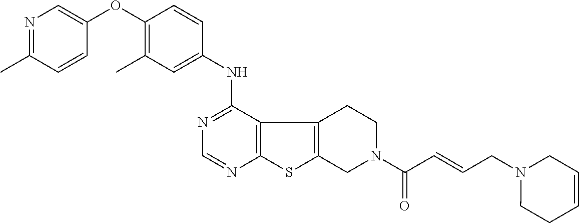 [SMILES: Cc1ccc(Oc2ccc(Nc3ncnc4sc5c(c34)CCN(C(=O)/C=C/CN3CC=CCC3)C5)cc2C)cn1] |
| 56 | 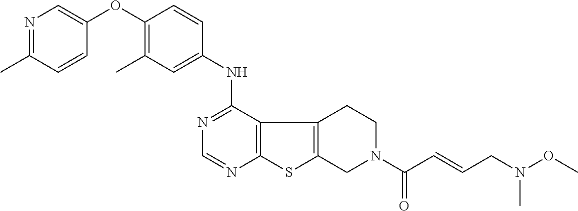 |
| 57 | 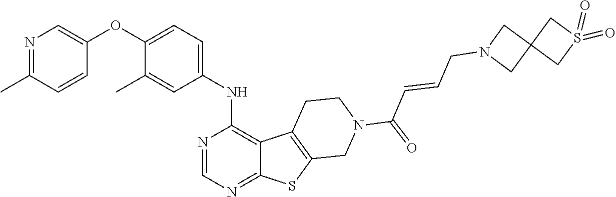 [SMILES: Cc1ccc(Oc2ccc(Nc3ncnc4sc5c(c34)CCN(C(=O)/C=C/CN3CC4(C3)CS(=O)(=O)C4)C5)cc2C)cn1] |
| 58 | 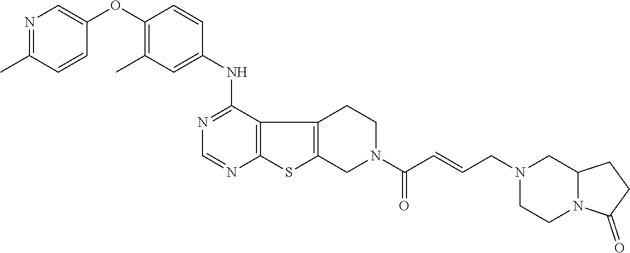 [SMILES: Cc1ccc(Oc2ccc(Nc3ncnc4sc5c(c34)CCN(C(=O)/C=C/CN3CCN4C(=O)CCC4C3)C5)cc2C)cn1] |
| 59 | 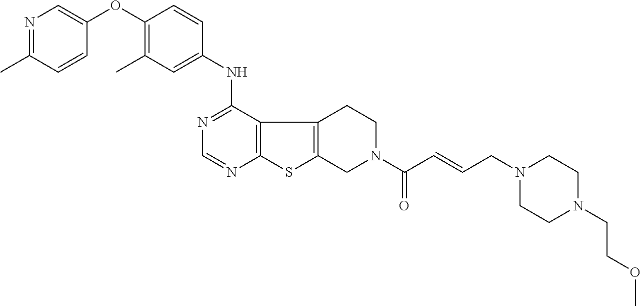 |
| 60 | 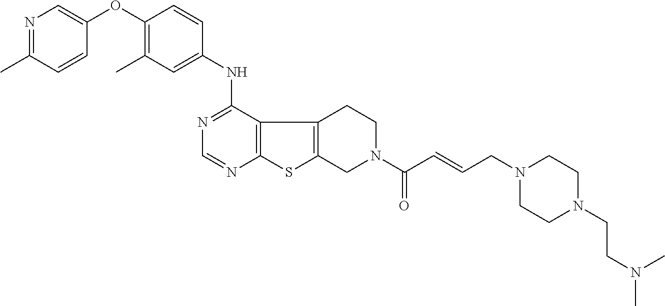 |
| 61 | 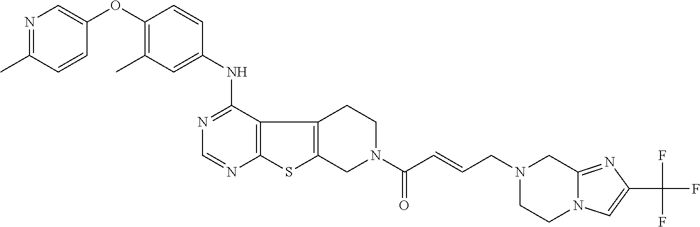 [SMILES: Cc1ccc(Oc2ccc(Nc3ncnc4sc5c(c34)CCN(C(=O)/C=C/CN3CCn4cc(C(F)(F)F)nc4C3)C5)cc2C)cn1] |
| 62 | 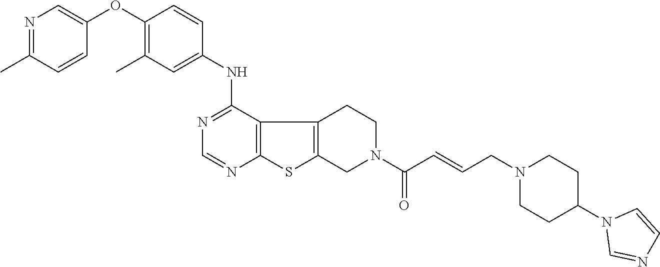 [SMILES: Cc1ccc(Oc2ccc(Nc3ncnc4sc5c(c34)CCN(C(=O)/C=C/CN3CCC(n4ccnc4)CC3)C5)cc2C)cn1] |
| 64 | 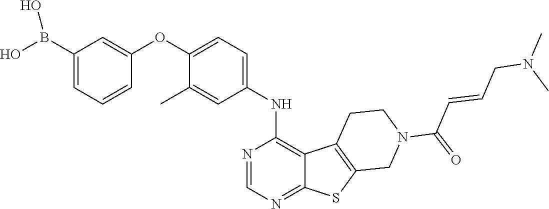 [SMILES: Cc1cc(Nc2ncnc3sc4c(c23)CCN(C(=O)/C=C/CN(C)C)C4)ccc1Oc1cccc(B(O)O)c1] |
| 65 | 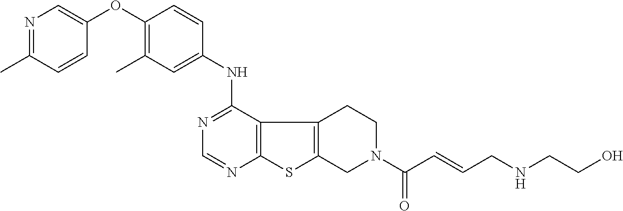 [SMILES: Cc1ccc(Oc2ccc(Nc3ncnc4sc5c(c34)CCN(C(=O)/C=C/CNCCO)C5)cc2C)cn1] |
| 66 | 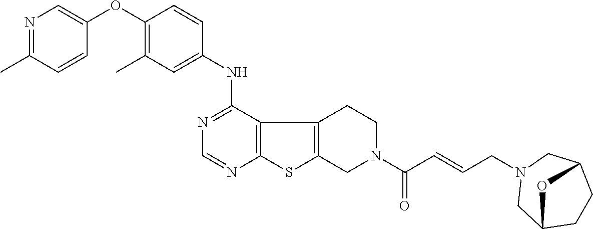 [SMILES: Cc1ccc(Oc2ccc(Nc3ncnc4sc5c(c34)CCN(C(=O)/C=C/CN3C[C@H]4CC[C@@H](C3)O4)C5)cc2C)cn1] |
| 67 | 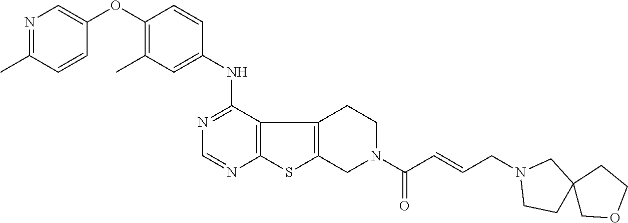 [SMILES: Cc1ccc(Oc2ccc(Nc3ncnc4sc5c(c34)CCN(C(=O)/C=C/CN3CCC4(CCOC4)C3)C5)cc2C)cn1] |
| 68 | 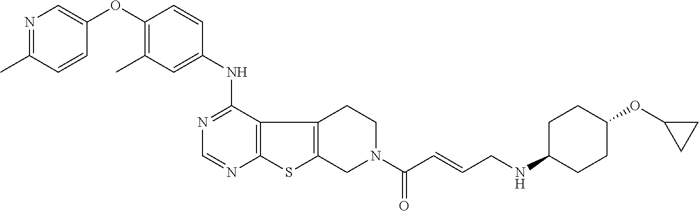 [SMILES: Cc1ccc(Oc2ccc(Nc3ncnc4sc5c(c34)CCN(C(=O)/C=C/CN[C@H]3CC[C@H](OC4CC4)CC3)C5)cc2C)cn1] |
| 69 | 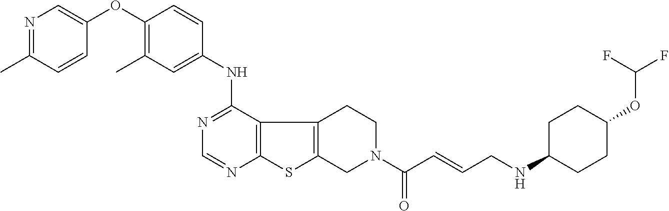 [SMILES: Cc1ccc(Oc2ccc(Nc3ncnc4sc5c(c34)CCN(C(=O)/C=C/CN[C@H]3CC[C@H](OC(F)F)CC3)C5)cc2C)cn1] |
| 70 | 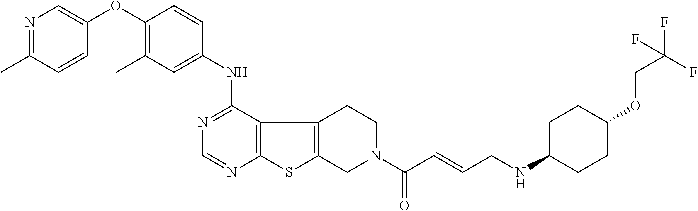 [SMILES: Cc1ccc(Oc2ccc(Nc3ncnc4sc5c(c34)CCN(C(=O)/C=C/CN[C@H]3CC[C@H](OCC(F)(F)F)CC3)C5)cc2C)cn1] |
| 71 | 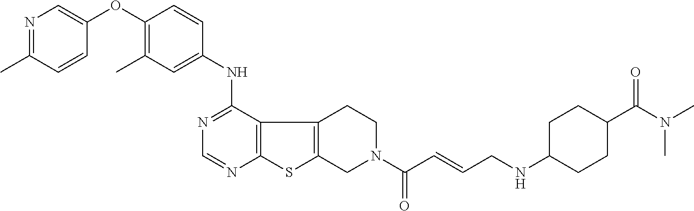 |
| 72 | 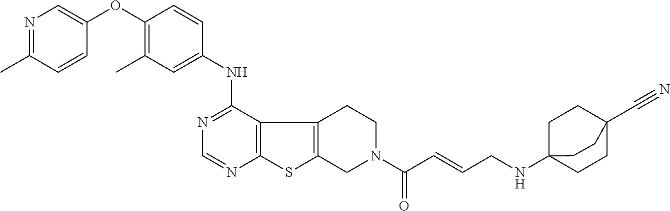 [SMILES: Cc1ccc(Oc2ccc(Nc3ncnc4sc5c(c34)CCN(C(=O)/C=C/CNC34CCC(C#N)(CC3)CC4)C5)cc2C)cn1] |
| 73 | 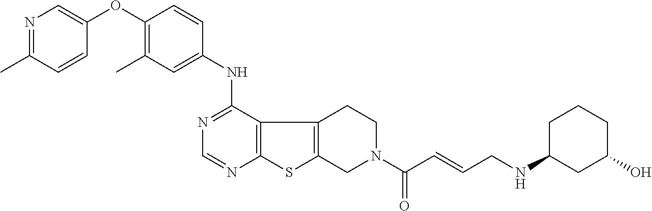 [SMILES: Cc1ccc(Oc2ccc(Nc3ncnc4sc5c(c34)CCN(C(=O)/C=C/CN[C@H]3CCC[C@H](O)C3)C5)cc2C)cn1] |
| 74 | 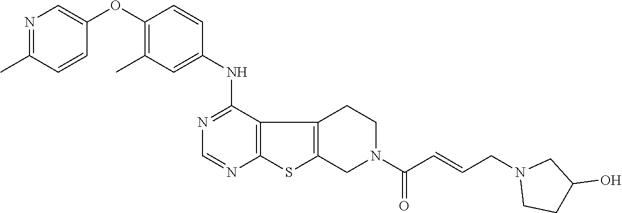 [SMILES: Cc1ccc(Oc2ccc(Nc3ncnc4sc5c(c34)CCN(C(=O)/C=C/CN3CCC(O)C3)C5)cc2C)cn1] |
| 75 | 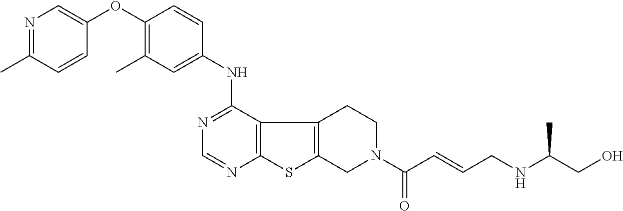 [SMILES: Cc1ccc(Oc2ccc(Nc3ncnc4sc5c(c34)CCN(C(=O)/C=C/CN[C@@H](C)CO)C5)cc2C)cn1] |
| 76 | 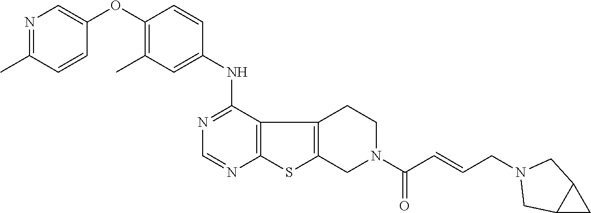 [SMILES: Cc1ccc(Oc2ccc(Nc3ncnc4sc5c(c34)CCN(C(=O)/C=C/CN3CC4CC4C3)C5)cc2C)cn1] |
| 77 | 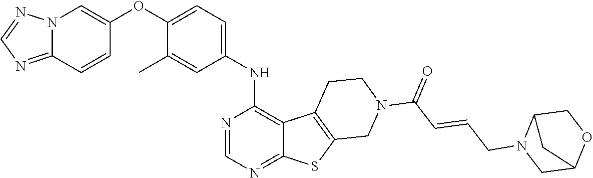 [SMILES: Cc1cc(Nc2ncnc3sc4c(c23)CCN(C(=O)/C=C/CN2CC3CC2CO3)C4)ccc1Oc1ccc2ncnn2c1] |
| 79 | 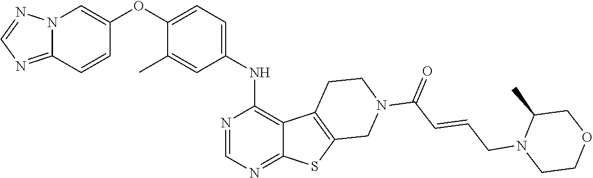 [SMILES: Cc1cc(Nc2ncnc3sc4c(c23)CCN(C(=O)/C=C/CN2CCOC[C@@H]2C)C4)ccc1Oc1ccc2ncnn2c1] |
| 80 | 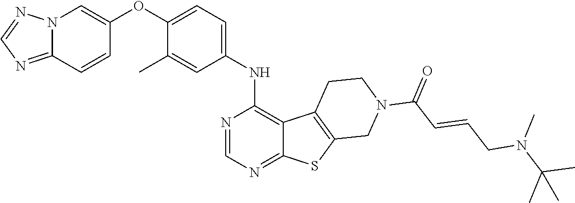 [SMILES: Cc1cc(Nc2ncnc3sc4c(c23)CCN(C(=O)/C=C/CN(C)C(C)(C)C)C4)ccc1Oc1ccc2ncnn2c1] |
| 81 | 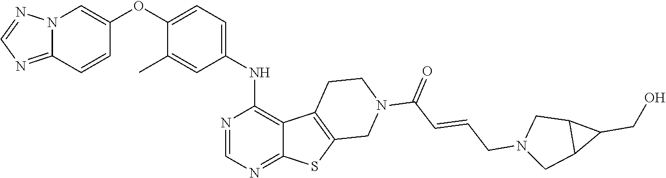 [SMILES: Cc1cc(Nc2ncnc3sc4c(c23)CCN(C(=O)/C=C/CN2CC3C(CO)C3C2)C4)ccc1Oc1ccc2ncnn2c1] |
| 82 | 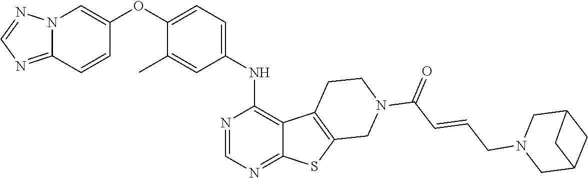 [SMILES: Cc1cc(Nc2ncnc3sc4c(c23)CCN(C(=O)/C=C/CN2CC3CC(C3)C2)C4)ccc1Oc1ccc2ncnn2c1] |
| 83 | 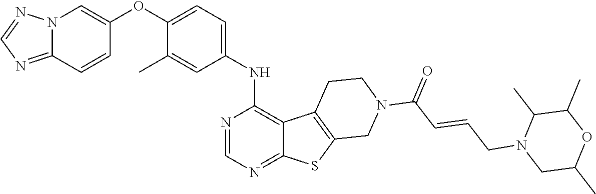 [SMILES: Cc1cc(Nc2ncnc3sc4c(c23)CCN(C(=O)/C=C/CN2CC(C)OC(C)C2C)C4)ccc1Oc1ccc2ncnn2c1] |
| 84 | 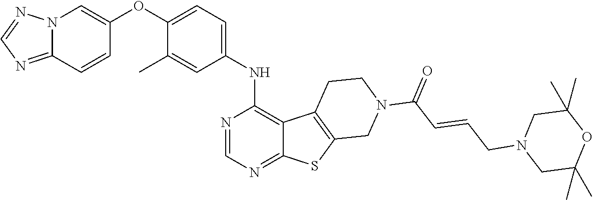 [SMILES: Cc1cc(Nc2ncnc3sc4c(c23)CCN(C(=O)/C=C/CN2CC(C)(C)OC(C)(C)C2)C4)ccc1Oc1ccc2ncnn2c1] |
| 85 | 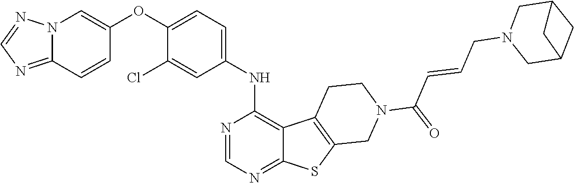 [SMILES: O=C(/C=C/CN1CC2CC(C2)C1)N1CCc2c(sc3ncnc(Nc4ccc(Oc5ccc6ncnn6c5)c(Cl)c4)c23)C1] |
| 86 | 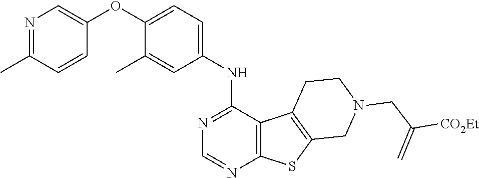 [SMILES: C=C(CN1CCc2c(sc3ncnc(Nc4ccc(Oc5ccc(C)nc5)c(C)c4)c23)C1)C(=O)OCC] |
| 87 | 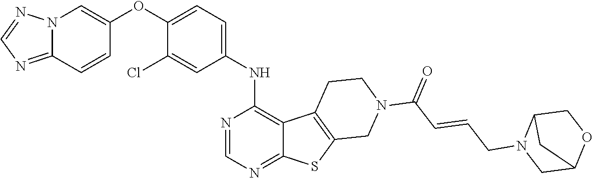 [SMILES: O=C(/C=C/CN1CC2CC1CO2)N1CCc2c(sc3ncnc(Nc4ccc(Oc5ccc6ncnn6c5)c(Cl)c4)c23)C1] |
| 88 | 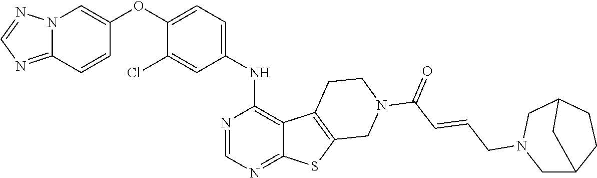 [SMILES: O=C(/C=C/CN1CC2CCC(C2)C1)N1CCc2c(sc3ncnc(Nc4ccc(Oc5ccc6ncnn6c5)c(Cl)c4)c23)C1] |
| 90 | 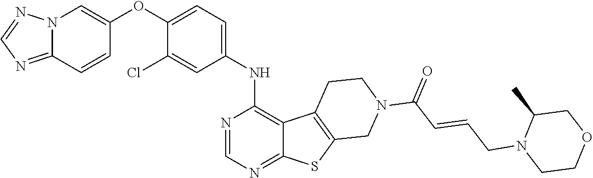 [SMILES: C[C@H]1COCCN1C/C=C/C(=O)N1CCc2c(sc3ncnc(Nc4ccc(Oc5ccc6ncnn6c5)c(Cl)c4)c23)C1] |
| 91 | 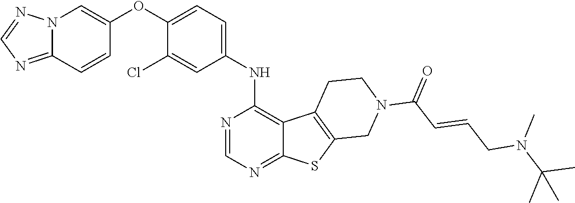 [SMILES: CN(C/C=C/C(=O)N1CCc2c(sc3ncnc(Nc4ccc(Oc5ccc6ncnn6c5)c(Cl)c4)c23)C1)C(C)(C)C] |
| 92 | 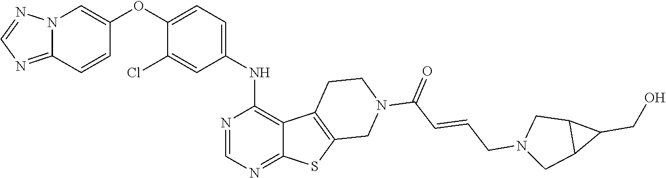 [SMILES: O=C(/C=C/CN1CC2C(CO)C2C1)N1CCc2c(sc3ncnc(Nc4ccc(Oc5ccc6ncnn6c5)c(Cl)c4)c23)C1] |
| 94 | 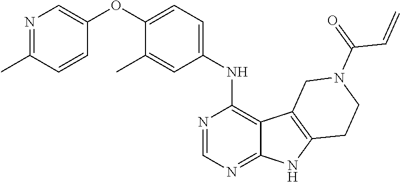 [SMILES: C=CC(=O)N1CCc2[nH]c3ncnc(Nc4ccc(Oc5ccc(C)nc5)c(C)c4)c3c2C1] |
| 95 | 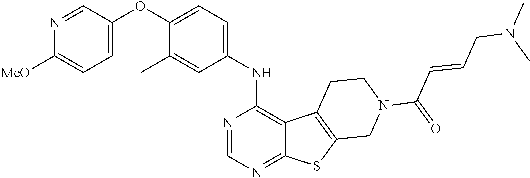 [SMILES: COc1ccc(Oc2ccc(Nc3ncnc4sc5c(c34)CCN(C(=O)/C=C/CN(C)C)C5)cc2C)cn1] |
| 96 | 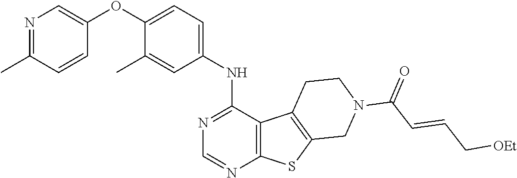 [SMILES: CCOC/C=C/C(=O)N1CCc2c(sc3ncnc(Nc4ccc(Oc5ccc(C)nc5)c(C)c4)c23)C1] |
| 97 | 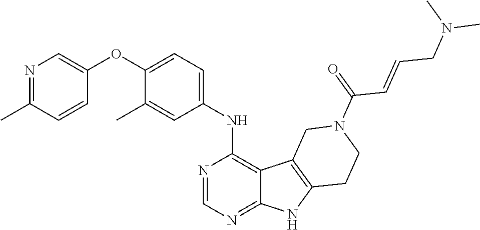 |
| 98 | 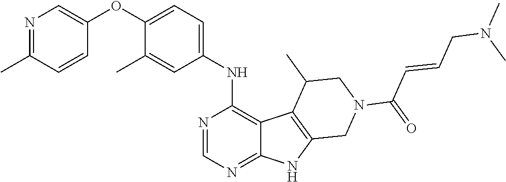 |
| 99 | 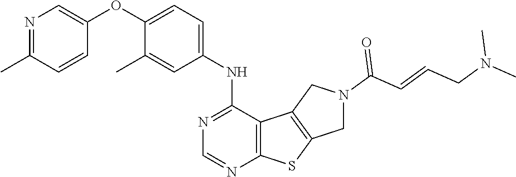 |
| 100 | 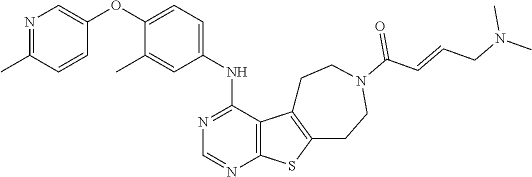 |
| 101 | 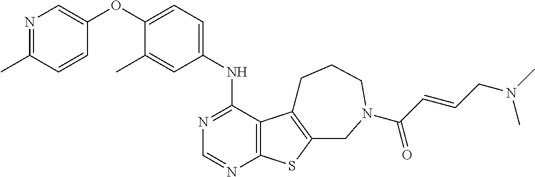 |
| 102 | 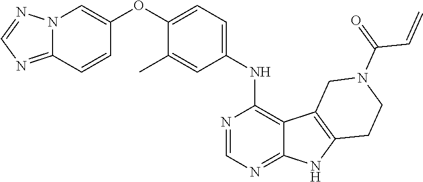 [SMILES: C=CC(=O)N1CCc2[nH]c3ncnc(Nc4ccc(Oc5ccc6ncnn6c5)c(C)c4)c3c2C1] |
| 103 | 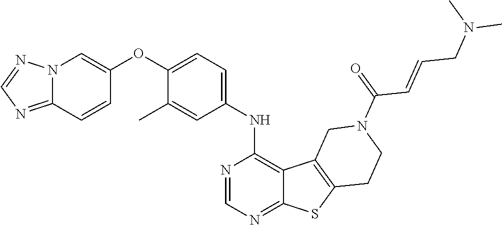 [SMILES: Cc1cc(Nc2ncnc3sc4c(c23)CN(C(=O)/C=C/CN(C)C)CC4)ccc1Oc1ccc2ncnn2c1] |
| 104 | 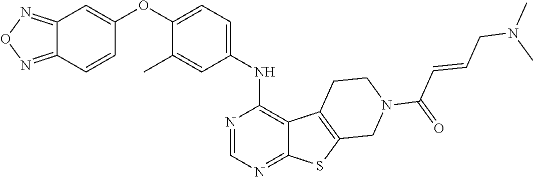 [SMILES: Cc1cc(Nc2ncnc3sc4c(c23)CCN(C(=O)/C=C/CN(C)C)C4)ccc1Oc1ccc2nonc2c1] |
| 105/106 | 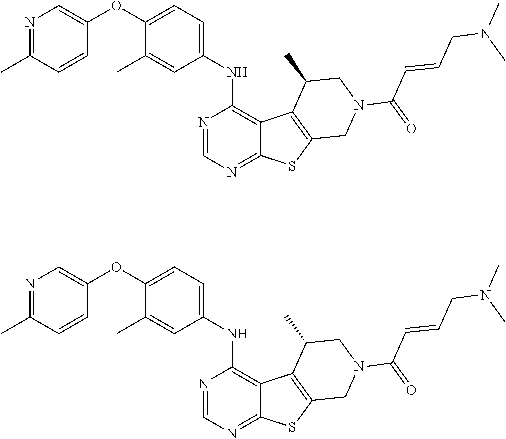 |
| 107 | 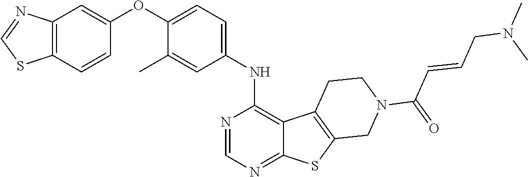 [SMILES: Cc1cc(Nc2ncnc3sc4c(c23)CCN(C(=O)/C=C/CN(C)C)C4)ccc1Oc1ccc2scnc2c1] |
| 108 | 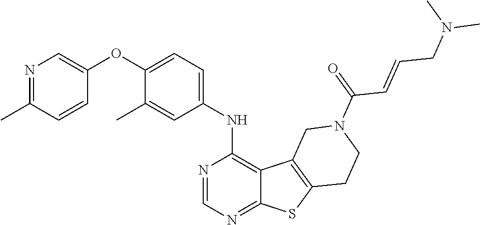 |
| 109 | 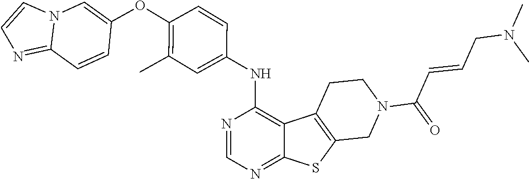 [SMILES: Cc1cc(Nc2ncnc3sc4c(c23)CCN(C(=O)/C=C/CN(C)C)C4)ccc1Oc1ccc2nccn2c1] |
| 110 | 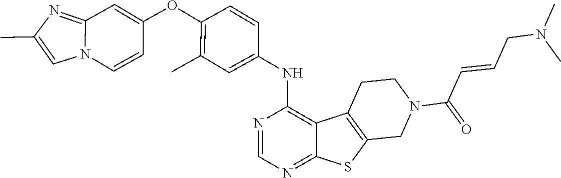 |
| 111 | 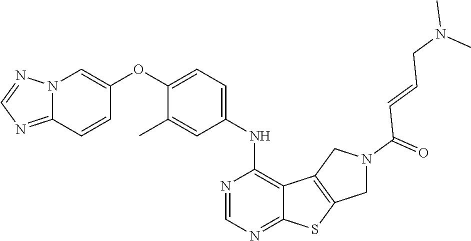 [SMILES: Cc1cc(Nc2ncnc3sc4c(c23)CN(C(=O)/C=C/CN(C)C)C4)ccc1Oc1ccc2ncnn2c1] |
| 112 | 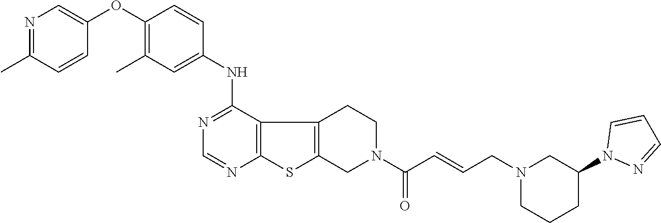 [SMILES: Cc1ccc(Oc2ccc(Nc3ncnc4sc5c(c34)CCN(C(=O)/C=C/CN3CCC[C@H](n4cccn4)C3)C5)cc2C)cn1] |
| 113 | 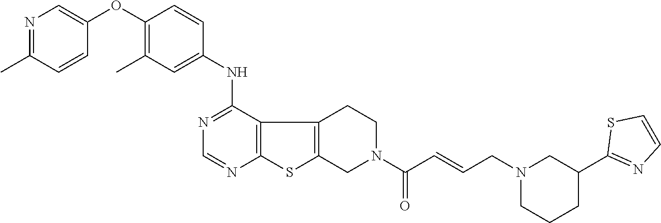 [SMILES: Cc1ccc(Oc2ccc(Nc3ncnc4sc5c(c34)CCN(C(=O)/C=C/CN3CCCC(c4nccs4)C3)C5)cc2C)cn1] |
| 114 | 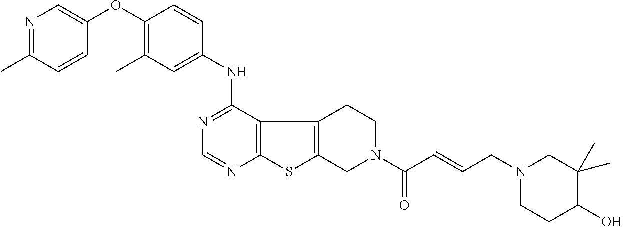 |
| 115 | 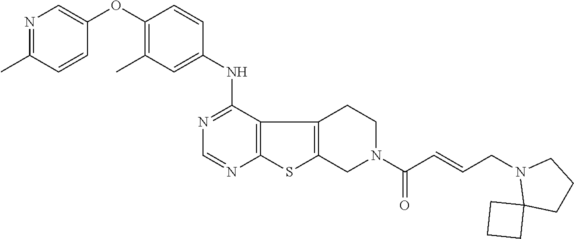 [SMILES: Cc1ccc(Oc2ccc(Nc3ncnc4sc5c(c34)CCN(C(=O)/C=C/CN3CCCC34CCC4)C5)cc2C)cn1] |
| 116 | 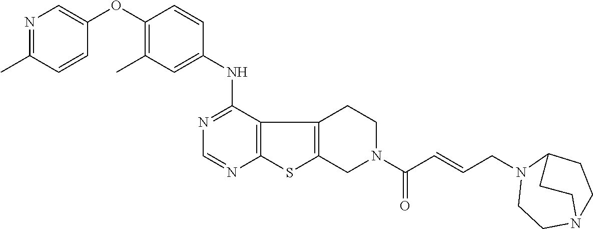 [SMILES: Cc1ccc(Oc2ccc(Nc3ncnc4sc5c(c34)CCN(C(=O)/C=C/CN3CCN4CCC3CC4)C5)cc2C)cn1] |
| 117 | 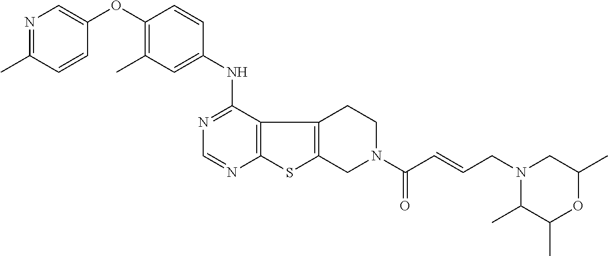 |
| 118 | 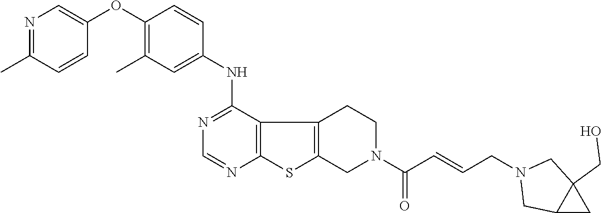 [SMILES: Cc1ccc(Oc2ccc(Nc3ncnc4sc5c(c34)CCN(C(=O)/C=C/CN3CC4CC4(CO)C3)C5)cc2C)cn1] |
| 119 | 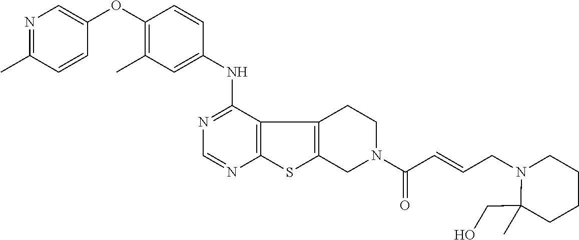 [SMILES: Cc1ccc(Oc2ccc(Nc3ncnc4sc5c(c34)CCN(C(=O)/C=C/CN3CCCCC3(C)CO)C5)cc2C)cn1] |
| 120 | 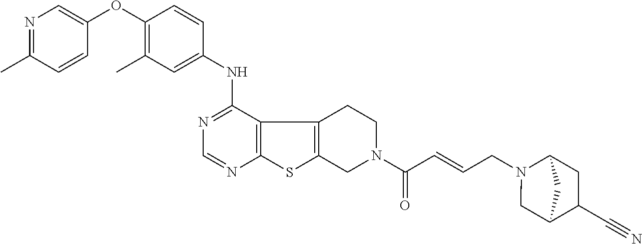 [SMILES: Cc1ccc(Oc2ccc(Nc3ncnc4sc5c(c34)CCN(C(=O)/C=C/CN3C[C@H]4C[C@@H]3CC4C#N)C5)cc2C)cn1] |
| 121 | 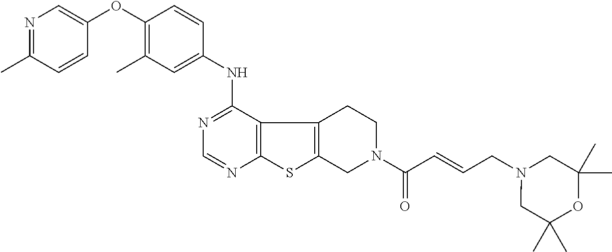 |
| 122 | 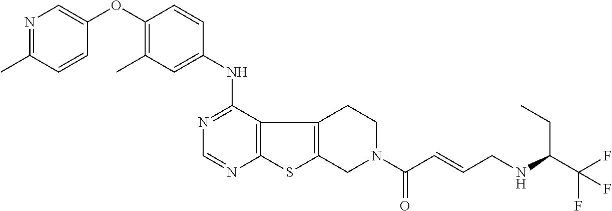 [SMILES: CC[C@H](NC/C=C/C(=O)N1CCc2c(sc3ncnc(Nc4ccc(Oc5ccc(C)nc5)c(C)c4)c23)C1)C(F)(F)F] |
| 123 | 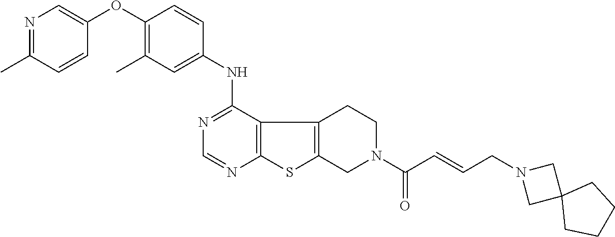 [SMILES: Cc1ccc(Oc2ccc(Nc3ncnc4sc5c(c34)CCN(C(=O)/C=C/CN3CC4(CCCC4)C3)C5)cc2C)cn1] |
| 124 | 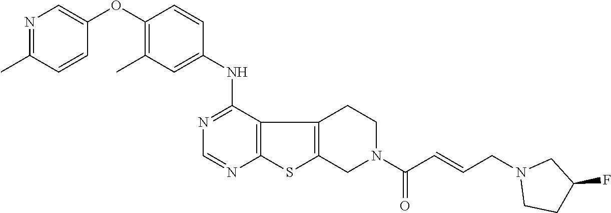 [SMILES: Cc1ccc(Oc2ccc(Nc3ncnc4sc5c(c34)CCN(C(=O)/C=C/CN3CC[C@H](F)C3)C5)cc2C)cn1] |
| 126 | 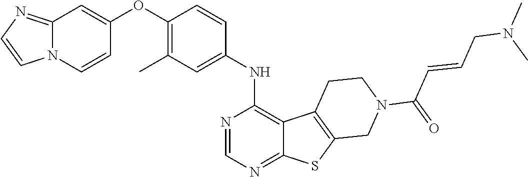 [SMILES: Cc1cc(Nc2ncnc3sc4c(c23)CCN(C(=O)/C=C/CN(C)C)C4)ccc1Oc1ccn2ccnc2c1] |
| 127 | 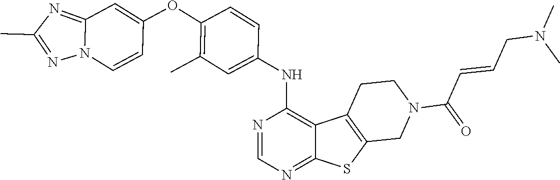 |
| 128 | 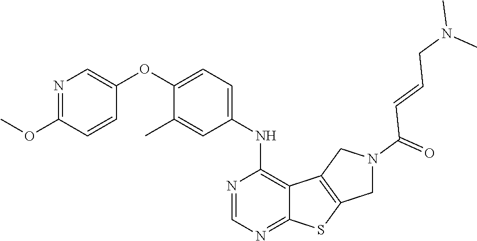 |
| 129 | 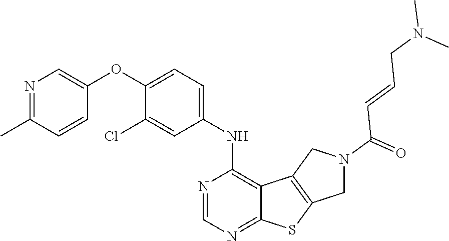 |
| 130 | 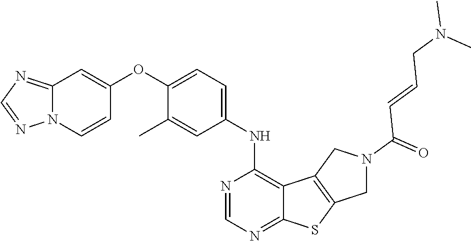 [SMILES: Cc1cc(Nc2ncnc3sc4c(c23)CN(C(=O)/C=C/CN(C)C)C4)ccc1Oc1ccn2ncnc2c1] |
| 131 | 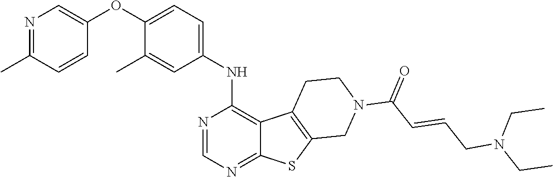 [SMILES: CCN(CC)C/C=C/C(=O)N1CCc2c(sc3ncnc(Nc4ccc(Oc5ccc(C)nc5)c(C)c4)c23)C1] |
| 132 | 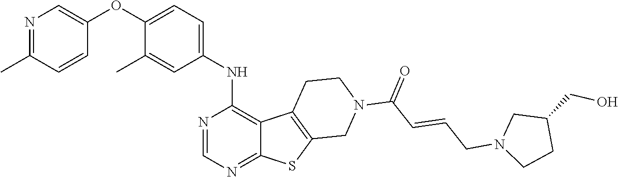 [SMILES: Cc1ccc(Oc2ccc(Nc3ncnc4sc5c(c34)CCN(C(=O)/C=C/CN3CC[C@@H](CO)C3)C5)cc2C)cn1] |
| 133 | 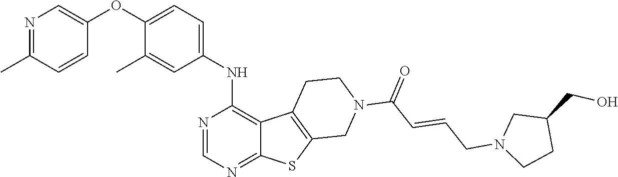 [SMILES: Cc1ccc(Oc2ccc(Nc3ncnc4sc5c(c34)CCN(C(=O)/C=C/CN3CC[C@H](CO)C3)C5)cc2C)cn1] |
| 134 | 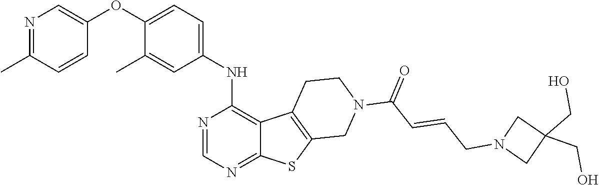 [SMILES: Cc1ccc(Oc2ccc(Nc3ncnc4sc5c(c34)CCN(C(=O)/C=C/CN3CC(CO)(CO)C3)C5)cc2C)cn1] |
| 135 | 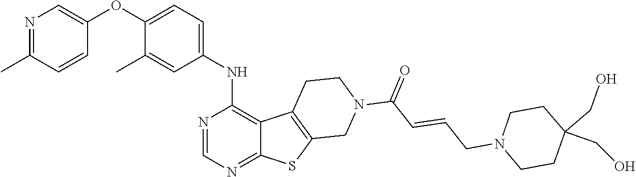 [SMILES: Cc1ccc(Oc2ccc(Nc3ncnc4sc5c(c34)CCN(C(=O)/C=C/CN3CCC(CO)(CO)CC3)C5)cc2C)cn1] |
| 136 | 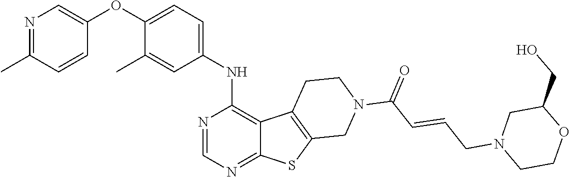 [SMILES: Cc1ccc(Oc2ccc(Nc3ncnc4sc5c(c34)CCN(C(=O)/C=C/CN3CCO[C@H](CO)C3)C5)cc2C)cn1] |
| 137 | 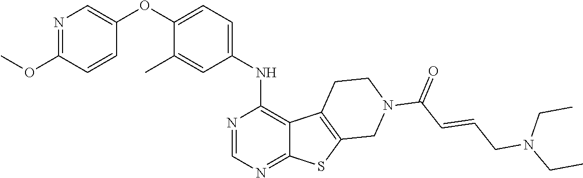 [SMILES: CCN(CC)C/C=C/C(=O)N1CCc2c(sc3ncnc(Nc4ccc(Oc5ccc(OC)nc5)c(C)c4)c23)C1] |
| 138 | 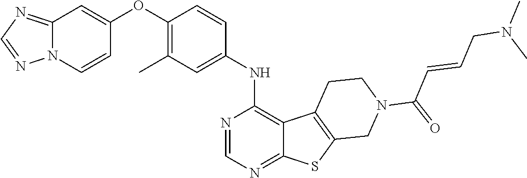 [SMILES: Cc1cc(Nc2ncnc3sc4c(c23)CCN(C(=O)/C=C/CN(C)C)C4)ccc1Oc1ccn2ncnc2c1] |
| 139 | 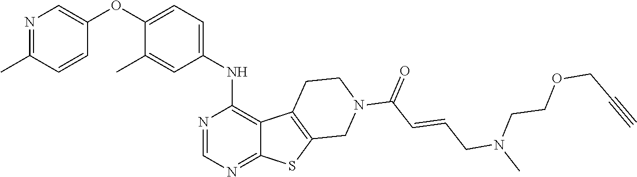 [SMILES: C#CCOCCN(C)C/C=C/C(=O)N1CCc2c(sc3ncnc(Nc4ccc(Oc5ccc(C)nc5)c(C)c4)c23)C1] |
| 140 | 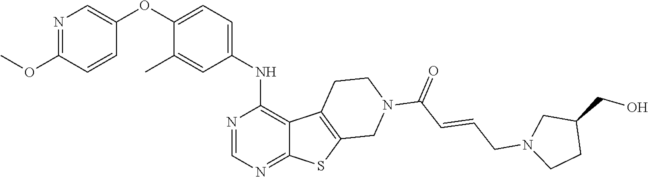 [SMILES: COc1ccc(Oc2ccc(Nc3ncnc4sc5c(c34)CCN(C(=O)/C=C/CN3CC[C@H](CO)C3)C5)cc2C)cn1] |
| 141 | 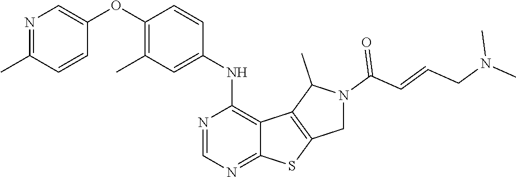 |
| 142 | 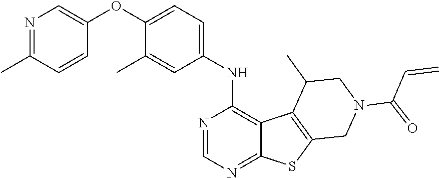 [SMILES: C=CC(=O)N1Cc2sc3ncnc(Nc4ccc(Oc5ccc(C)nc5)c(C)c4)c3c2C(C)C1] |
| 143 | 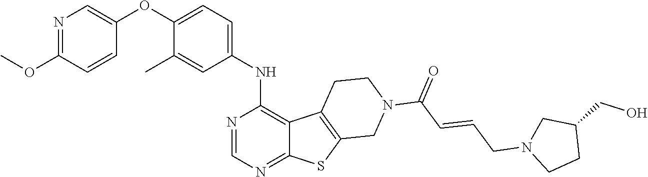 [SMILES: COc1ccc(Oc2ccc(Nc3ncnc4sc5c(c34)CCN(C(=O)/C=C/CN3CC[C@@H](CO)C3)C5)cc2C)cn1] |
| 144 | 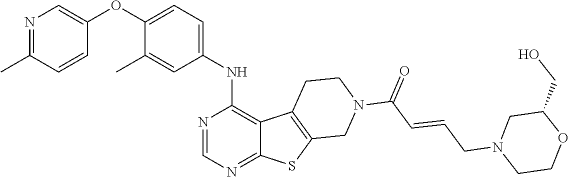 [SMILES: Cc1ccc(Oc2ccc(Nc3ncnc4sc5c(c34)CCN(C(=O)/C=C/CN3CCO[C@@H](CO)C3)C5)cc2C)cn1] |
| 145/146 | 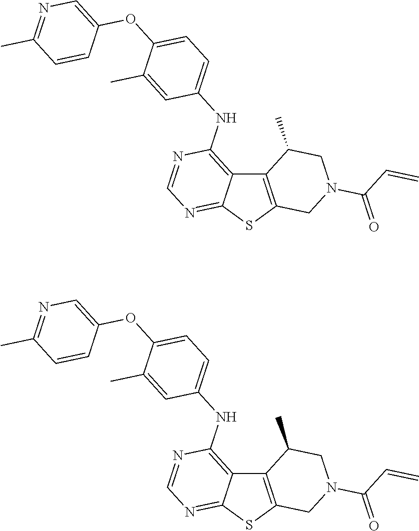 |
| 147/148 | 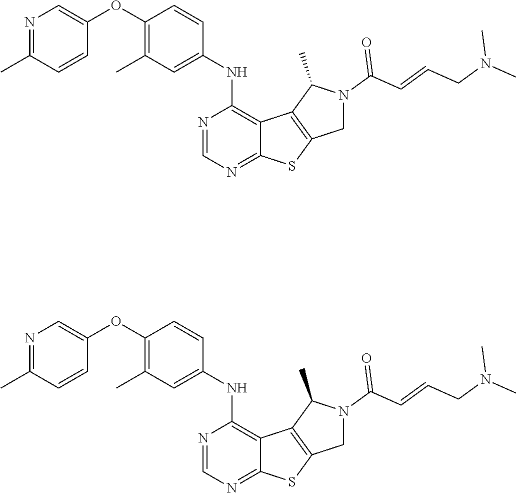 |
| 149 | 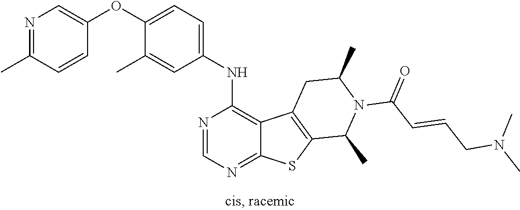 |
| 150 | 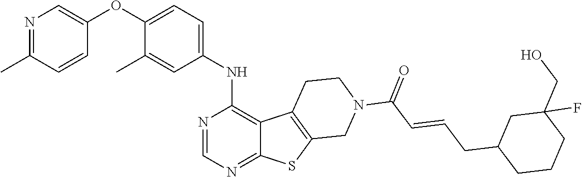 [SMILES: Cc1ccc(Oc2ccc(Nc3ncnc4sc5c(c34)CCN(C(=O)/C=C/CC3CCCC(F)(CO)C3)C5)cc2C)cn1] |
| 151 | 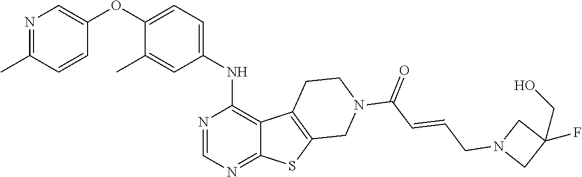 [SMILES: Cc1ccc(Oc2ccc(Nc3ncnc4sc5c(c34)CCN(C(=O)/C=C/CN3CC(F)(CO)C3)C5)cc2C)cn1] |
| 152 | 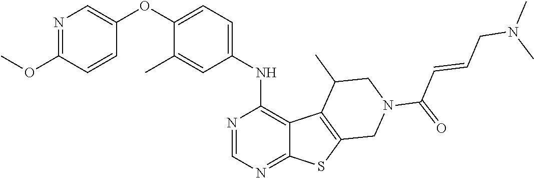 |
| 153 | 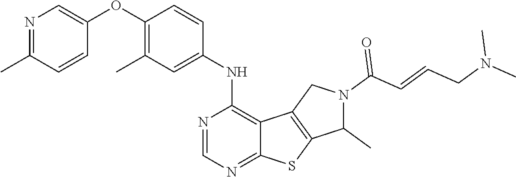 |
| 154 | 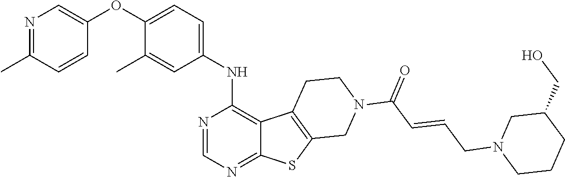 [SMILES: Cc1ccc(Oc2ccc(Nc3ncnc4sc5c(c34)CCN(C(=O)/C=C/CN3CCC[C@@H](CO)C3)C5)cc2C)cn1] |
| 155 | 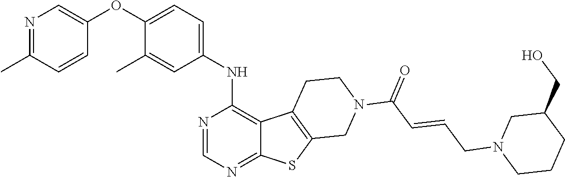 [SMILES: Cc1ccc(Oc2ccc(Nc3ncnc4sc5c(c34)CCN(C(=O)/C=C/CN3CCC[C@H](CO)C3)C5)cc2C)cn1] |
| 158 | 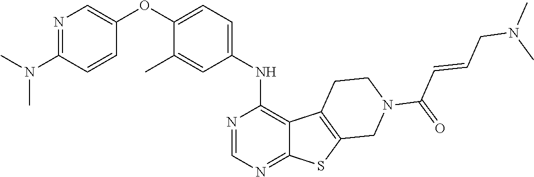 |
| 159/160 | 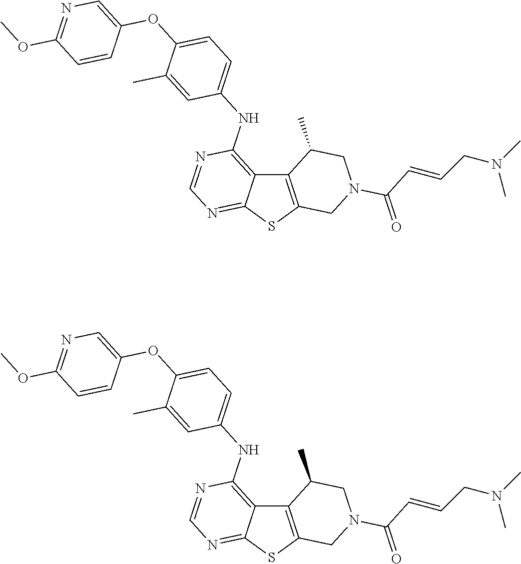 |
| 161/162 | 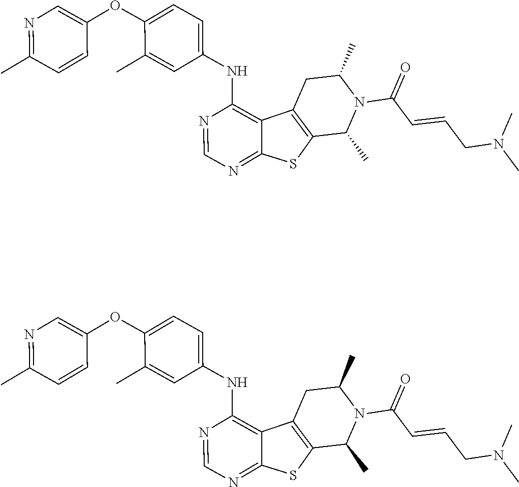 |
| 163 | 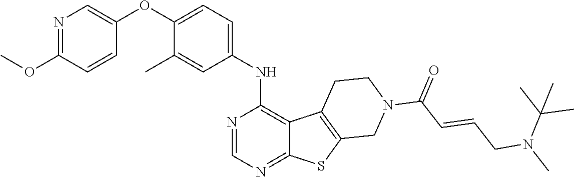 [SMILES: COc1ccc(Oc2ccc(Nc3ncnc4sc5c(c34)CCN(C(=O)/C=C/CN(C)C(C)(C)C)C5)cc2C)cn1] |
| 164 | 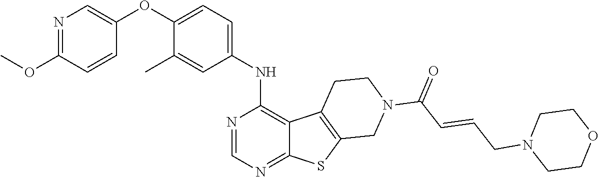 [SMILES: COc1ccc(Oc2ccc(Nc3ncnc4sc5c(c34)CCN(C(=O)/C=C/CN3CCOCC3)C5)cc2C)cn1] |
| 165 | 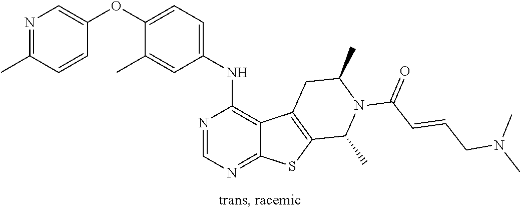 |
| 166 | 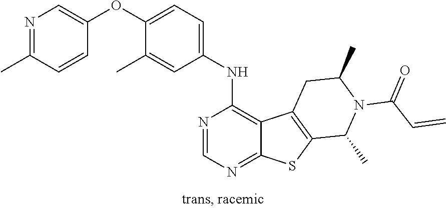 [SMILES: C=CC(=O)N1[C@H](C)Cc2c(sc3ncnc(Nc4ccc(Oc5ccc(C)nc5)c(C)c4)c23)[C@H]1C] |
| 167 | 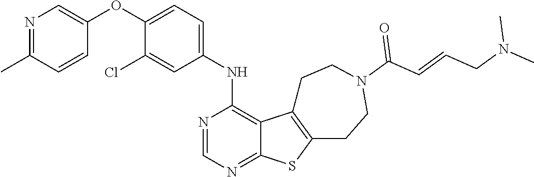 |
| 168 | 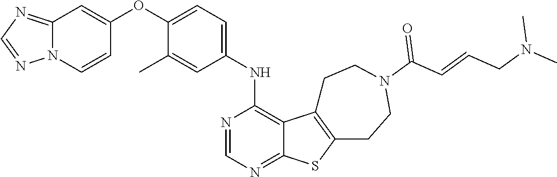 [SMILES: Cc1cc(Nc2ncnc3sc4c(c23)CCN(C(=O)/C=C/CN(C)C)CC4)ccc1Oc1ccn2ncnc2c1] |
| 169/170 | 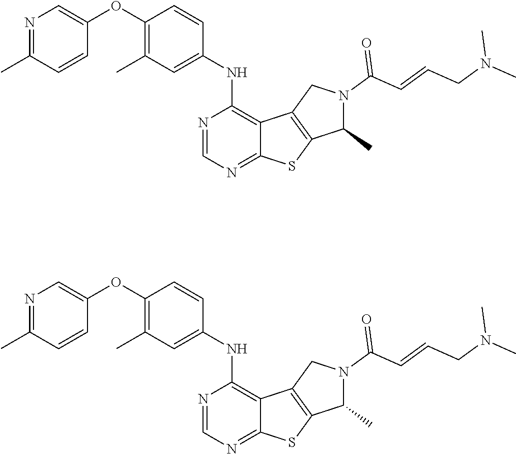 |
| 171 | 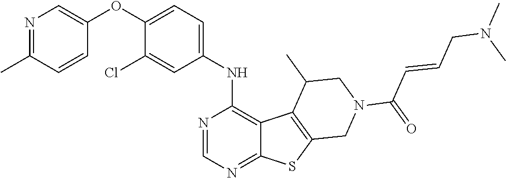 |
| 172 | 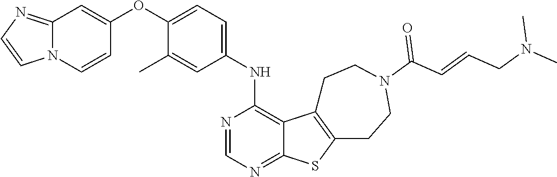 [SMILES: Cc1cc(Nc2ncnc3sc4c(c23)CCN(C(=O)/C=C/CN(C)C)CC4)ccc1Oc1ccn2ccnc2c1] |
| 173 | 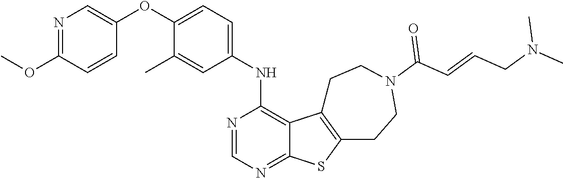 |
| 174 | 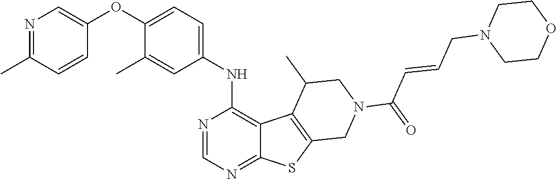 [SMILES: Cc1ccc(Oc2ccc(Nc3ncnc4sc5c(c34)C(C)CN(C(=O)/C=C/CN3CCOCC3)C5)cc2C)cn1] |
| 175 | 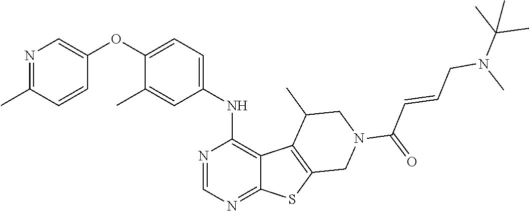 [SMILES: Cc1ccc(Oc2ccc(Nc3ncnc4sc5c(c34)C(C)CN(C(=O)/C=C/CN(C)C(C)(C)C)C5)cc2C)cn1] |
| 176/177 | 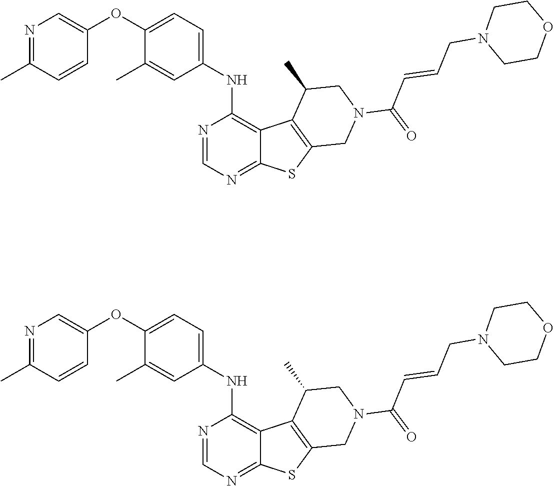 [SMILES: Cc1ccc(Oc2ccc(Nc3ncnc4sc5c(c34)[C@@H](C)CN(C(=O)/C=C/CN3CCOCC3)C5)cc2C)cn1 ; Cc1ccc(Oc2ccc(Nc3ncnc4sc5c(c34)[C@H](C)CN(C(=O)/C=C/CN3CCOCC3)C5)cc2C)cn1] |
| 178/179 | 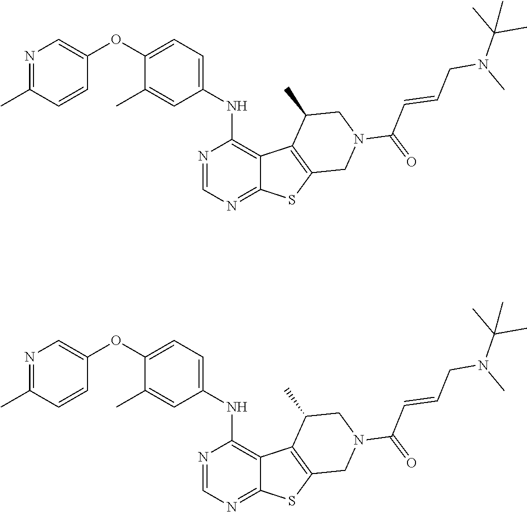 |

Methods of Treatment, Prevention and/or Management

Compounds provided herein (e.g., a compound of Formula (I), or a pharmaceutically acceptable salt, tautomer, stereoisomer, enantiomer, or isotopologue thereof) have utility as therapeutic agents to treat, prevent or manage conditions in animals or humans.

In certain embodiments, without being bound by a particular theory, compounds provided herein (e.g., a compound of Formula (I), or a pharmaceutically acceptable salt, tautomer, stereoisomer, enantiomer, or isotopologue thereof) bind to a tyrosine kinase. In one embodiment, compounds provided herein bind to an epidermal growth factor receptor. In one embodiment, compounds provided herein bind to HER2. In one embodiment, compounds provided herein inhibit the activity of HER2. In one embodiment, compounds provided herein inhibit the signaling and proliferation of cells with overexpression or amplification of HER2.

In certain embodiments, without being bound by a particular theory, compounds provided herein (e.g., a compound of Formula (I), or a pharmaceutically acceptable salt, tautomer, stereoisomer, enantiomer, or isotopologue thereof) bind to wildtype HER2.

In certain embodiments, without being bound by a particular theory, compounds provided herein (e.g., a compound of Formula (I), or a pharmaceutically acceptable salt, tautomer, stereoisomer, enantiomer, or isotopologue thereof) bind to one or more of HER2 mutants. In one embodiment, compounds provided herein inhibit the activity of a HER2 mutant. In one embodiment, compounds provided herein inhibit the signaling and proliferation of cells with overexpression or amplification of a HER2 mutant.

In certain embodiments, the HER2 mutant contains a deletion, insertion, or substitution. In certain embodiments, the HER2 mutant contains one or more deletions, insertions, or substitutions at the amino acid positions of 309, 310, 630, 678, 717, 719, 724, 726, 733, 755, 755-759, 760, 767, 769, 775-778, 777, 780, 781, 783, 784, 785, 798, 803, 812, 821, 835, 839, 842, 866, 896, and 915. In certain embodiments, the HER2 mutant contains one, two, or more deletions, insertions, and/or substitutions, each independently selected from G309A, G309E, S310F, C630Y, R678Q, E717K, E719G, E719K, K724N, L726F, T733I, L755P, L755S, L755W, L755_T759del, S760A, I767F, I767M, D769H, D769Y, A755_G776 ins YVMA (or “YVMA” as referred herein), G776delinsVC (or “VC” as referred herein), G776delinsLC, V777_G778insCG, G778_P780dup, V777L, V777M, P780L, P780_Y781insGSP, S783P, R784C, L785F, T798I, Y803N, E812K, D821N, Y835F, V839G, V842I, T862A, L866M, R896C, and L915M, provided that there is only one deletion and/or insertion, or substitution at a given amino acid position in the HER2 mutant. In certain embodiments, the HER2 mutant contains one, two, or more deletions, insertions, and/or substitutions, each independently selected from G309A, L755S, L755_T759del, A775_G776insYVMA, V777L, P780_Y781insGSP, R678Q, L755W, V842I, and R896C.

In one embodiment, the HER2 mutant is HER2YVMA. In another embodiment, the HER2 mutant is HER2VC. In another embodiment, the HER2 mutant is HER2 L755S. In another embodiment, the HER2 mutant is HER2 G776C. In another embodiment, the HER2 mutant is HER2 V777_G778insCG.

In certain embodiments, without being bound by a particular theory, compounds provided herein (e.g., a compound of Formula (I), or a pharmaceutically acceptable salt, tautomer, stereoisomer, enantiomer, or isotopologue thereof) selectively bind to HER2 over EGFR.

In certain embodiments, without being bound by a particular theory, compounds provided herein (e.g., a compound of Formula (I), or a pharmaceutically acceptable salt, tautomer, stereoisomer, enantiomer, or isotopologue thereof) may pass blood-brain barrier.

Compounds provided herein (e.g., a compound of Formula (I), or a pharmaceutically acceptable salt, tautomer, stereoisomer, enantiomer, or isotopologue thereof) have utility as therapeutic agents to treat, prevent or manage diseases or disorders mediated by HER2 or a HER2 mutant.

In one embodiment, provided herein is a method of treating, preventing, or managing a disorder mediated by HER2 or a HER2 mutant, comprising administering to a subject in need thereof a therapeutically or prophylactically effective amount of a compound provided herein (e.g., a compound of Formula (I), or a pharmaceutically acceptable salt, tautomer, stereoisomer, enantiomer, or isotopologue thereof).

Examples of “a disorder mediated by HER2 or a HER2 mutant” include, but are not limited to, angiogenesis disorders and cancers.

Examples of angiogenesis disorders include, but are not limited to, angiogenesis associated with the growth of cancer or sarcoma, angiogenesis associated with cancer metastasis, angiogenesis associated with diabetic retinopathy, arteriosclerosis, restenosis, psoriasis, and the like.

Examples of cancers include, but are not limited to, brain tumor, pharyngeal cancer, laryngeal cancer, tongue cancer, esophageal cancer, gastric cancer, colorectal cancer, lung cancer, pancreatic cancer, bile duct cancer, gallbladder cancer, liver cancer, renal cancer, bladder cancer, prostate cancer, breast cancer, ovarian cancer, cervical cancer, endometrial cancer, skin cancer, childhood solid cancer, bone tumor, hemangioma and the like.

Examples of specific cancers include, but are not limited to, advanced malignancy, amyloidosis, neuroblastoma, meningioma, hemangiopericytoma, multiple brain metastases, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

In one embodiment, the disorder is mediated by expression, overexpression, amplification, or activation of HER2. Overexpression, amplification, or activation of HER2 can lead to unwanted cell proliferation. Examples of such cell proliferation disorders include, but are not limited to, cancer, angiogenesis associated with the growth of cancer or sarcoma, angiogenesis associated with cancer metastasis, angiogenesis associated with diabetic retinopathy, arteriosclerosis, restenosis, or psoriasis.

In one embodiment, the disorder is a cancer mediated by expression, overexpression, amplification, or activation of HER2. In one embodiment, the cancer is breast cancer, gastric cancer, esophageal cancer, ovarian cancer, endometrial cancer, endometrial serous carcinoma, cervix cancer, bladder cancer, lung cancer, colorectal cancer, head and neck cancer, cholangial cancer, germ cell cancer, glioblastoma, liver cancer, melanoma, osteosarcoma, pancreatic cancer, renal cell cancinoma, salivary duct carcinoma, and soft tissue cancer. In one embodiment, the cancer is breast cancer. In one embodiment, the cancer is a metastatic breast cancer that spreads to brain. In one embodiment, the cancer is gastric cancer. In one embodiment, the cancer is esophageal cancer. In one embodiment, the cancer is ovarian cancer. In one embodiment, the cancer is endometrial cancer. In one embodiment, the cancer is endometrial serous carcinoma.

In one embodiment, the disorder is mediated by expression, overexpression, amplification, or activation of one or more HER2 mutants. In one embodiment, the disorder is a cancer mediated by expression, overexpression, amplification, or activation of one or more HER2 mutants such as HER2YVMA, HER2VC, HER2 L755S, HER2 G776C, and HER2 V777_G778insCG. In one embodiment, the cancer is bladder cancer, cervix cancer, colorectal cancer, endometrial cancer, germ cell cancer, glioblastoma, head and neck cancer, liver cancer, lung cancer, ovarian cancer, pancreatic cancer, and salivary duct carcinoma. In one embodiment, the cancer is lung cancer. In one embodiment, the cancer is non-small cell lung cancer (NSCLC).

In one embodiment, provided herein is a method of treating, preventing, or managing cancer, comprising administering to a subject in need thereof a therapeutically or prophylactically effective amount of a compound provided herein (e.g., a compound of Formula (I), or a pharmaceutically acceptable salt, tautomer, stereoisomer, enantiomer, or isotopologue thereof).

In one embodiment, provided herein is a method of treating or managing cancer, comprising administering to a subject in need thereof a therapeutically effective amount of a compound provided herein (e.g., a compound of Formula (I), or a pharmaceutically acceptable salt, tautomer, stereoisomer, enantiomer, or isotopologue thereof).

As used herein, the term “cancer” includes, but is not limited to, solid tumors and blood borne tumors. The term “cancer” refers to disease of skin tissues, organs, blood, and vessels, including, but not limited to, cancers of the bladder, bone, blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. In some embodiments, exemplary cancers include multiple myeloma, leukemias (for example, acute lymphocytic leukemia, acute and chronic myelogenous leukemia, chronic lymphocytic leukemia, acute lymphoblastic leukemia, or promyelocytic leukemia), lymphomas (for example, B-cell lymphoma, T-cell lymphoma, mantle cell lymphoma, hairy cell lymphoma, Burkitt's lymphoma, mast cell tumors, Hodgkin's disease or non-Hodgkin's disease), myelodysplastic syndrome, fibrosarcoma, rhabdomyosarcoma; astrocytoma, neuroblastoma, glioma and schwannomas; melanoma, seminoma, teratocarcinoma, osteosarcoma, xenoderma pigmentosum, keratoctanthoma, thyroid follicular cancer, Kaposi's sarcoma, melanoma, teratoma, rhabdomyosarcoma, metastatic and bone disorders, as well as cancer of the bone, mouth/pharynx, esophagus, larynx, stomach, intestine, colon, rectum, lung (for example, non-small cell lung cancer or small cell lung cancer), liver, pancreas, nerve, brain (for example, glioma or glioblastoma multiforme), head and neck, throat, ovary, uterus, prostate, testis, bladder, kidney, breast, gall bladder, cervix, thyroid, prostate, and skin.

In certain embodiments, the cancer is a blood cancer. In certain embodiments, the blood cancer is metastatic. In certain embodiments, the blood cancer is drug resistant. In certain embodiments, the cancer is myeloma, lymphoma, or leukemia.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Leukemias include, but are not limited to, acute myeloid leukemia, acute lymphoblastic leukemia, chronic lymphocytic leukemia, chronic myelogenous leukemia, and hairy cell leukemia.

Myelomas include, but are not limited to, multiple myeloma.

In certain embodiments, the cancer is a solid tumor. In certain embodiments, the solid tumor is metastatic. In certain embodiments, the solid tumor is drug-resistant. In certain embodiments, the solid tumor is breast cancer, lung cancer, colorectal cancer, gastric cancer, esophageal cancer, ovarian cancer, or endometrial cancer.

In one embodiment, the solid tumor is breast cancer. Examples of breast cancer include, but are not limited to, invasive ductal carcinoma, invasive lobular carcinoma, ductal carcinoma in situ, and lobular carcinoma in situ. In one embodiment, the breast cancer is a HER2 positive breast cancer. In one embodiment, the breast cancer is a metastatic breast cancer. In one embodiment, the breast cancer is a HER2 amplified metastatic breast cancer. In one embodiment, the breast cancer is metastatic breast cancer that spreads to CNS (e.g., brain). In one embodiment, the breast cancer is characterized by the presence of one or more HER2 mutants. In one embodiment, the HER2 mutant is HER2 L755_T759del. In another embodiment, the HER2 mutant is HER2 L755S. In another embodiment, the HER2 mutant is HER2 V777L. In another embodiment, the HER2 mutant is HER2 R896C. In another embodiment, the HER2 mutant is HER2 D769H. In another embodiment, the HER2 mutant is HER2 D769Y. In another embodiment, the HER2 mutant is HER2 G309A. In another embodiment, the HER2 mutant is HER2 V842I. In another embodiment, the HER2 mutant is HER2 P780_Y781insGSP.

In one embodiment, the solid tumor is gastric cancer. Examples of gastric cancer include, but are not limited to, Epstein-Barr virus (EBV) positive gastric cancer, gastric cancer with high microsatellite instability, genomically stable gastric cancer, and chromosomally unstable gastric cancer. In one embodiment, the gastric cancer is a HER2 positive gastric cancer. one embodiment, the gastric cancer is characterized by the presence of one or more HER2 mutants.

In one embodiment, the solid tumor is ovarian cancer. Examples of ovarian cancer include, but are not limited to, epithelial ovarian cancer, primary peritoneal cancer, borderline tumors, germ cell tumors, Sex cord stromal cell tumors, choriocarcinoma, dysgerminoma, endodermal sinus tumors, embryonal carcinoma, granulosa cell tumors, sarcomas, Sertoli-Leydig tumors, teratoma, and the like. Subtypes of epithelial ovarian cancer include, but are not limited to, serous, mucinous, endometrioid, clear cell, and undifferentiated or unclassified epithelial ovarian cancer. In one embodiment, the ovarian cancer is a HER2 positive ovarian cancer. one embodiment, the ovarian cancer is characterized by the presence of one or more HER2 mutants. In one embodiment, the HER2 mutant is HER2YVMA. In another embodiment, the HER2 mutant is HER2 D769Y. In another embodiment, the HER2 mutant is HER2 T862A.

In one embodiment, the solid tumor is endometrial cancer. Examples of endometrial cancer include, but are not limited to, endometrial carcinomas (including Type I and Type II subtypes), endometrial serous carcinoma, endometrioid adenocarcinoma, uterine papillary serous carcinoma, and uterine clear-cell carcinoma. In one embodiment, the endometrial cancer is endometrial serous carcinoma. In one embodiment, the endometrial cancer is a HER2 positive endometrial cancer. In one embodiment, the endometrial cancer is characterized by the presence of one or more HER2 mutants.

In one embodiment, the solid tumor is esophageal cancer. Examples of esophageal cancer include, but are not limited to, esophageal squamous-cell carcinoma and esophageal adenocarcinoma. In one embodiment, the esophageal cancer is a HER2 positive esophageal cancer. In one embodiment, the esophageal cancer is characterized by the presence of one or more HER2 mutants. In one embodiment, the mutant is HER2 T862A.

In one embodiment, the solid tumor is lung cancer. Examples of lung cancer include, but are not limited to, small-cell lung carcinoma and non-small-cell lung carcinoma (NSCLC). In one embodiment, the lung cancer is non-small-cell lung carcinoma. In one embodiment, the lung cancer or NSCLC is characterized by the presence of one or more HER2 mutants. In one embodiment, the HER2 mutant is HER2YVMA. In another embodiment, the HER2 mutant is HER2VC. In another embodiment, the HER2 mutant is HER2 L755S. In another embodiment, the HER2 mutant is HER2 G776C. In another embodiment, the HER2 mutant is HER2 V777_G778insCG.

In one embodiment, the solid tumor is colorectal cancer. In one embodiment, the colorectal cancer is characterized by the presence of one or more HER2 mutants. In one embodiment, the HER2 mutant is HER2 L755S. In another embodiment, the HER2 mutant is HER2 V777L. In another embodiment, the HER2 mutant is HER2 V777M. In another embodiment, the HER2 mutant is HER2 V842I. In another embodiment, the HER2 mutant is HER2 S310F. In another embodiment, the HER2 mutant is HER2 L866M.

In one embodiment, provided herein is a method of treating, preventing, or managing breast cancer, comprising administering to a subject in need thereof a therapeutically or prophylactically effective amount of a compound of Formula (I), or a pharmaceutically acceptable salt, tautomer, stereoisomer, enantiomer, or isotopologue thereof. In one embodiment, the breast cancer is metastatic breast cancer that spreads to brain.

In one embodiment, provided herein is a method of treating or managing breast cancer, comprising administering to a subject in need thereof a therapeutically effective amount of a compound of Formula (I), or a pharmaceutically acceptable salt, tautomer, stereoisomer, enantiomer, or isotopologue thereof. In one embodiment, the breast cancer is metastatic breast cancer that spreads to brain.

In one embodiment, provided herein is a method of treating, preventing, or managing gastric cancer, comprising administering to a subject in need thereof a therapeutically or prophylactically effective amount of a compound of Formula (I), or a pharmaceutically acceptable salt, tautomer, stereoisomer, enantiomer, or isotopologue thereof.

In one embodiment, provided herein is a method of treating or managing gastric cancer, comprising administering to a subject in need thereof a therapeutically effective amount of a compound of Formula (I), or a pharmaceutically acceptable salt, tautomer, stereoisomer, enantiomer, or isotopologue thereof.

In one embodiment, provided herein is a method of treating, preventing, or managing ovarian cancer, comprising administering to a subject in need thereof a therapeutically or prophylactically effective amount of a compound of Formula (I), or a pharmaceutically acceptable salt, tautomer, stereoisomer, enantiomer, or isotopologue thereof.

In one embodiment, provided herein is a method of treating or managing ovarian cancer, comprising administering to a subject in need thereof a therapeutically effective amount of a compound of Formula (I), or a pharmaceutically acceptable salt, tautomer, stereoisomer, enantiomer, or isotopologue thereof.

In one embodiment, provided herein is a method of treating, preventing, or managing endometrial cancer, comprising administering to a subject in need thereof a therapeutically or prophylactically effective amount of a compound of Formula (I), or a pharmaceutically acceptable salt, tautomer, stereoisomer, enantiomer, or isotopologue thereof. In one embodiment, the endometrial cancer is endometrial serous carcinoma.

In one embodiment, provided herein is a method of treating or managing endometrial cancer, comprising administering to a subject in need thereof a therapeutically effective amount of a compound of Formula (I), or a pharmaceutically acceptable salt, tautomer, stereoisomer, enantiomer, or isotopologue thereof. In one embodiment, the endometrial cancer is endometrial serous carcinoma.

In one embodiment, provided herein is a method of treating, preventing, or managing esophageal cancer, comprising administering to a subject in need thereof a therapeutically or prophylactically effective amount of a compound of Formula (I), or a pharmaceutically acceptable salt, tautomer, stereoisomer, enantiomer, or isotopologue thereof.

In one embodiment, provided herein is a method of treating or managing esophageal cancer, comprising administering to a subject in need thereof a therapeutically effective amount of a compound of Formula (I), or a pharmaceutically acceptable salt, tautomer, stereoisomer, enantiomer, or isotopologue thereof.

In one embodiment, provided herein is a method of treating, preventing, or managing lung cancer, comprising administering to a subject in need thereof a therapeutically or prophylactically effective amount of a compound of Formula (I), or a pharmaceutically acceptable salt, tautomer, stereoisomer, enantiomer, or isotopologue thereof. In one embodiment, the lung cancer is non-small-cell lung carcinoma.

In one embodiment, provided herein is a method of treating or managing lung cancer, comprising administering to a subject in need thereof a therapeutically effective amount of a compound of Formula (I), or a pharmaceutically acceptable salt, tautomer, stereoisomer, enantiomer, or isotopologue thereof. In one embodiment, the lung cancer is non-small-cell lung carcinoma.

In one embodiment, provided herein is a method of treating, preventing, or managing colorectal cancer, comprising administering to a subject in need thereof a therapeutically or prophylactically effective amount of a compound of Formula (I), or a pharmaceutically acceptable salt, tautomer, stereoisomer, enantiomer, or isotopologue thereof.

In one embodiment, provided herein is a method of treating or managing colorectal cancer, comprising administering to a subject in need thereof a therapeutically effective amount of a compound of Formula (I), or a pharmaceutically acceptable salt, tautomer, stereoisomer, enantiomer, or isotopologue thereof.

In one embodiment, the cancer is newly diagnosed, relapsed, refractory, or relapsed and refractory.

In one embodiment, the cancer is metastatic. In one embodiment, the cancer is non-metastatic.

In one embodiment, the subject is a mammal. In one embodiment, the subject is a human.

In certain embodiments, a therapeutically or prophylactically effective amount of a compound provided herein is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, or from about 0.05 to about 10 mg per day.

In certain embodiments, the therapeutically or prophylactically effective amount of a compound provided herein is about 0.1, about 0.2, about 0.3. about 0.5, about 1, about 2, about 5, about 10, about 15, about 20, about 25, about 30, about 40, about 45, about 50, about 60, about 70, about 80, about 90, about 100, or about 150 mg per day.

In one embodiment, the recommended daily dose range of a compound provided herein for the conditions described herein lie within the range of from about 0.5 mg to about 50 mg per day, preferably given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.01, 0.05, 0.1, 0.2, 0.3, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.01, 0.05, 0.1, 0.2, 0.3, 0.4, 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, or from about 0.01 to about 1 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein. Once steady state is reached, there are minor peaks and troughs on the time dependent curve of the plasma concentration of the compound.

In certain embodiments, the amount of the compound administered is sufficient to provide a maximum plasma concentration (peak concentration) of the compound, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In certain embodiments, the amount of the compound administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

In certain embodiments, the patient to be treated with one of the methods provided herein has not been treated with anticancer therapy prior to the administration of a compound provided herein. In certain embodiments, the patient to be treated with one of the methods provided herein has been treated with anticancer therapy prior to the administration of a compound provided herein. In certain embodiments, the patient to be treated with one of the methods provided herein has developed drug resistance to the anticancer therapy.

The methods provided herein encompass treating a patient regardless of patient's age, although some diseases or disorders are more common in certain age groups. Further provided herein is a method for treating a patient who has undergone surgery in an attempt to treat the disease or condition at issue, as well in one who has not. Because the subjects with cancer have heterogeneous clinical manifestations and varying clinical outcomes, the treatment given to a particular subject may vary, depending on his/her prognosis. The skilled clinician will be able to readily determine without undue experimentation, specific secondary agents, types of surgery, and types of non-drug based standard therapy that can be effectively used to treat an individual subject with cancer.

Depending on the disease to be treated and the subject's condition, a compound provided herein, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. A compound provided herein may be formulated, alone or together, in suitable dosage unit with pharmaceutically acceptable excipients, carriers, adjuvants and vehicles, appropriate for each route of administration.

In one embodiment, a compound provided herein is administered orally. In another embodiment, a compound provided herein is administered parenterally. In yet another embodiment, a compound provided herein is administered intravenously.

In one embodiment, a compound provided herein can be delivered as a single dose such as, e.g., a single bolus injection, or oral tablets or pills; or over time, such as, e.g., continuous infusion over time or divided bolus doses over time. The compound can be administered repeatedly if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205-216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

In one embodiment, a compound provided herein can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound is administered once or more than once each day, for example, for a period of time. The term “continuous” is intended to mean that a therapeutic compound is administered daily for an uninterrupted period of at least 10 days to 52 weeks. The term “intermittent” or “intermittently” as used herein is intended to mean stopping and starting at either regular or irregular intervals. For example, intermittent administration of a compound provided herein is administration for one to six days per week, administration in cycles (e.g., daily administration for two to eight consecutive weeks, then a rest period with no administration for up to one week), or administration on alternate days. The term “cycling” as used herein is intended to mean that a therapeutic compound is administered daily or continuously but with a rest period.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, a compound provided herein is administered once a day. In another embodiment, a compound provided herein is administered twice a day. In yet another embodiment, a compound provided herein is administered three times a day. In still another embodiment, a compound provided herein is administered four times a day.

In certain embodiments, a compound provided herein is administered once per day from one day to six months, from one week to three months, from one week to four weeks, from one week to three weeks, or from one week to two weeks. In certain embodiments, a compound provided herein is administered once per day for one week, two weeks, three weeks, or four weeks. In one embodiment, a compound provided herein is administered once per day for one week. In another embodiment, a compound provided herein is administered once per day for two weeks. In yet another embodiment, a compound provided herein is administered once per day for three weeks. In still another embodiment, a compound provided herein is administered once per day for four weeks.

### Cycling Therapy

In certain embodiments, a compound provided herein (e.g., a compound of Formula (I), or a pharmaceutically acceptable salt, tautomer, stereoisomer, enantiomer, or isotopologue thereof) is cyclically administered to a patient. Cycling therapy involves the administration of an active agent for a period of time, followed by a rest (i.e., discontinuation of the administration) for a period of time, and repeating this sequential administration. Cycling therapy can reduce the development of resistance to one or more of the therapies, avoid or reduce the side effects of one of the therapies, and/or improve the efficacy of the treatment.

Consequently, in certain embodiments, a compound provided herein is administered daily in a single or divided doses in a four to six week cycle with a rest period of about a week or two weeks. The cycling method further allows the frequency, number, and length of dosing cycles to be increased. Thus, encompassed herein in certain embodiments is the administration of a compound provided herein for more cycles than are typical when it is administered alone. In certain embodiments, a compound provided herein is administered for a greater number of cycles that would typically cause dose-limiting toxicity in a patient to whom a second active ingredient is not also being administered.

In one embodiment, a compound provided herein is administered daily and continuously for three or four weeks at a dose of from about 0.1 to about 150 mg/d followed by a break of one or two weeks.

### Pharmaceutical Compositions and Dosage Forms

Pharmaceutical compositions can be used in the preparation of individual, single unit dosage forms. Pharmaceutical compositions and dosage forms provided herein comprise a compound provided herein (e.g., a compound of Formula (I), or a pharmaceutically acceptable salt, tautomer, stereoisomer, enantiomer, or isotopologue thereof). Pharmaceutical compositions and dosage forms provided herein can further comprise one or more excipients.

Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms provided herein will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms provided herein will vary from one another will be readily apparent to those skilled in the art. See, e.g., Remington's Pharmaceutical Sciences, 18th ed., Mack Publishing, Easton PA (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided herein are pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions provided herein can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

Further provided herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms provided herein comprise a compound provided herein in an amount of from about 0.10 to about 500 mg. Typical dosage forms comprise a compound provided herein in an amount of about 0.1, 1, 2, 5, 7.5, 10, 12.5, 15, 17.5, 20, 25, 50, 100, 150, 200, 250, 300, 350, 400, 450, or 500 mg.

### Oral Dosage Forms

Pharmaceutical compositions provided herein that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton PA (1990).

Typical oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, PA), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions provided herein is typically present in from about 50 to about 99 weight percent of the pharmaceutical composition or dosage form.

Disintegrants are used in the compositions provided herein to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W. R. Grace Co. of Baltimore, MD), a coagulated aerosol of synthetic silica (marketed by Degussa Co. of Plano, TX), CAB-O-SIL (a pyrogenic silicon dioxide product sold by Cabot Co. of Boston, MA), and mixtures thereof. If used at all, lubricants are typically used in an amount of less than about 1 weight percent of the pharmaceutical compositions or dosage forms into which they are incorporated.

A solid oral dosage form provided herein comprises a compound provided herein, anhydrous lactose, microcrystalline cellulose, polyvinylpyrrolidone, stearic acid, colloidal anhydrous silica, and gelatin.

### Controlled Release Dosage Forms

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein. Thus, provided herein are single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms provided herein are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

In one embodiment, provided herein are pharmaceutical composition comprising a compound provided herein, which is suitable for intravenous administration. In one embodiment, provided herein is a method of treating, preventing, and/or managing a disease or disorder provided herein elsewhere comprising administering to a patient a compound provided herein via intravenous administration.

Compounds that increase the solubility of one or more of the active ingredients disclosed herein can also be incorporated into the parenteral dosage forms provided herein. For example, cyclodextrin and its derivatives can be used to increase the solubility of an immunomodulatory compound provided herein and its derivatives. See, e.g., U.S. Pat. No. 5,134,127, which is incorporated herein by reference.

### Topical and Mucosal Dosage Forms

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton PA (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms provided herein are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton PA (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Kits

In one embodiment, active ingredients provided herein are preferably not administered to a patient at the same time or by the same route of administration. Thus, provided herein are kits which, when used by the medical practitioner, can simplify the administration of appropriate amounts of active ingredients to a patient.

Kits provided herein can further comprise devices that are used to administer the active ingredients. Examples of such devices include, but are not limited to, syringes, drip bags, patches, and inhalers.

Kits provided herein can further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### EXAMPLES

Certain embodiments provided herein are illustrated by the following non-limiting examples.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

Enantioenriched compounds of the invention were prepared in enantioenriched form using chiral starting materials, or were separated after reaction with a racemic starting material, using chiral chromatography. For compounds prepared as racemic or diastereomeric mixtures, the single isomers can be prepared in optically pure form by either employing chiral starting materials or performing chiral chromatography.

In the illustrative examples that follow, reactions were carried out at room or ambient temperature, in the range of 18-25° C. unless otherwise stated. Organic solutions were dried over anhydrous magnesium sulfate or sodium sulfate and evaporation of solvent was carried out using a rotary evaporator under reduced pressure. In general, the courses of reactions were followed by TLC or LCMS and reaction times are representative. Yields are given for illustration only and are not necessarily those which can be obtained by diligent process development.

¹HNMR data is in delta values for major diagnostic protons, given in parts per million (ppm) relative to tetramethylsilane (TMS) or residual solvent. ¹HNMR spectra were determined at 400 MHz. Solvent ratios are given in volume:volume (v/v) terms. Mass spectra (MS) data was generated on an LCMS system where the HPLC component comprised generally either an Agilent or Shimadzu LCMS-2020 Instrument and was run on a Sepax BR-C18 (4.6×50 mm, 3 m) column or similar, eluting with acidic eluent (for example, using a gradient between 0-95% water acetonitrile with 0.1% formic acid or trifluoroacetic acid). Chromatograms were in electrospray (ESI) positive, negative and/or UV. LCMS values for m/z are provided throughout and generally, only ions which indicate the parent mass are reported. Unless otherwise stated the value quoted is the (M+H) or (M+1) for positive ion mode. Preparative HPLC was performed on C₁₈ reversed-phase silica using decreasingly polar mixtures as eluent, for example decreasingly polar mixtures of water and acetonitrile containing 1% trifluoroacetic acid. Column chromatography refers to normal phase silica gel chromatography.

Enantioenriched intermediates and final compounds were synthesized using commercially available chiral materials and their stereochemistry as recorded is absolute. Unless otherwise specified, starting materials were commercially available or synthesized according to known methods.

### Abbreviations

| | |
|---|---|
| TFA | trifluoroacetic acid |
| NCS | N-chlorosuccinimide |
| TFAA | trifluoroacetic anhydride |
| THF | tetrahydrofuran |
| DMF | N,N-dimethylformamide |
| DMA | N,N-dimethylacetamide |
| NMP | 1-methyl-2-pyrrolidinone |
| EtOAc | ethyl acetate |
| DCM | dichloromethane |
| DMSO | dimethylsulfoxide |
| ACN/MeCN | acetonitrile |
| t-AmOH, | 2-methyl-2-butanol |
| t-amyl | |
| alcohol | |
| mCPBA | m-chloroperoxybenzoic acid |
| DIPEA | N,N-diisopropylethylamine |
| (Hünig's | |
| base) | |
| TBAF | tetra-N-butylammonium fluoride |
| PMB | p-methoxybenzyl |
| rac | racemic |
| DMAP | 4-dimethylaminopyridine |
| dba | dibenzylideneacetone |
| dppf | 1,1′-bis(diphenylphosphino) ferrocene |
| Xantphos | 4,5-Bis(diphenylphosphino)-9,9-dimethylxanthene |
| DavePhos | 2-Dicyclohexylphosphino-2′-(N,N-dimethylamino)biphenyl |
| SPhos | 2-Dicyclohexylphosphino-2′,6′-dimethoxybiphenyl |
| BINAP | (±)-2,2′-Bis(diphenylphosphino)-1,1′-binaphthyl |
| BrettPhos- | Chloro[2-(dicyclohexylphosphino)-3,6-dimethoxy-2′,4′,6′- |
| G1 | triisopropyl-1,1′-biphenyl][2-(2- |
| | aminoethyl)phenyl]palladium(II) |
| B₂pin₂ | Bis(pinacolato)diboron |
| h | hour |
| min | minute |
| aq | aqueous |
| g | gram |
| sat | saturated |
| Boc | tert-butoxy carbamate |
| THP | tetrahydropyranyl ether |
| eq | equivalent |
| r.t. | room temperature |

Compound numbers utilized in the Examples below correspond to compound numbers set forth in Table 1, supra.

### Synthesis of Common Intermediates

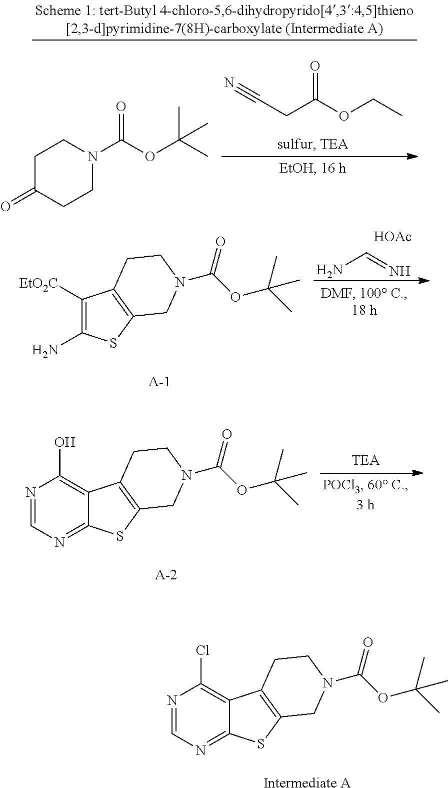

### Step 1: 6-tert-Butyl 3-ethyl 2-amino-4,5-dihydrothieno[2,3-c]pyridine-3,6(7H)-dicarboxylate (A-1)

To a mixture of tert-butyl 4-oxopiperidine-1-carboxylate (100 g, 0.50 mol), ethyl cyanoacetate (57 g, 0.50 mol), and sulfur (16 g, 0.50 mol) in absolute ethanol (400 mL) was added triethylamine (70 ml, 0.50 mol). The reaction mixture was allowed to stir at ambient temperature for 16 h after which it was filtered and the resultant solid was washed with ethanol and dried to provide the title compound which was used directly in the next step (136 g, 83%). LCMS m/z 327.1 [M+H]⁺. ¹HNMR (400 MHz, CDCl₃): δ 1.34 (t, 3H), 1.48 (s, 9H), 2.8 (s, 2H), 3.62 (t, 2H), 4.26 (q, 2H), 4.35 (s, 2H), 6.00 (s, 2H).

### Step 2: tert-Butyl 4-hydroxy-5,6-dihydropyrido[4′,3′:4,5]thieno[2,3-d]pyrimidine-7(8H)-carboxylate (A-2)

A mixture of A-1 (136 g, 0.4 mol) and formamidine acetate (65 g, 0.6 mol) in DMF (400 mL) was heated at 100° C. for 18 h. The reaction mixture was cooled to ambient temperature. Ice water was slowly added. The solid was collected by filtration, washed with water and ether and dried to afford the title compound (119 g, 92%). LCMS m/z 308.2 [M+H]^{+ 1}HNMR (400 MHz, CDCl₃): δ 1.50 (s, 9H), 3.13 (s, 2H), 3.74 (t, 2H), 4.66 (s, 2H), 7.98 (s, 1H).

### Step 3: tert-Butyl 4-chloro-5,6-dihydropyrido[4′,3′:4,5]thieno[2,3-d]pyrimidine-7(8H)-carboxylate (Intermediate A)

To a mixture of POCl₃ (130 mL) and triethylamine (130 mL) at 0° C. was added A-2 (100 g, 326 mmol). The mixture was heated at 60° C. for 3 h, after which the reaction was allowed cool to ambient temperature, POCl₃ was removed under reduced pressure and the resultant residue was neutralized through slow addition of saturated aqueous sodium bicarbonate solution. The mixture was partitioned between DCM and H₂O. The organic layer was washed with water, brine, dried over Na₂SO₄, concentrated. The resultant residue was purified by column (hexane:acetate=8:1) to afford the title compound (50 g, 47%). LCMS m/z 326.0 [M+H]⁺. ¹HNMR (400 MHz, CDCl₃): δ 1.51 (s, 9H), 3.21 (s, 2H), 3.80 (t, 2H), 4.74 (s, 2H), 8.76 (s, 1H).

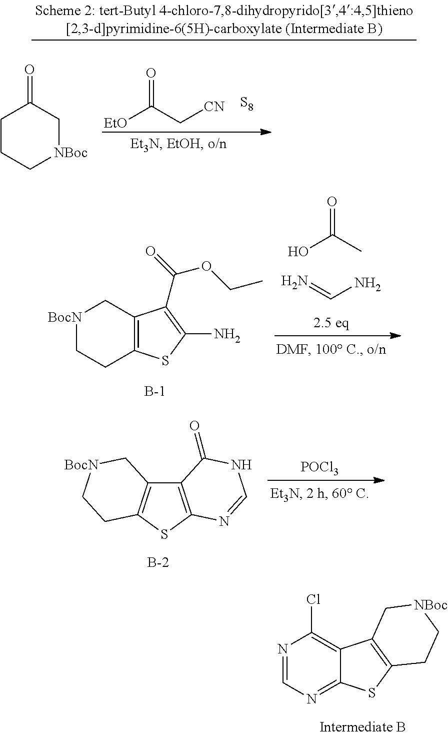

### Step 1: tert-Butyl 5-tert-butyl 3-ethyl 2-amino-6, 7-dihydrothieno[3,2-c]pyridine-3,5 (4H)-dicarboxylate (B-1)

A mixture of tert-butyl 3-oxopiperidine-1-carboxylate (5.0 g, 25.1 mmol), ethyl but-3-ynoate (2.81 g, 25.1 mmol), S (0.8 g, 25.1 mmol), Et₃N (3.5 mL, 25.1 mmol) and ethanol (15 mL) was stirred at room temperature for 16 h. The precipitate was collected by filtration and washed with ethanol to afford the title compound as yellow solid (1.41 g, 17%). LCMS m/z [M+H]⁺: 327.1.

### Step 2: tert-Butyl 4-oxo-3,4,7,8-tetrahydropyrido[3′,4′:4,5]thieno[2,3-d]pyrimidine-6(5H)-carboxylate (B-2)

A mixture of B-1 (1.41 g, 4.32 mmol), formimidamide acetate (1.12 g, 10.8 mmol) and DMF (20 mL) was stirred at 100° C. overnight. The reaction mixture was cooled to ambient temperature and concentrated to remove most DMF. To the mixture was added water. The precipitate was collected by filtration to afford the title compound as yellow solid (1.08 g, 81.3%). LCMS m/z [M+H]⁺: 308.0.

### Step 3: tert-Butyl 4-chloro-7, 8-dihydropyrido[3′, 4:4, 5]thieno[2,3-d]pyrimidine-6(5H)-carboxylate (Intermediate B)

To a mixture of POCl₃ (1.5 g, 9.76 mmol), Et₃N (985 mg, 9.76 mmol) at 0° C. was added B-2 (1.0 g, 3.25 mmol). The resulting mixture was heated at 60° C. for 2 h. The mixture was cooled to ambient temperature and neutralized with Et₃N. Toluene and brine were added. The resulting mixture was extracted with EtOAc, dried and concentrated. The resultant residue was purified by column chromatography (eluting with 25% EtOAc in hexane) to afford the title compound as off-white solid (500 mg, 47%). LCMS m/z [M+H]⁺: 326.0. ¹H NMR (400 MHz, DMSO-d₆): δ 8.87 (s, 1H), 4.87 (s, 2H), 3.73 (t, 2H), 2.99 (t, 2H), 1.45 (s, 9H).

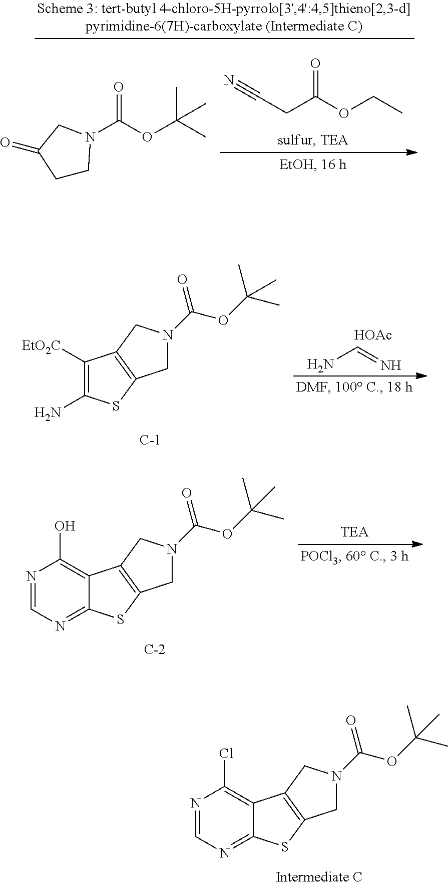

### Step 1: 5-tert-butyl 3-ethyl 2-amino-4H-thieno[2,3-c]pyrrole-3,5(6H)-dicarboxylate (C-1)

To a mixture of tert-butyl 3-oxopyrrolidine-1-carboxylate (20 g, 0.11 mol), ethyl cyanoacetate (12.4 g, 0.11 mol) and sulfur (3.52 g, 0.11 mol) in absolute ethanol (80 mL) was added triethylamine (11.1 g, 0.11 mol). The resulting mixture was stirred at ambient temperature for 16 h. The precipitate was collected by filtration, washed with ethanol, and dried to afford the title compound (15 g, 42%). ¹HNMR (400 MHz, CD₃OD): δ 4.48-4.42 (m, 4H), 4.25-4.23 (m, 2H), 1.53 (d, 9H), 1.35-1.32 (m, 3H).

### Step 2: tert-butyl 4-hydroxy-5H-pyrrolo[3′,4′:4,5]thieno[2,3-d]pyrimidine-6(7H)-carboxylate (C-2)

A mixture of C-1 (14.6 g, 0.047 mol) and formamidine acetate (7.4 g, 0.071 mol) in DMF (50 mL) was heated at 100° C. for 18 h. Reaction mixture was concentrated to remove most DMF under reduced pressure. To the residue was added water. The precipitate was collected by filtration and washed with water and ether and dried to afford the title compound (11 g, 81%). ¹HNMR (400 MHz, DMSO-d₆): δ 12.6 (s, 1H), 8.1 (s, 1H), 4.65-4.55 (m, 4H), 1.46 (d, 9H).

### Step 3: tert-butyl 4-chloro-5H-pyrrolo[3′,4′:4,5]thieno[2,3-d]pyrimidine-6(7H)-carboxylate (Intermediate C)

To a mixture of POCl₃ (15 mL) and triethylamine (15 mL) at 0° C. was added C-2 (9.6 g, 0.03 mol). The resulting mixture was heated at 60° C. for 3 h. The reaction mixture was concentrated to remove POCl₃. The resultant residue was taken up in H₂O and neutralized through slow addition of saturated aqueous NaHCO₃ solution. DCM was added and the organic layer was washed with water, brine, dried over Na₂SO₄, and concentrated. The resultant residue was purified through column chromatography to afford the title compound (5.0 g, 49%). ¹H NMR (400 MHz, CDCl₃): δ 8.81 (s, 1H), 4.93-4.81 (m, 4H), 1.55 (d, 9H).

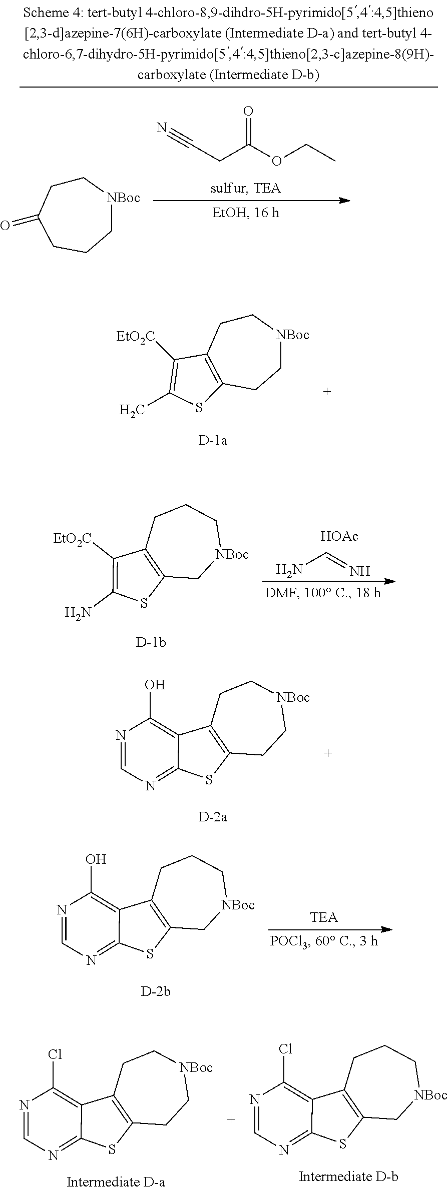

### Step 1: 6-tert-Butyl 3-ethyl 2-amino-7,8-dihydro-4H-thieno[2,3-d]azepine-3,6(5H)-dicarboxylate & 7-tert-butyl 3-ethyl (D-1a) and 2-amino-5,6-dihydro-4H-thieno[2,3-c]azepine-3,7(8H)-dicarboxylate (D-1b)

A mixture of tert-butyl 4-oxoazepane-1-carboxylate (13.7 g, 64.2 mmol), ethyl 2-cyanoacetate (7.27 g, 64.2 mmol), S₈ (2.06 g, 64.24 mmol) and Et₃N (6.49 g, 64.2 mmol) in ethanol (32 mL) was allowed to stir at room temperature for 22 h. The mixture was concentrated and the resultant residue dissolved in EtOAc, washed with brine, concentrated, and purified by silica gel column chromatography (hexane/EtOAc=20/1). The resultant light yellow solid was washed with hexane/ethyl acetate=10/1 to afford a mixture of D-1a/D-1b˜1:6 as white solid (12.2 g, 59%). LCMS: 341.1[M+H]⁺.

### Step 2: tert-Butyl 4-chloro-8,9-dihydro-5H-pyrimido[5′,4′:4,5]thieno[2,3-d]azepine-7(6H)-carboxylate (D-2a) and tert-butyl 4-oxo-3,4,5,6,7,9-hexahydro-1H-pyrimido[5′,4′:4,5]thieno[2,3-c]azepine-8(2H)-carboxylate (D-2b)

A mixture of D-1a/D-1b (2.0 g, 5.87 mmol) and methanimidamide acetic acid (0.92 g, 8.81 mmol) in DMF (20 mL) was allowed to stir at 100° C. for 24 h. The mixture was quenched with water, extracted with EtOAc and the organic layer was washed with brine and concentrated to afford a mixture of D-2a/D-2b˜1:5 as light yellow solid. LCMS: 322.0 [M+H]⁺.

### Step 3: tert-Butyl 4-chloro-8,9-dihydro-5H-pyrimido[5′,4′:4,5]thieno[2,3-d]azepine-7(6H)-carboxylate (Intermediate D-a) and tert-butyl 4-chloro-6,7-dihydro-5H-pyrimido[5′,4′:4,5]thieno[2,3-c]azepine-8(9H)-carboxylate (Intermediate D-b)

A mixture of POCl₃ (715 mg, 4.67 mmol) and TEA (471 mg, 4.67 mmol) in sulfolane (12 mL) was allowed to stir at room temperature for 30 min. D-2a/D-2b (500 mg, 1.56 mmol) was added and the mixture was heated at 65° C. for 2.5 h. The resultant mixture was allowed to cool to 0° C. and TEA (1 mL) was added. The reaction mixture was diluted with toluene and cooled to 0° C. Saturated aqueous sodium chloride solution was added and the mixture was allowed to stir for 10 min. The organic layer was separated, washed with water, concentrated and the resultant residue was purified by silica gel column chromatography (hexane/EtOAc=30/1) to afford two isolated products Intermediate D-a (60 mg) and Intermediate D-b (320 mg).

D-a: LCMS m/z [M+H]⁺: 340.1. ¹H NMR (400 MHz, CDCl₃): δ 8.75 (s, 1H), 3.74 (s, 4H), 3.52 (s, 2H), 3.17 (d, 2H), 1.49 (s, 9H).

D-b: LCMS m/z [M+H]⁺: 340.0. ¹H NMR (400 MHz, CDCl₃): δ 8.74 and 8.76 (d, 1H), 4.60 and 4.69 (d, 2H), 3.72-3.81 (m, 2H), 3.46-3.49 (m, 2H), 2.01-2.04 (m, 2H) 1.39 and 1.44 (s, 9H).

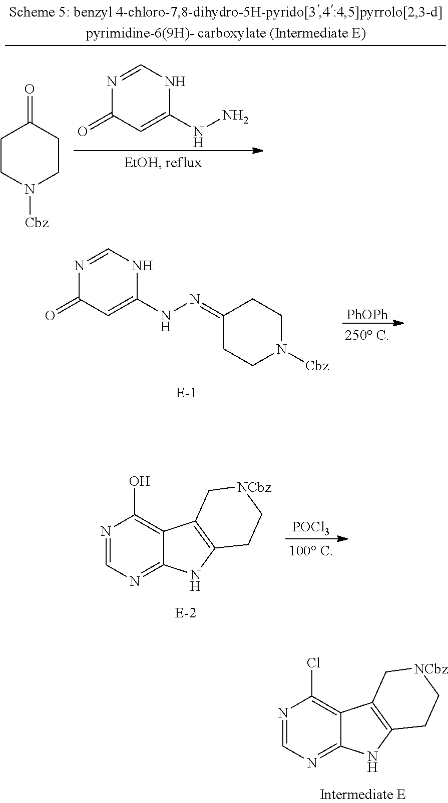

### Step 1: benzyl 4-(2-(6-oxo-3,6-dihydropyrimidin-4-yl)hydrazono)piperidine-1-carboxylate (E-1)

A suspension of benzyl 4-oxopiperidine-1-carboxylate (8.9 g, 38 mmol) and 6-hydrazino-1H-pyrimidin-4-one (4.0 g, 32 mmol) in ethanol (40 mL) was stirred at reflux for 2 h. The mixture was cooled to ambient temperature. The precipitate was collected by filtration and dried to afford the title compound as white solid (8.0 g, 74%). LCMS m/z [M+H]⁺: 342.4.

### Step 2: benzyl 4-hydroxy-5,7,8,9-tetrahydro-6H-pyrido[3′,4′:4,5]pyrrolo[2,3-d]pyrimidine-6-carboxylate (E-2)

A mixture of E-1 (4.0 g, 12 mmol) and PhOPh (25 g) was heated to 250° C. for 2 h under N₂ after which the reaction mixture was cooled to ambient temperature. Hexane (20 mL) was added and the precipitate was collected by filtration and dried. The resultant residue was purified by column chromatography (eluted with 5% MeOH in DCM) to afford the title compound as light yellow solid (1.0 g, 26%). ¹H NMR (400 MHz, DMSO-d₆): δ 11.8 (s, 1H), 11.7 (s, 1H), 7.76 (d, 1H), 7.38-7.33 (m, 5H), 5.12 (s, 2H), 4.60 (d, 2H), 3.72 (d, 2H), 2.68 (t, 2H).

### Step 3: benzyl 4-chloro-5,7,8,9-tetrahydro-6H-pyrido[3′,4′:4,5]pyrrolo[2,3-d]pyrimidine-6-carboxylate (Intermediate E)

A suspension of E-2 (1.0 g, 3.1 mmol) in POCl₃ (5.0 mL, 54.5 mmol) was heated to 100° C. for 30 min. The reaction mixture was cooled to ambient temperature and POCl₃ was removed in vacuo. The resultant residue was taken up in DCM and washed with saturated NaHCO₃ solution, then purified by column chromatography (eluted with 3-5% MeOH in DCM) to afford the title compound as white solid (0.7 g, 66%). LCMS m/z [M+H]⁺: 342.4. ¹H NMR (400 MHz, DMSO-d₆): δ 12.5 (s, 1H), 8.51 (s, 1H), 7.38-7.30 (m, 5H), 5.16 (s, 2H), 4.80 (s, 2H), 3.81 (t, 2H), 3.17 (t, 2H).

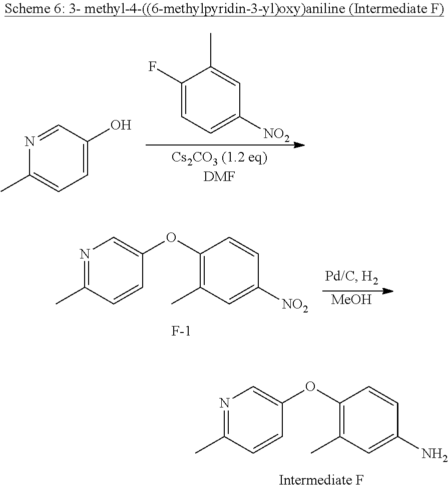

### Step 1: 2-methyl-5-(2-methyl-4-nitrophenoxy)pyridine (F-1)

To a solution of 6-methylpyridin-3-ol (5.0 g, 45.9 mmol) in DMF (40 mL) was added 1-fluoro-2-methyl-4-nitrobenzene (7.0 g, 45.2 mmol) and Cs₂CO₃ (14.9 g, 45.9 mmol) and the mixture was allowed to stir at ambient temperature for 12 h after which it was partitioned between EtOAc and water, the organic phase was separated, washed with water, brine, dried over anhydrous Na₂SO₄, and concentrated to afford the title compound (9.62 g, 86%) as light yellow solid. LCMS (m/z): 245.1 [M+1]⁺.

### Step 2: 3-methyl-4-((6-methylpyridin-3-yl)oxy)aniline (Intermediate F)

To a solution of F-1 (9.62 g, 39.4 mmol) in MeOH (80 mL) was added 10% Pd/C. The mixture was allowed to stir at ambient temperature under H₂ atmosphere for 16 h after which it was filtered, concentrated in vacuo and the resultant residue purified by column chromatography (50% EtOAc in hexane) to afford the title compound (6.2 g, 75%). LCMS (m/z): 215.1 [M+1]⁺. ¹H NMR (400 MHz, DMSO-d₆): δ 8.05 (d, 1H), 7.02 (dd, 1H), 7.15 (d, 1H), 6.71-6.67 (m, 1H), 6.49 (d, 1H), 6.43 (dd, 1H), 4.97 (s, 2H), 2.39 (s, 3H), 1.98 (s, 3H).

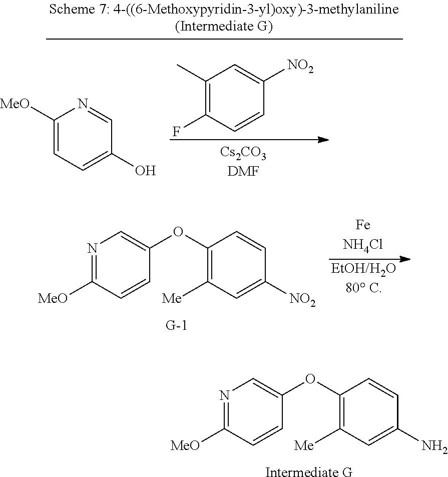

### Step 1: 2-Methoxy-5-(2-methyl-4-nitrophenoxy)pyridine (G-1)

6-Methoxypyridin-3-ol (500 mg, 4.0 mmol), 1-fluoro-2-methyl-4-nitro-benzene (620 mg, 4.0 mmol) and Cs₂CO₃ (2.9 g, 8.8 mmol) were taken in DMF (10 mL) and the mixture was allowed to stir at ambient temperature for 2 h. Water was added and the resultant mixture extracted with EtOAc. The combined organic layers were washed with brine, dried over anhydrous Na₂SO₄, filtered and concentrated. The resultant residue was purified by column chromatography on silica gel (EtOAc/hexanes=1/3) to afford the title compound as yellow solid (900 mg, 87%). LCMS m/z [M+H]⁺: 261.0.

### Step 2: 4-((6-Methoxypyridin-3-yl)oxy)-3-methylaniline (Intermediate G)

G-1 (900 mg, 3.46 mmol), NH₄Cl (1.12 g, 20.8 mmol) and Fe (1.16 g, 20.8 mmol) were taken in EtOH (30 mL) and water (15 mL). The resultant mixture was allowed to stir at 80° C. for 2 h after which the reaction mixture was filtered, the filtrate concentrated, the resultant residue dissolved in DCM/water and extracted with DCM. The combined organic layers were dried over anhydrous Na₂SO₄ and concentrated to afford the title compound as light yellow solid (680 mg, 85%). LCMS m/z [M+H]⁺: 231.1.

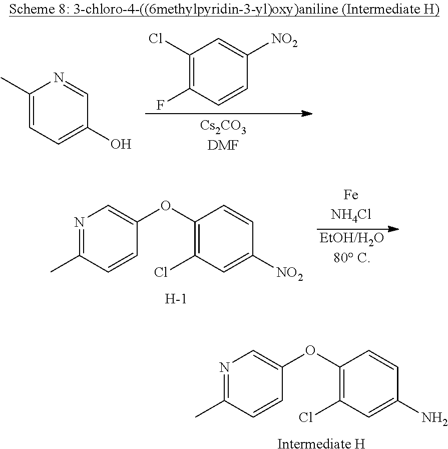

The title compound was prepared as described for Intermediate G in Scheme 7, using 6-methylpyridin-3-ol instead of 6-methoxypyridin-3-ol, and 2-chloro-1-fluoro-4-nitro-benzene instead of 1-fluoro-2-methyl-4-nitrobenzene. The title compound was obtained as light yellow solid (520 mg). LCMS m/z [M+H]⁺: 235.0.

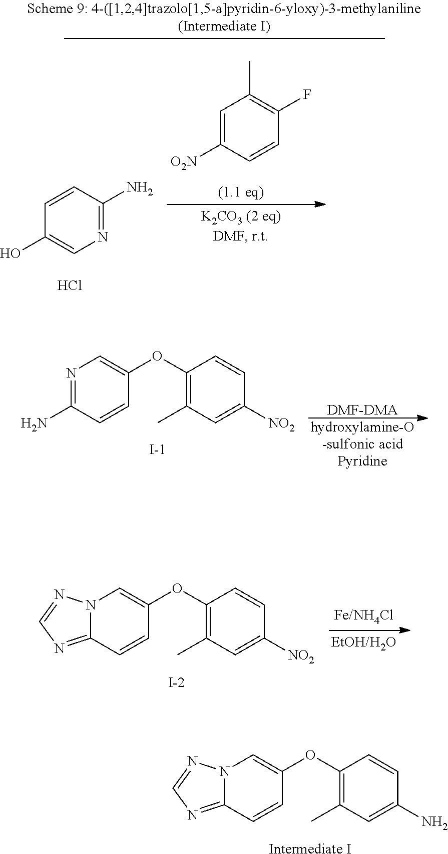

### Step 1: 5-(2-Methyl-4-nitrophenoxy) pyridin-2-amine (I-1)

A mixture of 6-aminopyridin-3-ol hydrochloride (29.3 g, 0.20 mol), 1-fluoro-2-methyl-4-nitrobenzene (31 g, 0.20 mol) and Cs₂CO₃ (144 g, 0.44 mmol) in DMF (400 mL) was allowed to stir at ambient temperature overnight. Water was added and the reaction mixture was allowed to stir for an additional 1 h. The resultant suspension was filtered and the precipitate collected to afford the title compound as light yellow solid (41.1 g, 84%). LCMS m/z [M+H]⁺: 246.3. ¹H NMR (400 MHz, CDCl₃): δ 8.13 (d, 1H), 7.97 (dd, 1H), 7.92 (d, 1H), 7.20 (d, 1H), 6.68 (d, 1H), 6.57 (d, 1H), 4.49 (br, 2H), 2.43 (s, 3H).

### Step 2: 6-(2-Methyl-4-nitrophenoxy)-[1,2,4]triazolo[1,5-a]pyridine (I-2)

A mixture of I-1 (10 g, 41 mmol) and DMF-DMA (9.7 g, 82 mmol) in EtOH (100 mL) was stirred at 90° C. for 7 h. The mixture was concentrated in vacuo and the resultant residue was dissolved in MeOH. (Aminooxy)sulfonic acid (6.5 g, 82 mmol) and pyridine (5.1 g, 45 mmol) were added at 0° C. and the mixture was allowed to stir at ambient temperature for 16 h after which it was concentrated and purified by column chromatography (eluted DCM) to afford the title compound as light yellow solid (7.0 g, 66%). LCMS m/z [M+H]⁺: 271.2.

### Step 3: 4-([1,2,4]triazolo[1,5-a]pyridin-6-yloxy)-3-methylaniline (Intermediate I)

A suspension of I-2 (7.0 g, 26 mmol), Fe (8.7 g, 155 mmol) and NH₄Cl (8.3 g, 155 mmol) in EtOH/H₂O (100 mL/5 mL) was allowed to stir at 80° C. for 3 h. The mixture was filtered and the filtrate diluted with water, and extracted with EtOAc. The organic layers were dried over anhydrous Na₂SO₄, concentrated and purified by column chromatography (DCM/MeOH=150/1) to afford the title compound as light yellow solid (5.3 g, 85%). LCMS m/z [M+H]⁺: 241.1. ¹H NMR (400 MHz, CDCl₃): δ 8.26 (s, 1H), 7.99 (d, 1H), 7.69 (d, 1H), 7.41 (dd, 1H), 6.82 (d, 1H), 6.61 (d, 1H), 6.55 (dd, 1H), 3.65 (br, 2H), 2.14 (s, 3H).

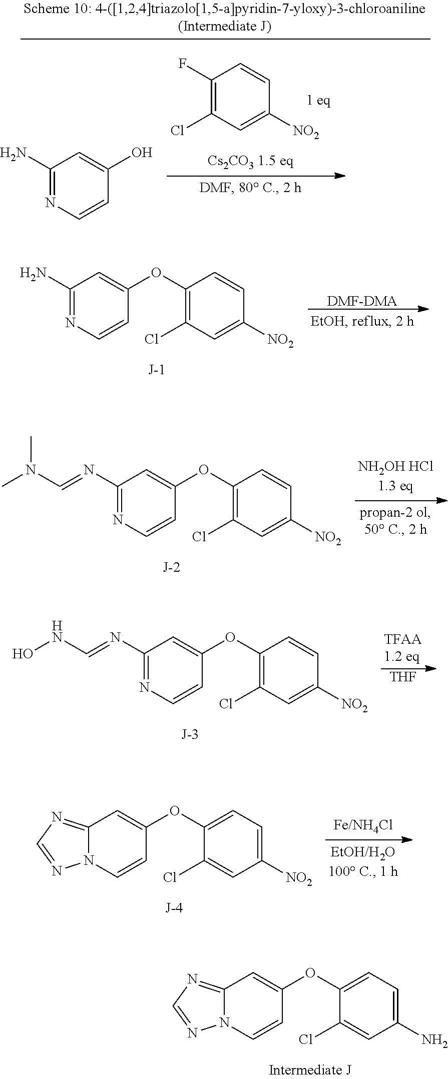

### Step 1: 4-(2-chloro-4-nitrophenoxy)pyridin-2-amine (J-1)

To a solution of 2-aminopyridin-4-ol (3.0 g, 27 mmol), 2-chloro-1-fluoro-4-nitro-benzene (4.8 g, 27 mmol) in DMF (10 mL) was added Cs₂CO₃ (13 g, 41 mmol). The resultant mixture was allowed to stir at 80° C. for 2 h. The reaction mixture was diluted with water and extracted with ethyl acetate. The combined organic layers were washed with water, brine, dried over sodium sulfate and concentrated in vacuo to afford the title compound as yellow solid (5.3 g, 73%). LCMS m/z [M+H]⁺ 266.0.

### Step 2: (E)-N′-(4-(2-chloro-4-nitrophenoxy)pyridin-2-yl)-N,N-dimethylformimidamide (J-2)

A solution of J-1 (5.3 g, 20 mmol) and DMF-DMA (4.8 g, 40 mmol) in ethanol (20 mL) was heated to reflux for 2 h. The reaction solution was concentrated to afford the title compound as yellow solid (5.0 g, 78%).

### Step 3: (E)-N′-(4-(2-chloro-4-nitrophenoxy)pyridin-2-yl)-N-hydroxyformimidamide (J-3)

To a solution of J-2 (1.0 g, 3.1 mmol) in IPA (10 mL) was added hydroxylamine hydrochloride (282 mg, 4.0 mmol). The resulting mixture was allowed to stir at 50° C. for 3 h after which it was filtered and the precipitate collected to afford the title compound as yellow solid (900 mg, 94%). LCMS m/z [M+H]⁺: 308.9.

### Step 4: 7-(2-chloro-4-nitrophenoxy)-[1,2,4]triazolo[1,5-a]pyridine (J-4)

To a solution of J-3 (500 mg, 1.6 mmol) in THF (10 mL) was added TFAA (680 mg, 3.24 mmol) at 0° C. The resultant mixture was allowed to stir under nitrogen atmosphere at ambient temperature for 3 h. The mixture was treated with aqueous NaHCO₃ to pH 8 then extracted with EtOAc. The organic layers were combined, dried over sodium sulfate, concentrated and purified by column chromatography to afford the title compound as white solid (200 mg, 42%). LCMS m/z [M+H]⁺: 291.1.

### Step 5: 4-([1,2,4]triazolo[1,5-a]pyridin-7-yloxy)-3-chloroaniline (Intermediate J)

Fe (193 mg, 3.44 mmol) and NH₄Cl (186 mg, 3.44 mmol) were added to a solution of J-4 (200 mg, 0.69 mmol) in ethanol (10 mL) and H₂O (5 mL). The resultant mixture was heated to reflux for 2 h after which the reaction solution was filtered through Celite and concentrated. The resultant residue was dissolved in dichloromethane, washed with saturated sodium bicarbonate solution. The organic layer was separated, dried over anhydrous sodium sulfate and concentrated to afford the title compound as yellow solid (160 mg, 89%). LCMS m/z [M+H]⁺: 261.0.

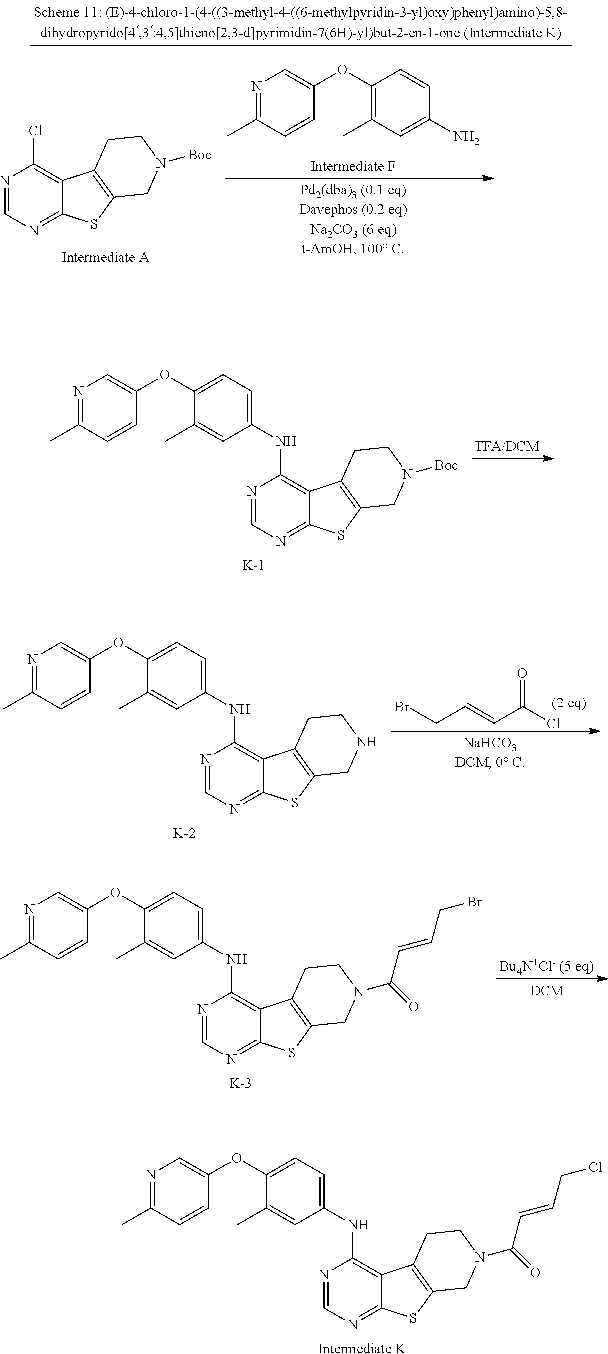

### Step 1: tert-butyl 4-((3-methyl-4-((6-methylpyridin-3-yl)oxy)phenyl)amino)-5,8-dihydropyrido[4′,3′:4,5]thieno[2,3-d]pyrimidine-7(6H)-carboxylate (K-1)

To a solution of Intermediate A (3.0 g, 9.2 mmol) in tert-amyl alcohol (30 mL) was added Intermediate F (2.0 g, 10 mmol), tris(dibenzylideneacetone)dipalladium (843 mg, 0.92 mmol), 2-dicyclohexylphosphino-2′-(N,N-dimethylamino)biphenyl (724 mg, 1.84 mmol), and sodium carbonate (5.8 g, 55 mmol). The resulting mixture was allowed to stir at 100° C. for 2 h. The mixture was washed with water, extracted with EtOAc, dried and evaporated to dryness. The resultant residue was purified by column chromatography eluting with 5% MeOH in DCM to afford the title compound (2.8 g, 59%). LCMS m/z [M+H]⁺: 504.2.

### Step 2: N-(3-methyl-4-((6-methylpyridin-3-yl)oxy)phenyl)-5,6,7,8-tetrahydropyrido[4′,3′:4,5]thieno[2,3-d]pyrimidin-4-amine (K-2)

To a solution of K-1 (2.75 g, 5.46 mmol) in DCM (10 mL) was added TFA (8 mL). The resulting mixture was allowed to stir at room temperature for 1 h. The mixture was quenched with saturated NaHCO₃ solution, extracted with DCM, dried and evaporated to dryness. The resultant residue was purified by column chromatography eluting with 5% MeOH in DCM to afford the title compound (1.8 g, 81%). LCMS m/z [M+H]⁺: 404.2.

### Step 3: (E)-4-bromo-1-(4-((3-methyl-4-((6-methylpyridin-3-yl)oxy)phenyl)amino)-5,8-dihydropyrido[4′,3′:4,5]thieno[2,3-d]pyrimidin-7(6H)-yl)but-2-en-1-one (K-3)

To a solution of K-2 (1.8 g, 4.4 mmol) in DCM/H₂O (25 mL/10 mL) was added sodium bicarbonate (1.41 g, 13.3 mmol) and (E)-4-bromobut-2-enoyl chloride (1.6 g, 8.9 mmol). The resulting mixture was allowed to stir at 0° C. for 2 h. The mixture was washed with water, extracted with EtOAc, dried and evaporated to dryness to afford the title compound (1.0 g, 41%). LCMS m/z [M+H]⁺: 551.8.

### Step 4: (E)-4-Chloro-1-(4-((3-methyl-4-((6-methylpyridin-3-yl)oxy)phenyl)amino)-5,6-dihydropyrido[4′,3′:4,5]thieno[2,3-d]pyrimidin-7(8H)-yl)but-2-en-1-one (Intermediate K)

To a solution of K-3 (1.0 g, 1.8 mmol) in DCM (20 mL) was added tetrabutylammonium chloride (2.5 g, 9.0 mmol). The resulting mixture was allowed to stir at ambient temperature for 1 h. The mixture was washed with water, extracted with EtOAc, dried and evaporated to dryness. The resultant residue was then purified by column chromatography eluting with 5% MeOH in DCM to afford the title compound (504 mg, 54%). LCMS m/z [M+H]*: 506.5. ¹H NMR (400 MHz, CDCl₃): δ 8.52 (s, 1H), 8.24 (s, 1H), 7.50 (d, 1H), 7.44 (d, 1H), 7.14 (t, 2H), 7.06-6.97 (m, 1H), 6.91 (d, 2H), 6.73-6.59 (m, 1H), 4.92 (d, 2H), 4.25 (s, 2H), 4.10-3.99 (m, 2H), 3.24 (d, 2H), 2.56 (s, 3H), 2.28 (s, 3H).

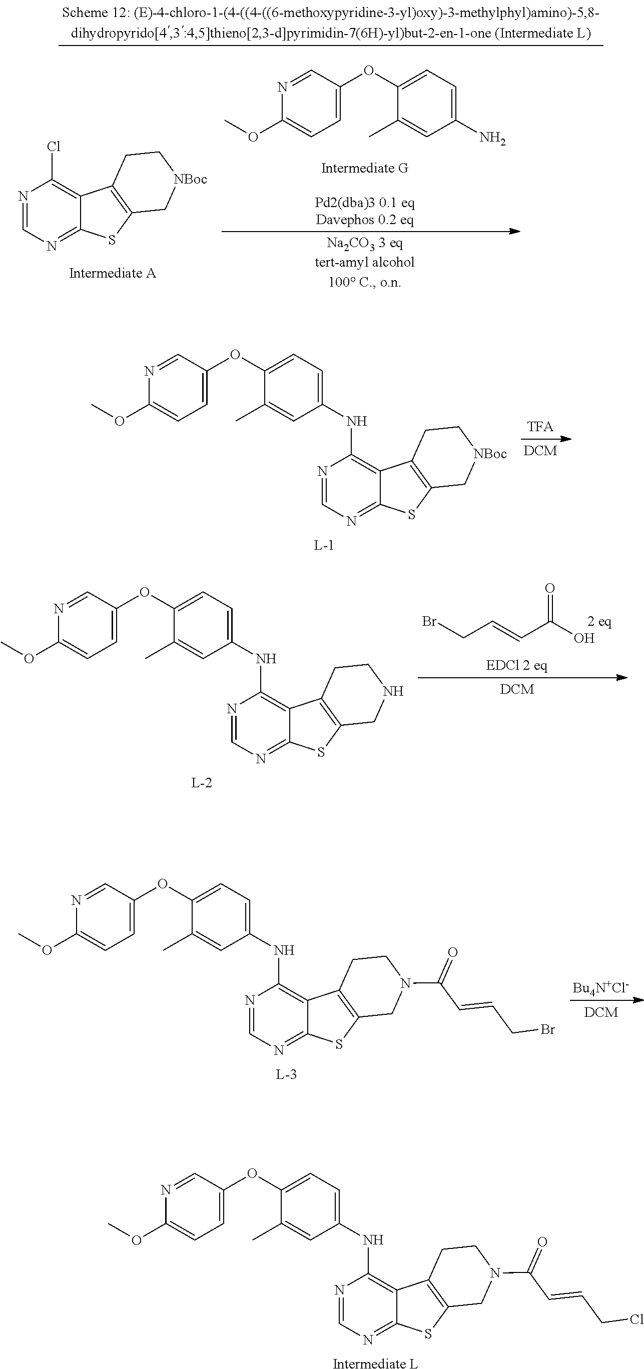

### Step 1: tert-butyl 4-((4-((6-methoxypyridin-3-yl)oxy)-3-methylphenyl)amino)-5,8-dihydropyrido[4′,3′:4,5]thieno[2,3-d]pyrimidine-7(6H)-carboxylate (L-1)

Intermediate A (7.6 g, 20.9 mmol) and Na₂CO₃ (3.7 g, 37.4 mmol) were added to a solution of Intermediate G (4.0 g, 17.4 mmol) in tert-amyl alcohol (30 mL). Pd₂(dba)₃ (800 mg, 0.87 mmol) and DavePhos (682 mg, 1.74 mmol) were added to the mixture under N₂. The resultant mixture was heated at 100° C. for 4 h then allowed cool to ambient temperature. The reaction mixture was diluted with water, extracted with ethyl acetate and the combined organic layers were washed with water, brine, dried over sodium sulfate and concentrated in vacuo. The resultant residue was purified by silica gel column chromatography (hexane/EtOAc=1/2) to afford the title compound as yellow solid (8.0 g, 88.9%). LCMS m/z [M+H]⁺: 520.4.

### Step 2: N-(4-((6-methoxypyridin-3-yl)oxy)-3-methylphenyl)-5,6,7,8-tetrahydropyrido[4′,3′:4,5]thieno[2,3-d]pyrimidin-4-amine (L-2)

To a solution of L-1 (1.5 g, 2.9 mmol) in DCM (5 mL) was added TFA (8 mL) and the resultant mixture was allowed to stir at ambient temperature for 1 h after which it was concentrated, diluted with water and the pH adjusted to >7 with saturated NaHCO₃. The resultant mixture was extracted with ethyl acetate, the combined organic layers were washed with water and brine, dried over sodium sulfate and concentrated in vacuo to afford the title compound (1.2 g, 95%).

### Step 3: (E)-4-bromo-1-(4-((4-((6-methoxypyridin-3-yl)oxy)-3-methylphenyl)amino)-5,8-dihydropyrido[4′,3′:4,5]thieno[2,3-d]pyrimidin-7(6H)-yl)but-2-en-1-one (L-3)

To a solution of L-2 (1.15 g, 2.7 mmol) and (E)-4-bromobut-2-enoic acid (890 mg, 5.4 mmol) in DMF (10 mL) was added EDCI (1.0 g, 5.4 mmol). The resultant mixture was allowed to stir at ambient temperature for 1 h after which the reaction mixture was diluted with water and extracted with ethyl acetate. The combined organic layers were washed with water, brine, dried over sodium sulfate and concentrated in vacuo. The resultant residue was purified by column chromatography eluting with 5% MeOH in DCM to afford the title compound as yellow solid (1.26 g, 84%).

### Step 4: (E)-4-Chloro-1-(4-((4-((6-methoxypyridin-3-yl)oxy)-3-methylphenyl)amino)-5,6-dihydropyrido[4′,3′:4,5]thieno[2,3-d]pyrimidin-7(8H)-yl)but-2-en-1-one (Intermediate L)

Tetrabutylammonium chloride (3.1 g, 11.2 mmol) was added to a solution of L-3 (1.26 g, 2.2 mmol) in DCM (10 mL). The resultant mixture was allowed to stir at ambient temperature overnight. The mixture was washed with water; the organic layer was separated, dried and concentrated. The resultant residue was purified by silica gel column chromatography (eluted with 3% MeOH in DCM) to afford the title compound as yellow solid (886 mg, 76%). LCMS m/z [M+H]⁺: 522.3. ¹H NMR (400 MHz, DMSO-d₆): δ 8.40 (s, 1H), 8.17 (s, 1H), 7.90 (d, 1H), 7.54 (brs, 1H), 7.47 (dd, 1H), 7.39 (dd, 1H), 6.98-6.88 (m, 1H), 6.85 (d, 1H), 6.83 (d, 1H), 6.79-6.74 (m, 1H), 4.97 (d, 1H), 4.43-4.38 (m, 2H), 3.95-3.87 (m, 2H), 3.84 (s, 3H), 3.30-3.24 (m, 2H), 2.24 (s, 3H).

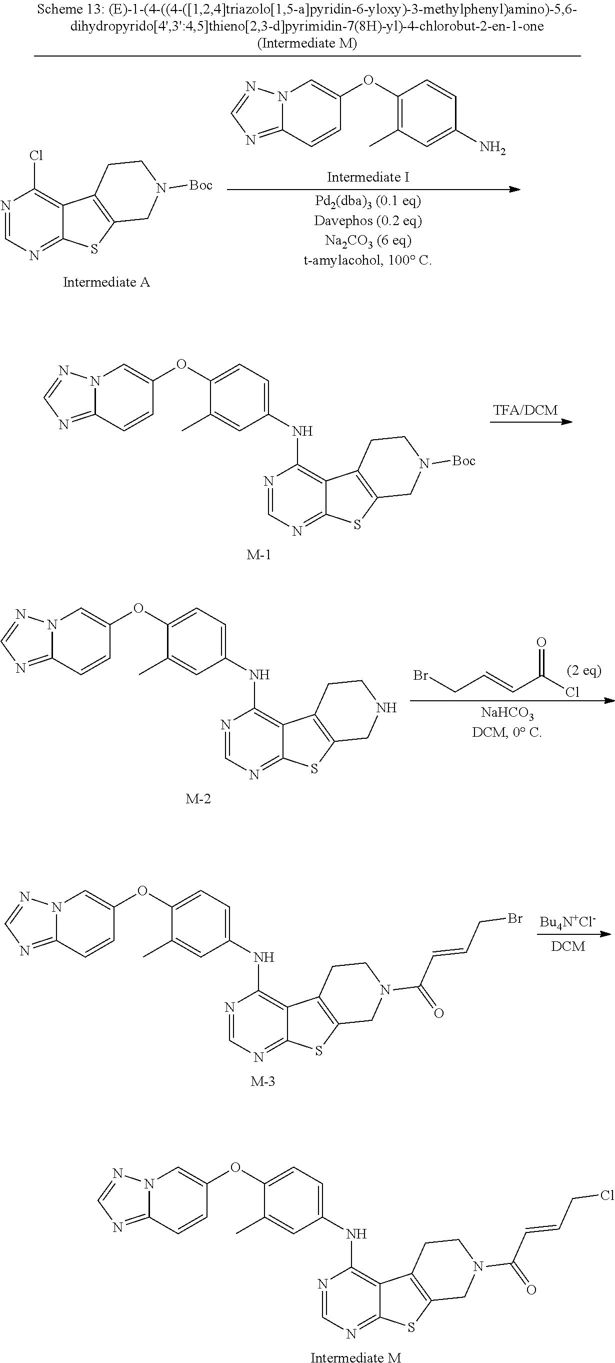

### Step 1: tert-butyl 4-((4-([1,2,4]triazolo[1,5-a]pyridin-6-yloxy)-3-methylphenyl)amino)-5,8-dihydropyrido[4′,3′:4,5]thieno[2,3-d]pyrimidine-7(6H)-carboxylate (M-1)

To a solution of Intermediate I (2 g, 8.32 mmol) in tert-amyl alcohol (30 mL) was added Intermediate A (2.71 g, 8.32 mmol), tris(dibenzylideneacetone)dipalladium (761 mg, 0.832 mmol), 2-dicyclohexylphosphino-2′-(N,N-dimethylamino)biphenyl (654 mg, 1.66 mmol), and sodium carbonate (4.4 g, 41.6 mmol). The resulting mixture was stirred at 100° C. for 2 h. The mixture was washed with water, extracted with EtOAc, and evaporated to dryness. The resultant residue was purified by column chromatography eluting 5% MeOH in DCM to afford the title compound (2.5 g, 56%). LCMS m/z [M+H]⁺: 530.4.

### Step 2: N-(4-([1,2,4]triazolo[1,5-a]pyridin-6-yloxy)-3-methylphenyl)-5,6,7,8-tetrahydropyrido[4′,3′:4,5]thieno[2,3-d]pyrimidin-4-amine (M-2)

To a solution of M-1 (2.5 g, 4.72 mmol) in DCM (15 mL) was added TFA (10 mL). The resulting mixture was allowed to stir at ambient temperature for 1 h. The mixture was quenched with saturated NaHCO₃ solution, extracted with DCM, dried concentrated, and the resultant residue purified by silica gel column chromatography eluting with 5% MeOH in DCM to afford the title compound (0.97 g, 90%). LCMS m/z [M+H]⁺: 430.2.

### Step 3: (E)-1-(4-((4-([1,2,4]triazolo[1,5-a]pyridin-6-yloxy)-3-methylphenyl)amino)-5,8-dihydropyrido[4′,3′:4,5]thieno[2,3-d]pyrimidin-7(6H)-yl)-4-bromobut-2-en-1-one (M-3)

To a solution of M-2 (0.97 g, 2.25 mmol) in DCM/H₂O (15 mL/5 mL) was added sodium bicarbonate (0.72 g, 6.7 mmol) and (E)-4-bromobut-2-enoyl chloride (0.82 g, 4.5 mmol). The resulting mixture was stirred at 0° C. for 2 h then washed with water, extracted with EtOAc, dried and evaporated to dryness to afford the title compound which was used directly in the next step.

### Step 4: (E)-1-(4-((4-([1,2,4]Triazolo[1,5-a]pyridin-6-yloxy)-3-methylphenyl)amino)-5,6-dihydropyrido[4′,3′:4,5]thieno[2,3-d]pyrimidin-7(8H)-yl)-4-chlorobut-2-en-1-one (Intermediate M)

To a solution of M-3 (1.0 g, 1.73 mmol) in DCM (20 mL) was added tetrabutylammonium chloride (2.4 g, 8.67 mmol). The resulting mixture was allowed to stir at ambient temperature for 1 h. The mixture was washed with water, extracted with EtOAc, dried and evaporated to dryness. The resultant residue was purified by silica gel column chromatography eluting with 5% MeOH in DCM to afford the title compound (452 mg, 49%). LCMS m/z [M+H]⁺ 532.1. 1H NMR (400 MHz, DMSO-d₆): δ 8.64 (d, 1H), 8.49 (s, 1H), 8.43 (s, 1H), 8.25 (s, 1H), 7.90 (d, 1H), 7.60 (s, 1H), 7.57-7.51 (m, 2H), 6.97 (dd, 2H), 6.81-6.73 (m, 1H), 4.93 (d, 2H), 4.41 (dd, 2H), 3.97-3.87 (m, 2H), 3.28 (d, 2H), 2.27 (s, 3H).

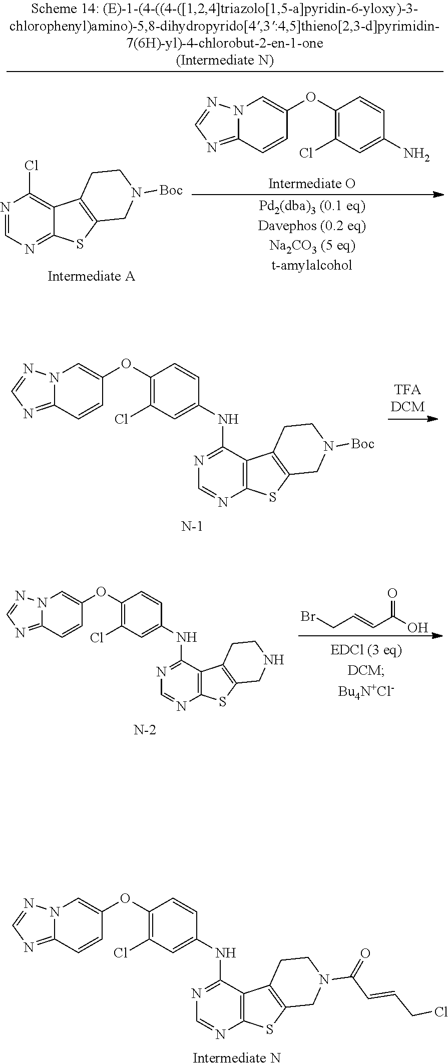

The title compound was prepared as described for Intermediate K in Scheme 11, using Intermediate 0 instead of Intermediate F. The title compound was obtained as light yellow solid (220 mg). LCMS m/z [M+H]⁺: 552.1. H NMR (400 MHz, DMSO-d₆): δ 8.85 (d, 1H), 8.50 (d, 2H), 8.46-8.38 (m, 1H), 8.02-7.89 (m, 2H), 7.68-7.55 (m, 2H), 7.25 (d, 1H), 7.01-6.88 (m, 1H), 6.84-6.73 (m, 1H), 4.89 (s, 2H), 4.42 (s, 2H), 4.02-3.85 (m, 2H), 3.32 (d, 2H).

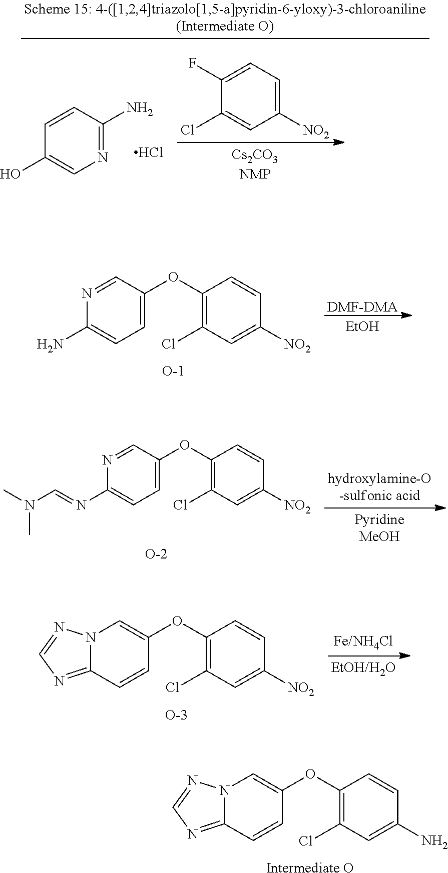

### Step 1: 5-(2-chloro-4-nitrophenoxy)pyridin-2-amine (0-1)

To a solution of 6-aminopyridin-3-ol hydrochloride (5.00 g, 34.1 mmol) and 2-chloro-1-fluoro-4-nitrobenzene (5.99 g, 34.1 mmol) in DMF (40 mL) was added Cs₂CO₃ (16.6 g, 51.0 mmol). The mixture was allowed to stir at ambient temperature overnight after which it was diluted with EtOAc, washed with water, and the organic layer was separated, dried and concentrated to afford the title compound as brown solid (8.20 g, 90%). LCMS m/z [M+H]⁺: 266.0. ¹HNMR (400 MHz, DMSO-d₆): δ 8.41 (d, 1H), 8.15 (dd, 1H), 7.86 (d, 1H), 7.33 (d, 1H), 6.95 (d, 1H), 6.56 (d, 1H), 6.10 (s, 2H).

### Step 2: (E)-N′-(5-(2-chloro-4-nitrophenoxy)pyridin-2-yl)-N,N-dimethylformimidamide (0-2)

To a solution of 0-1 (8.2 g, 30.9 mmol) in EtOH (100 mL) was added DMF-DMA (18 g, 154 mmol). The mixture was heated to reflux for 1 h. The mixture was concentrated to afford the title compound, which was used directly in the next step (9.3 g, 94%). LCMS m/z [M+H]⁺: 321.0.

### Step 3: 6-(2-chloro-4-nitrophenoxy)-[1,2,4]triazolo[1,5-a]pyridine (O-3)

To a solution of 0-2 (9.3 g, 29.1 mmol) and pyridine (6 mL, 33.96 mmol) in MeOH (80 mL) was added (aminooxy)sulfonic acid (8.8 g, 77.9 mmol) at 0° C. The mixture was stirred at ambient temperature overnight. The reaction mixture was filtered, and the resultant solid washed with MeOH and dried to afford the title compound as light yellow solid (3.5 g, 41%). LCMS m/z [M+H]⁺: 291.0.

### Step 4: 6-(2-chloro-4-nitrophenoxy)-[1,2,4]triazolo[1,5-a]pyridine (Intermediate O)

A solution of 0-3 (3.5 g, 12.0 mmol), Fe (3.37 g, 60.2 mmol) and NH₄Cl (3.19 g, 60.2 mmol) in EtOH/H₂O (40 mL/10 mL) was allowed to stir at 80° C. for 2 h. The mixture was filtered while hot and the filtrate diluted with water, and extracted with EtOAc. The organic layers were dried over anhydrous Na₂SO₄ and concentrated to afford the title compound as yellow solid (3.1 g, 98%). LCMS m/z [M+H]⁺: 261.1.

### Synthesis of Example Compounds

### Example 1: 1-(4-((4-([1,2,4]triazolo[1,5-a]pyridin-6-yloxy)-3-methylphenyl)amino)-5,8-dihydropyrido[4′,3′:4,5]thieno[2,3-d]pyrimidin-7(6H)-yl)prop-2-en-1-one (Compound 2)

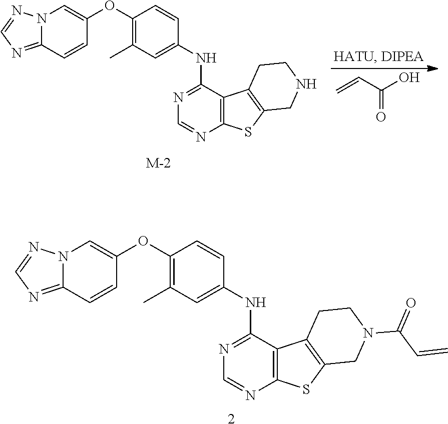

Intermediate M-2 (from Scheme 13, 60 mg, 0.091 mmol), HATU (41.6 mg, 0.109 mmol) and acrylic acid (7.51 μl, 0.109 mmol) were combined in DMF (1 mL), and DIPEA (0.048 mL, 0.274 mmol) was added at 23° C. The resultant mixture was allowed to stir for 1 h then purified by reverse phase prep HPLC to provide the title compound as white solid. LCMS m/z [M+H]⁺: 483.8.

### Example 2: (E)-1-(4-((4-([1,2,4]triazolo[1,5-a]pyridin-6-yloxy)-3-methylphenyl)amino)-5,8-dihydropyrido[4′,3′:4,5]thieno[2,3-d]pyrimidin-7(6H)-yl)-4-(dimethylamino)but-2-en-1-one (Compound 3)

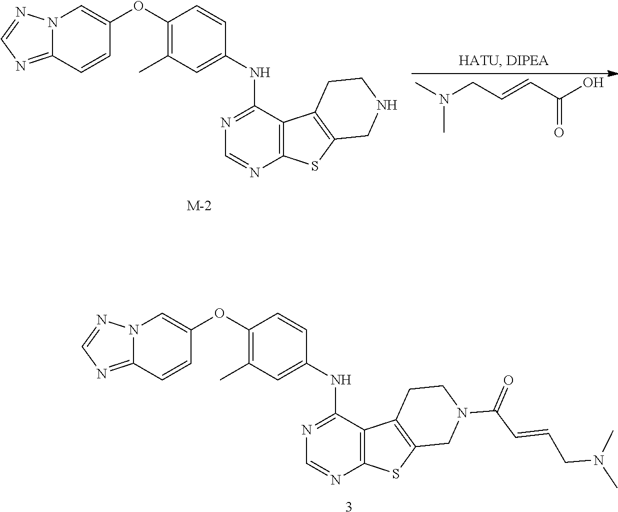

The title compound was prepared as described for Compound 2, using (E)-4-(dimethylamino)but-2-enoic acid in place of acrylic acid. The title compound was obtained as white solid. LCMS m/z [M+H]⁺: 541.0.

### Example 3: 1-(4-((4-([1,2,4]triazolo[1,5-a]pyridin-6-yloxy)-3-chlorophenyl)amino)-5,8-dihydropyrido[4′,3′:4,5]thieno[2,3-d]pyrimidin-7(6H)-yl)prop-2-en-1-one (Compound 4)

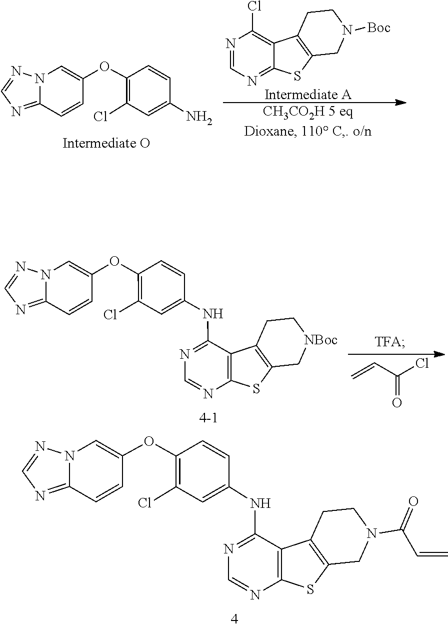

### Step 1: tert-Butyl 4-((4-([1,2,4]triazolo[1,5-a]pyridin-6-yloxy)-3-chlorophenyl)amino)-5,6-dihydropyrido[4′,3′:4,5]thieno[2,3-d]pyrimidine-7(8H)-carboxylate (4-1)

A mixture of Intermediate A (100 mg, 0.31 mmol), Intermediate 0 (80 mg, 0.31 mmol), CH₃CO₂H (184 mg, 3.07 mmol) and 1,4-dioxane (2 mL) was stirred at 110° C. for 20 h. The reaction mixture was neutralized with aqueous NaHCO₃ and extracted with EtOAc. The combined organic layer was dried over anhydrous sodium sulfate, and concentrated. The residue was purified by column chromatography (DCM:MeOH=20:1) to afford the title compound as off-white solid (25 mg, 15%). LCMS m/z [M+H]⁺: 550.2.

### Step 2: 1-(4-((4-([1,2,4]Triazolo[1,5-a]pyridin-6-yloxy)-3-chlorophenyl)amino)-5,6-dihydropyrido[4′, ′:4,5]thieno[2,3-d]pyrimidin-7(8H)-yl)prop-2-en-1-one (Compound 4)

Compound 4-1 (25 mg, 0.05 mmol) was dissolved in DCM (4 mL). TFA (2 mL) was added. The reaction mixture was stirred at ambient temperature for 30 min. The reaction mixture was neutralized with aqueous NaHCO₃ and extracted with DCM. The combined organic layer was concentrated and the resultant residue (20 mg, 0.04 mmol) and K₂CO₃ (12 mg, 0.09 mmol) were taken in DCM (10 mL)/water (10 mL) and cooled to 0° C. Acryloyl chloride (4 mg, 0.04 mmol) was added to the mixture. The reaction mixture was stirred for 30 min. The mixture was extracted with DCM, washed with bine and dried over Na₂SO₄. The resultant residue was purified by prep-TLC (DCM:MeOH=20:1) to afford the title compound as off-white solid (6.3 mg, 28%). LCMS m/z [M+H]⁺: 504.1; ¹H NMR (400 MHz, DMSO-d₆): δ 8.84 (d, 1H), 8.51 (s, 1H), 8.48 (s, 1H), 8.39 (d, 1H), 7.99 (d, 1H), 7.92 (d, 1H), 7.65 (t, 1H), 7.58 (dd, 1H), 7.25 (d, 1H), 7.02-6.88 (m, 1H), 6.23-6.18 (m, 1H), 5.78 (t, 1H), 4.95 (d, 2H), 3.98-3.90 (m, 2H), 3.29-3.25 (m, 2H).

### Example 4: (E)-1-(4-((4-([1,2,4]triazolo[1,5-a]pyridin-6-yloxy)-3-chlorophenyl)amino)-5,8-dihydropyrido[4′,3′:4,5]thieno[2,3-d]pyrimidin-7(6H)-yl)-4-(dimethylamino)but-2-en-1-one (Compound 5)

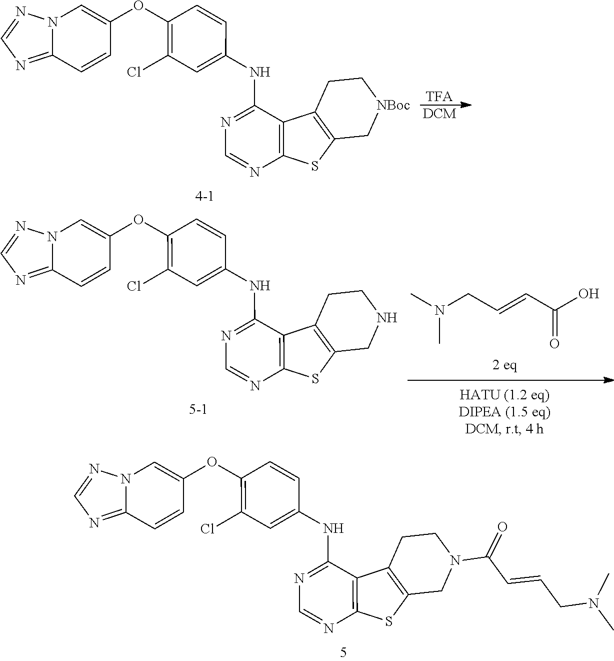

### Step 1: N-(4-([1,2,4]Triazolo[1,5-a]pyridin-6-yloxy)-3-chlorophenyl)-5,6,7,8-tetrahydropyrido[4′,3′:4,5]thieno[2,3-d]pyrimidin-4-amine (5-1)

Compound 4-1 (38 mg, 0.07 mmol) was dissolved in DCM (4 mL) followed by addition of TFA (2 mL). The reaction mixture was stirred at ambient temperature for 30 min after which it was neutralized with aqueous NaHCO₃ and extracted with DCM. The combined organic layer was evaporated to afford the title compound as off-white solid (31 mg, 99.7%). LCMS m/z [M+H]⁺: 450.0.

### Step 2: (E)-1-(4-((4-([1,2,4]Triazolo[1,5-a]pyridin-6-yloxy)-3-chlorophenyl)amino)-5,6-dihydropyrido[4′,3′:4,5]thieno[2,3-d]pyrimidin-7(8H)-yl)-4-(dimethylamino)but-2-en-1-one (Compound 5)

A mixture of compound 5-1 (31 mg, 0.07 mmol), HATU (39 mg, 0.10 mmol), and DIPEA (27 mg, 0.21 mmol) in DCM (3 mL) was stirred at ambient temperature for 2 h. The reaction mixture was partitioned between DCM and H₂O. The combined organic layer was dried over anhydrous sodium sulfate and concentrated. The resultant residue was purified by prep-TLC (DCM:MeOH=20:1) to afford the title compound as off-white solid (5.6 mg, 15%). LCMS m/z [M+H]⁺: 561.1; ¹H NMR (400 MHz, DMSO-d₆): δ 8.84 (d, 1H), 8.52 (s, 1H), 8.49 (s, 1H), 8.39 (d, 1H), 7.99 (s, 1H), 7.92 (d, 1H), 7.65 (t, 1H), 7.58 (dd, 1H), 7.25 (d, 1H), 6.88-6.79 (m, 1H), 6.72-6.67 (m, 1H), 4.93 (d, 2H), 3.99-3.89 (m, 2H), 3.34-3.28 (m, 4H), 2.33 (s, 6H).

### Example 5: 1-(4-((3-chloro-4-(pyridin-3-yloxy)phenyl)amino)-5,8-dihydropyrido[4′,3′:4,5]thieno[2,3-d]pyrimidin-7(6H)-yl)prop-2-en-1-one (Compound 6)

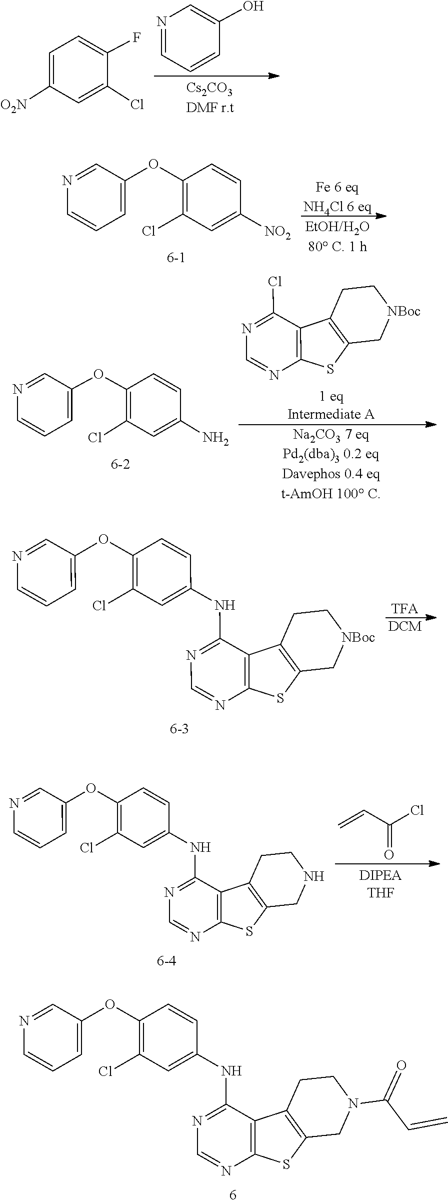

### Step 1: 3-(2-Chloro-4-nitrophenoxy)pyridine (6-1)

A suspension of 2-chloro-1-fluoro-4-nitro-benzene (500 mg, 2.85 mmol), pyridin-3-ol (298 mg, 3.13 mmol), Cs₂CO₃ (1.86 g, 5.7 mmol) in DMF (15 mL) was stirred at ambient temperature for 2 h. TLC indicated reaction was complete. The reaction mixture was diluted with water and the mixture was stirred for 30 min. The resulting suspension was filtered and washed with water and hexane to afford the title compound (710 mg, 99%). LCMS m/z [M+H]⁺: 251.0; ¹HNMR (400 MHz, CDCl₃): δ 8.54 (t, 1H), 8.48 (t, 1H), 8.41 (d, 1H), 8.11 (q, 1H), 7.41-7.40 (t, 2H), 6.94 (d, 1H).

### Step 2: 3-Chloro-4-(pyridin-3-yloxy) aniline (6-2)

A suspension of 6-1 (700 mg, 2.80 mmol), Fe (942 mg, 16.8 mmol), NH₄Cl (908 mg, 16.8 mmol) in water (10 mL)/ethanol (20 mL) was stirred at 80° C. for 2 h. The mixture was filtered and the filtrate was concentrated. The residue was dissolved in DCM, pH was adjusted to 10 with sodium carbonate solution and the resulting mixture was extracted with DCM. The combined organic layer was dried over Na₂SO₄, filtered and concentrated to afford the title compound as yellow solid (615 mg, 99%). LCMS m/z [M+H]⁺: 221.1.

### Step 3: tert-Butyl 4-((3-chloro-4-(pyridin-3-yloxy)phenyl)amino)-5,6-dihydropyrido[4′,3′:4,5]thieno[2,3-d]pyrimidine-7(8H)-carboxylate (6-3)

A suspension of Intermediate A (103 mg, 0.31 mmol), compound 6-2 (68 mg, 0.31 mmol), Pd₂(dba)₃ (57.8 mg, 0.063 mmol), and Davephos (49.8 mg, 0.12 mmol) in t-amyl alcohol (10 mL) was stirred at 100° C. for 2 h. The mixture was concentrated and the resultant residue purified by column chromatography (DCM:MeOH=80:1) to afford the title compound as yellow solid (150 mg, 93%). LCMS m/z [M+H]⁺: 510.2.

### Step 4: N-(3-Chloro-4-(pyridin-3-yloxy)phenyl)-5,6,7,8-tetrahydropyrido[4′,3′:4,5]thieno[2,3-d]pyrimidin-4-amine (6-4)

To a solution of compound 6-3 (75 mg, 0.15 mmol) in DCM (8 mL) was added TFA (3 mL). The mixture was stirred at ambient temperature for 1 h after which the mixture was concentrated. The resultant residue was dissolved in DCM, the pH adjusted to 10 with sodium carbonate solution and the resulting mixture extracted with DCM. The combined organic layer was dried over anhydrous sodium sulfate and concentrated to afford the title compound as yellow solid (59 mg, 97%).

### Step 5: 1-(4-((3-Chloro-4-(pyridin-3-yloxy)phenyl)amino)-5,6-dihydropyrido[4′,3′:4,5]thieno[2,3-d]pyrimidin-7(8H)-yl)prop-2-en-1-one (Compound 6)

To a solution of compound 6-4 (50 mg, 0.12 mmol) and DIPEA (32 mg, 0.25 mmol) in THF (5 mL) was added acryloyl chloride (11 mg, 0.12 mmol) in DCM (1 mL) dropwise at 0° C. The solution was extracted with DCM and the combined organic layer dried over anhydrous sodium sulfate and concentrated. The resultant residue was purified by prep-TLC to afford the title compound as light yellow solid (20.8 mg, 36%). LCMS m/z [M+H]⁺: 464.1; ¹HNMR (400 MHz, DMSO-d₆): δ 8.50 (s, 1H), 8.42 (d, 1H), 8.35 (dd, 2H), 8.03-7.96 (m, 1H), 7.73-7.67 (m, 1H), 7.42 (dd, 1H), 7.34-7.30 (m, 1H), 7.28 (d, 1H), 7.02-6.85 (m, 1H), 6.26-6.16 (m, 1H), 5.83-5.74 (m, 1H), 4.94 (d, 2H), 4.00-3.87 (m, 2H), 3.33-3.24 (m, 2H).

### Example 6: (E)-4-(dimethylamino)-1-(4-((3-methoxy-4-phenoxyphenyl)amino)-5,8-dihydropyrido[4′,3′:4,5]thieno[2,3-d]pyrimidin-7(6H)-yl)but-2-en-1-one (Compound 7)

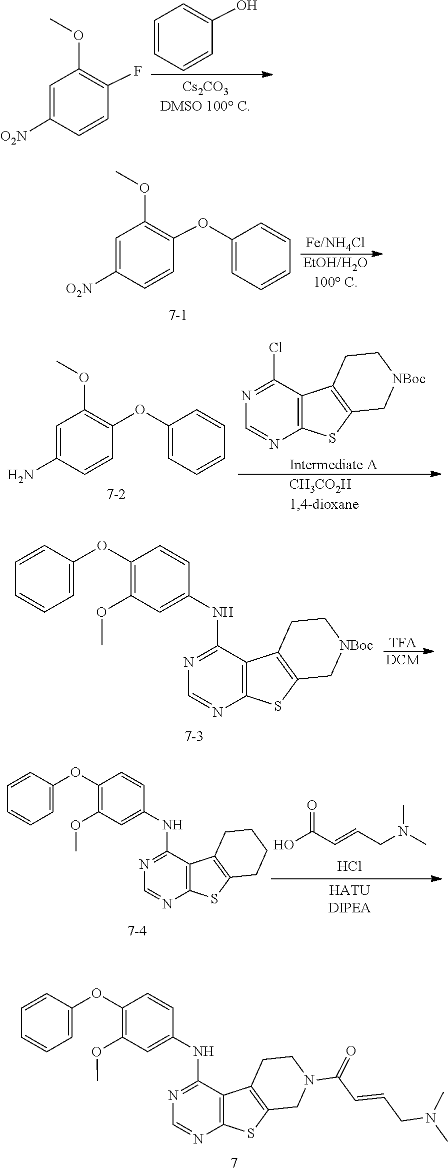

### Step 1: 2-Methoxy-4-nitro-1-phenoxybenzene (7-1)

To a solution of 1-fluoro-2-methoxy-4-nitrobenzene (5.0 g, 0.03 mol) and phenol (2.75 g, 0.03 mol) in DMSO (40 mL) was added Cs₂CO₃ (18.9 g, 0.058 mol). The resulted mixture was stirred at 100° C. for 2 h. The reaction mixture was diluted with water and extracted with ethyl acetate. The combined organic layers were washed with water and brine, dried over sodium sulfate and concentrated in vacuo to afford the title compound as yellow solid (7.0 g, 98%). LCMS m/z [M+H]⁺: 246.1.

### Step 2: 3-Methoxy-4-phenoxyaniline (7-2)

To a solution of compound 7-1 (7.0 g, 0.03 mol) in ethanol (30 mL) and H₂O (15 mL) was added Fe (8.0 g, 0.15 mol) and NH₄Cl (7.8 g, 0.145 mol). The resultant mixture was heated to reflux for 2 h. The reaction solution was filtered through celite and concentrated. The filtrate was diluted with DCM, washed with saturated aqueous sodium bicarbonate, and the organic layer was separated, dried over anhydrous sodium sulfate, filtered and concentrated to afford the title compound as yellow solid (6.0 g, 98% yield). LCMS m/z [M+H]⁺: 216.3.

### Step 3: tert-Butyl 4-((3-methoxy-4-phenoxyphenyl)amino)-5,6-dihydropyrido[4′,3′:4,5]thieno[2,3-d]pyrimidine-7(8H)-carboxylate (7-3)

Intermediate A (100 mg, 0.31 mmol), CH₃CO₂H (46.1 mg, 0.77 mmol) and compound 7-2 (99.1 mg, 0.46 mmol) were taken up in 1,4-dioxane (3 mL) and allowed to stirred at 110° C. overnight. The reaction mixture was concentrated to dryness and the resultant residue was taken up in EtOAc. The organic phase was washed with NaHCO₃ and brine then separated and dried over MgSO₄, filtered, and evaporated to dryness. The resultant residue was purified by column chromatography (hexanes/EtOAc=5/1) to afford the title compound as white solid (56 mg, 36%). LCMS m/z [M+H]⁺: 505.2.

### Step 4: N-(3-Methoxy-4-phenoxyphenyl)-5,6,7,8-tetrahydropyrido[4′,3′:4,5]thieno[2,3-d]pyrimidin-4-amine (7-4)

A solution of compound 7-3 (56 mg, 0.11 mmol) and TFA (2 mL) in DCM (4 mL) was stirred at ambient temperature for 30 min. The mixture was concentrated. The resultant residue was dissolved in DCM, pH adjusted to 11 with saturated Na₂CO₃ and the organic phase was washed with brine and concentrated to afford the title compound which was used for the next step without further purification.

### Step 5: (E)-4-(Dimethylamino)-1-(4-((3-methoxy-4-phenoxyphenyl)amino)-5,6-dihydropyrido[4′,3′:4,5]thieno[2,3-d]pyrimidin-7(8H)-yl)but-2-en-1-one (Compound 7)

A mixture of compound 7-4 (50 mg, 0.12 mmol), (E)-4-(dimethylamino)but-2-enoic acid hydrochloride (30.7 mg, 0.19 mmol), DIPEA (47.8 mg, 0.37 mmol), and HATU (56.4 mg, 0.15 mmol) in DCM (10 mL) was stirred at ambient temperature for 1 h. The mixture was concentrated and the resultant residue was dissolved in DCM, washed with brine, and concentrated. The resultant residue was purified by prep-TLC (DCM:MeOH=25:1) to afford the title compound as white solid (36 mg, 56%). LCMS m/z [M+H]⁺: 516.2. ¹H NMR (400 MHz, DMSO-d₆): δ 8.45 (s, 1H), 8.28-8.26 (m, 1H), 7.49 (d, 1H), 7.33-7.29 (m, 3H), 7.05-7.01 (m, 2H), 6.85-6.70 (m, 4H), 4.93 (d, 2H), 3.96-3.90 (m, 2H), 3.74 (s, 3H), 3.30-3.18 (m, 4H), 2.29 (s, 6H).

### Example 7: (E)-4-(Dimethylamino)-1-(4-((3-methyl-4-((6-methylpyridin-3-yl)oxy)phenyl)amino)-5,6-dihydropyrido[4′,3′:4,5]thieno[2,3-d]pyrimidin-7(8H)-yl)but-2-en-1-one (Compound 8)

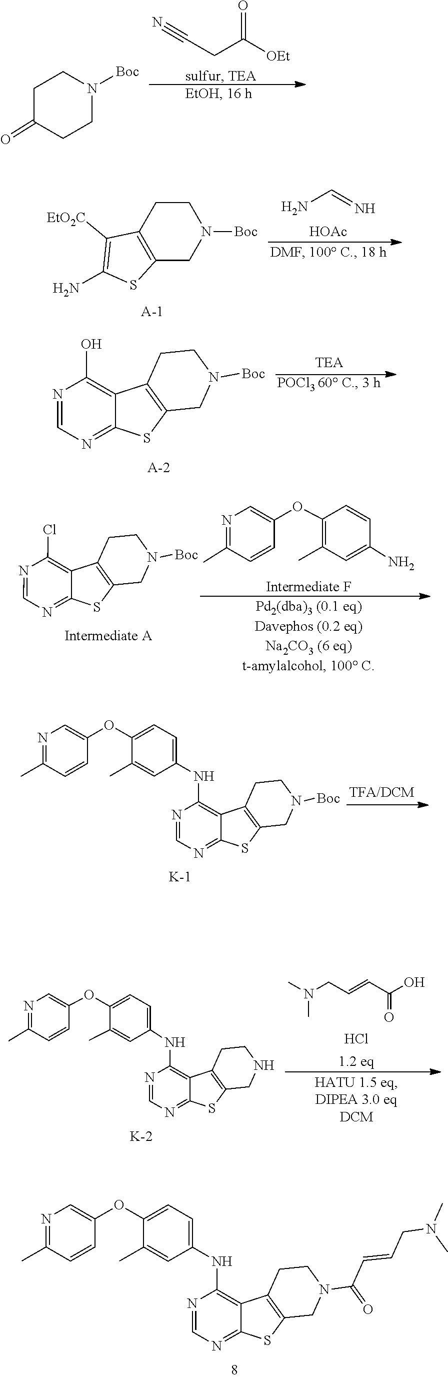

A mixture of N-(3-methyl-4-((6-methylpyridin-3-yl)oxy)phenyl)-5,6,7,8-tetrahydropyrido[4′,3′:4,5]thieno[2,3-d]pyrimidin-4-amine (K-2) (110 mg, 0.27 mmol, prepared as described in Schemes 1 and 11 for Intermediates A and K respectively), (E)-4-(dimethylamino)but-2-enoic acid hydrochloride (67.7 mg, 0.41 mmol), DIPEA (106 mg, 0.82 mmol) and HATU (124 mg, 0.33 mmol) in DCM (10 mL) was allowed to stir at ambient temperature for 1 h. The reaction mixture was concentrated. The resultant residue was dissolved in DCM, washed with brine, concentrated, and purified by silica gel column chromatography (DCM/MeOH=25/1) to afford the title compound as white solid (56 mg, 39%). LCMS m/z [M+H]⁺:515.2. ¹H NMR (400 MHz, DMSO-d₆): δ ppm 8.42 (s, 1H), 8.23-8.16 (m, 2H), 7.57-7.52 (m, 2H), 7.25-7.18 (m, 2H), 6.96-6.89 (m, 2H), 6.72-6.69 (m, 1H), 4.92 (d, 2H), 3.95-3.88 (m, 2H), 3.40 (s, 2H), 3.27-3.25 (m, 2H), 2.43 (m, 9H), 2.19 (s, 3H).

### Example 8: 1-(4-((3-methyl-4-(pyridin-3-yloxy)phenyl)amino)-5,8-dihydropyrido[4′,3′:4,5]thieno[2,3-d]pyrimidin-7(6H)-yl)prop-2-en-1-one (Compound 11)

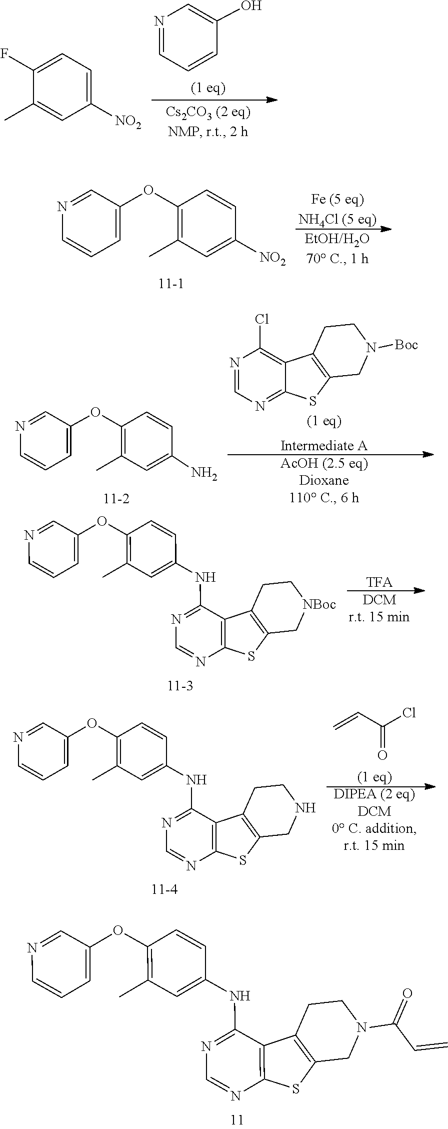

### Step 1: 3-(2-Methyl-4-nitrophenoxy)pyridine (11-1)

To a solution of pyridin-3-ol (300 mg, 3.15 mmol) and 1-fluoro-2-methyl-4-nitro-benzene (489 mg, 3.15 mmol) in NMP (10 mL) was added cesium carbonate (2.08 g, 6.31 mmol). The resulting mixture was stirred at ambient temperature for 3 h. The mixture was taken up in EtOAc and the organic solution was washed with water, dried and evaporated to afford the title compound (720 mg). LCMS m/z [M+H]⁺: 231.2.

### Step 2: 3-Methyl-4-(pyridin-3-yloxy)aniline (11-2)

To a solution of 11-1 (720 mg, 3.13 mmol) in ethanol (40 mL) and water (10 mL) was added Fe (874 mg, 15.6 mmol) and ammonium chloride (836 mg, 15.6 mmol). The resulting mixture was stirred at 70° C. for 2 h. To the mixture was added EtOAc. The resulting mixture was washed with water, dried and evaporated to afford the titled compound (500 mg).

### Step 3: tert-Butyl 4-((3-methyl-4-(pyridin-3-yloxy)phenyl)amino)-5,6-dihydropyrido[4′,3′:4,5]thieno[2,3-d]pyrimidine-7(8H)-carboxylate (11-3)

AcOH (36.8 mg, 0.61 mmol) was added to a solution of Intermediate A (80 mg, 0.25 mmol) and 11-2 (49.2 mg, 0.25 mmol) in 1,4-dioxane (2 mL). The resulting mixture was heated at 110° C. for 5 h. The mixture was quenched with saturated NaHCO₃. The reaction mixture was extracted with EtOAc. The organic phase was dried over Na₂SO₄, filtered, and evaporated to dryness. The resultant residue was purified by column chromatography (DCM:MeOH=50:1) to afford the title compound (65 mg). LCMS m/z [M+H]⁺: 490.2.

### Step 4: N-(3-Methyl-4-(pyridin-3-yloxy)phenyl)-5,6,7,8-tetrahydropyrido[4′,3′:4,5]thieno[2,3-d]pyrimidin-4-amine (11-4)

To a solution of 11-3 (65 mg, 0.13 mmol) in DCM (3 mL) was added TFA (2 mL). The resulting mixture was stirred at ambient temperature for 30 min. The mixture was evaporated and the resultant residue taken up in saturated NaHCO₃ then extracted with DCM, dried and concentrated to afford the title compound (50 mg). LCMS m/z [M+H]⁺: 390.1.

### Step 5: 1-(4-((3-Methyl-4-(pyridin-3-yloxy)phenyl)amino)-5,6-dihydropyrido[4′,3′:4,5]thieno[2,3-d]pyrimidin-7(8H)-yl)prop-2-en-1-one (Compound 11)

To a solution of 11-4 (45 mg, 0.12 mmol) and DIPEA (30 mg, 0.23 mmol) in DCM (5 mL) was added acryloyl chloride (10 mg, 0.11 mmol) at 0° C. The resulting mixture was stirred at 0° C. for 15 min. The reaction mixture was quenched with saturated NaHCO₃ and extracted with DCM. The organic extract was dried over Na₂SO₄, filtered, and evaporated to dryness. The resultant residue was purified by prep-TLC (DCM:MeOH=20:1) to afford the title compound (20.5 mg). LCMS m/z (M+H)+: 444.4. ¹HNMR (400 MHz, DMSO-d₆) δ 8.43 (s, 1H), 8.31-8.28 (m, 2H), 8.26 (s, 1H), 7.57 (d, 2H), 7.39 (dd, 1H), 7.26 (d, 1H), 7.01 (d, 1H), 6.84-6.98 (m, 1H), 6.21 (d, 1H), 5.78 (t, 1H), 4.93 (d, 2H), 3.93 (d, 2H), 3.28 (d, 2H), 2.18 (s, 3H).

### Example 9: (E)-4-(3-azabicyclo[3.2.1]octan-3-yl)-1-(4-((3-methyl-4-((6-methylpyridin-3-yl)oxy)phenyl)amino)-5,8-dihydropyrido[4′,3′:4,5]thieno[2,3-d]pyrimidin-7(6H)-yl)but-2-en-1-one benzenesulfonate salt (Compound 16 besylate salt)

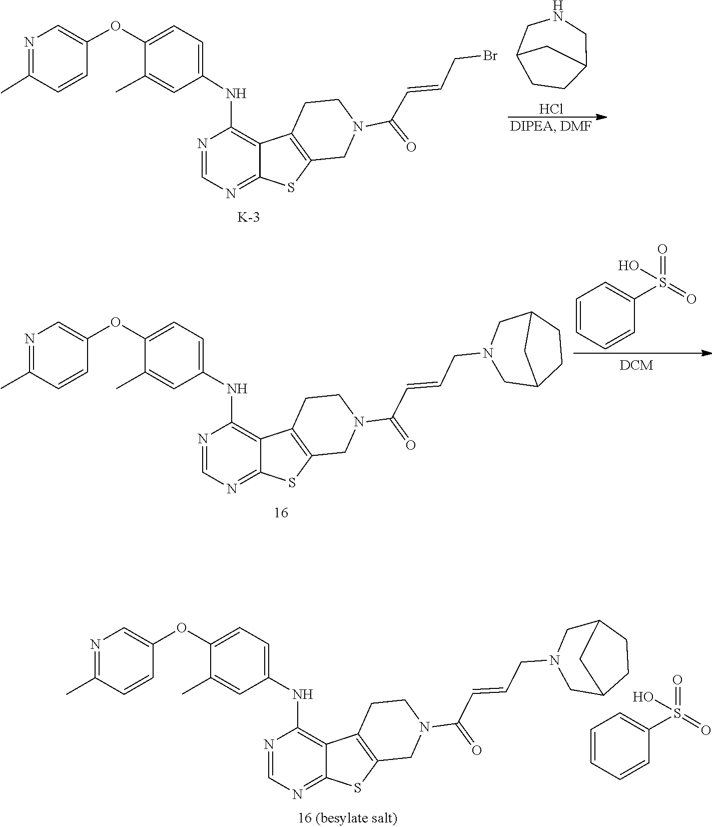

To a solution of (E)-4-bromo-1-(4-((3-methyl-4-((6-methylpyridin-3-yl)oxy)phenyl)amino)-5,8-dihydropyrido[4′,3′:4,5]thieno[2,3-d]pyrimidin-7(6H)-yl)but-2-en-1-one (K-3 from Scheme 11) (200 mg, 0.4 mmol) in DMF (6 mL) was added 3-azabicyclo[3.2.1]octane hydrochloride (58 mg, 0.40 mmol) and DIPEA (153 mg, 1.19 mmol). The resulting mixture was allowed to stir at 40° C. for 4 h. The reaction mixture was washed with water, extracted with DCM, dried and evaporated to dryness. The resultant residue was purified by column chromatography eluting with 8% MeOH in DCM to afford Compound 16 (32 mg). LCMS m/z [M+H]⁺: 581.2.

To a solution of Compound 16 (32 mg, 0.06 mmol) in DCM (4 mL) was added benzenesulfonic acid (9.0 mg, 0.06 mmol). The mixture was allowed to stir at 25° C. for 5 min after which it was concentrated in vacuo to afford the title compound (37 mg, 99%) as light yellow solid. LCMS m/z [M+H]⁺: 581.2. ¹H NMR (400 MHz, CDCl₃): δ 10.68-10.13 (m, 1H), 8.49 (s, 1H), 8.27 (s, 1H), 7.86 (d, 2H), 7.63-7.46 (m, 2H), 7.34 (m, 4H), 7.23 (d, 1H), 7.05 (d, 1H), 6.91-6.72 (m, 2H), 4.91 (s, 2H), 4.16-3.83 (m, 4H), 3.61-3.16 (m, 4H), 2.85 (d, 2H), 2.65 (s, 3H), 2.42 (s, 2H), 2.26-2.15 (m, 1H), 2.07 (m, 4H), 1.83-1.71 (m, 2H), 1.61 (s, 2H).

### Example 10: (E)-1-(4-((3-Methyl-4-((6-methylpyridin-3-yl)oxy)phenyl)amino)-5,6-dihydropyrido[4′,3′:4,5]thieno[2,3-d]pyrimidin-7(8H)-yl)-4-morpholinobut-2-en-1-one (Compound 17)

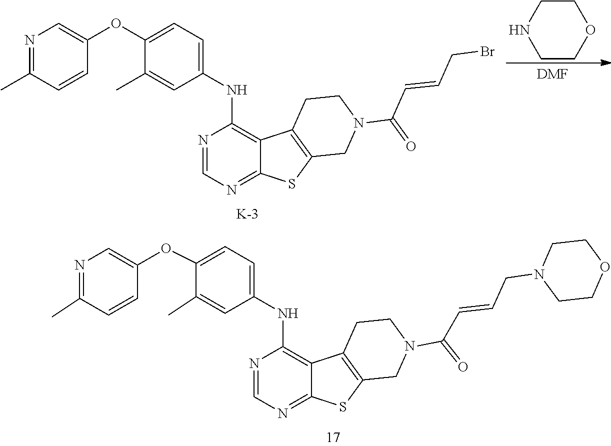

To a solution of (E)-4-bromo-1-(4-((3-methyl-4-((6-methylpyridin-3-yl)oxy)phenyl)amino)-5,6-dihydropyrido[4′,3′:4,5]thieno[2,3-d]pyrimidin-7(8H)-yl)but-2-en-1-one (K-3 from Scheme 11) (100 mg, 0.18 mmol) in DMF (6 mL) was added morpholine (79 mg, 0.91 mmol). The resulting mixture was allowed to stir at ambient temperature for 3 h. The reaction mixture was washed with water, extracted with EtOAc, dried and evaporated to dryness. The resultant residue was purified by column chromatography eluting with 10% MeOH in DCM to afford the title compound (39 mg, 37%) as light yellow solid. LCMS m/z [M+H]⁺: 557.4. ¹H NMR (400 MHz, CDCl₃): δ 8.52 (s, 1H), 8.25 (d, 1H), 7.50 (s, 1H), 7.43 (d, 1H), 7.14-7.06 (m, 2H), 6.97-6.85 (m, 3H), 6.65-6.48 (m, 1H), 4.90 (d, 2H), 4.05 (d, 2H), 3.76 (s, 4H), 3.29-3.17 (m, 4H), 2.56-2.52 (m, 7H), 2.28 (s, 3H).

### Example 11: (E)-4-(dimethylamino)-1-(4-((3-methyl-4-((2-methylpyrimidin-5-yl)oxy)phenyl)amino)-5,8-dihydropyrido[4′,3′:4,5]thieno[2,3-d]pyrimidin-7(6H)-yl)but-2-en-1-one (Compound 21)

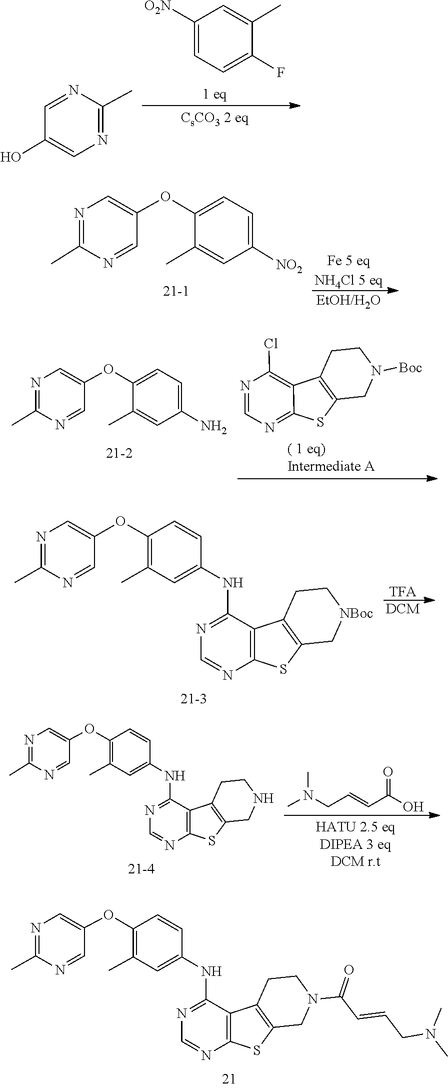

### Step 1: 2-Methyl-5-(2-methyl-4-nitrophenoxy)pyrimidine (21-1)

A suspension of 2-methylpyrimidin-5-ol (500 mg, 4.54 mmol), 1-fluoro-2-methyl-4-nitro-benzene (704 mg 4.54 mmol), and Cs₂CO₃ (2.96 g, 9.08 mmol) in DMF (10 mL) was stirred at ambient temperature for 1 h. TLC analysis indicated reaction was complete. The mixture was diluted with water, extracted with EtOAc. The combined organic layer was washed with brine, dried over Na₂SO₄ and concentrated to afford the title compound as yellow solid (753 mg, 67%). LCMS m/z [M+H]⁺: 246.0; ¹HNMR (400 MHz, CDCl₃): δ 8.45 (s, 2H), 8.20 (d, 1H), 8.05 (q, 1H), 6.80 (d, 1H), 2.77 (s, 3H), 2.43 (s, 3H).

### Step 2: 3-Methyl-4-((2-methylpyrimidin-5-yl)oxy)aniline (21-2)

A suspension of 21-1 (750 mg, 3.06 mmol), NH₄Cl (817 mg, 15.2 mmol), Fe (856 mg, 15.3 mmol) in ethanol (15 mL)/H₂O (15 mL) was stirred at 80° C. for 1 h. The reaction mixture was filtered and the filtrate was concentrated. The resultant residue was dissolved in DCM, the pH adjusted to 10 with sodium carbonate solution and extracted with DCM. The combined organic layer was dried over anhydrous sodium sulfate, filtered and concentrated to afford the title compound as light yellow solid (646 mg, 96%). LCMS m/z [M+H]⁺: 216.1.

### Step 3: tert-Butyl 4-((3-methyl-4-((2-methylpyrimidin-5-yl)oxy)phenyl)amino)-5,6-dihydropyrido[4′,3′:4,5]thieno[2,3-d]pyrimidine-7(8H)-carboxylate (21-3)

A suspension of Intermediate A (100 mg. 0.31 mmol), 21-2 (99 mg, 0.46 mmol), Na₂CO₃ (227 mg, 2.15 mmol), Davephos (48 mg, 0.12 mmol) Pd₂(dba)₃ (56 mg, 0.061 mmol) in t-amyl alcohol (10 mL) was stirred at 100° C. in N₂ for 2 h. The mixture was concentrated. The resultant residue was purified by column chromatography (DCM:MeOH=80:1) to afford the title compound as brown solid (101 mg, 65%). LCMS m/z [M+H]⁺: 505.2.

### Step 4: N-(3-Methyl-4-((2-methylpyrimidin-5-yl)oxy)phenyl)-5,6,7,8-tetrahydropyrido[4′,3′:4,5]thieno[2,3-d]pyrimidin-4-amine (21-4)

To a solution of 21-3 (100 mg, 0.2 mmol) in DCM (8 mL) was added TFA (3 mL). The mixture was stirred at ambient temperature for 1 h. The mixture was concentrated and the residue dissolved in DCM, the pH adjusted to 10 with sodium carbonate solution, and the resulting mixture extracted with DCM. The combined organic layer was dried over anhydrous sodium sulfate, filtered and concentrated to afford the title compound as brown solid (80 mg, 99%). LCMS m/z [M+H]⁺: 405.1.

### Step 5: (E)-4-(Dimethylamino)-1-(4-((3-methyl-4-((2-methylpyrimidin-5-yl)oxy)phenyl)amino)-5,6-dihydropyrido[4′,3′:4,5]thieno[2,3-d]pyrimidin-7(8H)-yl)but-2-en-1-one (Compound 21)

A suspension of 21-4 (80 mg 0.2 mmol), (E)-4-(dimethylamino)but-2-enoic acid hydrochloride (65.5 mg, 0.4 mmol), DIPEA (76.5 mg, 0.59 mmol), HATU (188 mg, 0.49 mmol) was stirred at ambient temperature for 1 h. The mixture was concentrated and the resultant residue was purified by prep-TLC to afford the title compound as colorless solid (20 mg, 19%). LCMS m z [M+H]⁺: 516.2; ¹HNMR (400 MHz, CDCl₃): δ 8.52 (s, 1H), 8.33 (s, 2H), 7.55-7.48 (m, 2H), 6.93 (d, 3H), 6.67-6.51 (m, 1H), 4.90 (d, 2H), 4.05 (d, 2H), 3.26-3.14 (m, 4H), 2.71 (s, 3H), 2.37-2.28 (m, 9H).

### Example 12: (E)-1-(4-((4-((1H-indazol-5-yl)oxy)-3-methylphenyl)amino)-5,8-dihydropyrido[4′,3′:4,5]thieno[2,3-d]pyrimidin-7(6H)-yl)-4-(dimethylamino)but-2-en-1-one (Compound 22)

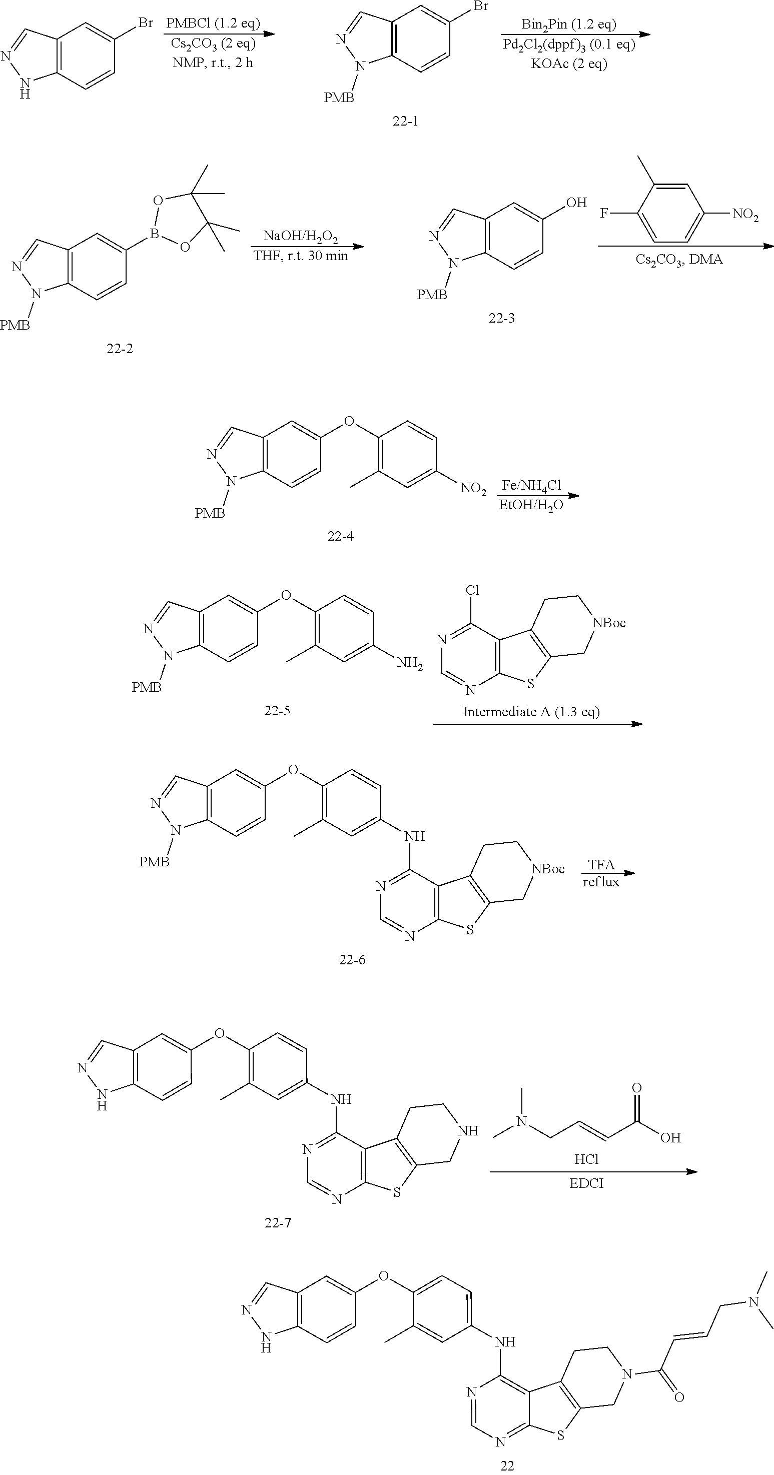

### Step 1: 5-Bromo-1-(4-methoxybenzyl)-1H-indazole (22-1)

PMBCl (4.69 g, 0.03 mol) was added to a mixture of 5-bromo-1H-indazole (5.0 g, 0.025 mol) and Cs₂CO₃ (12.4 g, 0.04 mol) in DMF (50 mL) at ambient temperature. The resulting mixture was stirred at ambient temperature for 2 h and then diluted with EtOAc. The organics were washed with brine, dried over anhydrous Na₂SO₄ and evaporated to dryness. The resultant residue was purified by column chromatography (hexanes:EtOAc=10:1) to afford the title compound as white solid (8.0 g, 99%). LCMS m/z [M+H]⁺: 317.0.

### Step 2: 1-(4-Methoxybenzyl)-5-(4,4,5,5-tetramethyl-1,3,2-dioxaborolan-2-yl)-1H-indazole (22-2)

A solution of 22-1 (8 g, 0.025 mol), Bin₂Pin (6.99 g, 0.028 mol), Pd(dppf)Cl₂ (914 mg, 1.25 mmol) and KOAc (7.44 g, 0.075 mol) in dioxane (50 mL) was stirred at 90° C. for 4 h under nitrogen atmosphere. The mixture was cooled, diluted with water, and extracted by EtOAc. The organic layers were dried over anhydrous Na₂SO₄ and evaporated to dryness to provide the title compound, which was used without further purification. LCMS m/z [M+H]⁺: 365.0.

### Step 3: 1-(4-Methoxybenzyl)-1H-indazol-5-ol (22-3)

A mixture of 22-2 (0.025 mol), NaOH (4 g, 0.1 mol), THF (10 mL) and H₂O₂ (30%, 8.5 mL) was stirred at ambient temperature for 0.5 h. The mixture was diluted with water then extracted with EtOAc. The organic layers were dried over anhydrous Na₂SO₄ and evaporated to dryness. The resultant residue was purified by column chromatography (hexanes:EtOAc=1:1) to afford the title compound as orange oil (5.0 g, 78% two steps). LCMS m/z [M+H]⁺: 255.1.

### Step 4: 1-(4-Methoxybenzyl)-5-(2-methyl-4-nitrophenoxy)-1H-indazole (22-4)

A mixture of 22-3 (1 g, 3.94 mmol), 1-fluoro-2-methyl-4-nitrobenzene (610 mg, 3.94 mmol), Cs₂CO₃ (1.93 g, 5.93 mmol) in DMA (20 mL) was stirred at 90° C. for 1 h. The mixture was diluted with water and extracted with EtOAc. The organic layers were dried over anhydrous Na₂SO₄ and evaporated to dryness. The resultant residue was purified through column chromatography (hexanes:EtOAc=4:1) to afford the title compound as brown oil (1.1 g, 72%). LCMS m/z [M+H]⁺: 390.1; ¹H NMR (400 MHz, CDCl₃): δ 8.15 (d, 1H), 8.00 (d, 1H), 7.94 (dd, 1H), 7.40 (d, 1H), 7.36 (d, 1H), 7.22-7.18 (m, 2H), 7.07 (dd, 1H), 6.87-6.84 (m, 2H), 6.67 (d, 1H), 5.55 (s, 2H), 3.78 (s, 3H), 2.44 (s, 3H).

### Step 5: 4-(1-(4-Methoxybenzyl)-1H-indazol-5-yloxy)-3-methylaniline (22-5)

A solution of 22-4 (1.1 g, 2.82 mmol), iron (791 mg, 14 mmol) and NH₄Cl (791 mg, 14 mmol) in EtOH/H₂O (20 mL/10 mL) was stirred at 80° C. for 3 h. The mixture was cooled and filtered. The filtrate was diluted with water, and extracted with EtOAc. The organic layers were dried over anhydrous Na₂SO₄ and evaporated to dryness. The resultant residue was used in the next step without further purification. LCMS m/z [M+H]⁺: 360.3; ¹H NMR (400 MHz, CDCl₃): δ 7.84 (s, 1H), 7.25 (d, 1H), 7.16 (d, 2H), 7.08 (dd, 1H), 6.93 (d, 1H), 6.84-6.81 (m, 2H), 6.75 (d, 1H), 6.60 (d, 1H), 6.52 (dd, 1H), 5.49 (s, 2H), 3.76 (s, 3H), 3.50 (b s, 2H), 2.13 (s, 3H).

### Step 6: tert-Butyl 4-((4-((1-(4-methoxybenzyl)-1H-indazol-5-yl)oxy)-3-methylphenyl) amino)-5,6-dihydropyrido[4′,3′:4,5]thieno[2,3-d]pyrimidine-7(8H)-carboxylate (22-6)

A mixture of 22-5 (300 mg, 0.84 mmol), Intermediate A (353 mg, 1.09 mmol), DavePhos (67 mg, 0.17 mmol), Pd₂(dba)₃ (153 mg, 0.17 mmol), and Na₂CO₃ (620 mg, 5.85 mmol) in t-amylalcohol (10 mL) was stirred at 100° C. for 3 h under nitrogen atmosphere. The reaction mixture was concentrated and purified through column chromatography (DCM:MeOH=100:1) to afford the title compound as light-yellow solid (400 mg, 74%). LCMS m/z [M+H]⁺: 649.4; ¹H NMR (400 MHz, CDCl₃): δ 8.50 (s, 1H), 7.90 (d, 1H), 7.47 (d, 1H), 7.39 (dd, 1H), 7.33-7.29 (m, 1H), 7.18 (d, 2H), 7.11-7.09 (m, 2H), 6.92-6.83 (m, 4H), 5.52 (s, 2H), 4.72 (b s, 2H), 3.86 (t, 2H), 3.78 (s, 3H), 3.15 (t, 2H), 2.31 (s, 3H), 1.51 (s, 9H).

### Step 7: N-(4-((1H-Indazol-5-yl)oxy)-3-methylphenyl)-5,6,7,8-tetrahydropyrido[4′,3′:4,5]thieno[2,3-d]pyrimidin-4-amine (22-7)

A solution of 22-6 (200 mg, 0.31 mmol) in TFA (10 mL) was stirred at 100° C. for 12 h. The reaction solution was concentrated to remove most of TFA. The residue was adjusted to pH 10 with aqueous Na₂CO₃ and extracted with DCM. The organic layers were dried over anhydrous Na₂SO₄ and evaporated to dryness. The resultant residue was purified through column chromatography (DCM:MeOH=10:1) to afford the title compound as white solid (80 mg, 60%). ¹H NMR (400 MHz, CDCl₃): δ 8.51 (s, 1H), 7.97 (d, 1H), 7.51 (d, 1H), 7.47 (d, 1H), 7.43 (dd, 1H), 7.20 (dd, 1H), 7.16 (d, 1H), 6.98 (s, 1H), 6.90 (d, 1H), 4.14 (s, 2H), 3.32 (t, 2H), 3.10 (t, 2H), 2.32 (s, 3H).

### Step 8: (E)-1-(4-((4-((1H-Indazol-5-yl)oxy)-3-methylphenyl)amino)-5,6-dihydropyrido[4′,3′:4,5]thieno[2,3-d]pyrimidin-7(8H)-yl)-4-(dimethylamino)but-2-en-1-one (Compound 22)

A mixture of 22-7 (30 mg, 0.07 mmol) and (E)-4-(dimethylamino) but-2-enoic acid hydrochloride (14 mg, 0.084 mmol) and EDCI (27 mg, 0.14 mmol) in DCM/DMA (5 mL/3 mL) was stirred at ambient temperature for 3 h. The mixture was adjusted to pH 11 with aqueous NaOH and then extracted with DCM. The organic phae was separated and washed by brine, dried over anhydrous Na₂SO₄ and evaporated to dryness. The resultant residue was purified by prep-TLC (DCM:MeOH=6:1) to afford the title compound as off-white solid (12.5 mg, 33%). LCMS m z [M+H]⁺: 540.2; ¹H NMR (400 MHz, DMSO-d₆): δ 13.06 (s, 1H), 8.41 (s, 1H), 8.19 (s, 1H), 7.98 (s, 1H), 7.57-7.55 (m, 2H), 7.51-7.47 (m, 1H), 7.12 (d, 2H), 6.88 (d, 2H), 6.74-6.67 (m, 1H), 4.92 (d, 2H), 3.96-3.89 (m, 2H), 3.33-3.25 (m, 4H), 2.41 (s, 6H), 2.22 (s, 3H).

### Example 13: (E)-1-(4-((4-((1H-indazol-6-yl)oxy)-3-methylphenyl)amino)-5,8-dihydropyrido[4′,3′:4,5]thieno[2,3-d]pyrimidin-7(6H)-yl)-4-(dimethylamino)but-2-en-1-one (Compound 23)

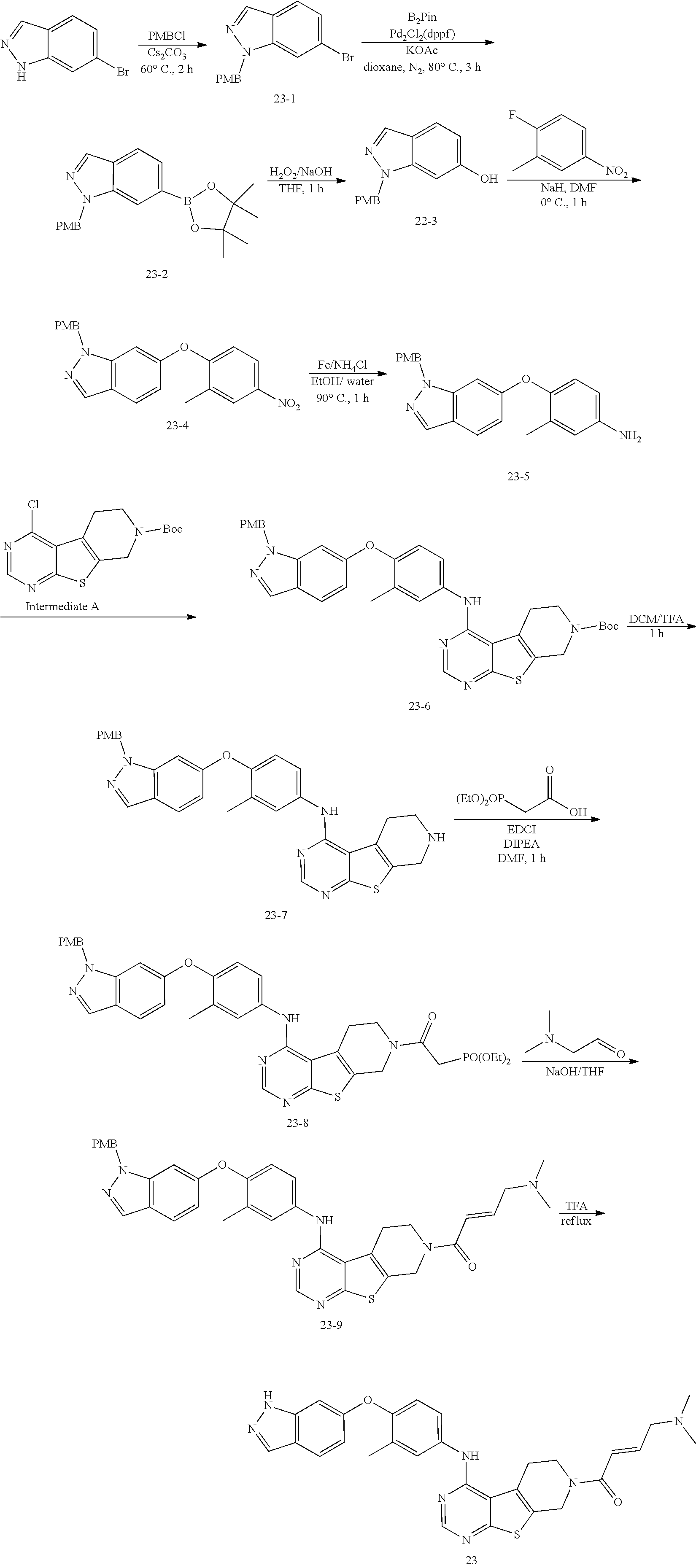

### Step 1: 6-Bromo-1-(4-methoxybenzyl)-1H-indazole (23-1)

PMBCl (4.69 g, 0.03 mol) was added to a mixture of 6-bromo-1H-indazole (5 g, 0.025 mol) and Cs₂CO₃ (12.4 g, 0.038 mol) in DMF (50 mL) at ambient temperature. The resulting mixture was stirred at ambient temperature for 2 h. The reaction solution was diluted with EtOAc, the organics separated, washed with brine, dried over anhydrous Na₂SO₄ and evaporated to dryness. The resultant residue was purified through column chromatography (hexanes:EtOAc=10:1) to afford the title compound white solid (8 g, 99%). LCMS m/z [M+H]⁺: 317.0.

### Step 2: 1-(4-Methoxybenzyl)-6-(4,4,5,5-tetramethyl-1,3,2-dioxaborolan-2-yl)-1H-indazole (23-2)

A solution of 23-1 (1.5 g, 4.73 mmol), B₂Pin₂ (1.56 g, 6.14 mmol), Pd(dppf)Cl₂ (350 mg, 0.47 mmol), and KOAc (0.93 g, 9.5 mmol) in dioxane (20 mL) was stirred at 80° C. for 3 h under nitrogen atmosphere. The mixture was diluted with water and extracted with EtOAc. The combined organic layers were dried over anhydrous Na₂SO₄ and evaporated to dryness to afford the title compound (1.6 g).

### Step 3: 1-(4-Methoxybenzyl)-1H-indazol-6-ol (23-3)

A mixture of 23-2 (1.6 g, 4.38 mmol), NaOH (527 mg, 13.1 mmol) THF (10 mL) and H₂O₂ (30%, 8.5 mL) was stirred at ambient temperature for 0.5 h. The mixture was diluted with water and extracted with EtOAc. The combined organic layers were dried over anhydrous Na₂SO₄ and evaporated to dryness. The resultant residue was purified through column chromatography (hexanes:EtOAc=1:1) to afford the title compound as orange oil (580 mg, 55% two steps). LCMS m/z [M+H]⁺: 255.1.

### Step 4: 1-(4-methoxybenzyl)-6-(2-methyl-4-nitrophenoxy)-1H-indazole (23-4)

To a solution of 23-3 (574 mg, 2.25 mmol) in DMF (20 mL) was added NaH (200 mg, 4.5 mmol). The mixture was stirred at 0° C. for 15 min. 1-Fluoro-2-methyl-4-nitrobenzene (383 mg, 2.47 mmol) was added and the mixture was stirred at 0° C. for 30 min after which it was diluted with water and extracted with EtOAc. The organic layers were dried over anhydrous Na₂SO₄ and evaporated to dryness. The resultant residue was purified with column chromatography (hexane:EtOAc=4:1) to afford the title compound as brown oil (280 mg, 32%). LCMS m/z [M+H]⁺: 390.1.

### Step 5: 4-((1-(4-methoxybenzyl)-1H-indazol-6-yl)oxy)-3-methylaniline (23-5)

A solution of 23-4 (270 mg, 0.69 mmol), Fe (190 mg, 3.46 mmol) and NH₄Cl (190 mg, 3.58 mmol) in EtOH/H₂O (20 mL/5 mL) was stirred at 90° C. for 2 h. The mixture was filtered and the filtrate was diluted with water and extracted with DCM. The combined organic layers were dried over anhydrous Na₂SO₄ and evaporated to dryness to afford the title compound (240 mg, 88%). LCMS m/z [M+H]⁺: 360.3.

### Step 6: tert-butyl 4-((4-((1-(4-methoxybenzyl)-1H-indazol-6-yl)oxy)-3-methylphenyl)amino)-5,8-dihydropyrido[4′,3′:4,5]thieno[2,3-d]pyrimidine-7(6H)-carboxylate (23-6)

A mixture of 23-5 (240 mg, 0.67 mmol), Intermediate A (240 mg, 0.73 mmol), DavePhos (52 mg, 0.13 mmol), Pd₂(dba)₃ (61 mg, 0.07 mmol) and Na₂CO₃ (425 mg, 4.02 mmol) in t-amylalcohol (10 mL) was stirred at 100° C. for 3 h under nitrogen atmosphere. The reaction mixture was concentrated the resultant residue was purified through column chromatography (DCM:MeOH=100:1) to afford the title compound as light yellow solid (270 mg, 63%).

### Step 7: N-(4-((1-(4-methoxybenzyl)-1H-indazol-6-yl)oxy)-3-methylphenyl)-5,6,7,8-tetrahydropyrido[4′,3′:4,5]thieno[2,3-d]pyrimidin-4-amine (23-7)

A solution of 23-6 (270 mg, 0.42 mmol) in TFA (3 mL) and DCM (3 mL) was stirred at 20° C. for 30 min. The mixture was concentrated. The resultant residue was basified with aqueous Na₂CO₃ and extracted with DCM. The combined organic layers were dried over anhydrous Na₂SO₄ and evaporated to dryness. The resultant residue was purified with column chromatography (DCM:MeOH=10:1) to afford the title compound as white solid (200 mg, 88%). LCMS m/z [M+H]⁺: 549.4.

### Step 8: diethyl (2-(4-((4-((1-(4-methoxybenzyl)-1H-indazol-6-yl)oxy)-3-methylphenyl)amino)-5,8-dihydropyrido[4′,3′:4,5]thieno[2,3-d]pyrimidin-7(6H)-yl)-2-oxoethyl)phosphonate (23-8)

To a solution of 23-7 (90 mg, 0.2 mmol) in DMF (8 mL) was added 2-(diethoxy phosphoryl)acetic acid (80 mg, 0.4 mmol), DIPEA (52 mg, 0.4 mmol), and EDCI (80 mg, 0.4 mmol). The mixture was stirred at ambient temperature for 1 h after which it was diluted with water, extracted with EtOAc and the combined organic layers were dried over anhydrous Na₂SO₄ and evaporated to dryness to afford the title compound as brown oil (100 mg, 84%).

### Step 9: (E)-4-(dimethylamino)-1-(4-((4-((1-(4-methoxybenzyl)-1H-indazol-6-yl)oxy)-3-methylphenyl)amino)-5,8-dihydropyrido[4′,3′:4,5]thieno[2,3-d]pyrimidin-7(6H)-yl)but-2-en-1-one (23-9)

To a solution of 23-8 (100 mg, 0.14 mmol) in THF (10 mL) was added NaOH (4 g/10 mL water) and 2-(dimethylamino)acetaldehyde (1.0 g in 10 mL 6 M hydrochloric acid) at 0° C. The mixture was stirred at ambient temperature overnight after which it was diluted with water and extracted with EtOAc. The combined organic layers were dried over anhydrous Na₂SO₄ and evaporated to dryness to afford the title compound (70 mg, 77%), LCMS m/z [M+H]⁺: 660.2.

### Step 10: (E)-1-(4-((4-((1H-Indazol-6-yl)oxy)-3-methylphenyl)amino)-5,6-dihydropyrido[4′,3′:4,5]thieno[2,3-d]pyrimidin-7(8H)-yl)-4-(dimethylamino)but-2-en-1-one (Compound 23)

A solution of 23-9 (70 mg, 0.1 mmol) in TFA (10 mL) was stirred at 80° C. for 48 h. The mixture was concentrated. The residue was adjusted to pH 10 with aqueous Na₂CO₃ and the resulting mixture was extracted by DCM. The combined organic layers were dried over anhydrous Na₂SO₄ and evaporated to dryness. The resultant residue was purified through column chromatography (DCM:MeOH=10:1) to afford the title compound as white solid (6.1 mg, 11%). LCMS m/z [M+H]⁺: 540.2; ¹H NMR (400 MHz, CD₃OD): δ 8.36 (s, 1H), 7.98 (s, 1H), 7.55-7.53 (m, 1H), 7.56-7.47 (m, 2H), 7.03-7.00 (d, 1H), 6.95-6.92 (m, 1H), 6.84-6.72 (m, 3H), 5.00-4.95 (d, 2H), 4.07-4.04 (m, 2H), 3.28-3.22 (m, 4H), 2.32-2.30 (d, 6H), 2.25 (s, 3H).

### Example 14: (E)-(3-(4-((7-(4-(dimethylamino)but-2-enoyl)-5,6,7,8-tetrahydropyrido[4′,3′:4,5]thieno[2,3-d]pyrimidin-4-yl)amino)-2-methylphenoxy)phenyl)boronic acid (Compound 64)

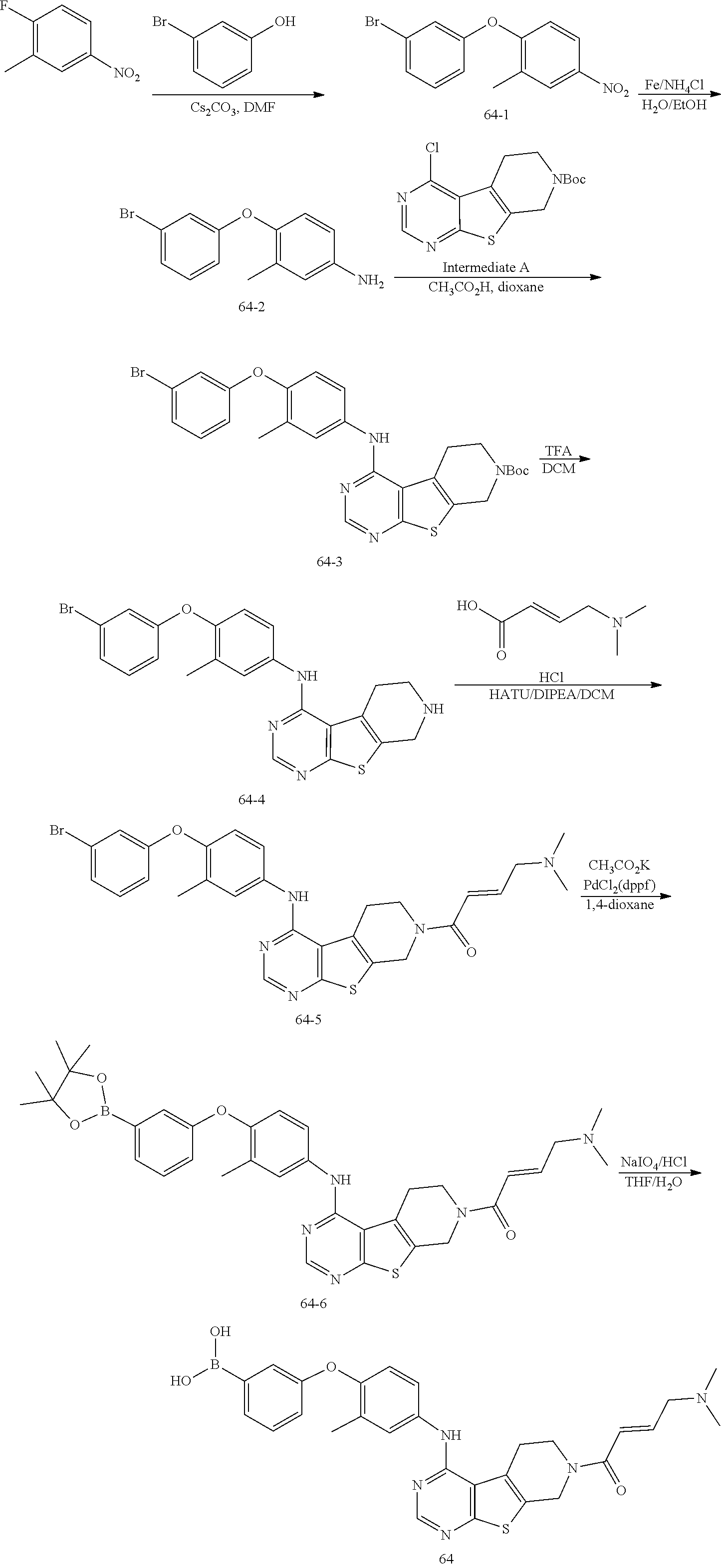

### Step 1: 1-(3-Bromophenoxy)-2-methyl-4-nitrobenzene (64-1)

A mixture of 3-bromophenol (1.2 g, 7.0 mmol), 1-fluoro-2-methyl-4-nitrobenzene (986 mg, 6.4 mmol), Cs₂CO₃ (3.8 g, 11.6 mmol) in DMF (14 mL) was stirred at ambient temperature for 4 h. Water was added to the reaction mixture. The resulting suspension was extracted with EtOAc and washed with water and brine. The organic layer was concentrated and the resultant residue purified through column chromatography (hexanes/EtOAc=100/1) to afford the title compound as white solid (1.1 g, 56%). ¹H NMR (400 MHz, CDCl₃): δ 8.17 (d, 1H), 8.03 (dd, 1H), 7.34-7.36 (m, 1H), 7.25-7.29 (m, 1H), 7.19 (t, 1H), 6.95-6.98 (m, 1H), 6.84 (d, 1H), 2.39 (s, 3H).

### Step 2: 4-(3-Bromophenoxy)-3-methylaniline (64-2)

A solution of 64-1 (1.1 g, 3.6 mmol), Fe (1.2 g, 21.4 mmol) and NH₄Cl (1.1 g, 20.5 mmol) in EtOH/H₂O (18 mL/9 mL) was stirred at 80° C. for 3 h. The mixture was filtered. The filtrate was diluted with water and extracted by EtOAc. The organic layers were dried over anhydrous Na₂SO₄ and evaporated to afford the title compound as brown oil (990 mg, crude). LCMS m/z [M+H]⁺: 279.9; ¹H NMR (400 MHz, CDCl₃): δ 7.01-7.13 (m, 2H), 6.96-6.96 (m, 1H), 6.77-6.79 (m, 2H), 6.59 (d, 1H), 6.53 (dd, 1H), 3.59 (br, 2H), 2.08 (s, 3H).

### Step 3: tert-Butyl 4-((4-(3-bromophenoxy)-3-methylphenyl)amino)-5,6-dihydropyrido[4′,3′:4,5]thieno[2,3-d]pyrimidine-7(8H)-carboxylate (64-3)

A mixture of 64-2 (400 mg, 1.44 mmol), Intermediate A (469 mg, 1.44 mmol) and CH₃CO₂H (216 mg, 3.6 mmol) in 1,4-dioxane (10 mL) was stirred at 110° C. for 4 h. The reaction mixture was quenched with water, adjusted pH to 9-10 with saturated Na₂CO₃. The resulting mixture was extracted with EtOAc. The organic layer was washed with brine, concentrated and purified on column chromatography (hexane/EtOAc=7/1) to afford the title compound as white solid (410 mg, 50%). LCMS m/z [M+H]⁺: 568.9; ¹H NMR (400 MHz, CDCl₃): δ 8.53 (s, 1H), 7.49 (dd, 1H), 7.16-7.19 (m, 2H), 7.04-7.05 (m, 1H), 6.98 (d, 1H), 6.94 (s, 1H), 6.86-6.88 (m, 1H), 4.72 (br, 2H), 3.77 (t, 2H), br (t, 2H), 2.24 (s, 3H), 1.51 (s, 9H).

### Step 4: N-(4-(3-Bromophenoxy)-3-methylphenyl)-5,6,7,8-tetrahydropyrido[4′,3′:4,5]thieno[2,3-d]pyrimidin-4-amine (64-4)

TFA (6 mL) was added to a solution of 64-3 (410 mg, 0.72 mmol) in DCM (14 mL). The mixture was stirred at ambient temperature for 2 h. The reaction solution was concentrated to remove TFA. The resultant residue was dissolved in DCM, washed with aqueous Na₂CO₃ and brine. The DCM layer was concentrated to afford the title compound as white solid (310 mg, crude). LCMS m/z [M+H]⁺: 468.8.

### Step 5: (E)-1-(4-((4-(3-Bromophenoxy)-3-methylphenyl)amino)-5,6-dihydropyrido[4′,3′:4,5]thieno[2,3-d]pyrimidin-7(8H)-yl)-4-(dimethylamino)but-2-en-1-one (64-5)

A mixture of 64-4 (440 mg, 0.94 mmol), (E)-4-(dimethylamino) but-2-enoic acid hydrochloride (187 mg, 1.13 mmol), HATU (716 mg, 1.88 mmol) and DIPEA (365 mg, 2.82 mmol) in DCM (20 mL) was stirred at ambient temperature for 2 h after which water was added and resultant mixture extracted with DCM. The organic layer was separated, washed with brine, dried over anhydrous Na₂SO₄ and evaporated to dryness. The resultant residue was purified through column chromatography (DCM:MeOH=30:1) to afford the title compound as white solid (350 mg, 64%). LCMS m/z [M+H]⁺: 579.9; ¹H NMR (400 MHz, CDCl₃): δ 8.53 (s, 1H), 7.46-7.52 (m, 2H), 7.15-7.19 (m, 2H), 7.05 (br, 1H), 6.86-7.00 (m, 4H), 6.46-6.59 (m, 1H), 4.92 (d, 2H), 4.05 (d, 2H), 3.23 (br, 2H), 3.13 (d, 2H), 2.29 (s, 6H), 2.24 (s, 3H).

### Step 6: (E)-4-(Dimethylamino)-1-(4-((3-methyl-4-(3-(4,4,5,5-tetramethyl-1,3,2-dioxaborolan-2-yl)phenoxy)phenyl)amino)-5,6-dihydropyrido[4′,3′:4,5]thieno[2,3-d]pyrimidin-7(8H)-yl)but-2-en-1-one (64-6)

A mixture of 64-5 (350 mg, 0.61 mmol), 4,4,4′,4′,5,5,5′,5′-octamethyl-2,2′-bi (1,3,2-dioxaborolane) (184 mg, 0.73 mmol), CH₃CO₂K (119 mg, 1.21 mmol) and PdCl₂(dppf) (44 mg, 0.061 mmol) in 1,4-dioxane (12 mL) was stirred at 85° C. for 7 h under nitrogen atmosphere. The mixture was concentrated and purified with column chromatography (DCM:MeOH=30:1) to afford the title compound as brown solid (200 mg, 53%). LCMS m/z [M+H]⁺: 626.1.

### Step 7: (E)-(3-(4-((7-(4-(Dimethylamino)but-2-enoyl)-5,6,7,8-tetrahydropyrido[4′,3′:4,5]thieno[2,3-d]pyrimidin-4-yl)amino)-2-methylphenoxy)phenyl)boronic acid (Compound 64)

A mixture of 64-6 (200 mg, 0.32 mmol) and NaIO₄ (205 mg, 0.96 mmol) in THF/H₂O (10 mL/2.5 mL) was stirred at ambient temperature for 0.5 h. HCl (1 N, 2.2 mL) was added and the reaction mixture was stirred for 3 h. Saturated Na₂CO₃ was added and the pH was adjusted to 8. The mixture was filtered and the filtrate concentrated and purified through column chromatography (DCM/MeOH/NH₄OH=40/4/1) to afford the title compound as brown solid (30 mg, 17%). LCMS m/z [M+H]⁺: 544.1; ¹H NMR (400 MHz, CDCl₃): δ 8.46 (s, 1H), 7.68 (d, 1H), 7.60 (s, 1H), 7.42 (s, 1H), 7.33-7.36 (m, 2H), 6.86-6.94 (m, 4H), 6.55 (dd, 1H), 4.83 (d, 2H), 3.97 (d, 2H), 3.35 (sr, 1H), 3.14-3.32 (m, 3H), 2.43 (s, 3H), 2.33 (s, 3H), 2.22 (s, 3H).

### Example 15: Ethyl 2-((4-((3-methyl-4-((6-methylpyridin-3-yl)oxy)phenyl)amino)-5,8-dihydropyrido[4′,3′:4,5]thieno[2,3-d]pyrimidin-7(6H)-yl)methyl)acrylate (Compound 86)

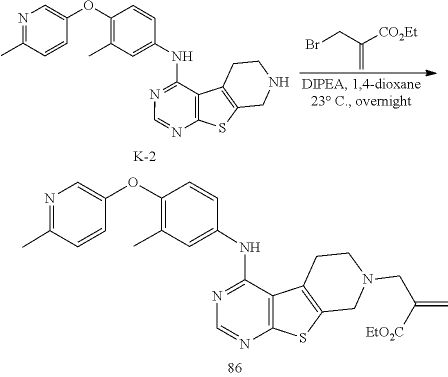

A mixture of K-2 (from Scheme 11) (45.0 mg, 0.11 mmol), ethyl 2-(bromomethyl)acrylate (28.0 mg, 0.14 mmol), and DIPEA (23.1 mg, 0.18 mmol) in 1,4-dioxane (1.0 mL) was allowed to stir overnight at ambient temperature. It was diluted with DCM, washed with saturated aqueous NaHCO₃, dried (MgSO₄), and filtered. The filtrate was concentrated in vacuo and purified through column chromatography on silica gel (eluting with 10% MeOH in DCM) to afford the title compound as yellow solid (15 mg, 26%). MS m/z: 515.8 (M+H)+. ¹H NMR (400 MHz, DMSO-d₆): δ 8.38 (s, 1H), 8.14 (d, 1H), 8.12 (b s, 1H), 7.57-7.52 (m, 2H), 7.23-7.15 (m, 2H), 6.92 (d, 1H), 6.20 (d, 1H), 5.85 (d, 1H), 4.15 (q, 2H), 3.74 (s, 2H), 3.38 (s, 2H), 3.20 (t, 2H), 2.82 (t, 2H), 2.41 (s, 3H), 2.16 (s, 3H), 1.22 (t, 3H).

### Example 16: 1-(4-((3-methyl-4-((6-methylpyridin-3-yl)oxy)phenyl)amino)-7,8-dihydro-5H-pyrido[3′,4′:4,5]pyrrolo[2,3-d]pyrimidin-6(9H)-yl)prop-2-en-1-one (Compound 94)

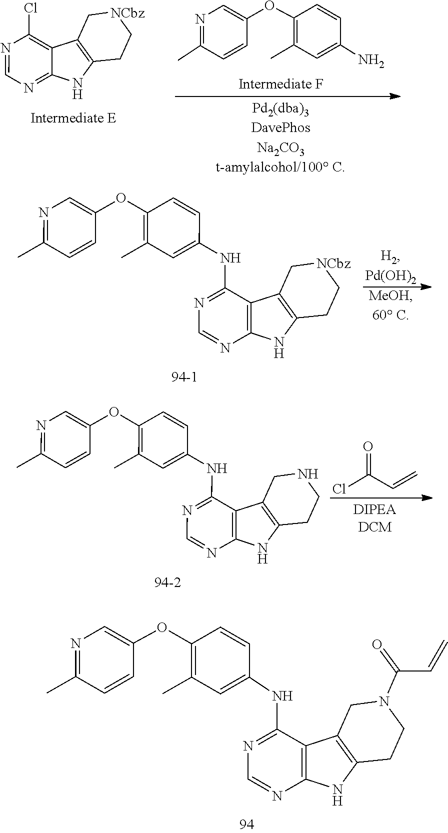

### Step 1: Benzyl 4-((3-methyl-4-((6-methylpyridin-3-yl)oxy)phenyl)amino)-7,8-dihydro-5H-pyrido[3′,4′:4,5]pyrrolo[2,3-d]pyrimidine-6(9H)-carboxylate (94-1)

To a solution of Intermediate F (390 mg, 1.82 mmol) and Intermediate E (686 mg, 2 mmol) in t-amyl alcohol (10 mL) was added Pd₂(dba)₃ (167 mg, 0.18 mmol), Davephos (143 mg, 0.36 mmol) and Na₂CO₃ (1.1 g, 10.9 mmol). The resulting mixture was allowed to stir at 100° C. for 2 h. The mixture was washed with water, extracted with EtOAc, dried over anhydrous sodium suflate and concentrated. The resultant residue was purified by column chromatography eluting with 5% MeOH in DCM to afford the title compound (500 mg, 53%). LCMS m/z [M+H]+ 521.5.

### Step 2: N-(3-Methyl-4-((6-methylpyridin-3-yl)oxy)phenyl)-6,7,8,9-tetrahydro-5H-pyrido[3′,4′:4,5]pyrrolo[2,3-d]pyrimidin-4-amine, 94-2 (94-2)

To a solution of 94-1 (100 mg, 0.19 mmol) in methanol (5 mL) was added palladium hydroxide (50 mg, 0.19 mmol) under hydrogen atmosphere. The resulting mixture was allowed to stir at 60° C. for 2 h. The reaction mixture was filtered and the filtrate concentrated to afford the title compound. (60 mg, 80.8%). LCMS m/z [M+H]+ 387.1.

### Step 3: 1-(4-((3-Methyl-4-((6-methylpyridin-3-yl)oxy)phenyl)amino)-7,8-dihydro-5H-pyrido[3′,4′:4,5]pyrrolo[2,3-d]pyrimidin-6(9H)-yl)prop-2-en-1-one (Compound 94)

To a solution of 94-2 (60 mg, 0.16 mmol) in DCM (3 mL) was added acryloyl chloride (14 mg, 0.16 mmol) and DIPEA (60 mg, 0.47 mmol). The resulting mixture was allowed to stir at room temperature for 1 h. The reaction mixture was washed with water, extracted with EtOAc, dried and concentrated. The resultant residue was purified by column chromatography eluting 8% MeOH in DCM to afford the title compound as white solid (7.9 mg, 11.2%). LCMS m/z [M+H]⁺ 441.2. ¹H NMR (400 MHz, CD₃OD): δ 8.03 (d, 2H), 7.41-7.27 (m, 2H), 7.21-7.14 (m, 2H), 6.89-6.77 (m, 2H), 6.18 (d, 1H), 5.74-5.68 (m, 1H), 4.89 (d, 2H), 3.93-3.88 (m, 2H), 2.85-2.76 (m, 2H), 2.39 (s, 3H), 2.12 (s, 3H).

### Example 17: (E)-4-(Dimethylamino)-1-(4-((4-((6-methoxypyridin-3-yl)oxy)-3-methylphenyl)amino)-5,6-dihydropyrido[4′,3′:4,5]thieno[2,3-d]pyrimidin-7(8H)-yl)but-2-en-1-one (Compound 95)

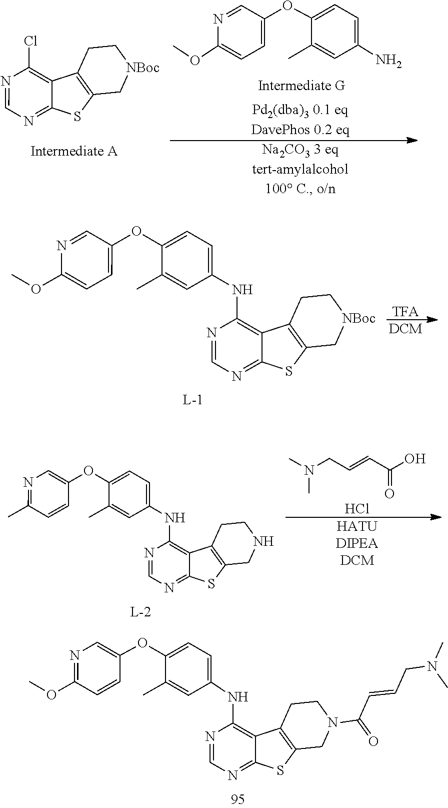

The title compound was prepared from L-2, described in Scheme 12. A mixture of L-2 (75 mg, 0.18 mmol), (E)-4-(dimethylamino)but-2-enoic acid hydrochloride (59 mg, 0.35 mmol), DIPEA (69 mg, 0.53 mmol) and HATU (170 mg, 0.44 mmol) in DCM (10 mL) was allowed to stir at ambient temperature for 1 h. The reaction mixture was concentrated, and the resultant residue dissolved in DCM, washed with brine, concentrated. The crude product was purified by prep-TLC (DCM/MeOH=25/1) to afford the title compound as light yellow solid (31.5 mg, 32%). LCMS m/z [M+H]⁺: 531.4. ¹HNMR (400 MHz, DMSO-d₆): δ 8.40 (s, 1H), 8.18 (s, 1H), 7.90 (d, 1H), 7.54 (s, 1H), 7.47 (q, 1H), 7.38 (q, 1H), 6.86 (q, 2H), 6.72 (br, 2H), 4.90 (d, 2H), 3.90 (d, 2H), 3.84 (s, 3H), 3.22 (d, 4H), 2.27 (s, 6H), 2.23 (s, 3H).

### Example 18: (E)-4-ethoxy-1-(4-((3-methyl-4-((6-methylpyridin-3-yl)oxy)phenyl)amino)-5,8-dihydropyrido[4′,3′:4,5]thieno[2,3-d]pyrimidin-7(6H)-yl)but-2-en-1-one (Compound 96)

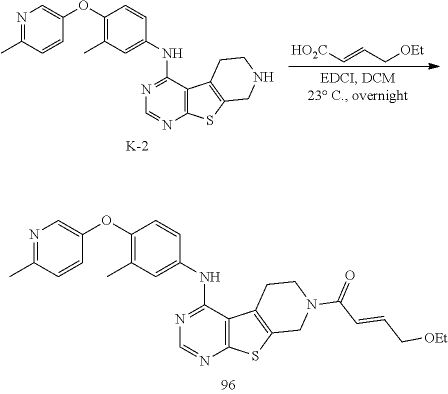

The title compound was prepared in analogous fashion to Compound 8, using (E)-4-ethoxybut-2-enoic acid instead of (E)-4-(dimethylamino)but-2-enoic acid in the last step reaction. The title compound was obtained as white solid. MS m/z: 516.3 (M+H)+. ¹H NMR (400 MHz, DMSO-d₆): δ 8.40 (s, 1H), 8.20 (s, 1H), 8.15 (s, 1H), 7.55-7.50 (m. 2H), 7.21-7.17 (m, 2H), 6.92 (d, 1H), 6.74-6.69 (m, 2H), 4.93 & 4.84 (2b s, 2H), 4.12-4.10 (m, 2H), 3.91-3.84 (m, 2H), 3.48 (q, 2H), 3.29-3.22 (m, 2H), 2.41 (s, 3H), 2.16 (s, 3H), 1.14 (t, 3H).

### Example 19: (E)-4-(dimethylamino)-1-(4-((3-methyl-4-((6-methylpyridin-3-yl)oxy)phenyl)amino)-5,7,8,9-tetrahydro-6H-pyrido[3′,4′:4,5]pyrrolo[2,3-d]pyrimidin-6-yl)but-2-en-1-one (Compound 97)

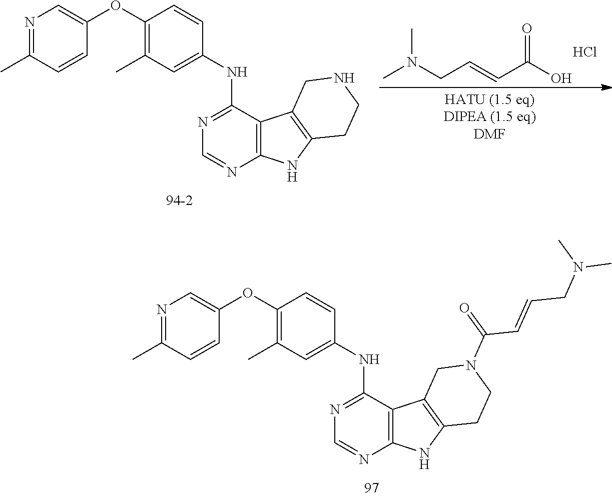

To a solution of N-(3-methyl-4-((6-methylpyridin-3-yl)oxy)phenyl)-6,7,8,9-tetrahydro-5H-pyrido[3′,4′:4,5]pyrrolo[2,3-d]pyrimidin-4-amine (94-2) (90 mg, 0.23 mmol) in DMF (5 mL) was added (E)-4-(dimethylamino)but-2-enoic acid (45.1 mg, 0.35 mmol), HATU (132.8 mg, 0.35 mmol) and DIPEA (90 mg, 0.7 mmol). The resulting mixture was allowed to stir at room temperature overnight. The mixture was washed with water, extracted with EtOAc, dried and evaporated to dryness. The resultant residue was purified by column chromatography eluting with 10% MeOH in DCM to afford the title compound (19.8 mg) as light yellow solid. LCMS m/z [M+H]⁺: 498.3. ¹H NMR (400 MHz, DMSO-d₆): δ 11.70 (d, 1H), 8.32-8.17 (m, 3H), 7.55 (t, 2H), 7.25-7.15 (m, 2H), 7.02-6.56 (m, 3H), 4.91 (d, 2H), 3.90-3.77 (m, 2H), 3.22-3.05 (m, 2H), 2.77 (d, 2H), 2.43 (s, 3H), 2.15 (m, 9H).

### Example 20: (E)-4-(dimethylamino)-1-(5-methyl-4-((3-methyl-4-((6-methylpyridin-3-yl)oxy)phenyl)amino)-5,8-dihydropyrido[4′,3′:4,5]thieno[2,3-d]pyrimidin-7(6H)-yl)but-2-en-1-one (Compound 98)

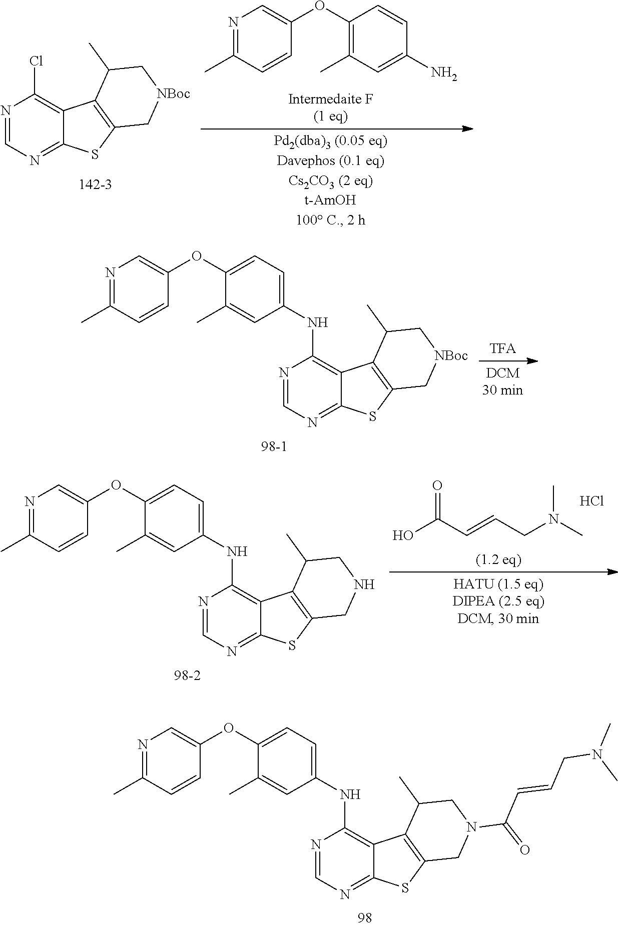

The title compound was prepared in analogous fashion to Compound 99, using tert-butyl 4-chloro-5-methyl-5,6-dihydropyrido[4′,3′:4,5]thieno[2,3-d]pyrimidine-7(8H)-carboxylate (142-3) instead of Intermediate C. The title compound was obtained as yellow solid (81 mg). LCMS: 529.4 (M+1). ¹HNMR (400 MHz, DMSO-d₆): 1.19 (m, 3H), 2.14-2.20 (m, 9H), 2.44 (s, 3H), 3.07-3.09 (m, 2H), 3.28 (s, 0.5H), 3.73 (dd, 0.5H), 3.87 (s, 1H), 4.13 (dd, 0.5H), 4.36 (d, 0.5H), 4.47 (d, 0.5H), 4.71 (d, 0.5H), 5.25 (d, 0.5H), 5.37 (d, 0.5H), 6.74-6.76 (m, 2H), 6.95 (d, 1H), 7.19-7.26 (m, 2H), 7.48 (d, 1H), 7.55 (s, 1H), 8.18-8.25 (m, 2H), 8.41 (d, 1H).

### Example 21: (E)-4-(dimethylamino)-1-(4-((3-methyl-4-((6-methylpyridin-3-yl)oxy)phenyl)amino)-5,7-dihydro-6H-pyrrolo[3′,4′:4,5]thieno[2,3-d]pyrimidin-6-yl)but-2-en-1-one (Compound 99)

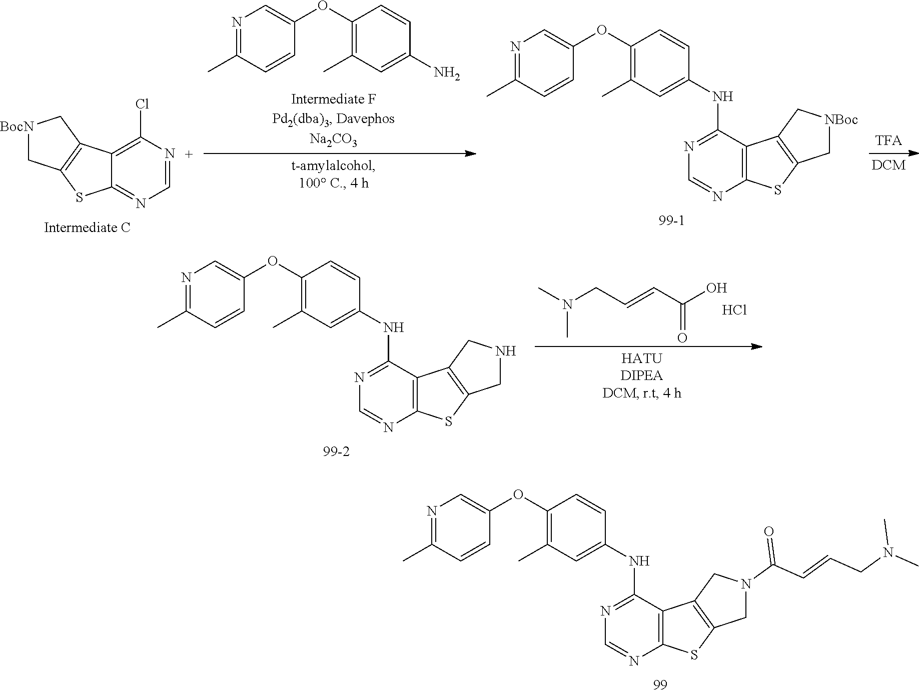

The title compound was prepared in analogous fashion to Compound 8, but using Intermediate C instead of Intermediate A. The title compound was obtained as off-white solid (26.2 mg). LCMS m/z [M+H]⁺: 501.2. ¹H NMR (400 MHz, DMSO-d₆): δ 8.64 (s, 0.5H), 8.45 (s, 0.5H), 8.43 (s, 1H), 8.17 (t, 1H), 7.55 (s, 1H), 7.48 (d, 1H), 7.23 (dd, 2H), 6.99-6.93 (m, 1H), 6.78 (s, 1H), 6.55 (d, 1H), 5.25 (s, 1H), 5.10-5.01 (m, 2H), 4.82 (s, 1H), 3.30 (s, 2H), 2.44 (s, 3H), 2.33 (d, 6H), 2.21 (d, 3H).

### Example 22: (E)-4-(dimethylamino)-1-(4-((3-methyl-4-((6-methylpyridin-3-yl)oxy)phenyl)amino)-8,9-dihydro-5H-pyrimido[5′,4′:4,5]thieno[2,3-d]azepin-7(6H)-yl)but-2-en-1-one (Compound 100)

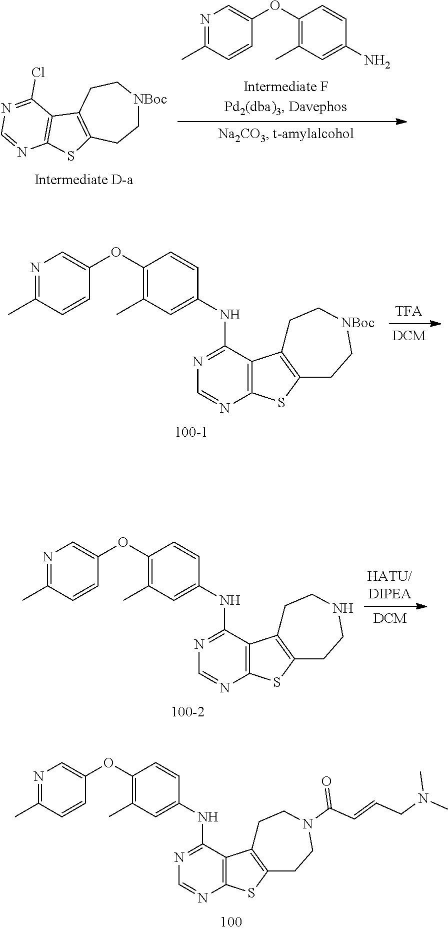

The title compound was prepared in analogous fashion to Compound 99, using Intermediate D-a instead of Intermediate C. The title compound was obtained as white solid (20 mg). LCMS m/z [M+H]⁺: 529.4. ¹H NMR (400 MHz, DMSO-d₆): δ 8.45 (s, 1H), 8.38 (d, 1H), 8.17 (t, 1H), 7.51 (s, 1H), 7.43-7.45 (m, 1H), 7.18-7.25 (m, 2H), 6.93-6.96 (m, 1H), 6.64-6.72 (m, 2H), 3.87-3.94 (m, 3H), 3.77 (t, 1H), 3.38-3.41 (m, 2H), 3.16-3.20 (m, 4H), 2.44 (s, 3H), 2.24 (s, 3H), 2.23 (s, 3H), 2.18 (s, 3H).

### Example 23: (E)-4-(Dimethylamino)-1-(4-((3-methyl-4-((6-methylpyridin-3-yl)oxy)phenyl)amino)-6,7-dihydro-5H-pyrimido[5′,4′:4,5]thieno[2,3-c]azepin-8(9H)-yl)but-2-en-1-one (Compound 101)

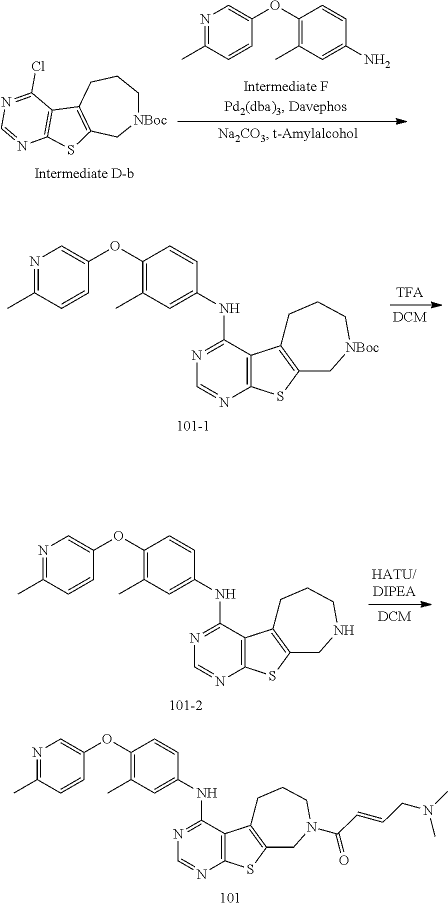

The title compound was prepared in analogous fashion to Compound 99, using Intermediate D-b instead of Intermediate C. The title compound was obtained as light yellow solid (20 mg). LCMS m/z [M+H]⁺: 529.2. ¹H NMR (400 MHz, CDCl₃): δ 8.49 (d, 1H), 8.26 (d, 1H), 7.45-7.46 (m, 1H), 7.37-7.40 (m, 1H), 7.14-7.08 (m, 2H), 6.80-7.01 (m, 3H), 6.64 (d, 0.5H), 6.44 (d, 0.5H), 4.87 (s, 1H), 4.78 (s, 1H), 3.95-4.01 (m, 2H), 3.22-3.33 (m, 3H), 3.11 (d, 1H), 2.53 (s, 3H), 2.32-2.38 (m, 5H), 2.28 (s, 6H).

### Example 24: 1-(4-((4-([1,2,4]triazolo[1,5-a]pyridin-6-yloxy)-3-methylphenyl)amino)-5,7,8,9-tetrahydro-6H-pyrido[3′,4′:4,5]pyrrolo[2,3-d]pyrimidin-6-yl)prop-2-en-1-one (Compound 102)

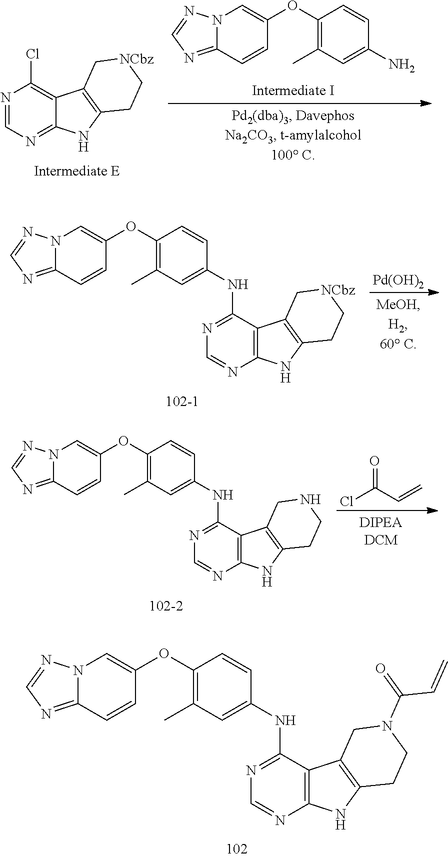

The title compound was prepared in analogous fashion to Compound 94, using Intermediate I instead of Intermediate F. The title compound was obtained as white solid (16 mg). LCMS m/z [M+H]⁺ 467.2. ¹H NMR (400 MHz, DMSO-d₆): δ 11.70 (d, 1H), 8.58 (d, 1H), 8.47 (s, 1H), 8.30 (d, 1H), 8.17 (s, 1H), 7.91-7.86 (m, 1H), 7.59 (d, 1H), 7.55 (d, 2H), 7.09-6.90 (m, 2H), 6.25-6.12 (m, 1H), 5.79-5.71 (m, 1H), 4.98 (d, 2H), 3.89 (t, 2H), 2.84-2.74 (m, 2H), 2.26 (d, 3H).

### Example 25: (E)-1-(4-((4-([1,2,4]triazolo[1,5-a]pyridin-6-yloxy)-3-methylphenyl)amino)-7,8-dihydropyrido[3′,4′:4,5]thieno[2,3-d]pyrimidin-6(5H)-yl)-4-(dimethylamino)but-2-en-1-one (Compound 103)

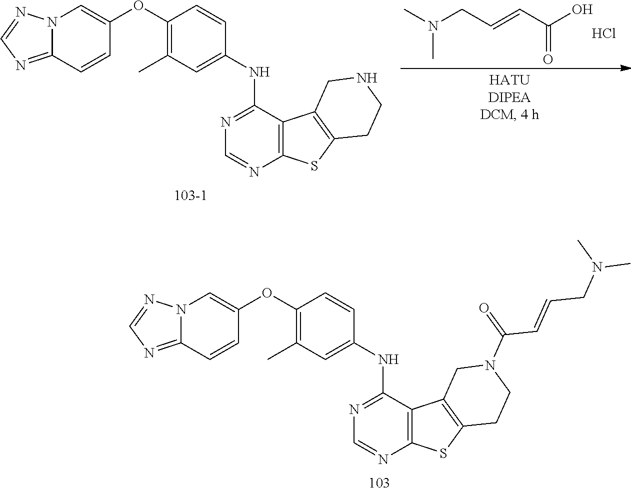

N-(4-([1,2,4]triazolo[1,5-a]pyridin-6-yloxy)-3-methylphenyl)-5,6,7,8-tetrahydropyrido[3′,4′:4,5]thieno[2,3-d]pyrimidin-4-amine (103-1) (160 mg, 0.37 mmol) was prepared in analogous fashion to 102-2, using Intermediate B in place of Intermediate E. 103-1 was combined with (E)-4-(dimethylamino)but-2-enoic acid hydrochloride (74 mg, 0.45 mmol), HATU (212 mg, 0.56 mmol), and DIPEA (144 mg, 1.12 mmol) in DCM (20 mL). Ther resultant mixture was allowed to stir at room temperature for 1 h. The reaction mixture was washed with water and the organic layer concentrated and purified by column chromatography (DCM:MeOH=20:1) to afford the title compound as off-white solid (12 mg, 6%). LCMS m/z [M/2+H]⁺: 542.5. ¹H NMR (400 MHz, CDCl₃): δ 8.48 (s, 1H), 7.08-6.91 (m, 2H), 6.74-6.61 (m, 1H), 5.16 (s, 2H), 4.03-3.85 (m, 2H), 3.63-3.47 (m, 2H), 3.05-2.90 (m, 2H), 2.28 (s, 3H), 7.94-7.87 (m, 1H), 7.56 (dd, 2H), 7.50-7.42 (m, 1H), 8.68-8.61 (m, 1H), 2.50 (s, 6H), 8.43 (s, 2H).

### Example 26: (E)-1-(4-((4-(Benzo[c][1,2,5]oxadiazol-5-yloxy)-3-methylphenyl)amino)-5,6-dihydropyrido[4′,3′:4,5]thieno[2,3-d]pyrimidin-7(8H)-yl)-4-(dimethylamino)but-2-en-1-one (Compound 104)

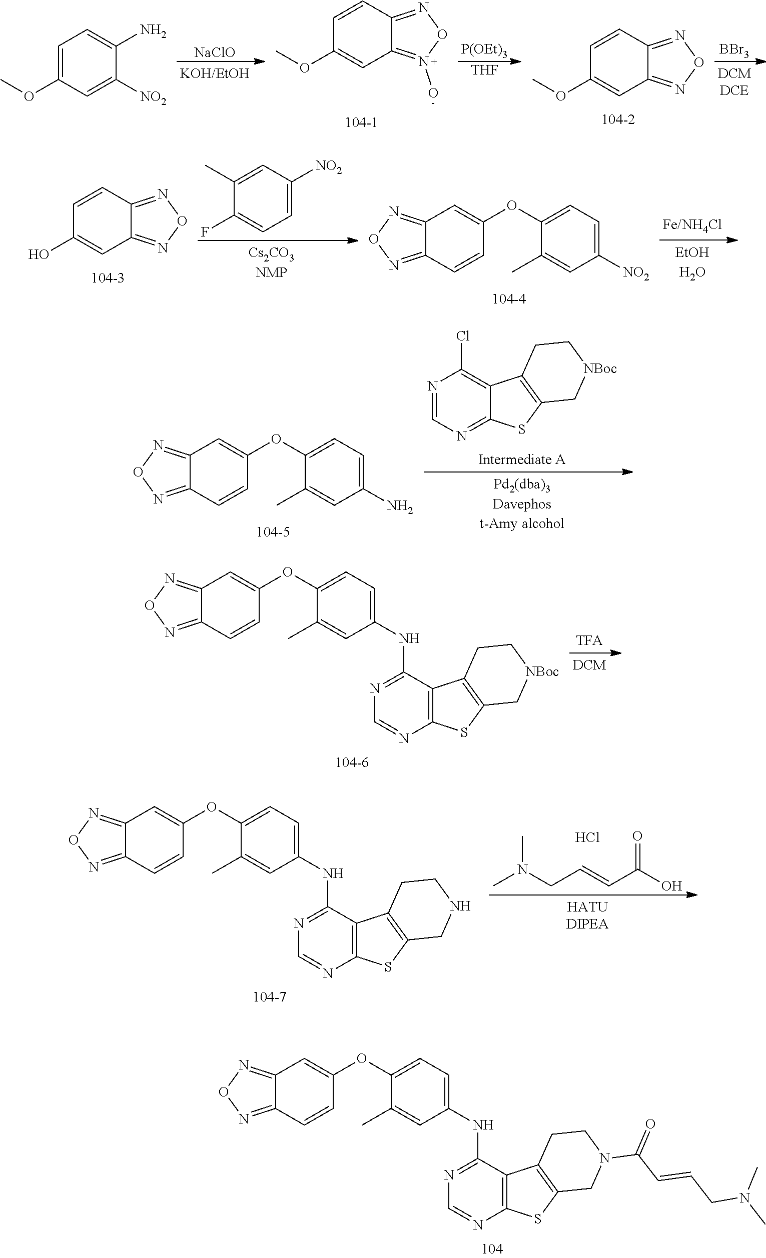

### Step 1: 6-Methoxybenzo[c][1,2,5]oxadiazole 1-oxide (104-1)

To a solution of KOH (16 g, 0.29 mol) in ethanol (80 mL) was added 4-methoxy-2-nitro-aniline (2.0 g, 12 mmol) at 5° C. The resulting mixture was allowed to stir for 10 min. NaClO (40 mL, 12 mmol) was added dropwise until disappearance of 4-methoxy-2-nitro-aniline was observed by TLC and the reaction solution turned from red to yellow. The resulting suspension was filtered and the solid collected, washed with cold water, and dried under vacuum to afford the title compound as yellow solid (1.4 g, 70%). LCMS m/z [M+H]⁺: 166.1. ¹HNMR (400 MHz, CDCl₃): δ 7.25-7.50 (m, 1H), 6.87-7.07 (m, 1H), 6.50 (d, 1H), 3.90 (d, 3H).

### Step 2: 5-Methoxybenzo[c][1,2,5]oxadiazole (104-2)

To a solution of 104-1 (1.2 g, 7.2 mmol) in THF (30 mL) was added (EtO)₃P (2.4 g, 15 mmol) slowly and the mixture was allowed to stir at 60° C. for 4 h. The reaction solution was concentrated and the resultant residue was purified by column chromatography to afford the title compound as light yellow solid (1.02 g, 94%). LCMS m/z [M+H]⁺: 151.1. ¹HNMR (400 MHz, CDCl₃): δ 7.70 (d, 1H), 7.11 (m, 1H), 6.85 (d, 1H), 3.92 (s, 3H).

### Step 3: Benzo[c][1,2,5]oxadiazol-5-ol (104-3)

To a solution of 104-2 (0.5 g, 3.33 mmol) in 1,2-dichloroethane (10 mL) was added BBr₃ (8.25 g, 33.3 mmol) and the mixture was allowed to stir at 85° C. for 24 h. The reaction solution was concentrated and the resultant residue was dissolved in water, extracted with DCM and the organic layer was dried and concentrated. The resultant residue was purified by column chromatography to afford the title compound as white solid (220 mg, 48%). LCMS m/z [M+H]⁺: 137.1. ¹HNMR (400 MHz, CDCl₃): δ 7.78 (m, 1H), 7.16 (m, 1H), 6.96 (m, 1H), 6.10 (d, 1H).

### Step 4: 5-(2-Methyl-4-nitrophenoxy)benzo[c][1,2,5]oxadiazole (104-4)

A suspension of 104-3 (210 mg, 1.54 mmol), 1-fluoro-2-methyl-4-nitro-benzene (263 mg, 1.7 mmol), and Cs₂CO₃ (754 mg, 2.31 mmol) in NMP (10 mL) was allowed to stir at 60° C. overnight. The reaction mixture was quenched with water, extracted with EtOAc and the organic layer was washed with brine, dried and concentrated. The resultant residue was purified by column chromatography to afford the title compound as light yellow solid (125 mg, 29%). ¹HNMR (400 MHz, CDCl₃): δ 8.26 (d, 1H), 8.16 (m, 1H), 7.92 (m, 1H), 7.32 (m, 1H), 7.15 (d, 1H), 6.86 (m, 1H), 2.37 (s, 3H).

### Step 5: 4-(Benzo[c][1,2,5]oxadiazol-5-yloxy)-3-methylaniline (104-5)

A suspension of 104-4 (120 mg, 0.44 mmol), Fe (124 mg, 2.21 mmol), and NH₄Cl (119 mg, 2.21 mmol) in ethanol (10 mL)/water (10 mL) was allowed to stir at 80° C. for 1 h after which the reaction mixture was concentrated and the resultant residue was taken into EtOAc. The organic layer was washed with water, brine and concentrated to afford the title compound as light-yellow solid (80 mg, 74%). LCMS m/z [M+H]⁺: 242.3. ¹H NMR (400 MHz, CDCl₃): δ 7.79 (d, 1H), 7.33 (m, 1H), 6.86 (d, 1H), 6.58-6.63 (m, 3H), 3.66 (br s, 2H), 2.09 (s, 3H).

### Step 6: tert-Butyl 4-((4-(benzo[c][1,2,5]oxadiazol-5-yloxy)-3-methylphenyl)amino)-5,6-dihydropyrido[4′,3′:4,5]thieno[2,3-d]pyrimidine-7(8H)-carboxylate (104-6)

A suspension of 104-5 (80 mg, 0.33 mmol), Intermediate A (227 mg, 0.70 mmol), Na₂CO₃ (246 mg, 2.32 mmol), Davephos (52 mg, 0.13 mmol), and Pd₂(dba)₃ (60 mg, 0.07 mmol) in t-amyl alcohol (10 mL) was allowed to stir at 100° C. under N₂ for 3 h. The reaction mixture was concentrated and the resultant residue was purified by column chromatography to afford the title compound as brown solid (160 mg, 90%). ¹H NMR (400 MHz, CDCl₃): δ 8.55 (s, 1H), 7.84 (m, 1H), 7.61 (t, 2H), 7.37 (m, 1H), 7.11 (d, 1H), 7.00 (s, 1H), 6.67 (d, 1H), 4.74 (s, 2H), 3.88 (t, 2H), 3.19 (br, 2H), 2.24 (s, 3H), 1.52 (s, 9H).

### Step 7: N-(4-(Benzo[c][1,2,5]oxadiazol-5-yloxy)-3-methylphenyl)-5,6,7,8-tetrahydropyrido[4′,3′:4,5]thieno[2,3-d]pyrimidin-4-amine (104-7)

To a solution of 104-6 (141 mg, 0.26 mmol) in DCM (8 mL) was added TFA (3 ml). The mixture was allowed to stir at ambient temperature for 1 h after which the mixture was concentrated and the resultant residue dissolved in DCM, the pH adjusted to 10 with sodium carbonate solution, then extracted with DCM. The combined organic layers were dried over anhydrous sodium sulfate, filtered and concentrated to afford the title compound as yellow solid (113 mg, 99%). LCMS m/z [M+H]⁺: 431.5.

### Step 8: (E)-1-(4-((4-(Benzo[c][1,2,5]oxadiazol-5-yloxy)-3-methylphenyl)amino)-5,6-dihydropyrido[4′,3′:4,5]thieno[2,3-d]pyrimidin-7(8H)-yl)-4-(dimethylamino)but-2-en-1-one (Compound 104)

A suspension of 104-7 (110 mg, 0.26 mmol), (E)-4-(dimethylamino)but-2-enoic acid hydrochloride (84 mg, 0.51 mmol), HATU (243 mg, 0.64 mmol), and DIPEA (99 mg, 0.77 mmol) in DCM (10 mL) was allowed to stir at 20° C. overnight. The mixture was concentrated and the resultant residue was purified by prep-TLC to afford the title compound as light yellow solid (20 mg, 14%). LCMS m/z [M+H]⁺: 542.5. ¹H NMR (400 MHz, DMSO d₆): δ 8.46 (s, 1H), 8.32 (s, 1H), 8.15 (d, 1H), 7.67-6.90 (m, 2H), 7.57 (m, 1H), 7.20 (d, 1H), 6.72-6.87 (m, 3H), 4.93 (d, 2H), 3.93 (d, 2H), 3.27 (br, 4H), 2.32 (s, 6H), 2.18 (s, 3H).

### Example 27: (R)-4-(dimethylamino)-1-(5-methyl-4-((3-methyl-4-((6-methylpyridin-3-yl)oxy)phenyl)amino)-5,6-dihydropyrido[4′,3′:4,5]thieno[2,3-d]pyrimidin-7(8H)-yl)but-2-en-1-one and (S)-4-(dimethylamino)-1-(5-methyl-4-((3-methyl-4-((6-methylpyridin-3-yl)oxy)phenyl)amino)-5,6-dihydropyrido[4′,3′:4,5]thieno[2,3-d]pyrimidin-7(8H)-yl)but-2-en-1-one

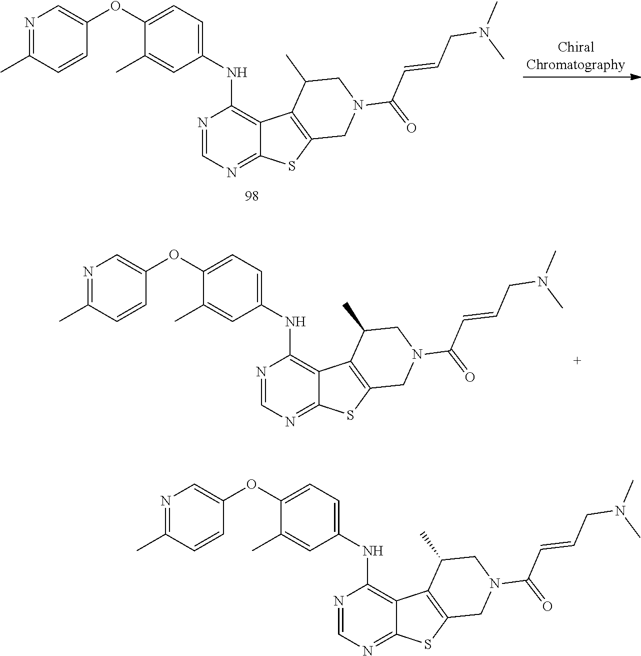

Enantiomers of Compound 98 were separated via HPLC using a Lux Cellulose-4 column. The faster eluting enantiomer was designated Compound 106 ([α]_{D}¹¹⁰=+109.09° (C=0.22 g/100 mL MeOH)) and slower eluting enantiomer designated Compound 105 ([α]_{D}¹¹⁰=−134.29° (C=0.21 g/100 mL MeOH)).

### Example 28: (E)-1-(4-((4-(benzo[d]thiazol-5-yloxy)-3-methylphenyl)amino)-5,8-dihydropyrido[4′,3′:4,5]thieno[2,3-d]pyrimidin-7(6H)-yl)-4-(dimethylamino)but-2-en-1-one (Compound 107)

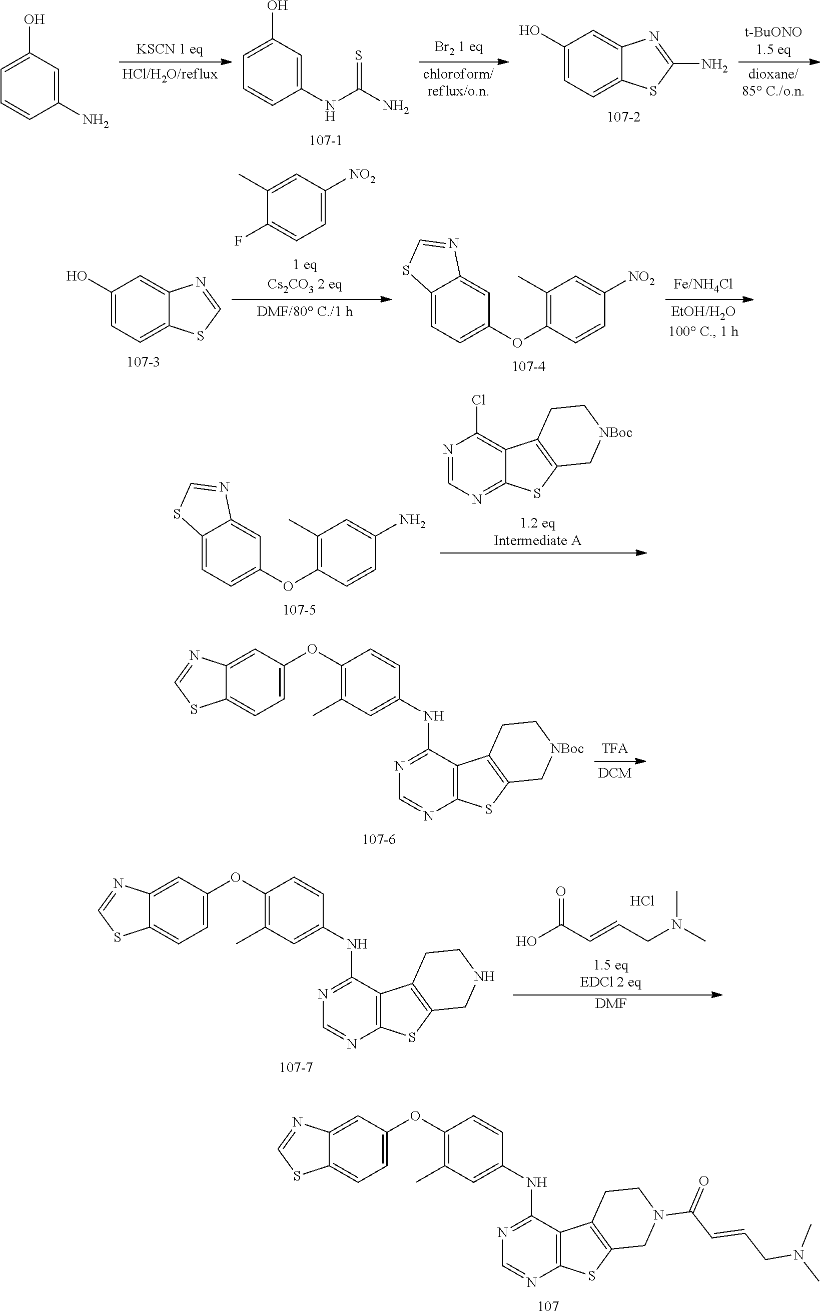

### Step 1: 1-(3-hydroxyphenyl)thiourea (107-1)

A mixture of 3-aminophenol (5.0 g, 45.8 mmol), HCl (conc., 4.5 mL) and water (12.5 mL) was heated at reflux for 0.5 h. The reaction mixture was cooled at ambient temperature and KSCN (8.9 g, 92 mmol) was added after which it was heated to reflux for 4 h. The resultant solution was poured onto crushed ice and filtered. The solid was collected, dried and re-crystallized from ethanol to afford the title compound as white crystals (2.3 g, 30%). LCMS m/z [M+H]⁺: 169.0.

### Step 2: 2-aminobenzo[d]thiazol-5-ol (107-2)

107-1 (2.3 g, 13.75 mmol) was dissolved in chloroform (30 mL). A solution of Br₂ (2.2 g, 13.8 mmol) in chloroform was added dropwise while keeping reaction mixture temperature below 5° C. The reaction was heated at reflux for 4 h until no HBr was generated after which it was taken up in EtOAc and the organic layer separated, washed with water, dried over sodium sulfate and purified through column chromatography to afford the title compound as yellow solid (1.08 g, 47%). LCMS m/z [M+H]⁺: 167.0.

### Step 3: benzo[d]thiazol-5-ol (107-3)

To a mixture of 107-2 (1.08 g, 6.5 mmol) in 1, 4-dioxane (15 mL) was added t-BuONO (1.01 g, 9.75 mmol). The reaction mixture was stirred at 85° C. for 2 h after which it was diluted with water and extracted with ethyl acetate. The combined organic layers were washed with water and brine, dried over sodium sulfate and concentrated in vacuo. The resultant residue was purified by column chromatography (hexanes/EtOAc: 5/1) to afford the title compound as yellow solid (500 mg, 51% yield). LCMS m/z [M+H]⁺: 152.0.

### Step 4: 5-(2-methyl-4-nitrophenoxy)benzo[d]thiazole (107-4)

Cs₂CO₃ (1.16 g, 3.57 mmol), 1-fluoro-2-methyl-4-nitro-benzene (277 mg, 1.79 mmol) was added to the solution of 107-3 (270 mg, 1.79 mmol) in DMF (10 mL). The resulted mixture was stirred at 80° C. for 2 h. The reaction mixture was diluted with water and extracted with ethyl acetate. The combined organic layers were washed with water and brine, dried over sodium sulfate and concentrated in vacuo to afford the title compound as yellow solid (200 mg, 39%). LCMS m/z [M+H]⁺: 287.1.

### Step 5: 4-(benzo[d]thiazol-5-yloxy)-3-methylaniline (107-5)

Fe (196 mg, 3.49 mmol), NH₄Cl (188 mg, 3.49 mmol) was added to a solution of 107-4 (200 mg, 0.7 mmol) in ethanol (10 mL) and H₂O (5 mL). The resultant mixture was heated to reflux for 2 h after which it was filtered through Celite and concentrated. The resultant residue was taken up in DCM and washed with an aqueous solution of saturated sodium bicarbonate. The organic layer was separated, dried over anhydrous sodium sulfate, filtered and concentrated to afford the title compound as yellow solid (170 mg, 95%). LCMS m/z [M+H]⁺: 257.4. Step 6: tert-butyl 4-((4-(benzo[d]thiazol-5-yloxy)-3-methylphenyl)amino)-5,8-dihydropyrido[4′,3′:4,5]thieno[2,3-d]pyrimidine-7(6H)-carboxylate (107-6)

Pd₂(dba)₃ (61 mg, 0.07 mmol) and Davophos (52 mg, 0.13 mmol) were added to a solution of 107-5 (170 mg, 0.66 mmol), Intermediate A (259 mg, 0.8 mmol), and Na₂CO₃ (211 mg, 1.99 mmol) in tert-amyl alcohol (10 mL). The resultant mixture was stirred at 100° C. for 3 h under nitrogen atmosphere after which it was diluted with water and extracted with ethyl acetate. The combined organic layers were washed with water and brine, dried over sodium sulfate and concentrated in vacuo. The resultant residue was purified by column chromatography (hexanes/EtOAc: 1/2) to afford the title compound as yellow solid (280 mg, 77%). LCMS m/z [M+H]⁺: 546.5.

### Step 7: N-(4-(benzo[d]thiazol-5-yloxy)-3-methylphenyl)-5,6,7,8-tetrahydropyrido[4′,3′:4,5]thieno[2,3-d]pyrimidin-4-amine (107-7)

TFA (3 mL) was added to the solution of 107-6 (200 mg, 0.37 mmol) in DCM (5 mL). The resultant mixture was stirred at ambient temperature for 0.5 h. The reaction mixture was concentrated, diluted with water and the pH adjusted to >7 with saturated NaHCO₃ solution. The mixture was extracted with ethyl acetate and the combined organic layers were washed with water and brine, dried over sodium sulfate and concentrated in vacuo to afford the title compound (123 mg, 75%).

### Step 8: (E)-1-(4-((4-(Benzo[d]thiazol-5-yloxy)-3-methylphenyl)amino)-5,6-dihydropyrido[4′,3′:4,5]thieno[2,3-d]pyrimidin-7(8H)-yl)-3-(dimethylamino)prop-2-en-1-one (Compound 107)

To a solution of 107-7 (123 mg, 0.27 mmol), (E)-4-(dimethylamino)but-2-enoic acid hydrochloride (68.3 mg, 0.41 mmol) in DMF (5 mL) was added EDCI (105 mg, 0.55 mmol). The resulted mixture was stirred at ambient temperature for 1 h. TLC showed the reaction completed. The reaction mixture was diluted with water and extracted with ethyl acetate. The combined organic layers were washed with water and brine, dried over sodium sulfate and concentrated in vacuo. The resultant residue was purified by prep-TLC (eluted with 4% MeOH in DCM) to afford the title compound as light yellow solid. LCMS m/z [M+H]⁺: 557.5; ¹H NMR (400 MHz, DMSO-d₆): δ 9.40 (s, 1H), 8.43 (s, 1H), 8.24 (s, 1H), 8.15 (d, 1H), 7.60-7.56 (m, 2H), 7.40 (d, 1H), 7.20 (dd, 1H), 7.03 (d, 1H), 6.79-6.71 (m, 2H), 4.92 (d, 2H), 3.95-3.89 (m, 2H), 3.28-3.24 (m, 2H), 3.10-3.06 (m, 2H), 2.19 (s, 9H).

### Example 29: (E)-4-(Dimethylamino)-1-(4-((3-methyl-4-((6-methylpyridin-3-yl)oxy)phenyl)amino)-7,8-dihydropyrido[3′,4′:4,5]thieno[2,3-d]pyrimidin-6(5H)-yl)but-2-en-1-one (Compound 108)

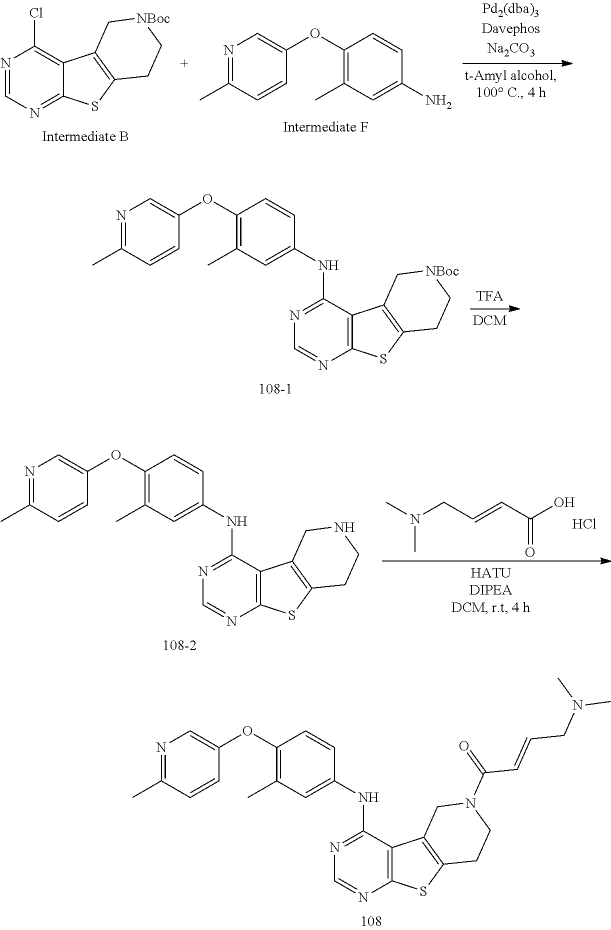

The title compound was prepared in analogous fashion to Compound 99, but using Intermediate B instead of Intermediate C. The title compound was obtained as off-white solid (8.7 mg). LCMS m/z [M+H]⁺: 515.2. ¹H NMR (400 MHz, CDCl₃): δ 8.47-8.14 (m, 3H), 7.57-7.40 (m, 2H), 7.29-7.14 (m, 2H), 7.01-6.80 (m, 2H), 6.73-6.62 (m, 1H), 5.28-5.06 (m, 2H), 4.05-3.84 (m, 2H), 3.40-3.33 (m, 2H), 3.07-2.91 (m, 2H), 2.44 (s, 3H), 2.20 (s, 3H), 2.29 (dd, 6H).

### Example 30: (E)-4-(dimethylamino)-1-(4-((4-(imidazo[1,2-a]pyridin-6-yloxy)-3-methylphenyl)amino)-5,6-dihydropyrido[4′,3′:4,5]thieno[2,3-d]pyrimidin-7(8H)-yl)but-2-en-1-one (Compound 109)

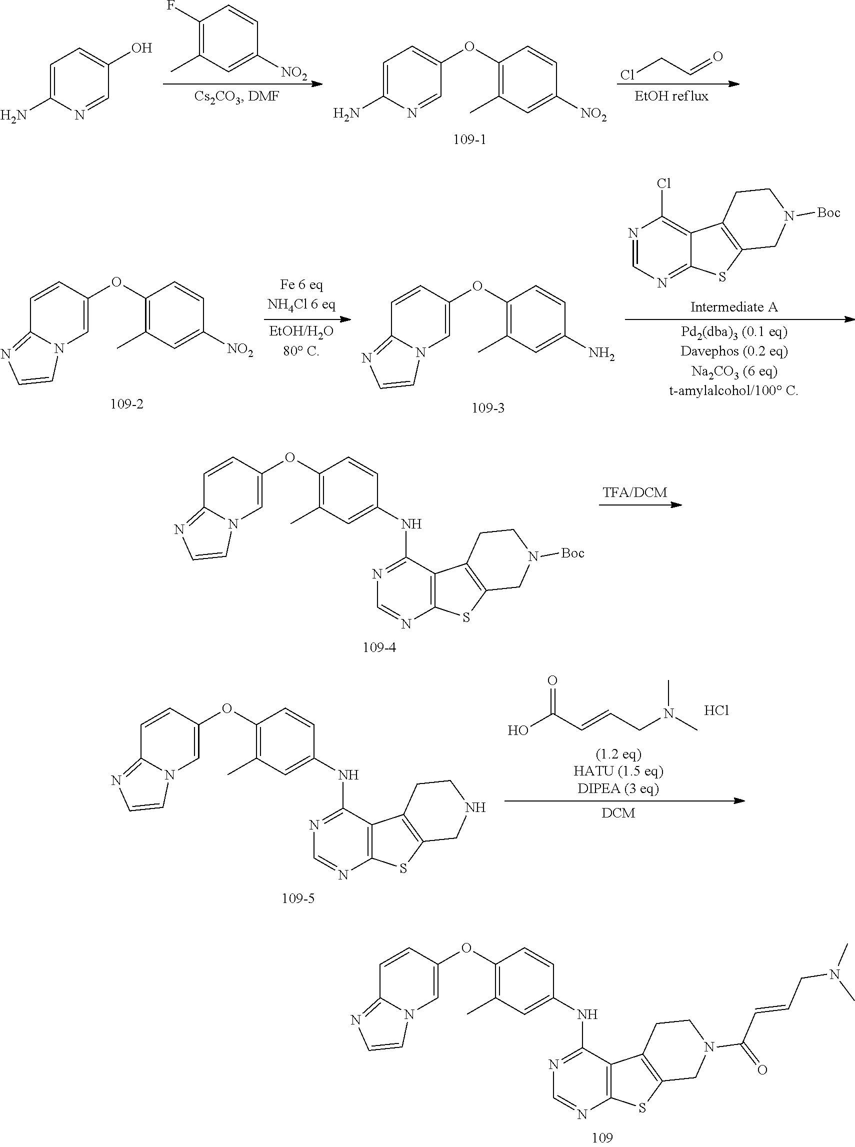

### Step 1: 5-(2-methyl-4-nitrophenoxy)pyridin-2-amine (109-1)

To a solution of 6-aminopyridin-3-ol (4.0 g, 0.04 mol) and 1-fluoro-2-methyl-4-nitrobenzene (3.1 g, 0.04 mol) in DMF (50 mL) was added Cs₂CO₃ (18 g, 0.054 mol). The mixture was allowed to stir at ambient temperature for 3 h, after which it was poured into water and the precipitate collected by filtration to afford the title compound as brown solid (5.0 g, 56%). LCMS m/z [M+H]⁺: 246.1.

### Step 2: 6-(2-methyl-4-nitrophenoxy)imidazo[1,2-a]pyridine (109-2)

A mixture of 109-1 (1.0 g, 4.1 mmol) and 2-chloroacetaldehyde (4 mL) was taken up in ethanol (20 mL). The resulting mixture was allowed to stir at 80° C. for 5 h after which the reaction mixture was concentrated and the resultant residue purified by column chromatography (DCM/MeOH=50/1) to afford the title compound as yellow solid (1.2 g, 99%). LCMS m/z [M+H]⁺: 270.1.

### Step 3: 4-(imidazo[1,2-a]pyridin-6-yloxy)-3-methylaniline (109-3)

A mixture of 109-2 (1.21 g, 4.49 mmol), NH₄Cl (1.46 g, 27 mmol) and Fe (1.51 g, 27 mmol) was taken up in ethanol (30 mL) and water (15 mL). The resulting mixture was allowed to stir at 80° C. for 2 h after which the reaction mixture was filtered and the filtrate was concentrated. The resultant residue was dissolved in DCM/water and extracted with DCM. The combined organic layers were dried over anhydrous Na₂SO₄ and concentrated to afford the title compound as light yellow solid (700 mg, 65%).

### Step 4: tert-butyl 4-((4-(imidazo[1,2-a]pyridin-6-yloxy)-3-methylphenyl)amino)-5,8-dihydropyrido[4′,3′:4,5]thieno[2,3-d]pyrimidine-7(6H)-carboxylate (109-4)

To a solution of 109-3 (200 mg, 0.84 mmol) in tert-amylalcohol (8 mL) was added Intermediate A (225 mg, 0.84 mmol), Pd₂(dba)₃ (76.5 mg, 0.08 mmol), Davephos (66 mg, 0.17 mmol) and Na₂CO₃ (532 mg, 5.0 mmol). The resulting mixture was allowed to stir at 100° C. for 2 h after which it was cooled to ambient temperature, washed with water, extracted with EtOAc, dried and concentrated. The resultant residue was purified by column chromatography eluting with 5% MeOH in DCM to afford the title compound (180 mg). LCMS m/z [M+H]⁺: 529.7.

### Step 5: N-(4-(imidazo[1,2-a]pyridin-6-yloxy)-3-methylphenyl)-5,6,7,8-tetrahydropyrido[4′,3′:4,5]thieno[2,3-d]pyrimidin-4-amine (109-5)

To a solution of 109-4 (180 mg, 0.34 mmol) in DCM (8 mL) was added TFA (3 mL). The resulting mixture was allowed to stir at ambient temperature for 1 h after which it was quenched through addition of saturated aqueous NaHCO₃, extracted with DCM, dried and concentrated. The resultant residue was purified by column chromatography eluting with 10% MeOH in DCM to afford the title compound (90 mg). LCMS m/z [M+H]⁺ 429.1.

### Step 6: (E)-4-(Dimethylamino)-1-(4-((4-(imidazo[1,2-a]pyridin-6-yloxy)-3-methylphenyl)amino)-5,6-dihydropyrido[4′,3′:4,5]thieno[2,3-d]pyrimidin-7(8H)-yl)but-2-en-1-one (Compound 109)

To a solution of 109-5 (90 mg, 0.21 mmol) in DCM (8 mL) was added (E)-4-(dimethylamino)but-2-enoic acid hydrochloride (42 mg, 025 mmol), HATU (120 mg, 0.31 mmol) and DIPEA (81 mg, 0.63 mmol). The resulting mixture was allowed to stir at 25° C. for 2 h after which it was washed with water, extracted with DCM, dried and concentrated. The resultant residue was purified by column chromatography eluting with 10% MeOH in DCM to afford the title compound (40 mg, 35%) as white solid. LCMS m/z [M+H]⁺: 540.3. ¹H NMR (400 MHz, CD₃OD): δ 8.34 (s, 1H), 8.09 (d, 1H), 7.77 (s, 1H), 7.59-7.55 (m, 3H), 7.48 (dd, 1H), 7.23 (dd, 1H), 7.00 (d, 1H), 6.90-6.71 (m, 2H), 4.98 (d, 2H), 4.06 (t, 2H), 3.32-3.25 (m, 2H), 3.23 (d, 2H), 2.32 (d, 9H).

### Example 31: (E)-4-(dimethylamino)-1-(4-((3-methyl-4-((2-methylimidazo[1,2-a]pyridin-7-yl)oxy)phenyl)amino)-5,8-dihydropyrido[4′,3′:4,5]thieno[2,3-d]pyrimidin-7(6H)-yl)but-2-en-1-one (Compound 110)

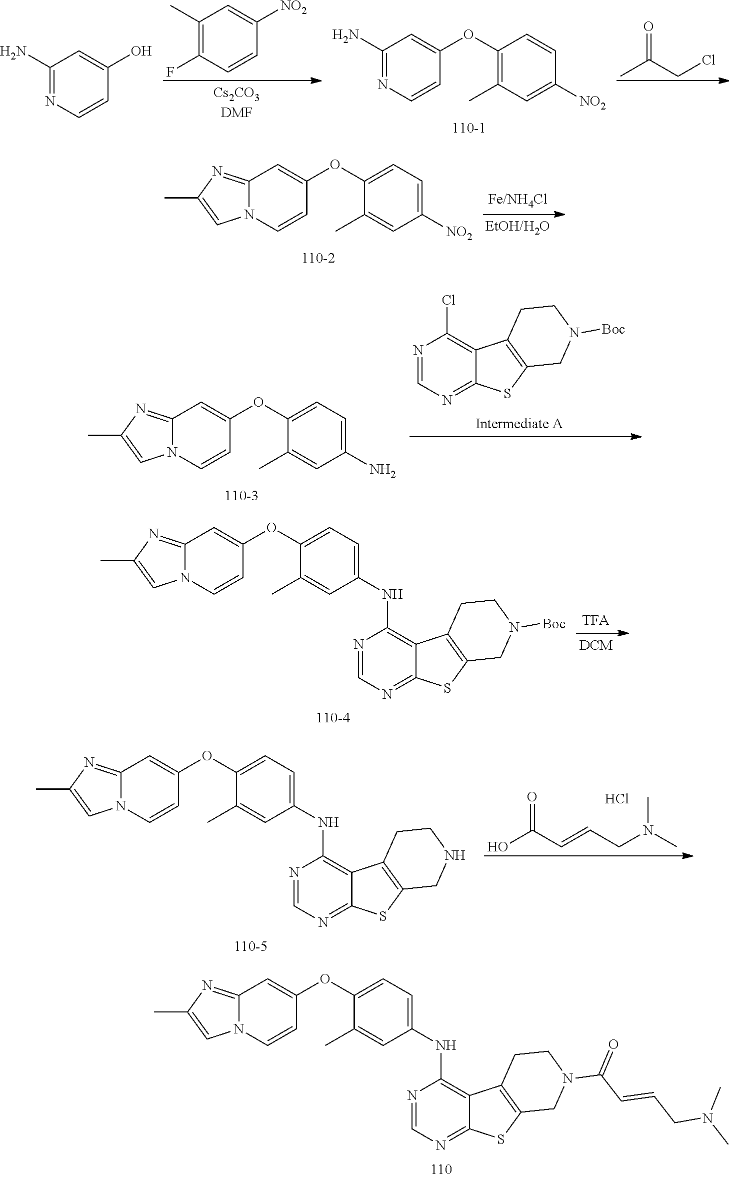

### Step 1: 4-(2-methyl-4-nitrophenoxy)pyridin-2-amine (110-1)

To a solution of 2-aminopyridin-4-ol (2.0 g, 18.2 mmol) and 1-fluoro-2-methyl-4-nitrobenzene (2.82 g, 18.2 mmol) in DMF (40 mL) was added cesium carbonate (7.1 g, 21.8 mmol). The reaction mixture was stirred at ambient temperature overnight. The mixture was diluted with EtOAc, the organic layer was washed with water and dried and evaporated to afford the title compound (3.82 g) as brown solid. LCMS m/z (M+H)*: 246.3.

### Step 2: 2-Methyl-7-(2-methyl-4-nitrophenoxy)imidazo[1,2-a]pyridine (110-2)

A mixture of 110-1 (800 mg, 3.26 mmol), and 1-chloropropan-2-one (150 mg, 0.58 mmol) in DCM (25 mL) and saturated NaHCO₃ (25 mL) was stirred at 45° C. for 2 days. The reaction mixture was extracted with DCM, washed with brine, concentrated and purified through column chromatography (hexanes:EtOAc=3:1) to afford the title compound as yellow solid (700 mg, 76%). LCMS m/z [M+H]⁺: 284.3. ¹H NMR (400 MHz, DMSO-d₆): δ. 8.52 (d, 1H), 8.28 (d, 1H), 8.08 (dd, 1H), 7.65 (s, 1H), 7.09 (d, 1H), 6.69 (d, 1H), 6.74 (dd, 1H), 2.37 (s, 3H), 2.30 (s. 3H).

### Step 3: 3-Methyl-4-((2-methylimidazo[1,2-a]pyridin-7-yl)oxy)aniline (110-3)

A mixture of 110-2 (700 mg, 2.47 mmol), Fe (692 mg, 12.4 mmol) and NH₄Cl (668 mg, 12.4 mmol) in EtOH/H₂O (20 mL/10 mL) was stirred at 80° C. for 2 h. The mixture was filtered and the filtrate was taken up in water and extracted with EtOAc. The organic layers were dried over anhydrous Na₂SO₄ and evaporated to afford the title compound as yellow solid (510 mg, 82%). LCMS m/z [M+H]⁺: 254.1; ¹H NMR (400 MHz, DMSO-d₆): δ. 8.35 (d, 1H), 7.49 (s, 1H), 6.75 (d, 1H), 6.62 (dd, 1H), 6.52 (d, 1H), 6.46 (dd, 1H), 6.27 (d, 1H), 5.01 (s, 2H), 2.23 (s, 3H), 1.98 (s. 3H).

### Step 4: tert-Butyl 4-((3-methyl-4-((2-methylimidazo[1,2-a]pyridin-7-yl)oxy)phenyl)amino)-5,6-dihydropyrido[4′,3′:4,5]thieno[2,3-d]pyrimidine-7(8H)-carboxylate (110-4)

A mixture of 110-3 (185 mg, 0.73 mmol), Intermediate A (285 mg, 0.87 mmol), DavePhos (114 mg, 0.3 mmol), Pd₂(dba)₃ (113 mg, 0.2 mmol) and Na₂CO₃ (541 mg, 5.1 mmol) in t-AmOH (15 mL) was stirred at 100° C. for 2 h under nitrogen atmosphere. The reaction mixture was quenched with water, extracted with EtOAc, washed with brine and concentrated. The resultant residue was purified through column chromatography (DCM:MeOH=50:1) to afford the title compound as white solid (110 mg, 28%). LCMS m/z [M+H]⁺: 543.2; ¹H NMR (400 MHz, DMSO-d₆): δ. 8.42-8.43 (m, 2H), 8.23 (s, 1H), 7.60-7.62 (m, 2H), 7.55 (s, 1H), 7.09 (d, 1H), 6.70 (dd, 1H), 6.45 (d, 1H), 4.69 (s, 2H), 3.72 (t, 2H), 3.24 (t, 2H), 2.25 (s, 3H), 2.17 (s, 3H), 1.46 (s, 9H).

### Step 5: N-(3-Methyl-4-((2-methylimidazo[1,2-a]pyridin-7-yl)oxy)phenyl)-5,6,7,8-tetrahydropyrido[4′,3′:4,5]thieno[2,3-d]pyrimidin-4-amine (110-5)

TFA (3 mL) was added to a solution of 110-4 (110 mg, 0.20 mmol) in DCM (3 mL). The resultant mixture was stirred at ambient temperature for 1 h then concentrated. The resultant residue was dissolved in DCM and the organic later washed by aqueous Na₂CO₃ and brine then concentrated to afford the title compound. LCMS m/z [M+H]⁺: 443.3.

### Step 6: (E)-4-(Dimethylamino)-1-(4-((3-methyl-4-((2-methylimidazo[1,2-a]pyridin-7-yl)oxy)phenyl)amino)-5,6-dihydropyrido[4′,3′:4,5]thieno[2,3-d]pyrimidin-7(8H)-yl)but-2-en-1-one (Compound 110)

A mixture of 110-5 (72 mg, 0.20 mmol), (E)-4-(dimethylamino)but-2-enoic acid hydrochloride (50 mg, 0.3 mmol), HATU (152 mg, 0.40 mmol) and DIPFA (65 mg, 0.5 mmol) in DCM (5 mL) was stirred at ambient temperature for 1 h after which water was added and the organic layer separated, washed with brine, dried over anhydrous Na₂SO₄ and evaporated to dryness. The resultant residue was purified through prep-HPLC to afford the title compound as white solid (23.3 mg, 21%). LCMS m/z [M+H]⁺:554.2; ¹H NMR (400 MHz, CD₃OD): δ 8.66 (d, 1H), 8.40 (s, 1H), 7.80 (s, 1H), 7.63-7.68 (m, 2H), 7.28 (dd, 1H), 7.22 (d, 1H), 7.03-7.15 (m, 1H), 6.94 (d, 1H), 6.76-6.85 (m, 1H), 5.02 (d, 2H), 3.99-4.11 (m, 4H), 3.35-3.41 (m, 2H), 2.95 and 2.96 (s, 6H), 2.49 (d, 3H), 2.25 (s, 3H).

### Example 32: (E)-1-(4-((4-([1,2,4]Triazolo[1,5-a]pyridin-6-yloxy)-3-methylphenyl)amino)-5H-pyrrolo[3′,4′:4,5]thieno[2,3-d]pyrimidin-6(7H)-yl)-4-(dimethylamino)but-2-en-1-one (Compound 111)

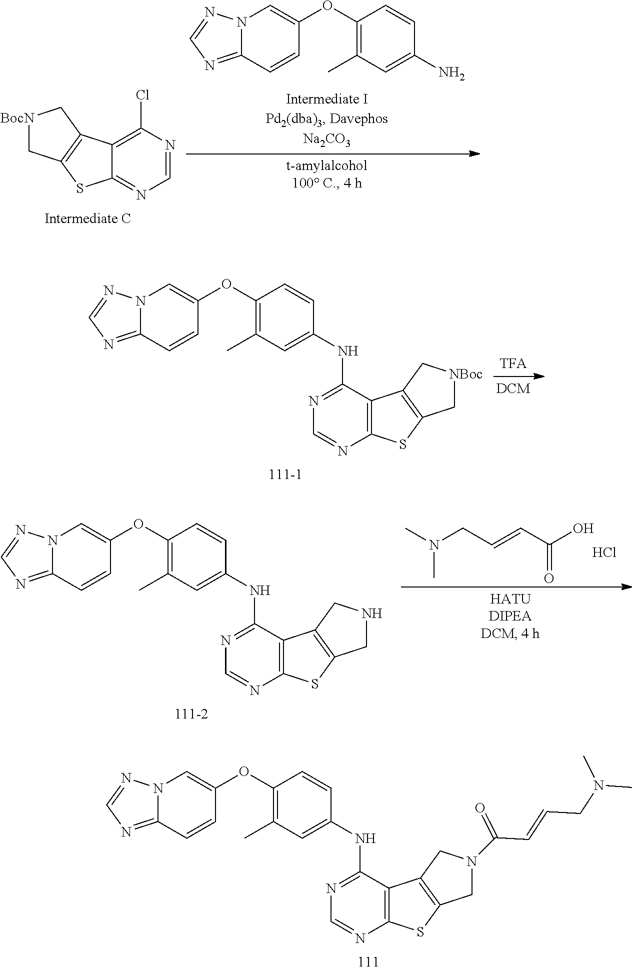

The title compound was prepared in analogous fashion to Compound 99, using Intermediate I instead of Intermediate F. The title compound was obtained as off-white solid (26 mg). LCMS m/z [M+H]⁺: 527.5. ¹H NMR (400 MHz, CDCl₃): δ 8.65 (dd, 1.5H), 8.50-8.41 (m, 2.5H), 7.90 (d, 1H), 7.57 (d, 2H), 7.51-7.45 (m, 1H), 7.05 (d, 1H), 6.84-6.73 (m, 1H), 6.57-6.47 (m, 1H), 5.26 (s, 1H), 5.11-5.00 (m, 2H), 4.82 (s, 1H), 3.19 (s, 2H), 2.27 (q, 9H).

### Example 33: (E)-4-(dimethylamino)-1-(4-((4-(imidazo[1,2-a]pyridin-7-yloxy)-3-methylphenyl)amino)-5,8-dihydropyrido[4′,3′:4,5]thieno[2,3-d]pyrimidin-7(6H)-yl)but-2-en-1-one (Compound 126)

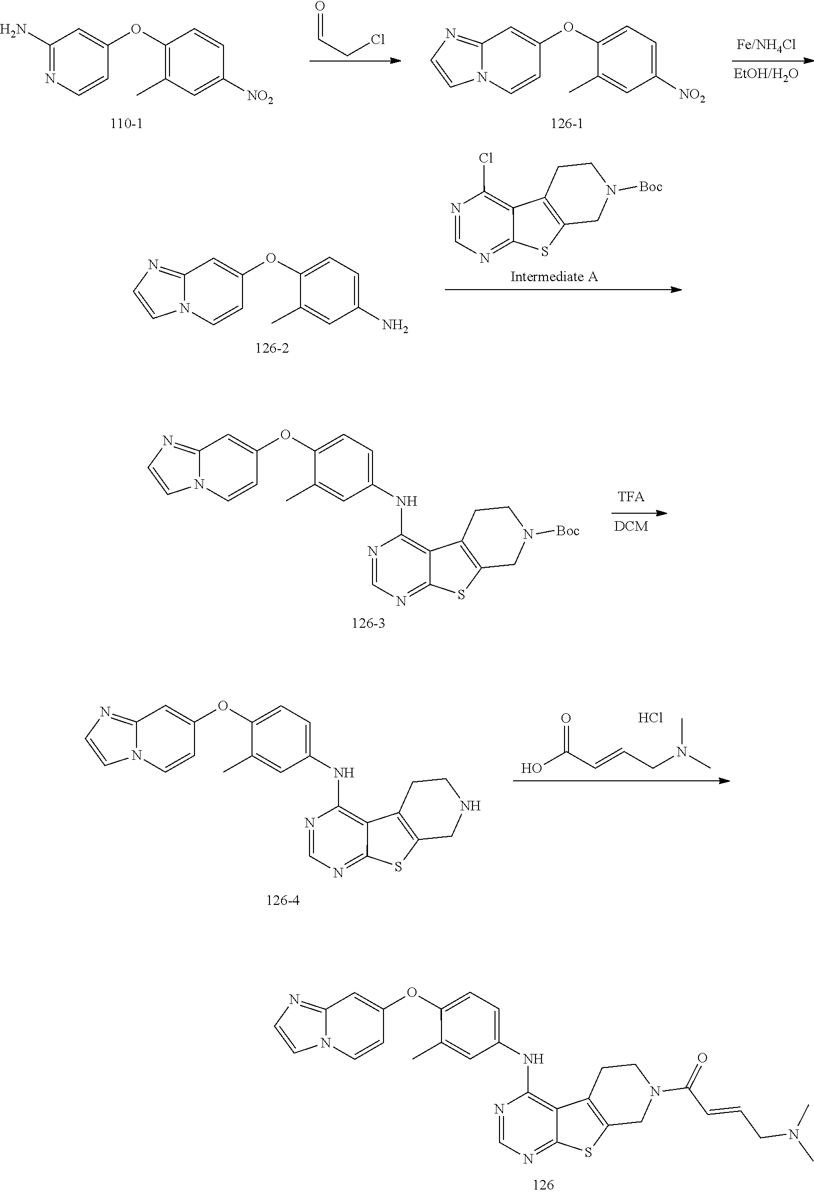

### Step 1: 7-(2-methyl-4-nitrophenoxy)imidazo[1,2-a]pyridine (126-1)

To a solution of 110-1 (3.82 g, 15.6 mmol) in ethanol (25 mL) was added chloroacetaldehyde (1.83 g, 23.3 mmol). The mixture was heated to 85° C. for 2 h after which it was evaporated and the resultant residue was extracted with EtOAc, washed with water, dried and evaporated to afford the title compound (2.15 g) as black oil. LCMS m/z [M+H⁺]⁺: 270.3.

### Step 2: 4-(Imidazo[1,2-a]pyridin-7-yloxy)-3-methylaniline (126-2)

To a solution of 126-1 (2.15 g, 8 mmol) in ethanol (20 mL) was added Fe (2.23 g, 40 mmol), ammonium chloride (2.14 g, 40 mmol) and water (5 mL). The mixture was heated to 80° C. for 1.5 h after which it was diluted with DCM, the organic layer was washed with water, dried and evaporated to afford the title compound (1.05 g).

### Step 3: tert-butyl 4-((4-(imidazo[1,2-a]pyridin-7-yloxy)-3-methylphenyl)amino)-5,8-dihydropyrido[4′,3′:4,5]thieno[2,3-d]pyrimidine-7(6H)-carboxylate (126-3)

To a solution of 126-2 (300 mg, 1.25 mmol), Intermediate A (409 mg, 1.26 mmol) in t-amylalcohol (6 mL) was added Pd₂(dba)₃ (115 mg, 0.13 mmol), Davephos (99 mg, 0.25 mmol) and sodium carbonate (930 mg, 8.8 mmol). The resulting mixture was heated to 100° C. for 3 h under nitrogen atmosphere after which it was evaporated and the resultant residue purified by column chromatography (DCM:MeOH=20:1) to afford the title compound (250 mg) as yellow solid. LCMS m/z [M+H]⁺:529.6.

### Step 4: N-(4-(imidazo[1,2-a]pyridin-7-yloxy)-3-methylphenyl)-5,6,7,8-tetrahydropyrido[4′,3′:4,5]thieno[2,3-d]pyrimidin-4-amine (126-4)

To a solution of 126-3 (250 mg, 0.47 mmol) in DCM (3 mL) was added TFA (3 mL). The resulting mixture was stirred at ambient temperature for 0.5 h after which it was quenched with saturated NaHCO₃, extracted with DCM, dried and evaporated to afford the title compound (140 mg). LCMS m/z [M+H]⁺: 429.2.

### Step 5: (E)-4-(Dimethylamino)-1-(4-((4-(imidazo[1,2-a]pyridin-7-yloxy)-3-methylphenyl)amino)-5,6-dihydropyrido[4′,3′:4,5]thieno[2,3-d]pyrimidin-7(8H)-yl)but-2-en-1-one (Compound 126)

To a solution of compound 126-4 (140 mg, 0.33 mmol) and (E)-4-(dimethylamino)but-2-enoic acid hydrochloride (65 mg, 0.39 mmol) in DCM (5 mL) was added EDCI (94 mg, 0.49 mmol). The resulting mixture was stirred at ambient temperature overnight. The reaction mixture was quenched with saturated NaHCO₃ extracted with DCM, dried and evaporated to dryness. The resultant residue was purified through prep-TLC (DCM:MeOH=20:1) to afford the title compound (16.5 mg). LCMS m/z [M+H]⁺: 540.2. ¹HNMR (400 MHz, CDCl₃) δ 8.54 (s, 1H), 8.05 (d, 1H), 7.55-7.47 (m, 4H), 7.06 (d, 1H), 6.90-6.98 (m, 2H), 6.76 (s, 1H), 6.71 (d, 1H), 6.48-6.68 (m, 1H), 4.80-4.99 (m, 2H), 3.95-4.13 (m, 2H), 3.08-3.36 (m, 4H), 2.19-2.45 (m, 9H).

### Example 34: (E)-4-(dimethylamino)-1-(4-((3-methyl-4-((2-methyl-[1,2,4]triazolo[1,5-a]pyridin-7-yl)oxy)phenyl)amino)-5,6-dihydropyrido[4′,3′:4,5]thieno[2,3-d]pyrimidin-7(8H)-yl)but-2-en-1-one (Compound 127)

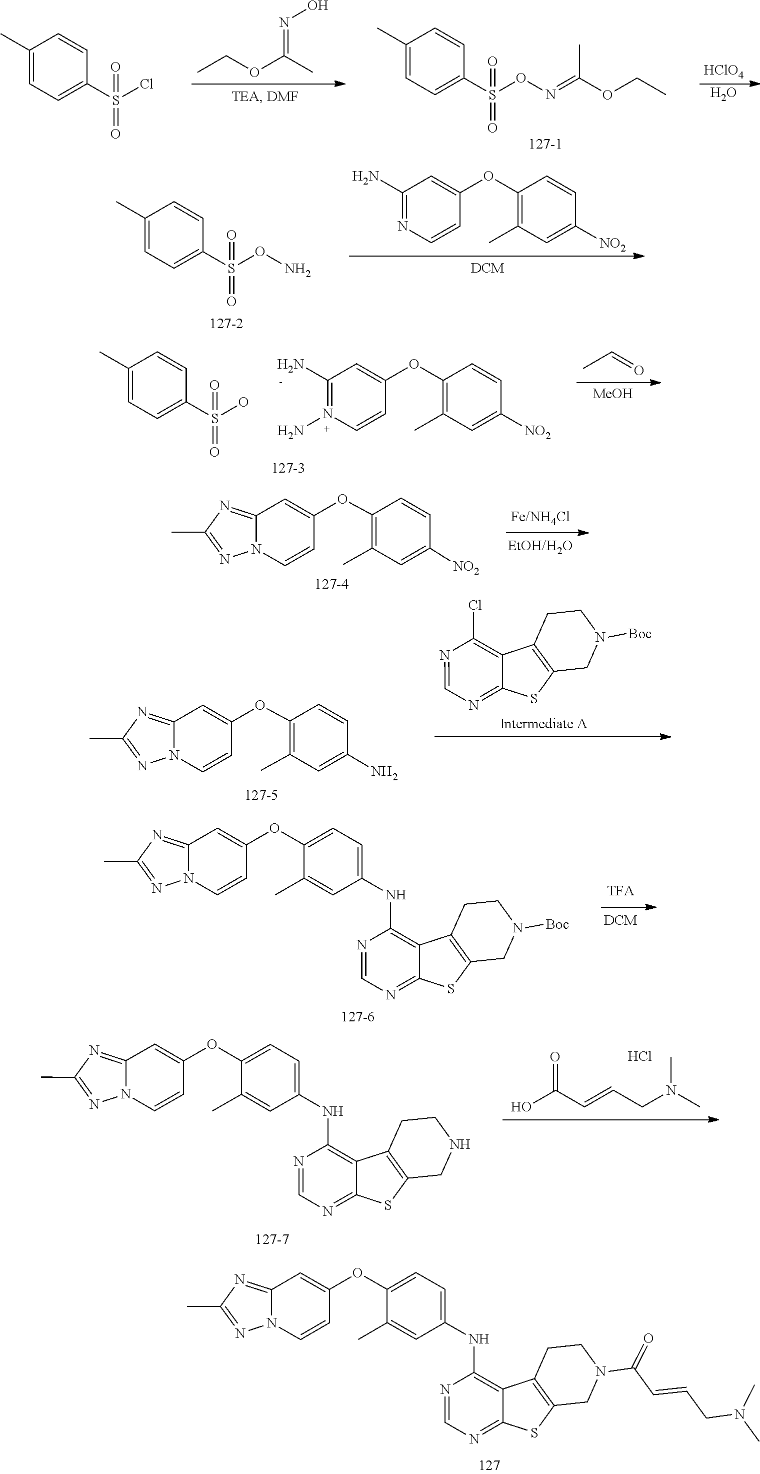

### Step 1: (E)-Ethyl N-tosyloxyacetimidate (127-1)

A solution of (E)-ethyl N-hydroxyacetimidate (1.3 g, 12 mmol) in DMF (2 mL) was added dropwise into a solution of 4-methylbenzene-1-sulfonyl chloride (2.0 g, 11 mmol) and TEA (1.6 g, 16 mmol) in DMF at 0° C. The resulting mixture was allowed to stir at 0° C. for 0.5 h then ambient temperature for 1 h. To the reaction mixture was added water (80 mL) with stirring for an additional 10 min. The resulting suspension was filtered to afford the title compound as white solid (1.2 g, 44%). LCMS m/z [M+H]⁺: 258.2.

### Step 2: Tosylhydroxylamine (127-2)

A mixture of 127-1 (1.2 g, 4.66 mmol) and HClO₄ (70%, 10 mL) in water (40 mL) was allowed to stir at ambient temperature for 10 min. To the reaction mixture was added water (100 mL) and continued to stir for 30 min. The solid was collected by filtration to afford the title compound as white solid (450 mg, 52%).

### Step 3: (127-3)

A mixture of 127-2 (450 mg, 2.4 mmol) and 4-(2-methyl-4-nitrophenoxy)pyridin-2-amine (530 mg, 2.2 mmol) in DCM (40 mL) was allowed to stir at ambient temperature for 3 min. The solid was collected by filtration to afford the title compound as white solid (900 mg, 95%).

### Step 4: 2-Methyl-7-(2-methyl-4-nitrophenoxy)-[1,2,4]triazolo[1,5-a]pyridine (127-4)

KOH (1.16 g, 20.8 mmol) in water (10 mL) was added into a solution of 127-3 (900 mg, 2.08 mmol) and acetaldehyde (115 mg, 2.6 mmol) in MeOH. The resulting mixture was allowed to stir at ambient temperature for 30 min after which it was extracted with DCM, washed with brine, and concentrated. The resultant residue was purified by column chromatography (hexanes:EtOAc=3:1) to afford the title compound as yellow solid (270 mg, 46%). LCMS m/z [M+H]⁺: 285.3. ¹H NMR (400 MHz, CDCl₃): δ. 8.45 (d, 1H), 8.22 (d, 1H), 8.11 (dd, 1H), 7.08 (d, 1H), 6.91 (d, 1H), 6.76 (dd, 1H), 2.56 (s, 3H), 2.37 (s, 3H).

### Step 5: 3-Methyl-4-((2-methyl-[1,2,4]triazolo[1,5-a]pyridin-7-yl)oxy)aniline (127-5)

A mixture of 127-4 (270 mg, 0.95 mmol), Fe (266 mg, 4.75 mmol) and NH₄Cl (255 mg, 4.75 mmol) in EtOH/H₂O (20 mL/10 mL) was allowed to stir at 80° C. for 2 h. The mixture was filtered and the filtrate diluted with water, extracted with EtOAc and the organic layers dried over anhydrous Na₂SO₄ and concentrated to afford the title compound as white solid (180 mg, 75%).

### Step 6: tert-Butyl 4-((3-methyl-4-((2-methyl-[1,2,4]triazolo[1,5-a]pyridin-7-yl)oxy)phenyl)amino)-7,8-dihydropyrido[4,3-d]pyrimidine-6(5H)-carboxylate (127-6)

A mixture of 127-5 (160 mg, 0.63 mmol), Intermediate A (248 mg, 0.76 mmol), DavePhos (12 mg, 0.032 mmol), Pd₂(dba)₃ (30 mg, 0.032 mmol) and Cs₂CO₃ (410 mg, 1.26 mmol) in t-amylalcohol (15 mL) was allowed to stir at 100° C. for 5 h under nitrogen atmosphere. The reaction mixture was quenched with water, extracted with EtOAc, washed with brine, and concentrated. The resultant residue was purified by column chromatography (DCM:MeOH=50:1) to afford the title compound as white solid (150 mg, 44%). LCMS m/z [M+H]⁺: 544.5. ¹H NMR (400 MHz, DMSO-d₆): δ. 8.85 (d, 1H), 8.51 (s, 1H), 8.33 (s, 1H), 7.71-7.72 (m, 2H), 7.22 (d, 1H), 6.96 (dd, 1H), 6.72 (d, 1H), 4.75 (s, 2H), 3.77 (t, 2H), 3.31 (m, 2H), 2.46 (s, 3H), 2.23 (s, 3H), 1.52 (s, 9H).

### Step 7: N-(3-Methyl-4-((2-methyl-[1,2,4]triazolo[1,5-a]pyridin-7-yl)oxy)phenyl)-5,6,7,8-tetrahydropyrido[4,3-d]pyrimidin-4-amine (127-7)

TFA (3 mL) was added to a solution of 127-6 (120 mg, 0.22 mmol) in DCM (3 mL). The mixture was allowed to stir at ambient temperature for 0.5 h. The reaction mixture was concentrated and the resultant residue dissolved in DCM and washed with aqueous Na₂CO₃ then brine. The organic layer was concentrated to afford the title compound which was used directly in the next step. LCMS m/z [M+H]⁺: 444.3.

### Step 8: (E)-4-(dimethylamino)-1-(4-((3-methyl-4-((2-methyl-[1,2,4]triazolo[1,5-a]pyridin-7-yl)oxy)phenyl)amino)-5,6-dihydropyrido[4′,3′:4,5]thieno[2,3-d]pyrimidin-7(8H)-yl)but-2-en-1-one (Compound 127)

A mixture of 127-7 (90 mg, 0.21 mmol), (E)-4-(dimethylamino)but-2-enoic acid hydrochloride (38 mg, 0.23 mmol), HATU (160 mg, 0.42 mmol) and DIPFA (62 mg, 0.53 mmol) in DCM (5 mL) was allowed to stir at ambient temperature for 1 h. The reaction mixture was quenched with water and the organic layer was separated, washed with brine, dried over anhydrous Na₂SO₄ and evaporated to dryness. The resultant residue was purified by column chromatography (DCM:MeOH=15:1) to afford the title compound as white solid (31.6 mg, 27%). LCMS m/z [M+H]⁺: 555.2. ¹H NMR (400 MHz, DMSO-d₆): δ 8.54 (s, 1H), 8.36 (d, 1H), 7.58-7.65 (m, 2H), 7.71-7.72 (m, 2H), 7.06 (d, 1H), 6.89 (m, 1H), 6.76-6.77 (m, 2H), 4.95 (s, 2H), 4.11 (t, 2H), 3.38-3.57 (m, 5H), 2.69 (s, 3H), 2.48-2.53 (s, 5H), 2.23 (s, 3H).

### Example 35: (E)-4-(dimethylamino)-1-(4-((4-((6-methoxypyridin-3-yl)oxy)-3-methylphenyl)amino)-5,7-dihydro-6H-pyrrolo[3′,4′:4,5]thieno[2,3-d]pyrimidin-6-yl)but-2-en-1-one (Compound 128)

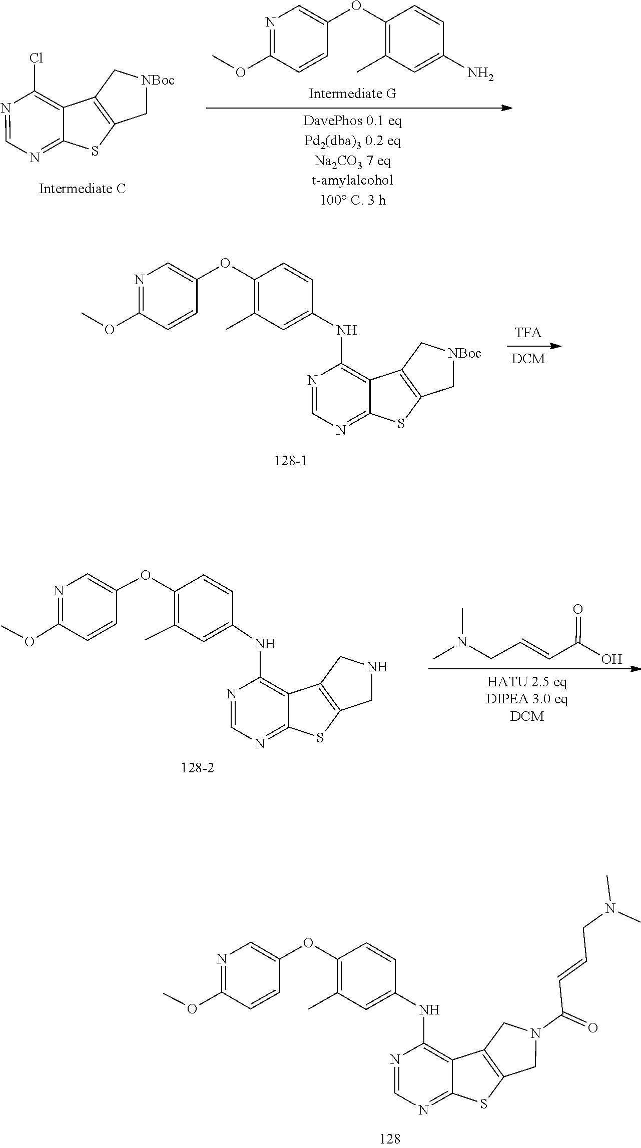

The title compound was prepared in analogous fashion to Compound 99, using Intermediate G instead of Intermediate F. The title compound was obtained as light yellow solid (38 mg). LCMS m/z [M+H]⁺: 517.5. ¹H NMR (400 MHz, DMSO-d₆): δ 8.52 (d, 1H), 8.40 (d, 1H), 7.92 (t, 1H), 7.50 (t, 1H), 7.38-7.44 (m, 2H), 6.82-6.88 (m, 2H), 6.73-6.80 (m, 1H), 6.48 (q, 1H), 5.21 (t, 1H), 5.03 (q, 2H), 4.80 (t, 1H), 3.84 (s, 3H), 3.09 (t, 2H), 2.25 (d, 3H), 2.19 (d, 6H).

### Example 36: (E)-1-(4-((3-Chloro-4-((6-methylpyridin-3-yl)oxy)phenyl)amino)-5H-pyrrolo[3′,4′:4,5]thieno[2,3-d]pyrimidin-6(7H)-yl)-4-(dimethylamino)but-2-en-1-one (Compound 129)

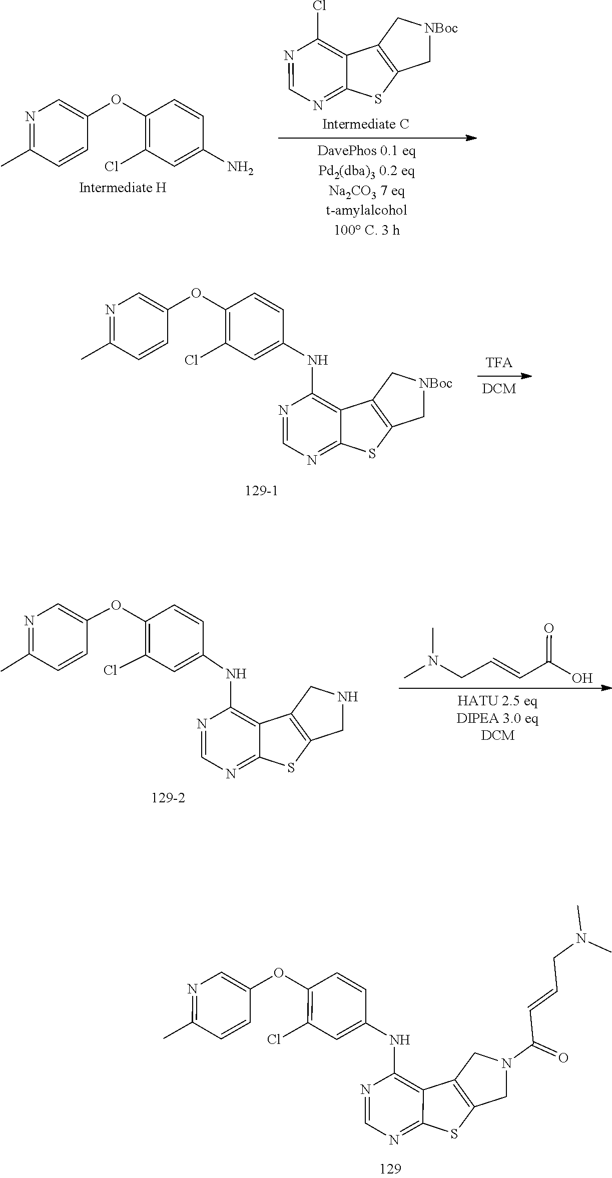

The title compound was prepared in analogous fashion to Compound 99, using Intermediate H instead of Intermediate F. The title compound was obtained as light yellow solid (29 mg). LCMS m/z [M+H]⁺: 521.2. ¹H NMR (400 MHz, DMSO-d₆): δ 8.64 (d, 1H), 8.49 (s, 1H), 8.21 (t, 1H), 7.94 (d, 1H), 7.63-7.69 (m, 1H), 7.27 (d, 2H), 7.20 (q, 1H), 6.74-6.83 (m, 1H), 6.50 (q, 1H), 5.27 (s, 1H), 5.07 (s, 2H), 4.82 (s, 1H), 3.11 (t, 2H), 2.46 (s, 3H), 2.20 (d, 6H).

### Example 37: (E)-1-(4-((4-([1,2,4]Triazolo[1,5-a]pyridin-7-yloxy)-3-methylphenyl)amino)-5H-pyrrolo[3′,4′:4,5]thieno[2,3-d]pyrimidin-6(7H)-yl)-4-(dimethylamino)but-2-en-1-one (Compound 130)

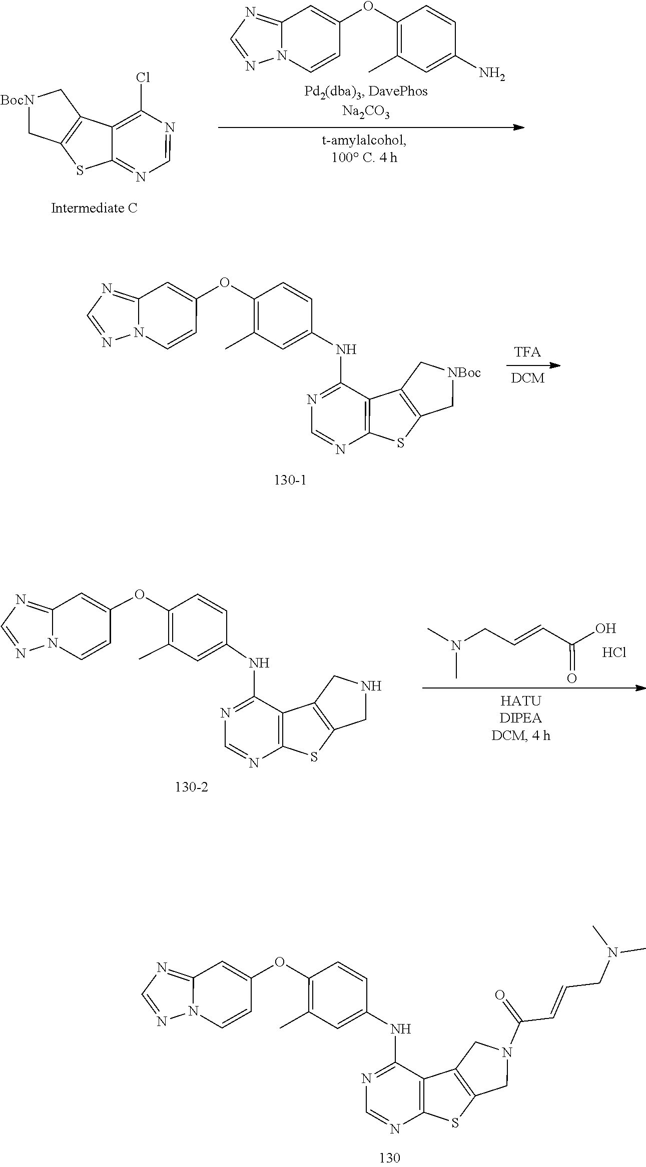

The title compound was prepared in analogous fashion to Compound 99, but using 4-([1,2,4]triazolo[1,5-a]pyridin-7-yloxy)-3-methylaniline (prepared as described for Intermediate J, using 1-fluoro-2-methyl-4-nitrobenzene instead of 2-chloro-1-fluoro-4-nitro-benzene) instead of Intermediate F. The title compound was obtained as off-white solid (15.4 mg. LCMS m/z [M+H]⁺: 527.2. ¹H NMR (400 MHz, DMSO-d₆): δ 8.73-8.68 (m, 0.5H), 8.55-8.52 (m, 0.5H), 6.79 (s, 2H), 6.60-6.46 (m, 1H), 3.21-3.15 (m, 2H), 2.22 (dd, 9H), 4.87-4.80 (m, 1H), 5.32-5.27 (m, 1H), 7.06-7.00 (m, 1H), 7.24-7.17 (m, 1H), 7.70-7.58 (m, 2H), 8.47 (s, 1H), 8.38 (s, 1H), 8.97-8.91 (m, 1H), 5.08 (s, 2H).

### Example 38: (E)-1-(4-((4-([1,2,4]triazolo[1,5-a]pyridin-7-yloxy)-3-methylphenyl)amino)-5,8-dihydropyrido[4′,3′:4,5]thieno[2,3-d]pyrimidin-7(6H)-yl)-4-(dimethylamino)but-2-en-1-one (Compound 138)

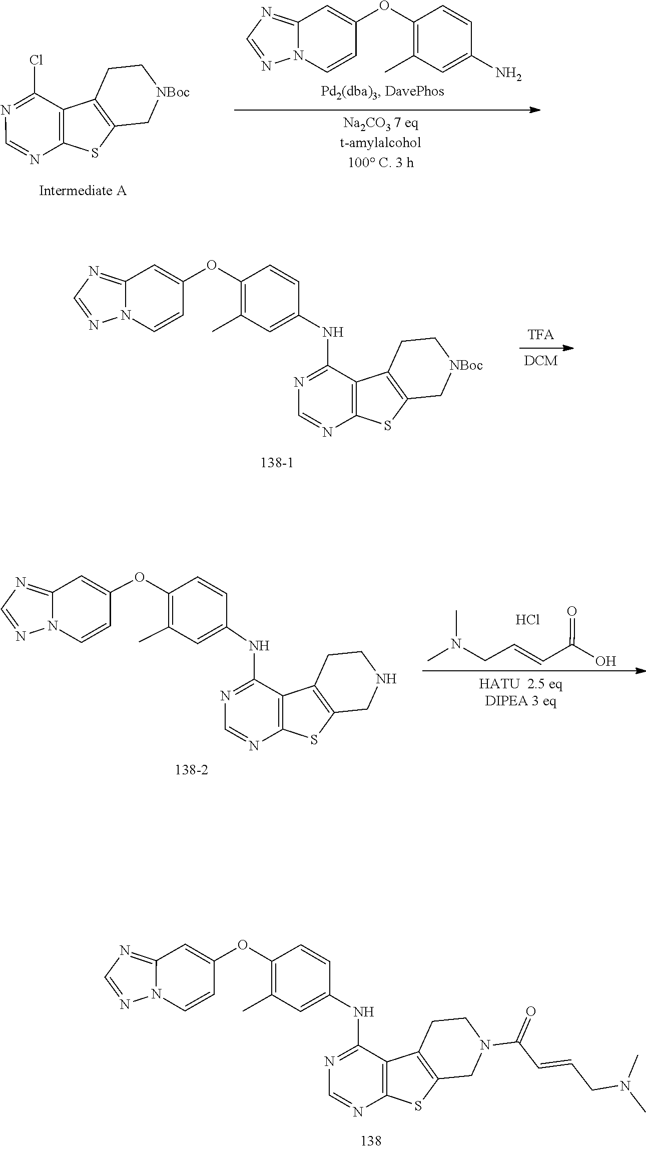

The title compound was prepared in analogous fashion to Compound 130, using Intermediate A instead of Intermediate C. The title compound was obtained as off-white solid (31 mg). LCMS m/z [M+H]⁺: 541.6. ¹H NMR (400 MHz, DMSO-d₆): δ 8.92 (d, 1H), 8.45 (s, 1H), 8.37 (s, 1H), 8.28 (s, 1H), 7.65 (d, 2H), 7.18 (d, 1H), 7.00-7.03 (m, 1H), 6.71-6.78 (m, 3H), 4.87-4.97 (m, 2H), 3.89-3.95 (m, 2H), 3.28 (s, 2H), 3.09 (br m, 2H), 2.18 (m, 9H).

### Example 39: (E)-4-(dimethylamino)-1-(5-methyl-4-((3-methyl-4-((6-methylpyridin-3-yl)oxy)phenyl)amino)-5,7-dihydro-6H-pyrrolo[3′,4′:4,5]thieno[2,3-d]pyrimidin-6-yl)but-2-en-1-one (Compound 141)

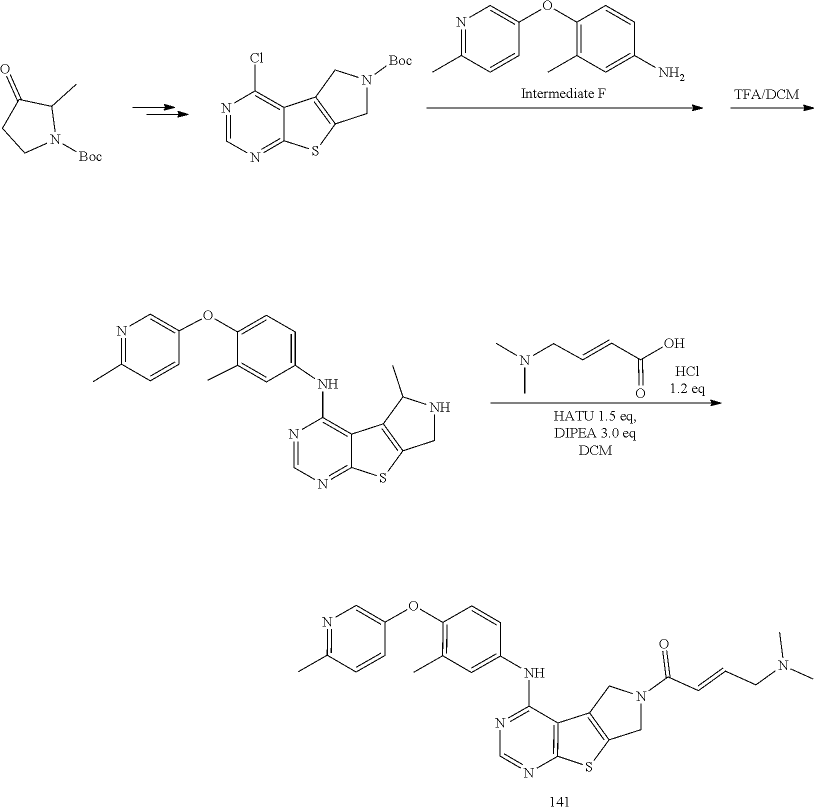

The title compound was prepared in analogous fashion to Compound 8, using tert-butyl 2-methyl-3-oxopyrrolidine-1-carboxylate instead of tert-butyl 4-oxopiperidine-1-carboxylate as starting material in the first step. The title compound was obtained as light yellow solid. MS m/z: 515.2 (M+H)+. ¹H NMR (400 MHz, DMSO-d₆): δ 8.72 (s, 0.5H), 8.54 (s, 0.5H), 8.42 (d, 1H), 8.17-8.16 (m, 1H), 7.52 (d, 1H), 7.44-7.41 (m, 1H), 7.23-7.19 (m, 2H), 6.93 (dd, 1H), 6.82-6.71 (m, 1H), 6.53-6.39 (m, 1H), 6.06-5.97 (m, 1H), 5.05-4.90 (m, 1.5H), 4.72-4.68 (m, 0.5H), 3.10-3.05 (m, 2H), 2.42 (d, 3H), 2.20-2.15 (m, 9H), 1.44-1.40 (m, 3H).

### Example 40: 1-(5-methyl-4-((3-methyl-4-((6-methylpyridin-3-yl)oxy)phenyl)amino)-5,6-dihydropyrido[4′,3′:4,5]thieno[2,3-d]pyrimidin-7(8H)-yl)prop-2-en-1-one (Compound 142)

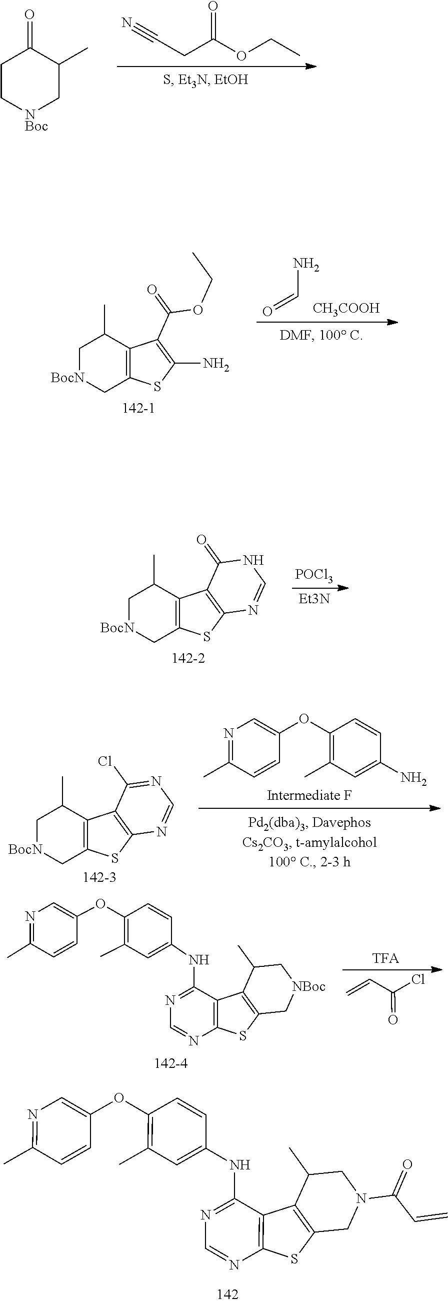

### Step 1: 6-tert-Butyl 3-ethyl 2-amino-4-methyl-4,5-dihydrothieno[2,3-c]pyridine-3,6(7H)-dicarboxylate (142-1)

To a solution of tert-butyl 3-methyl-4-oxopiperidine-1-carboxylate (2.0 g, 9.4 mmol), ethyl 2-cyanoacetate (1.06 g, 9.4 mmol), sulfur (0.3 g, 9.4 mmol) and TEA (0.95 g, 9.4 mmol) in EtOH (20 mL) was allowed to stir at room temperature overnight. The mixture was poured into water and extracted with EtOAc. The organic layer was washed with water and brine, dried over Na₂SO₄ and concentrated. The resultant residue was purified by column chromatography (hexane:EtOAc=2:1) to afford the title compound (1.2 g, 41%) as yellow solid. ¹H NMR (400 MHz, CDCl₃): δ 1.05 (d, 2H), 1.50 (s, 9H), 2.39-2.57 (m, 3H), 2.86 (br, 1H), 3.23-3.30 (m, 1H), 4.11-4.30 (m, 2H), 1.27 (d, 3H).

### Step 2: tert-Butyl 5-methyl-4-oxo-3,4,5,6-tetrahydropyrido[4′,3′:4,5]thieno[2,3-d]pyrimidine-7(8H)— carboxylate (142-2)

A solution of 142-1 (1.2 g, 3.9 mmol), and formamidine acetate (545 mg, 5.23 mmol) in DMF (10 ml) was heated at 100° C. overnight. The reaction mixture was allowed cool to ambient temperature, poured into water and extracted with EtOAc. The organic layer was washed with water and brine, dried over Na₂SO₄ and concentrated. The resultant residue was purified by column chromatography to afford the title compound (1.0 g, 81%) as yellow solid. ¹H NMR (400 MHz, DMSO-d6): δ 1.18 (d, 3H), 1.44 (s, 9H), 3.13 (br, 2H), 4.00 (br, 1H), 4.25 (br, 1H), 4.97 (br, 1H), 8.05 (s, 1H), 12.43 (br, 1H).

### Step 3: tert-Butyl 4-chloro-5-methyl-5,6-dihydropyrido[4′,3′:4,5]thieno[2,3-d]pyrimidine-7(8H)-carboxylate (142-3)

To a mixture of POCl₃ (5 mL) in TEA (5 mL) was added 142-2 (500 mg, 1.56 mmol) at 0° C. The mixture was heated at 60° C. for 2 h after which it was poured into cold saturated sodium carbonate solution and extracted with EtOAc. The organic layer was washed with water and brine, dried over Na₂SO₄ and concentrated. The resultant residue was purified by column chromatography to afford the title compound (250 mg, 53%) as yellow oil. ¹H NMR (400 MHz, DMSO-d6): 1.26 (d, 3H), 1.46 (s, 9H), 3.17 (br, 1H), 3.57 (br, 1H), 4.12 (br, 1H), 4.44 (br, 1H), 5.17 (br, 1H), 8.87 (s, 1H).

### Step 4: tert-Butyl 5-methyl-4-((3-methyl-4-((6-methylpyridin-3-yl)oxy)phenyl)amino)-5,6-dihydropyrido[4′,3′:4,5]thieno[2,3-d]pyrimidine-7(8H)-carboxylate (142-4)

A solution of 142-3 (280 mg, 0.83 mmol), Intermediate F (177 mg, 0.83 mmol), Cs₂CO₃ (269 mg, 1.65 mmol), Pd₂(dba)₃ (37 mg, 0.043 mmol) and Davephos (32 mg, 0.086 mmol) in 2-methyl-2-butanol (5 mL) was heated at 100° C. for 2 h. The mixture was poured into water and extracted with EtOAc. The organic layer was washed with water and brine, dried over Na₂SO₄ and concentrated. The resultant residue was purified by column chromatography to afford the title compound (210 mg, 49%) as yellow solid. ¹H NMR (400 MHz, DMSO-d₆): δ 1.22 (d, 3H), 1.46 (s, 9H), 2.20 (s, 3H), 2.44 s, 3H), 3.45 (br, 1H), 3.79 (br, 1H), 4.01 (br, 1H), 4.37 (br, 1H), 5.01 (br, 1H), 6.95 (d, 1H), 7.19-7.26 (m, 2H), 7.51 (d, 1H), 7.56 (d, 1H), 8.16 (s, 1H), 8.18 (d, 1H), 8.41 (s, 1H).

### Step 5: 1-(5-Methyl-4-((3-methyl-4-((6-methylpyridin-3-yl)oxy)phenyl)amino)-5,6-dihydropyrido[4′,3′:4,5]thieno[2,3-d]pyrimidin-7(8H)-yl)prop-2-en-1-one (Compound 142)

A solution of 142-4 (210 mg, 0.41 mmol) and TFA (2 mL) in DCM (2 mL) was allowed to stir at room temperature for 30 min. The solution was concentrated and dissolved in DCM (5 mL). Saturated sodium carbonate solution (2 mL) was added and the mixture was allowed to stir at 0° C. Acryloyl chloride (37 mg, 0.48 mmol) was added and the mixture was allowed to stir at 0° C. for an additional 5 min. The mixture was poured into water and extracted with EtOAc after which the organic layer was washed with water and brine, dried over Na₂SO₄ and concentrated. The resultant residue was purified by column chromatography to afford the title compound (130 mg, 67%) as white solid. LCMS (M+1): 472.6. ¹H NMR (400 MHz, DMSO-d₆): δ 1.20 (d, 3H), 2.20 (s, 3H), 2.44 (s, 3H), 3.34 (br, 0.5H), 3.73 (d, 0.5H), 3.87 (br, 1H), 4.15 (d, 0.5H), 4.35-4.89 (m, 1H), 4.72 (d, 0.5H), 5.23-5.40 (m, 1H), 5.78 (d, 1H), 6.24 (d, 1H), 6.94-7.03 (m, 2H), 7.19-7.26 (m, 2H), 7.47 (d, 1H), 7.55 (s, 1H), 8.21 (t, 2H), 8.41 (s, 1H).

### Example 41: (S)-1-(5-methyl-4-((3-methyl-4-((6-methylpyridin-3-yl)oxy)phenyl)amino)-5,6-dihydropyrido[4′,3′:4,5]thieno[2,3-d]pyrimidin-7(8H)-yl)prop-2-en-1-one and (R)-1-(5-methyl-4-((3-methyl-4-((6-methylpyridin-3-yl)oxy)phenyl)amino)-5,6-dihydropyrido[4′,3′:4,5]thieno[2,3-d]pyrimidin-7(8H)-yl)prop-2-en-1-one

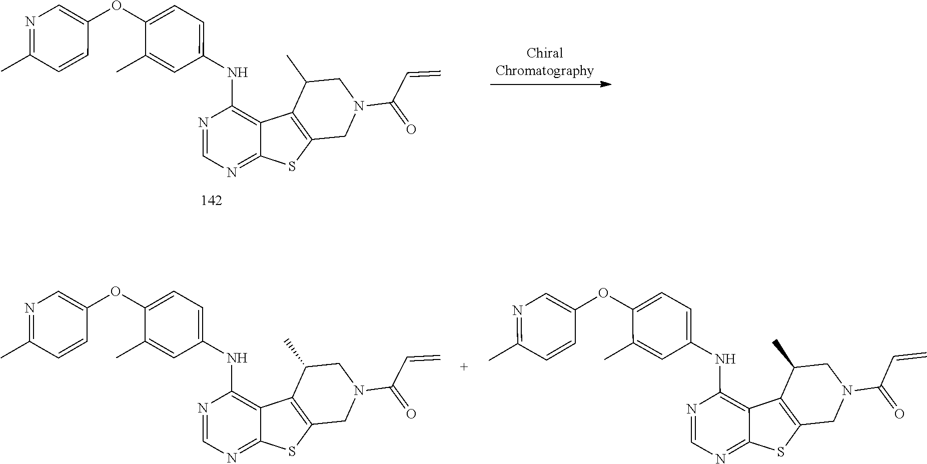 [SMILES: C=CC(=O)N1Cc2sc3ncnc(Nc4ccc(Oc5ccc(C)nc5)c(C)c4)c3c2C(C)C1 ; C=CC(=O)N1Cc2sc3ncnc(Nc4ccc(Oc5ccc(C)nc5)c(C)c4)c3c2[C@H](C)C1 ; C=CC(=O)N1Cc2sc3ncnc(Nc4ccc(Oc5ccc(C)nc5)c(C)c4)c3c2[C@@H](C)C1]

Enantiomers of Compound 142 were separated via HPLC using a Lux Cellulose-4 column. The faster eluting enantiomer was designated Compound 145 ([α]_{D}¹¹⁰=+168° (C=0.1 g/100 mL MeOH)) and slower eluting enantiomer designated Compound 146 ([α]_{D}¹¹⁰=−124° (C=0.1 g/100 mL MeOH)).

### Example 42: (S)-4-(dimethylamino)-1-(5-methyl-4-((3-methyl-4-((6-methylpyridin-3-yl)oxy)phenyl)amino)-5,7-dihydro-6H-pyrrolo[3′,4′:4,5]thieno[2,3-d]pyrimidin-6-yl)but-2-en-1-one and (R)-4-(dimethylamino)-1-(5-methyl-4-((3-methyl-4-((6-methylpyridin-3-yl)oxy)phenyl)amino)-5,7-dihydro-6H-pyrrolo[3′,4′:4,5]thieno[2,3-d]pyrimidin-6-yl)but-2-en-1-one

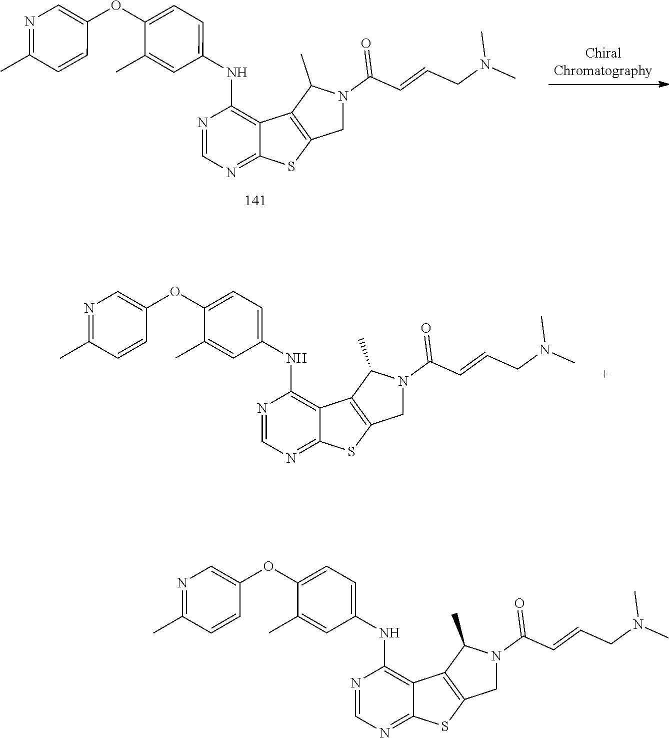

Enantiomers of Compound 141 were separated via HPLC using a Lux Cellulose-4 column. The faster eluting enantiomer was designated Compound 147 ([α]_{D}^{20.1}=−34° (C=0.1 g/100 mL MeOH)) and slower eluting enantiomer designated Compound 148 ([α]_{D}^{16.7}=+30° (C=0.1 g/100 mL MeOH)).

### Example 43: (E)-1-(6,8-cis-dimethyl-4-((3-methyl-4-((6-methylpyridin-3-yl)oxy)phenyl)amino)-5,8-dihydropyrido[4′,3′:4,5]thieno[2,3-d]pyrimidin-7(6H)-yl)-4-(dimethylamino)but-2-en-1-one (Racemic Compound 149)

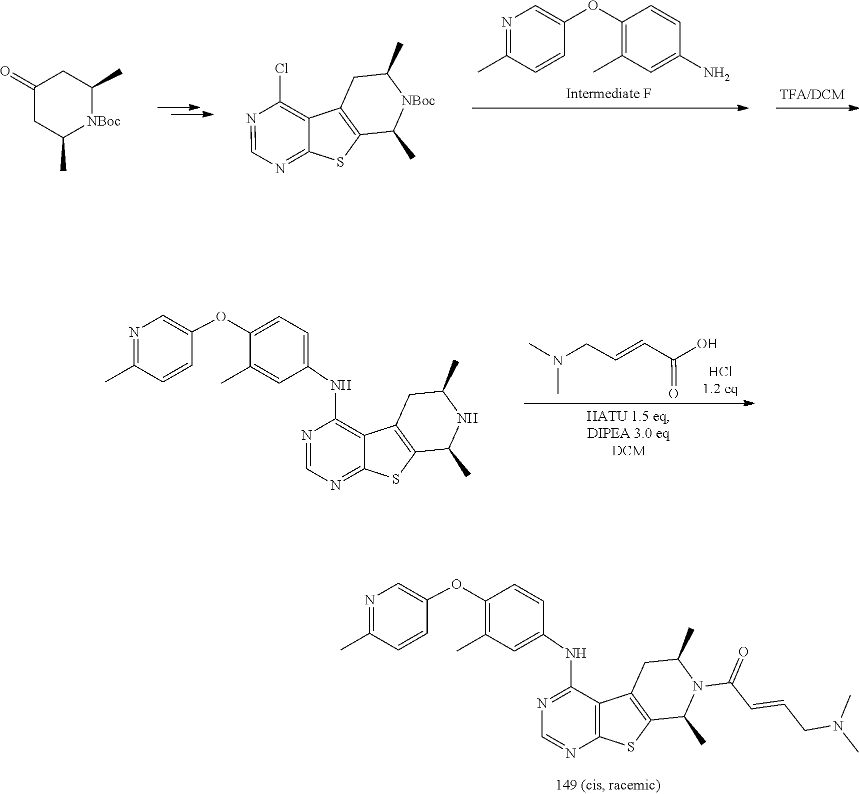

The title compound was prepared in analogous fashion to Compound 8, using tert-butyl 2,6-cis-dimethyl-4-oxopiperidine-1-carboxylate instead of tert-butyl 4-oxopiperidine-1-carboxylate as starting material in the first step. The title compound was obtained as light yellow solid. MS m/z: 543.2 (M+H)+. ¹H NMR (400 MHz, DMSO-d₆): δ 8.41 (s, 1H), 8.25 (s, 1H), 8.14 (s, 1H), 7.56-7.51 (m, 2H), 7.23-7.15 (m, 2H), 6.93 (d, 1H), 6.71-6.61 (m, 2H), 5.53 (b s, 1H), 5.32 (b s, 0.44H), 4.78 (b s, 0.56H), 3.44-3.35 (m, 1H), 3.20-3.10 (m, 1H), 3.05 (d, 2H), 2.41 (s, 3H), 2.16 (s, 3H), 2.15 (s, 6H), 1.56 (d, 3H), 1.21 (d, 3H).

### Example 44: (E)-4-(dimethylamino)-1-(4-((4-((6-methoxypyridin-3-yl)oxy)-3-methylphenyl)amino)-5-methyl-5,8-dihydropyrido[4′,3′:4,5]thieno[2,3-d]pyrimidin-7(6H)-yl)but-2-en-1-one (Compound 152)

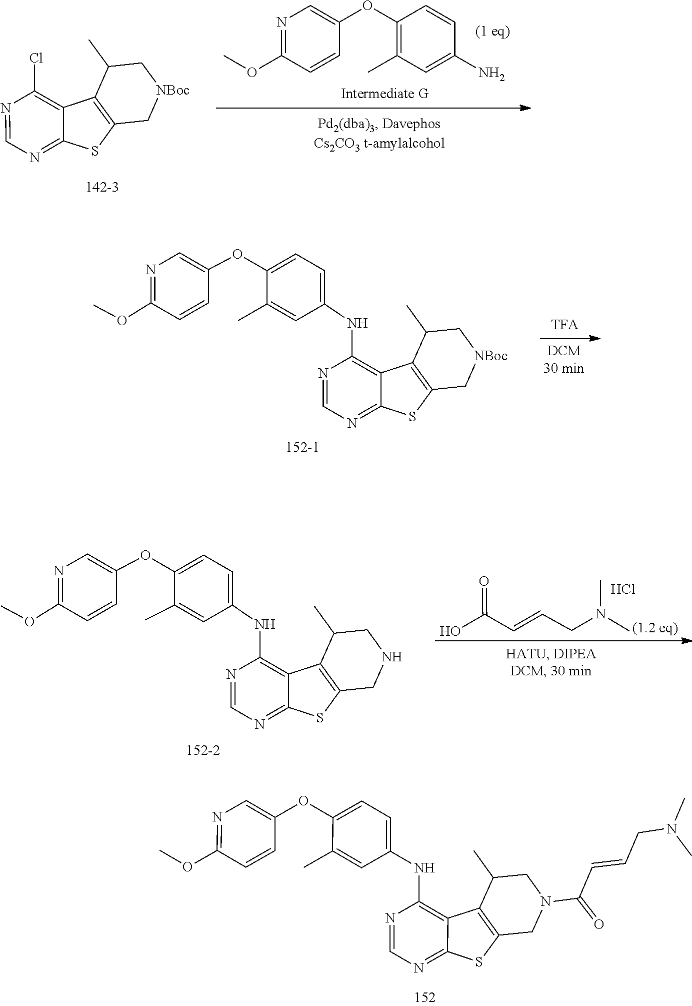

The title compound was prepared in analogous fashion to Compound 99, using tert-butyl 4-chloro-5-methyl-5,6-dihydropyrido[4′,3′:4,5]thieno[2,3-d]pyrimidine-7(8H)-carboxylate (142-3) instead of Intermediate C and using Intermediate G instead of Intermediate F. The title compound was obtained as white solid. LCMS m/z [M+H]⁺: 545.3; ¹H NMR (400 MHz, DMSO-d₆): δ 8.39 (s, 1H), 8.22 and 8.18 (2b s, 1H), 7.92 (d, 1H), 7.51 (b s, 1H), 7.43-7.39 (m, 2H), 6.87-6.83 (m, 2H), 6.76-6.73 (m, 2H), 5.37 (d, 0.63H), 5.25 (d, 0.37H), 4.70 (d, 0.37H), 4.47 (d, 0.37H), 4.36 (d, 0.63H), 4.13 (d, 0.63H), 3.89-3.83 (m, 4H), 3.75-3.70 (0.58H), 3.32-3.29 (m, 0.42H), 3.09-3.05 (m, 2H), 2.23 (s, 3H), 2.16 (s, 6H), 1.18 (t, 3H).

### Example 45: (E)-4-(dimethylamino)-1-(7-methyl-4-((3-methyl-4-((6-methylpyridin-3-yl)oxy)phenyl)amino)-5,7-dihydro-6H-pyrrolo[3′,4′:4,5]thieno[2,3-d]pyrimidin-6-yl)but-2-en-1-one (Compound 153)

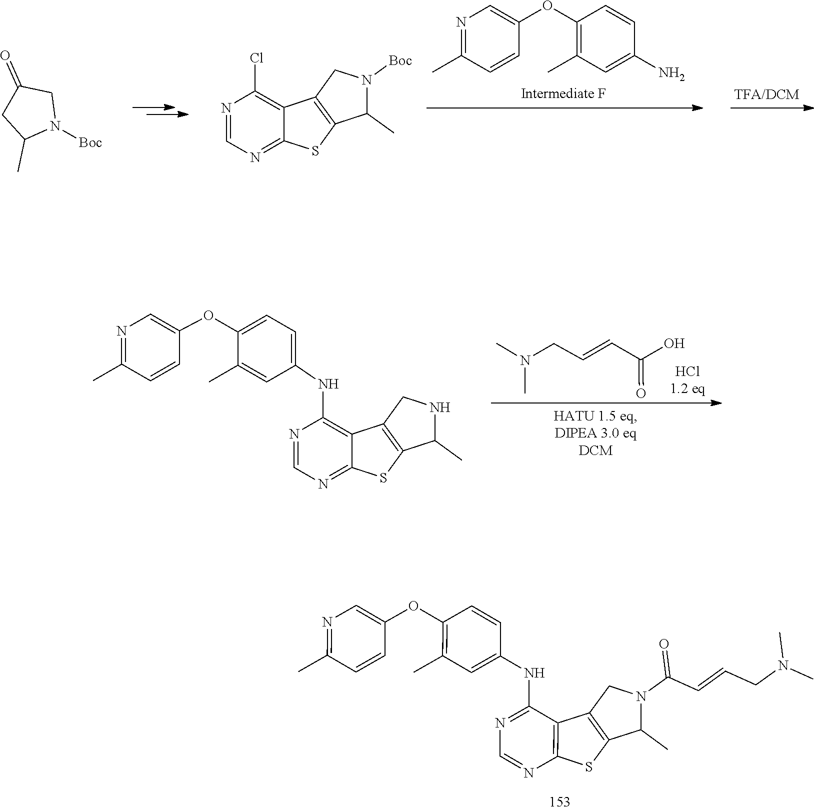

The title compound was prepared in analogous fashion to Compound 8, using tert-butyl 2-methyl-4-oxopyrrolidine-1-carboxylate instead of tert-butyl 4-oxopiperidine-1-carboxylate as starting material in the first step. The title compound was obtained as light yellow solid. MS m/z: 515.2 (M+H)⁺. ¹H NMR (400 MHz, DMSO-d₆): δ 8.67 (s, 0.32H), 8.48 (s, 0.68H), 8.40 (d, 1H), 8.15 (d, 1H), 7.50 (t, 1H), 7.47-7.41 (m, 1H), 7.22-7.18 (m, 2H), 6.97-6.91 (m, 1H), 6.81-6.73 (m, 1H), 6.53-6.45 (m, 1H), 5.59-5.58 (m, 0.32H), 5.42-5.38 (m, 0.68H), 5.29-5.16 (m, 1.68H), 4.89-4.84 (m, 0.32H), 3.07 (d, 2H), 2.42 (s, 3H), 2.19-2.15 (m, 9H), 1.50-1.48 (m, 3H).

### Example 46: (E)-4-(dimethylamino)-1-(4-((4-((6-(dimethylamino)pyridin-3-yl)oxy)-3-methylphenyl)amino)-5,8-dihydropyrido[4′,3′:4,5]thieno[2,3-d]pyrimidin-7(6H)-yl)but-2-en-1-one (Compound 158)

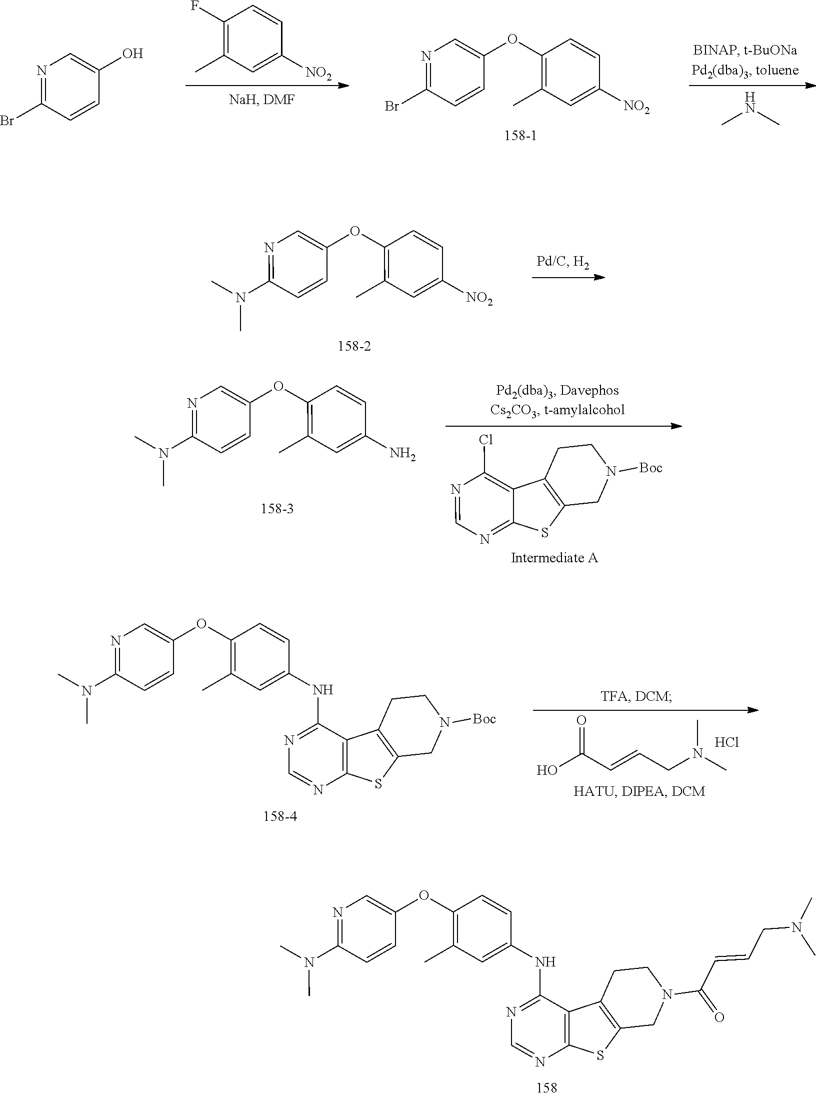

### Step 1: 2-Bromo-5-(2-methyl-4-nitrophenoxy)pyridine (158-1)

NaH (920 mg, 23 mmol) was added slowly to the solution of 6-bromopyridin-3-ol (2.0 g, 11.5 mmol) in DMF (15 mL) at 0° C. 1-Fluoro-2-methyl-4-nitrobenzene (1.8 g, 11.5 mmol) was added to the mixture above at 0° C. and the reaction mixture was stirred at ambient temperature overnight. The mixture was quenched with ice water and extracted with ethyl acetate. The combined organics were washed with water and brine, dried over sodium sulfate and concentrated to dryness. The resultant residue was purified by chromatography column (hexanes/ethyl acetate=5/1) to afford the title compound (1.3 g, 37%). ¹H NMR (400 MHz, DMSO-d₆): δ 2.38 (s, 3H), 7.04 (d, 1H), 7.58 (dd, 1H), 7.73 (d, 1H), 8.07 (dd, 1H), 8.27 (d, 1H), 8.32 (d, 1H).

### Step 2: N,N-Dimethyl-5-(2-methyl-4-nitrophenoxy)pyridin-2-amine (158-2)

A mixture of 158-1 (1.16 g, 3.7 mmol), dimethylamine (170 mg, 3.7 mmol), BINAP (467 mg, 0.75 mmol), t-BuONa (720 mg, 7.5 mmol) and Pd₂(dba)₃ (340 mg, 0.4 mmol) in toluene (20 mL) was stirred at 100° C. under nitrogen for 3 h. The mixture was quenched with water and extracted with ethyl acetate. The combined organics were washed with water and brine, dried over sodium sulfate and concentrated to dryness. The resultant residue was purified by chromatography column (hexanes/ethyl acetate=4/1) to afford the title compound (890 mg, 85%). ¹H NMR (400 MHz, DMSO-d₆): δ 2.41 (s, 3H), 3.05 (s, 6H), 6.71 (d, 1H), 6.73 (d, 1H), 7.41 (dd, 1H), 8.01 (d, 1H), 8.03 (d, 1H), 8.19 (d, 1H).

### Step 3: 5-(4-Amino-2-methylphenoxy)-N,N-dimethylpyridin-2-amine (158-3)

A mixture of 158-2 (890 mg, 3.25 mmol) and Pd/C (100 mg) in MeOH (10 mL) was stirred at ambient temperature under hydrogen overnight. The mixture was filtered and the filtrate was concentrated to dryness to afford the title compound (720 mg, 91%). LCMS m/z [M+H]⁺: 244.2.

### Step 4: tert-Butyl 4-((4-((6-(dimethylamino)pyridin-3-yl)oxy)-3-methylphenyl)amino)-5,6-dihydropyrido[4′,3′:4,5]thieno[2,3-d]pyrimidine-7(8H)-carboxylate (158-4)

A suspension of 158-3 (300 mg, 1.2 mmol), Intermediate A (442 mg, 1.4 mmol), Pd₂(dba)₃ (113 mg, 0.12 mmol), Cs₂CO₃ (800 mg, 2.5 mmol), and Davephos (48 mg, 0.12 mmol) in t-amyl alcohol (15 mL) was heated at 100° C. under nitrogen for 3 h. After cooling to ambient temperature, the reaction mixture was quenched with water and extracted with ethyl acetate. The combined organic layers were washed with water and brine, dried over sodium sulfate and concentrated in vacuo. The resultant residue was purified by column chromatography (hexanes/ethyl acetate=1/1) to afford the title compound (500 mg, 90%). LCMS m/z [M+H]⁺: 533.4.

### Step 5: (E)-4-(Dimethylamino)-1-(4-((4-((6-(dimethylamino)pyridin-3-yl)oxy)-3-methylphenyl)amino)-5,6-dihydropyrido[4′,3′:4,5]thieno[2,3-d]pyrimidin-7(8H)-yl)but-2-en-1-one (Compound 158)

A mixture of 158-4 (500 mg, 0.9 mmol) in TFA (5 mL)/DCM (5 mL) was stirred at ambient temperature for 30 min. The mixture was concentrated to dryness then taken up in DCM (10 mL)/DIPEA (242 mg, 1.9 mmol). To the resulting solution was added (E)-4-(dimethylamino)but-2-enoic acid hydrochloride (187 mg, 1.1 mmol) and HATU (536 mg, 1.4 mmol) at ambient temperature and the mixture was allowed to stir for 10 min after which it was quenched with water and extracted with DCM. The combined organics were washed with water and brine, dried over sodium sulfate and concentrated to dryness. The resultant residue was purified by column chromatography (DCM/MeOH=10/1) to afford the title compound (26 mg, 5%). LCMS m/z [M+H]*+: 544.4; ¹H NMR (400 MHz, DMSO-d₆): δ 2.17 (s, 6H), 2.26 (s, 3H), 3.00 (s, 6H), 3.06 (br, 1H), 3.27-3.29 (m, 3H), 3.86-3.93 (m, 2H), 4.85-4.95 (m, 2H), 6.66-6.74 (m, 4H), 7.25 (dd, 1H), 7.40 (d, 1H), 7.49 (s, 1H), 7.90 (d, 1H), 8.14 (s, 1H), 8.38 (s, 1H).

### Example 47: (R)-4-(dimethylamino)-1-(4-((4-((6-methoxypyridin-3-yl)oxy)-3-methylphenyl)amino)-5-methyl-5,8-dihydropyrido[4′,3′:4,5]thieno[2,3-d]pyrimidin-7(6H)-yl)but-2-en-1-one and (S)-4-(dimethylamino)-1-(4-((4-((6-methoxypyridin-3-yl)oxy)-3-methylphenyl)amino)-5-methyl-5,8-dihydropyrido[4′,3′:4,5]thieno[2,3-d]pyrimidin-7(6H)-yl)but-2-en-1-on

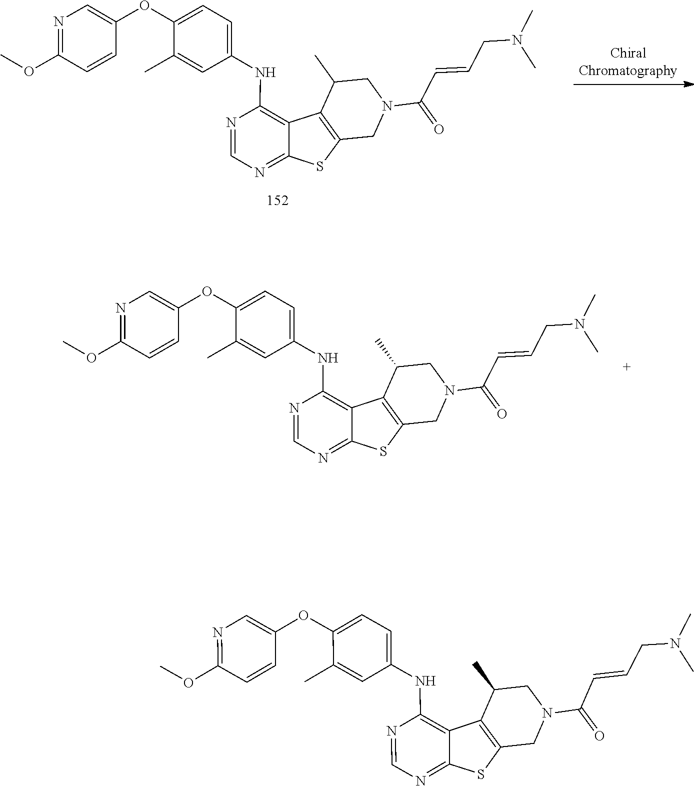

Enantiomers of Compound 152 were separated via HPLC using a Lux Cellulose-4 column. The faster eluting enantiomer was designated Compound 159 ([a]D=+134⁰ (C=0.1 g/100 mL MeOH)) and slower eluting enantiomer designated Compound 160 ([α]_{D}-140° (C=0.1 g/100 mL MeOH)).

### Example 48: (S,E)-4-(dimethylamino)-1-(7-methyl-4-((3-methyl-4-((6-methylpyridin-3-yl)oxy)phenyl)amino)-5,7-dihydro-6H-pyrrolo[3′,4′:4,5]thieno[2,3-d]pyrimidin-6-yl)but-2-en-1-one and (R,E)-4-(dimethylamino)-1-(7-methyl-4-((3-methyl-4-((6-methylpyridin-3-yl)oxy)phenyl)amino)-5,7-dihydro-6H-pyrrolo[3′,4′:4,5]thieno[2,3-d]pyrimidin-6-yl)but-2-en-1-one

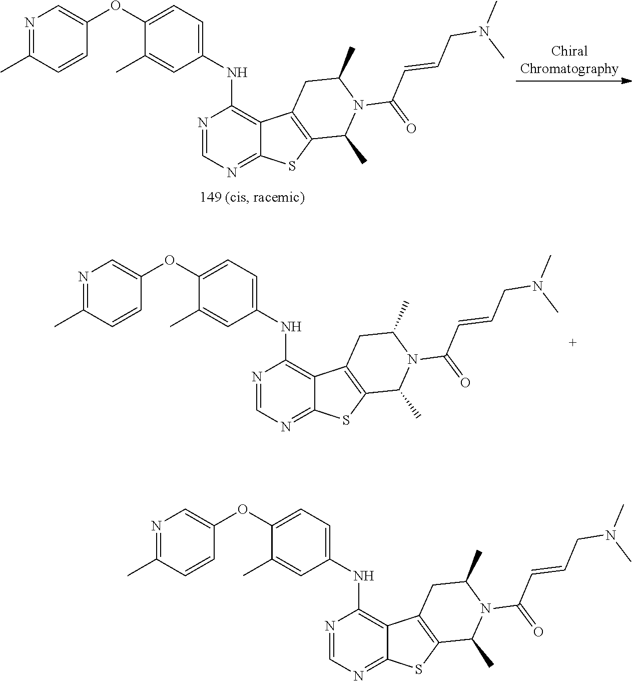

Enantiomers of Compound 149 were separated via HPLC using a Lux Cellulose-4 column. The faster eluting enantiomer was designated Compound 161 ([α]_{D}^{25.7}=+2.53° (C=0.405 g/100 mL MeOH)) and slower eluting enantiomer designated Compound 162 ([α]_{D}^{26.9}=−5.2° (C=0.384 g/100 mL MeOH)).

### Example 49: (E)-1-(6,8-trans-dimethyl-4-((3-methyl-4-((6-methylpyridin-3-yl)oxy)phenyl)amino)-5,8-dihydropyrido[4′,3′:4,5]thieno[2,3-d]pyrimidin-7(6H)-yl)-4-(dimethylamino)but-2-en-1-one (Racemic Compound 165)

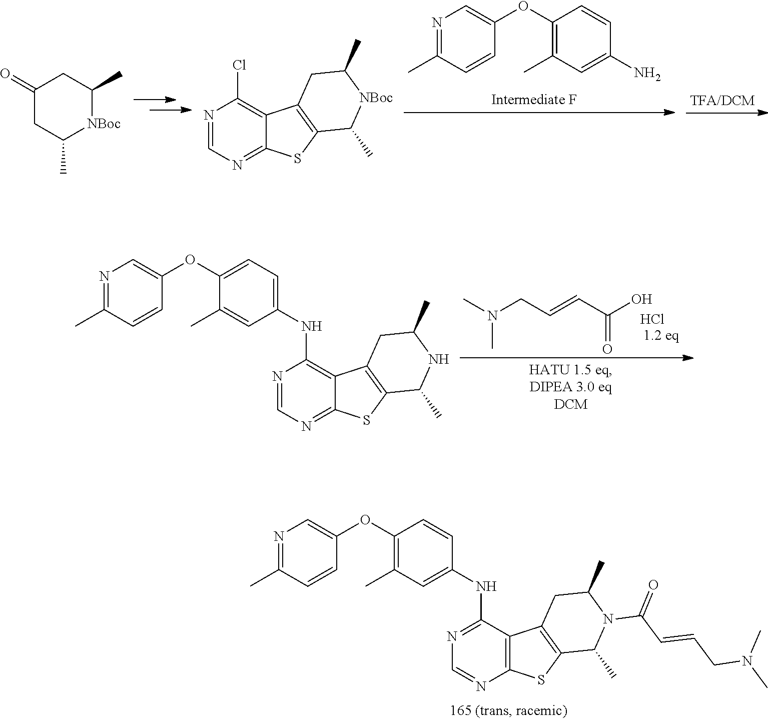

The title compound was prepared in analogous fashion to Compound 8, using tert-butyl 2,6-trans-dimethyl-4-oxopiperidine-1-carboxylate instead of tert-butyl 4-oxopiperidine-1-carboxylate as starting material in the first step. The title compound was obtained as white solid. MS m/z: 543.2 (M+H)+. ¹H NMR (400 MHz, DMSO-d₆): δ 8.54 (s, 1H), 8.40 (s, 1H), 8.15 (d, 1H), 7.53 (d, 1H), 7.47 (dd, 1H), 7.24-7.17 (m, 2H), 6.93 (d, 1H), 6.68 (b s, 2H), 5.31 (d, 1H), 4.56 (b s, 1H), 3.36-3.30 (m, 2H), 3.07-3.01 (m, 2H), 2.42 (s, 3H), 2.16 (s, 3H), 2.14 (s, 6H), 1.46 (d, 3H), 1.04 (b s, 3H).

### Example 50: 1-(6,8-trans-dimethyl-4-((3-methyl-4-((6-methylpyridin-3-yl)oxy)phenyl)amino)-5,8-dihydropyrido[4′,3′:4,5]thieno[2,3-d]pyrimidin-7(6H)-yl)prop-2-en-1-one (Compound 166)

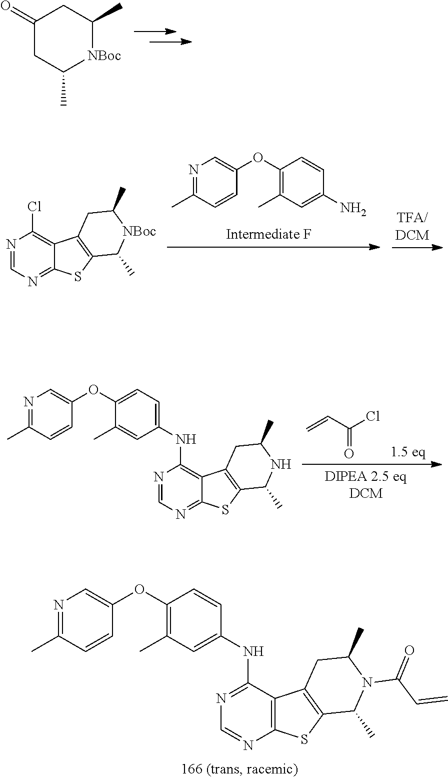

The intermediate, trans-6,8-dimethyl-N-(3-methyl-4-((6-methylpyridin-3-yl)oxy)phenyl)-5,6,7,8-tetrahydropyrido[4′,3′:4,5]thieno[2,3-d]pyrimidin-4-amine, was prepared in analogous fashion to intermediate K-2, using tert-butyl 2,6-trans-dimethyl-4-oxopiperidine-1-carboxylate instead of tert-butyl 4-oxopiperidine-1-carboxylate as starting material in the first step. Then to the mixture of this intermediate (50 mg, 0.116 mmol) and N-ethyl-N-isopropylpropan-2-amine (37 mg, 0.29 mmol) in DCM (2.0 mL) was added a solution of acryloyl chloride (13.6 mg, 0.15 mmol) in DCM (2.0 mL). After addition, the reaction mixture was stirred for another 0.5 h at ambient temperature. After which the reaction mixture was diluted with DCM, washed with saturated aqueous NaHCO₃, dried (Na₂SO₄), filtered, and evaporated to dryness. The resultant residue was purified through column chromatography on silica gel (eluting with 10% MeOH in DCM) to provide the title compound as white solid (49 mg, 87%). MS m/z: 486.2 (M+H)+. ¹H NMR (400 MHz, DMSO-d₆): δ 8.54 (s, 1H), 8.40 (s, 1H), 8.15 (d, 1H), 7.53 (d, 1H), 7.47 (dd, 1H), 7.24-7.17 (m, 2H), 6.94-6.85 (m, 2H), 6.19 (dd, 1H), 5.72 (dd, 1H), 5.33 (d, 1H), 4.59 (b s, 1H), 3.35-3.28 (m, 2H), 2.42 (s, 3H), 2.17 (s, 3H), 1.46 (d, 3H), 1.04 (b s, 3H).

### Example 51: (E)-1-(4-((3-Chloro-4-((6-methylpyridin-3-yl)oxy)phenyl)amino)-8,9-dihydro-5H-pyrimido[5′,4′:4,5]thieno[2,3-d]azepin-7(6H)-yl)-4-(dimethylamino)but-2-en-1-one (Compound 167)

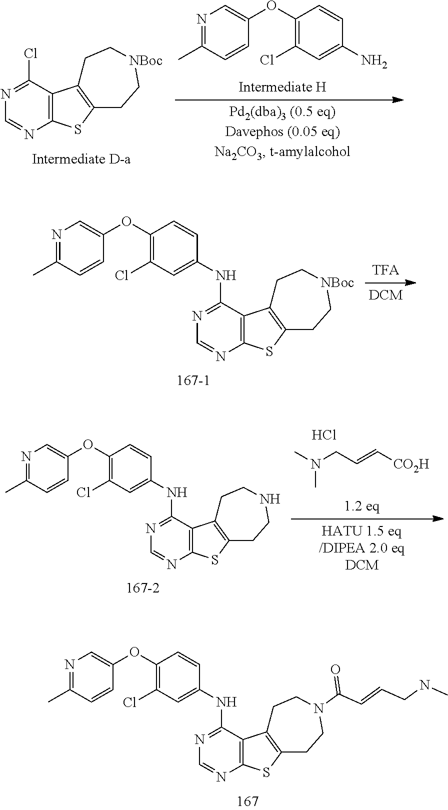

### Step 1: tert-Butyl 4-((3-chloro-4-((6-methylpyridin-3-yl)oxy)phenyl)amino)-8,9-dihydro-5H-pyrimido[5′,4′:4,5]thieno[2,3-d]azepine-7(6H)-carboxylate (167-1)

A mixture of Intermediate D-a (350 mg, 1.03 mmol), Intermediate H (290 mg, 1.24 mmol), Davephos (20 mg, 0.05 mmol), Pd₂(dba)₃ (47 mg, 0.05 mmol) and Na₂CO₃ (546 mg, 5.15 mmol) in t-amylalcohol (10 mL) was stirred at 100° C. for 3 h under nitrogen atmosphere. The reaction mixture was quenched with water and extracted with EtOAc. The organic layer was washed with brine, concentrated and purified through column chromatography (DCM:MeOH=100:1) to afford the title compound as yellow solid (400 mg, 72%). LCMS m/z [M+H]⁺: 538.5; ¹H NMR (400 MHz, CDCl₃): δ 8.52 (s, 1H), 8.29 (s, 1H), 7.83 and 7.92 (s, 1H), 7.42-7.44 (m, 1H), 7.08-7.18 (m, 2H), 6.98-7.04 (m, 2H), 3.87-3.97 (m, 2H), 3.74-3.76 (m, 2H), 3.32-3.38 (m, 2H), 3.17 (t, 2H), 2.54 (s, 3H), 1.48 and 1.51 (two singlets, 9H).

### Step 2: N-(3-Chloro-4-((6-methylpyridin-3-yl)oxy)phenyl)-6,7,8,9-tetrahydro-5H-pyrimido[5′,4′:4,5]thieno[2,3-d]azepin-4-amine (167-2)

TFA (4 mL) was added to a solution of 167-1 (250 mg, 0.46 mmol) in DCM (10 mL). The mixture was stirred at ambient temperature for 1 h. The reaction mixture was concentrated and the resultant residue was dissolved in DCM and washed with aqueous Na₂CO₃ and brine. The organic solution was dried over Na₂SO₄ and concentrated to afford the title compound as yellow solid (190 mg, 94%). LCMS m/z [M+H]⁺: 438.2.

### Step 3: (E)-1-(4-((3-Chloro-4-((6-methylpyridin-3-yl)oxy)phenyl)amino)-8,9-dihydro-5H-pyrimido[5′,4′:4,5]thieno[2,3-d]azepin-7(6H)-yl)-4-(dimethylamino)but-2-en-1-one (Compound 167)

A mixture of 167-2 (190 mg, 0.43 mmol), (E)-4-(dimethylamino)but-2-enoic acid hydrochloride (86 mg, 0.52 mmol), HATU (214 mg, 0.65 mmol), DIPEA (168 mg, 1.30 mmol) in DCM (12 mL) was stirred at room temperature for 1 h. The reaction mixture was quenched with water, extracted with DCM, washed with brine and concentrated. The resultant residue was purified by column chromatography (DCM:MeOH=15:1) to afford the title compound as yellow solid (190 mg, 80%). LCMS m/z [M+H]⁺: 549.5; ¹H NMR (400 MHz, DMSO-d₆): δ 8.61 (d, 1H), 8.44 (d, 1H), 8.21 (s, 1H), 7.89 (dd, 1H), 7.59-7.62 (m, 1H), 7.26-7.28 (m, 2H), 7.20 (t, 1H), 6.64-6.70 (m, 2H), 3.86-3.95 (m, 3H), 3.78 (t, 1H), 3.38-3.41 (m, 2H), 3.18-3.22 (m, 2H), 3.08-3.11 (m, 2H), 2.45 (s, 3H), 2.18 (s, 6H).

### Example 52: (E)-4-(Dimethylamino)-1-(4-((3-methyl-4-((6-methylpyridin-3-yl)oxy)phenyl)amino)-8,9-dihydro-5H-pyrimido[5′,4′:4,5]thieno[2,3-d]azepin-7(6H)-yl)but-2-en-1-one (Compound 168)

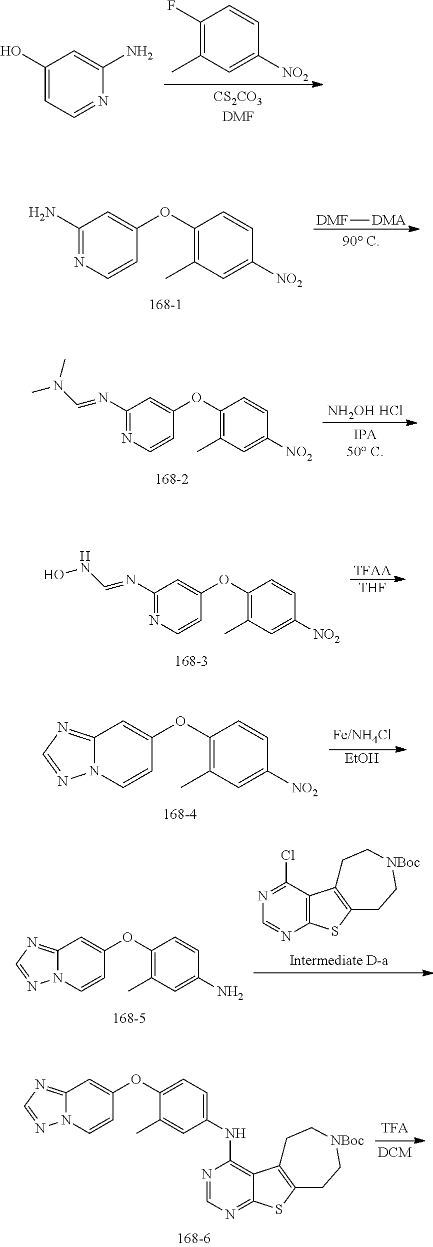

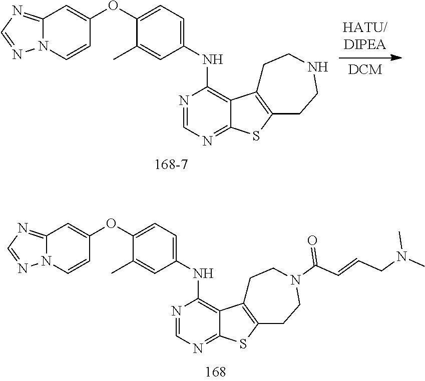 [SMILES: Cc1cc(Nc2ncnc3sc4c(c23)CCNCC4)ccc1Oc1ccn2ncnc2c1 ; Cc1cc(Nc2ncnc3sc4c(c23)CCN(C(=O)/C=C/CN(C)C)CC4)ccc1Oc1ccn2ncnc2c1]

### Step 1: 4-(2-Methyl-4-nitrophenoxy)pyridin-2-amine (168-1)

A mixture of 2-aminopyridin-4-ol (15 g, 136 mmol), 1-fluoro-2-meth yl-4-nitro benzene (21 g, 135 mmol), and Cs₂CO₃ (66 g, 203 mmol) in DMF (100 mL) was stirred at 50° C. overnight. The reaction mixture was quenched with water and the resultant solid collected by filtration and dried to afford the title compound as yellow solid (32 g, 96%).

### Step 2: (E)-N,N-Dimethyl-N′-(4-(2-methyl-4-nitrophenoxy)pyridin-2-yl)formimidamide (168-2)

A solution of 168-1 (30 g, 122 mmol) in DMF-DMA (60 mL) was stirred at 90° C. for 2 h. DMF-DMA was removed under reduced pressure and the resultant residue was taken up in EtOAc, washed with water and brine, and the organic layer was dried and concentrated to afford the title compound as brown solid (33 g, 90%). LCMS m/z [M+H]⁺: 301.3.

### Step 3: (E)-N-Hydroxy-N′-(4-(2-methyl-4-nitrophenoxy)pyridin-2-yl)formimidamide (168-3)

To a solution of 168-2 (33 g, 109.9 mmol) in isopropyl alcohol (100 mL) was added hydroxylamine hydrochloride (9.2 g, 131.8 mmol), and the mixture was heated at 50° C. overnight. The resultant solid was collected by filtration and dried to afford the title compound as white solid (30 g, 95%).

### Step 4: 7-(2-Methyl-4-nitrophenoxy)-[1,2,4]triazolo[1,5-a]pyridine (168-4)

To a solution of 168-3 (30 g, 104 mmol) in dry THF (120 mL) was added TFAA (26.2 g, 124.7 mmol) dropwise at 0° C. The mixture was stirred at ambient temperature overnight under nitrogen atmosphere. The solvent was removed in vacuo and the resultant residue was treated with aqueous NaHCO₃ to pH=8. The resulting mixture was extracted with EtOAc, and the organic layer was dried over sodium sulfate and concentrated. The resultant residue was purified by flash column chromatography (DCM/MeOH=150/1) to afford the title compound as white solid (16.8 g, 60%). LCMS m/z [M+H]⁺: 271.2; ¹HNMR (400 MHz, DMSO-d₆): δ 2.35 (s, 3H), 7.09-7.11 (m, 1H), 7.26-7.30 (m, 2H), 8.12-8.15 (m, 1H), 8.32 (d, 1H), 8.46 (s, 1H), 9.01 (s, 1H).

### Step 5: 4-([1,2,4]Triazolo[1,5-a]pyridin-7-yloxy)-3-methylaniline (168-5)

A mixture of 168-4 (16.8 g, 62.2 mmol), Fe (12.2 g, 218 mmol) and NH₄Cl (11.6 g, 218 mmol) in EtOH/H₂O (50 mL/25 mL) were stirred at 85° C. for 2 h. The solvent was removed in vacuo. The residue was dissolved in EtOAc, washed with water and brine, dried and concentrated. The resultant residue was purified through column chromatography (Hexanes/EtOAc=2/1) to afford the title compound as white solid (11.2 g, 75%). LCMS m/z [M+H]⁺: 241.1; ¹H NMR (400 MHz, DMSO-d₆): δ 1.99 (s, 3H), 5.10 (s, 2H), 6.62 (d, 1H), 6.55 (d, 1H), 6.49 (dd, 1H), 6.82 (d, 1H), 6.95 (dd, 1H), 8.33 (s, 1H), 8.86 (d, 1H).

### Step 6: tert-Butyl 4-((4-([1,2,4]triazolo[1,5-a]pyridin-7-yloxy)-3-methylphenyl)amino)-8,9-dihydro-5H-pyrimido[5′,4′:4,5]thieno[2,3-d]azepine-7(6H)-carboxylate (168-6)

A mixture of Intermediate D-a (156 mg, 0.46 mmol), 168-5 (100 mg, 0.42 mmol), DavePhos (9 mg, 0.02 mmol), Pd₂(dba)₃ (20 mg, 0.02 mmol) and Cs₂CO₃ (273 mg, 0.84 mmol) in t-amylalcohol (10 mL) was stirred at 100° C. for 2.5 h under nitrogen atmosphere. The reaction mixture was quenched with water, extracted with EtOAc, washed with brine and concentrated. The resultant residue was purified by column chromatography (DCM:MeOH=80:1) to afford the title compound as white solid (160 mg, 70%). LCMS m/z [M+H]⁺: 544.2; ¹H NMR (400 MHz, DMSO-d₆): δ. 8.94 (d, 1H), 8.56 (two singlets, 1H), 8.41-8.38 (m, 2H), 7.52-7.60 (m, 2H), 7.18 (d, 1H), 7.03 (dd, 1H), 6.78 (d, 1H), 3.60-3.73 (m, 4H), 3.38-3.41 (m, 2H), 3.09-3.17 (m, 2H), 2.17 (s, 3H), 1.43 (s, 9H).

### Step 7: N-(4-([1,2,4]Triazolo[1,5-a]pyridin-7-yloxy)-3-methylphenyl)-6,7,8,9-tetrahydro-5H-pyrimido[5′,4′:4,5]thieno[2,3-d]azepin-4-amine (168-7)

TFA (3 mL) was added to a solution of 168-6 (160 mg, 0.29 mmol) in DCM (3 mL). The mixture was stirred at ambient temperature for 1 h after which it was concentrated. The resultant residue was dissolved in DCM and washed with Na₂CO₃ and brine. The organic layer was concentrated to afford the title compound as white solid (105 mg, 82%). LCMS m/z [M+H]⁺: 444.3; ¹H NMR (400 MHz, DMSO-d₆): δ 8.94 (d, 1H), 8.66 (s, 1H), 8.40-8.38 (m, 2H), 7.54-7.58 (m, 2H), 7.17 (d, 1H), 7.02 (dd, 1H), 6.77 (d, 1H), 3.24-3.36 (m, 4H), 2.90-3.02 (m, 4H), 2.16 (s, 3H).

### Step 8: (E)-4-(Dimethylamino)-1-(4-((3-methyl-4-((6-methylpyridin-3-yl)oxy)phenyl)amino)-8,9-dihydro-5H-pyrimido[5′,4′:4,5]thieno[2,3-d]azepin-7(6H)-yl)but-2-en-1-one (compound 168)

A mixture of 168-7 (105 mg, 0.24 mmol), (E)-4-(dimethylamino)but-2-enoic acid hydrochloride (48 mg, 0.28 mmol), HATU (180 mg, 0.47 mmol), DIPEA (61 mg, 0.47 mmol) in DCM (10 mL) was stirred at ambient temperature for 1 h. The reaction mixture was quenched with water, extracted with DCM, washed with brine and concentrated. The resultant residue was purified by prep-TLC to afford the title compound as white solid (60 mg, 45%). LCMS m/z [M+H]⁺: 555.4; ¹H NMR (400 MHz, DMSO-d₆): δ. 8.95 (dd, 1H), 8.56 (d, 1H), 8.42 (d, 1H), 8.39 (s, 1H), 7.60 (t, 2H), 7.18 (dd, 1H), 7.03 (dd, 1H), 6.81-6.93 (m, 1H), 6.77 (dd, 1H), 6.63-6.72 (m, 1H), 3.97-3.99 (m, 1H), 3.88-3.93 (m, 2H), 3.80 (t, 1H), 3.42-3.51 (m, 4H), 3.22-3.23 (m, 2H), 2.48 (s, 6H), 2.17 (s, 3H).

### Example 53: (S)-4-(dimethylamino)-1-(7-methyl-4-((3-methyl-4-((6-methylpyridin-3-yl)oxy)phenyl)amino)-5,7-dihydro-6H-pyrrolo[3′,4′:4,5]thieno[2,3-d]pyrimidin-6-yl)but-2-en-1-one and (R)-4-(dimethylamino)-1-(7-methyl-4-((3-methyl-4-((6-methylpyridin-3-yl)oxy)phenyl)amino)-5,7-dihydro-6H-pyrrolo[3′,4′:4,5]thieno[2,3-d]pyrimidin-6-yl)but-2-en-1-one

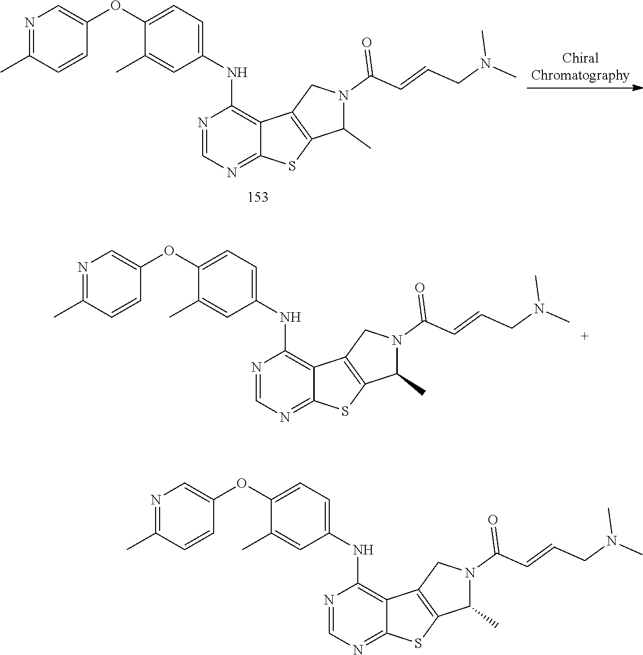

Enantiomers of Compound 153 were separated via HPLC using a Lux Cellulose-4 column. The faster eluting enantiomer was designated Compound 169 ([α]_{D}^{19.2}=−48° (C=0.1 g/100 mL MeOH)) and slower eluting enantiomer designated Compound 170 ([α]_{D}^{19.0}=+42° (C=0.1 g/100 mL MeOH)).

### Example 54: (E)-1-(4-((3-Chloro-4-((6-methylpyridin-3-yl)oxy)phenyl)amino)-5-methyl-5,6-dihydropyrido[4′,3′:4,5]thieno[2,3-d]pyrimidin-7(8H)-yl)-4-(dimethylamino)but-2-en-1-one (Compound 171)

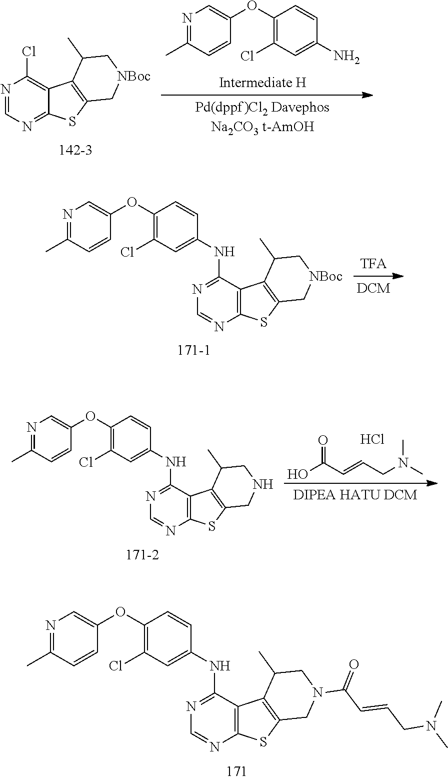

The title compound was prepared in analogous fashion to Compound 152, but using Intermediate H instead of Intermediate G in the first step. The title compound was obtained as yellow solid (278 mg, 26.7%). LCMS m/z [M+H]⁺: 549.2; HNMR (400 MHz, DMSO-d₆): δ 1.18 (d, 3H), 2.17 (s, 6H), 2.45 (s, 3H), 3.08 (br, 2H), 3.31 (br, 0.4H), 3.74 (d, 0.6H), 3.88 (br, 1H), 4.13 (d, 0.6H), 4.37 (d, 0.6H), 4.46 (d, 0.4H), 4.72 (d, 0.4H), 5.26 (d, 0.4H), 5.38 (d, 0.6H), 6.68-6.80 (m, 2H), 7.19 (d, 1H), 7.27 (d, 2H), 7.62 (br, 1H), 7.94 (br, 1H), 8.23 (t, 1H), 8.37-8.43 (m, 1H), 8.48 (s, 1H).

### Example 55: (E)-4-(Dimethylamino)-1-(4-((4-(imidazo[1,2-a]pyridin-7-yloxy)-3-methylphenyl)amino)-8,9-dihydro-5H-pyrimido[5′,4′:4,5]thieno[2,3-d]azepin-7(6H)-yl)but-2-en-1-one (Compound 172)

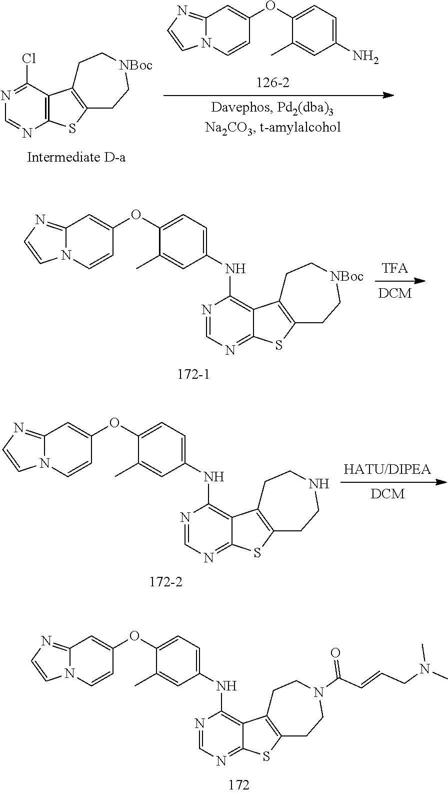 [SMILES: CC(C)(C)OC(=O)N1CCc2sc3ncnc(Cl)c3c2CC1 ; Cc1cc(N)ccc1Oc1ccn2ccnc2c1 ; Cc1cc(Nc2ncnc3sc4c(c23)CCN(C(=O)OC(C)(C)C)CC4)ccc1Oc1ccn2ccnc2c1 ; Cc1cc(Nc2ncnc3sc4c(c23)CCNCC4)ccc1Oc1ccn2ccnc2c1 ; Cc1cc(Nc2ncnc3sc4c(c23)CCN(C(=O)/C=C/CN(C)C)CC4)ccc1Oc1ccn2ccnc2c1]

### Step 1: tert-Butyl 4-((4-(imidazo[1,2-a]pyridin-7-yloxy)-3-m ethylphenyl) amino)-8, 9-dihydro-5H-pyrimido[5′,4′:4,5]thieno[2,3-d]azepine-7(6H)-carboxylate (172-1)

A mixture of Intermediate D-a (213 mg, 0.63 mmol), 126-2 (150 mg, 0.63 mmol), DavePhos (13 mg, 0.03 mmol), Pd₂(dba)₃ (30 mg, 0.03 mmol) and Cs₂CO₃ (410 mg, 1.26 mmol) in t-amylalcohol (15 mL) was stirred at 100° C. for 2.5 h under nitrogen atmosphere. The reaction mixture was quenched with water, extracted with EtOAc, washed with brine and concentrated. The resultant residue was purified by column chromatography (DCM:MeOH=80:1) to afford the title compound as white solid (110 mg, 32%). LCMS m/z [M+H]⁺: 543.3; ¹H NMR (400 MHz, DMSO-d₆): δ. 8.49-8.59 (m, 2H), 8.40 (s, 1H), 7.84 (s, 1H), 7.84-7.57 (m, 2H), 7.44 (d, 1H), 7.11 (d, 1H), 6.81 (dd, 1H), 6.52 (d, 1H), 3.62-3.71 (m, 4H), 3.36-3.38 (m, 2H), 3.12-3.17 (m, 2H), 2.18 (s, 3H), 1.43 (s, 9H).

### Step 2: N-(4-(Imidazo[1,2-a]pyridin-7-yloxy)-3-methylphenyl)-6,7,8,9-tetrahydro-5H-pyrimido[5′,4′:4,5]thieno[2,3-d]azepin-4-amine (172-2)

TFA (3 mL) was added to a solution of 172-1 (110 mg, 0.20 mmol) in DCM (6 mL). The mixture was stirred at ambient temperature for 1 h. The reaction solution was concentrated and the resultant residue dissolved in DCM and washed with aqueous Na₂CO₃ and brine. The organic layer was concentrated to afford the title compound as white solid (80 mg, 90%). LCMS m z [M+H]⁺: 443.2; ¹H NMR (400 MHz, DMSO-d₆): δ 8.62 (s, 1H), 8.55 (d, 1H), 8.39 (s, 1H), 7.84 (s, 1H), 7.55 (d, 1H), 7.52 (dd, 1H), 7.44 (d, 1H), 7.10 (d, 1H), 6.81 (dd, 1H), 6.52 (d, 1H), 3.24-3.29 (m, 2H), 2.99-3.01 (m, 2H), 2.89-2.93 (m, 4H), 2.17 (s, 3H).

### Step 3: (E)-4-(Dimethylamino)-1-(4-((4-(imidazo[1,2-a]pyridin-7-yloxy)-3-methylphenyl)amino)-8,9-dihydro-5H-pyrimido[5′,4′:4,5]thieno[2,3-d]azepin-7(6H)-yl)but-2-en-1-one (Compound 172)

A mixture of 172-2 (80 mg, 0.18 mmol), (E)-4-(dimethylamino)but-2-enoic acid hydrochloride (36 mg, 0.22 mmol), HATU (137 mg, 0.36 mmol), DIPEA (46 mg, 0.36 mmol) in DCM (10 mL) was stirred ambient temperature for 1 h. The reaction mixture was quenched with water, extracted with DCM, washed with brine and concentrated. The resultant residue was purified by prep-TLC to afford the title compound as white solid (38 mg, 38%). LCMS m/z [M+H]⁺: 554.2; ¹H NMR (400 MHz, DMSO-d₆): 6.8.52-8.57 (m, 2H), 8.41 (d, 1H), 7.85 (s, 1H), 7.53-7.57 (m, 2H), 7.44 (s, 1H), 7.10-7.13 (m, 1H), 6.53-6.90 (m, 4H), 3.78-3.97 (m, 4H), 3.43-3.50 (m, 4H), 3.18-3.22 (m, 2H), 2.35 (s, 6H), 2.17 (s, 3H).

### Example 56: (E)-4-(Dimethylamino)-1-(4-((4-((6-methoxypyridin-3-yl)oxy)-3-methylphenyl)amino)-8,9-dihydro-5H-pyrimido[5′,4′:4,5]thieno[2,3-d]azepin-7(6H)-yl)but-2-en-1-one (Compound 173)

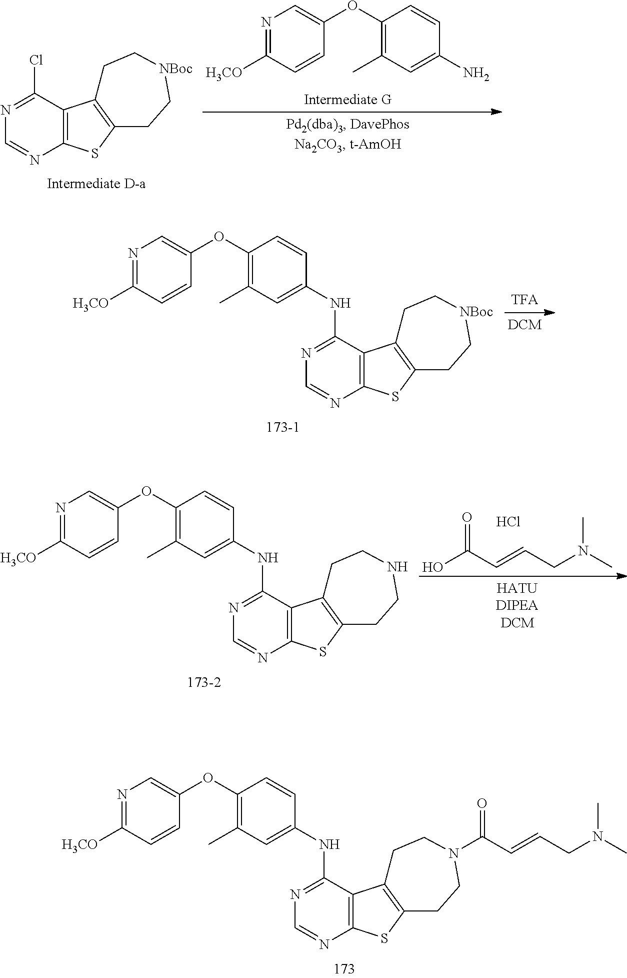

The title compound was prepared in analogous fashion to Compound 172, but using Intermediate G instead of 126-2 as starting material in the first step. The title compound was obtained as white solid (161 mg, 64%). LCMS m/z [M+H]⁺: 545.3; HNMR (400 MHz, DMSO-d₆): δ 8.40 (s, 1H), 8.35 (d, 1H), 7.91 (t, 1H), 7.47-7.48 (m, 1H), 7.37-7.41 (m, 2H), 6.82-6.87 (m, 2H), 6.64-6.68 (m, 2H), 3.76-3.94 (m, 7H), 3.37-3.40 (m, 2H), 3.16-3.21 (m, 2H), 3.06-3.07 (m, 2H), 2.23 (s, 3H), 2.15 and 2.16 (s, 6H).

### Example 57: (E)-1-(5-Methyl-4-((3-methyl-4-((6-methylpyridin-3-yl)oxy)phenyl)amino)-5,6-dihydropyrido[4′,3′:4,5]thieno[2,3-d]pyrimidin-7(8H)-yl)-4-morpholinobut-2-en-1-one (Compound 174)

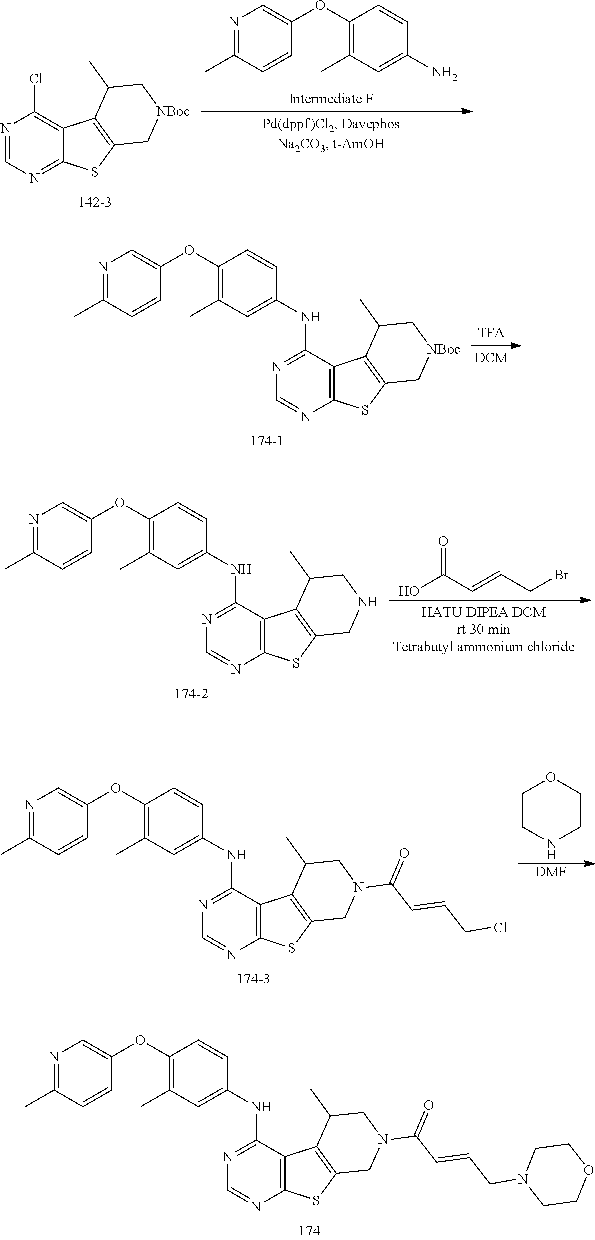

### Steps 1-3: (E)-4-Chloro-1-(5-methyl-4-((3-methyl-4-((6-methylpyridin-3-yl)oxy)phenyl)amino)-5,6-dihydropyrido[4′,3′:4,5]thieno[2,3-d]pyrimidin-7(8H)-yl)but-2-en-1-one (174-3)

Compound 174-2 was prepared in analogous fashion to compound 152-2, using Intermediate F instead of Intermediate G in the Buchwald coupling reaction. A solution of 174-2 (300 mg, 0.72 mmol), (E)-4-bromobut-2-enoic acid (237 mg, 1.44 mmol), EDCI (414 mg, 2.16 mmol) in DCM was stirred at ambient temperature for 1 h. Tetrabutyl ammonium chloride (600 mg, 2.16 mmol) was added and the mixture was stirred at ambient temperature for 2 h. The mixture was poured into water and extracted with EtOAc. The organic layer was washed with water and brine, dried over Na₂SO₄ and concentrated. The resultant residue was purified by column chromatography to afford the title compound as yellow solid (300 mg, 80%).

### Step 4: (E)-1-(5-Methyl-4-((3-methyl-4-((6-methylpyridin-3-yl)oxy)phenyl)amino)-5,6-dihydropyrido[4′,3′:4,5]thieno[2,3-d]pyrimidin-7(8H)-yl)-4-morpholinobut-2-en-1-one (Compound 174)

A solution of 174-3 (150 mg, 0.29 mmol) and morpholine (250 mg, 2.9 mmol) in DMF (5 mL) was stirred at 45° C. overnight. The resultant mixture was poured into water and extracted with EtOAc. The organic layer was washed with water and brine, dried over Na₂SO₄ and concentrated. The resultant residue was purified by column chromatography to afford the title compound as yellow solid (87 mg, 52%). LCMS m/z [M+H]⁺: 571.5; ¹HNMR (400 MHz, DMSO-d₆): δ 1.19 (d, 3H), 2.20 (s, 3H), 2.38 (br s, 4H), 2.44 (s, 3H), 3.14-3.18 (m, 2H), 3.27-3.31 (m, 0.4H), 3.59 (t, 4H), 3.72 (d, 0.6H), 3.87 (d, 1H), 4.13 (d, 0.6H), 4.35 (d, 0.6H), 4.47 (d, 0.4H), 4.70 (d, 0.4H), 5.26 (d, 0.4H), 5.37 (d, 0.6H), 6.68-6.83 (m, 2H), 6.95 (d, 1H), 7.19-7.26 (m, 2H), 7.47 (d, 1H), 7.55 (s, 1H), 8.18 (d, 1H), 8.23 (d, 1H), 8.41 (s, 1H).

### Example 58: (E)-1-(5-Methyl-4-((3-methyl-4-((6-methylpyridin-3-yl)oxy)phenyl)amino)-5,6-dihydropyrido[4′,3′:4,5]thieno[2,3-d]pyrimidin-7(8H)-yl)-4-morpholinobut-2-en-1-one

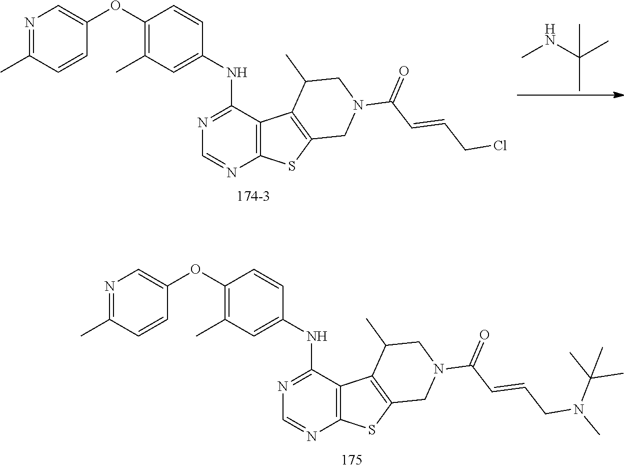

A solution of 174-3 (150 mg, 0.29 mmol), N,2-dimethylpropan-2-amine (50 mg, 0.58 mmol) and DIPEA (112 mg, 0.87 mmol) in DMF was heated at 45° C. overnight. The mixture was poured into water and extracted with EtOAc. The organic layer was washed with water and brine, dried over Na₂SO₄ and concentrated. The resultant residue was purified by column chromatography to afford the title compound as yellow solid (75 mg, 45.3%). LCMS m/z [M+H]⁺: 571.4; ¹HNMR (400 MHz, DMSO-d₆): δ 1.04 (s, 9H), 1.19 (d, 3H), 2.12 (s, 3H), 2.20 (s, 3H), 2.44 (s, 3H), 3.19 (br s, 2H), 3.31 (br s, 0.4H), 3.72 (d, 0.6H), 3.87 (br s, 1H), 4.13 (d, 0.6H), 4.34 (d, 0.6H), 4.46 (d, 0.4H), 4.71 (d, 0.4H), 5.24 (d, 0.4H), 5.39 (d, 0.6H), 6.67-6.80 (m, 2H), 6.95 (d, 1H), 7.19-7.26 (m, 2H), 7.48 (d, 1H), 7.55 (s, 1H), 8.18 (d, 1H), 8.23 (br, 1H), 8.41 (s, 1H).

### Example 59: (R)-1-(5-methyl-4-((3-methyl-4-((6-methylpyridin-3-yl)oxy)phenyl)amino)-5,8-dihydropyrido[4′,3′:4,5]thieno[2,3-d]pyrimidin-7(6H)-yl)-4-morpholinobut-2-en-1-one and (S)-1-(5-methyl-4-((3-methyl-4-((6-methylpyridin-3-yl)oxy)phenyl)amino)-5,8-dihydropyrido[4′,3′:4,5]thieno[2,3-d]pyrimidin-7(6H)-yl)-4-morpholinobut-2-en-1-one

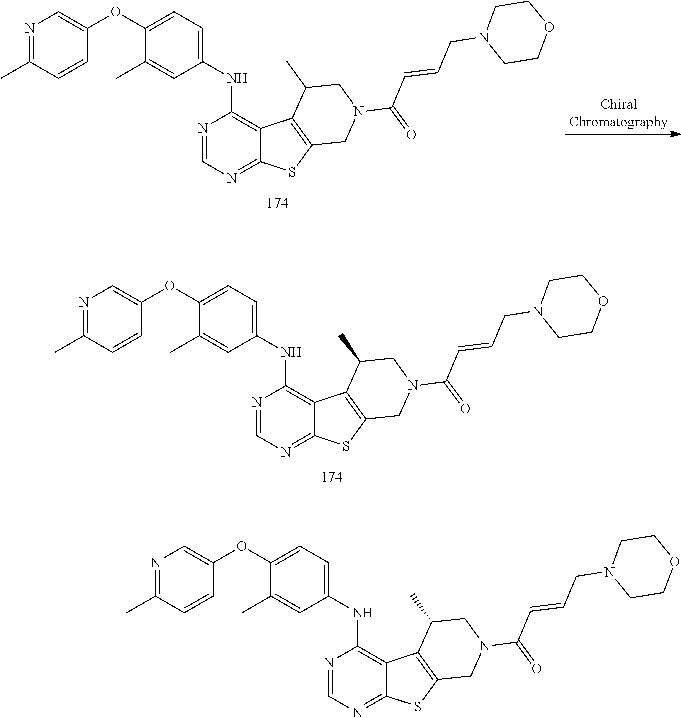

Enantiomers of Compound 174 were separated via HPLC using a Lux Cellulose-4 column. The faster eluting enantiomer was designated Compound 176 ([α]_{D}¹¹⁰=+126° (C=0.1 g/100 mL MeOH)) and slower eluting enantiomer designated Compound 177 ([α]_{d}¹¹⁰=−140° (C=0.1 g/100 mL MeOH)).

### Example 60: (R)-4-(tert-butyl(methyl)amino)-1-(5-methyl-4-((3-methyl-4-((6-methylpyridin-3-yl)oxy)phenyl)amino)-5,8-dihydropyrido[4′,3′:4,5]thieno[2,3-d]pyrimidin-7(6H)-yl)but-2-en-1-one and (S)-4-(tert-butyl(methyl)amino)-1-(5-methyl-4-((3-methyl-4-((6-methylpyridin-3-yl)oxy)phenyl)amino)-5,8-dihydropyrido[4′,3′:4,5]thieno[2,3-d]pyrimidin-7(6H)-yl)but-2-en-1-one

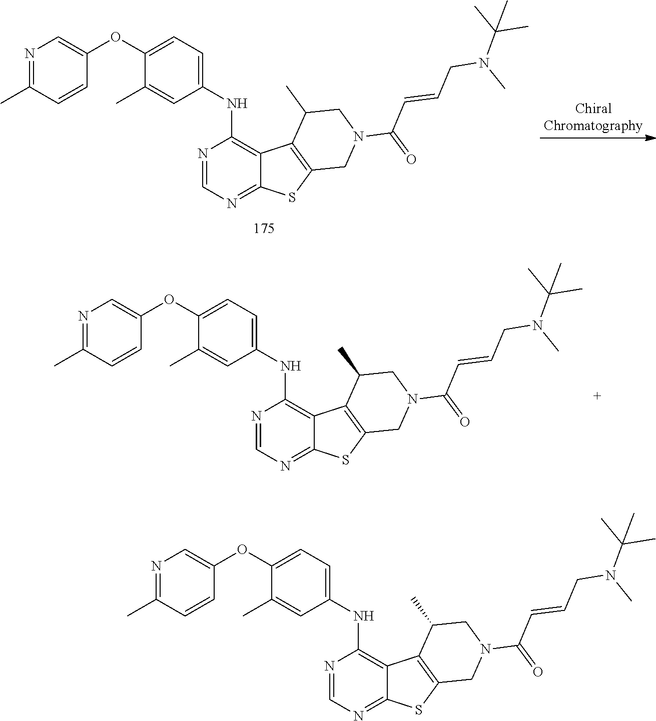

Enantiomers of Compound 175 were separated via HPLC using a Lux Cellulose-4 column. The faster eluting enantiomer was designated Compound 178 ([α]_{D}¹¹⁰=+115° (C=0.1 g/100 mL MeOH)) and slower eluting enantiomer designated Compound 179 ([α]_{D}¹¹⁰=−116° (C=0.1 g/100 mL MeOH)). Absolute configurations were assigned arbitrarily.

### Example 61: Parallel Synthesis of Aminocrotonamide Libraries

General Information. Regents and solvents were used as received from the supplier. DMA, DMSO and DIPEA were purchased from Sigma Aldrich. Reactions were performed in capped 2-dram vials on a small shaker. The separations of library products were performed on a Waters Automated Purification System using a single quadruple mass detector (SQD2, Waters); a 19×100 mm XBridge™ C-18 prep column from Waters corporation. Each separation of library compounds was eluted with a focused gradient from 10% to 90% CH₃CN in H₂O (containing v/v 0.1% formic acid) at a flow rate of 20 mL/min. All starting materials for reactions are prepared into a 0.25 M of DMA stock solution. All solvents were removed by EZ-2 from Genevec.

General Procedure. In 2-dram round bottom vials, 0.5 mL of DMA was first charged to the vials. To each vial, one of Intermediates K—N from Schemes 11-14 respectively (200 μL, 50 μmol) was added followed by the addition of amines (400 μL, 100 μmol) in a library format. DIPEA was added (50 μL neat) and the resultant solutions were heated and shaken at 40° C. for 16 h. Solvent was removed by EZ-2 and the resultant residues were dissolved into 1.0 mL to 2.0 mL of DMSO. The solutions were then subjected to reverse phase purification using a Waters Automated Purification System (conditions described previously in the General Information). The purity of library products was determined by LC absorbance at two wavelengths, typically 214 and 254 nm. All obtained compounds were >90% pure by peak area.

Compounds in Table 2 below were prepared as described above:

TABLE 2

| Com- | | Starting | M + H |
|---|---|---|---|
| pound | Structure | Material | observed |
| 12 | 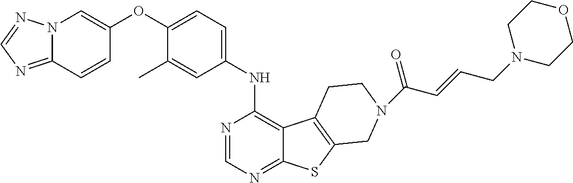 [SMILES: Cc1cc(Nc2ncnc3sc4c(c23)CCN(C(=O)/C=C/CN2CCOCC2)C4)ccc1Oc1ccc2ncnn2c1] | Intermediate M | 583.2 |
| 13 | 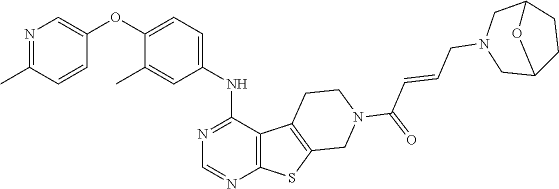 [SMILES: Cc1ccc(Oc2ccc(Nc3ncnc4sc5c(c34)CCN(C(=O)/C=C/CN3CC4CCC(C3)O4)C5)cc2C)cn1] | Intermediate K | 582.8 |
| 14 | 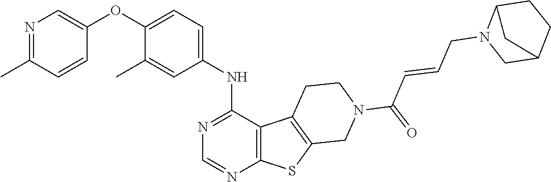 [SMILES: Cc1ccc(Oc2ccc(Nc3ncnc4sc5c(c34)CCN(C(=O)/C=C/CN3CC4CCC3C4)C5)cc2C)cn1] | Intermediate K | 566.9 |
| 15 | 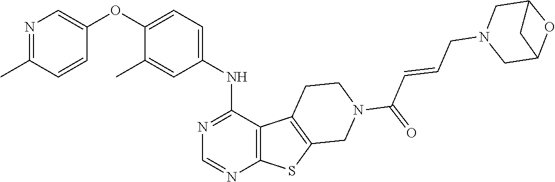 [SMILES: Cc1ccc(Oc2ccc(Nc3ncnc4sc5c(c34)CCN(C(=O)/C=C/CN3CC4CC(C3)O4)C5)cc2C)cn1] | Intermediate K | 568.8 |
| 18 | 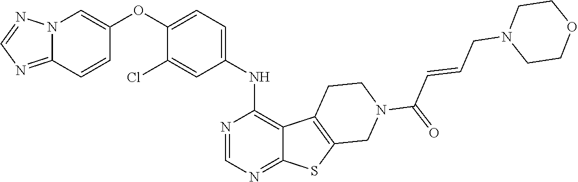 [SMILES: O=C(/C=C/CN1CCOCC1)N1CCc2c(sc3ncnc(Nc4ccc(Oc5ccc6ncnn6c5)c(Cl)c4)c23)C1] | Intermediate N | 603.1 |
| 20 | 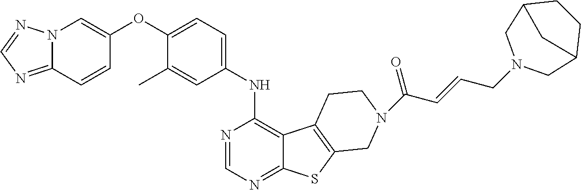 [SMILES: Cc1cc(Nc2ncnc3sc4c(c23)CCN(C(=O)/C=C/CN2CC3CCC(C3)C2)C4)ccc1Oc1ccc2ncnn2c1] | Intermediate M | 607.3 |
| 26 | 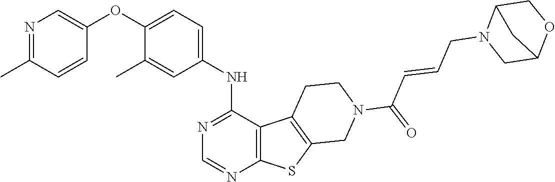 [SMILES: Cc1ccc(Oc2ccc(Nc3ncnc4sc5c(c34)CCN(C(=O)/C=C/CN3CC4CC3CO4)C5)cc2C)cn1] | Intermediate K | 569.3 |
| 27 | 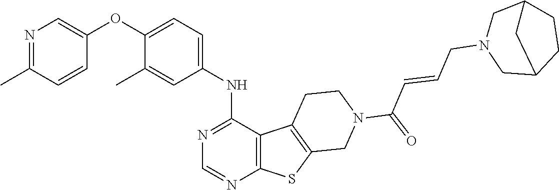 [SMILES: Cc1ccc(Oc2ccc(Nc3ncnc4sc5c(c34)CCN(C(=O)/C=C/CN3CC4CCC(C4)C3)C5)cc2C)cn1] | Intermediate K | 581.3 |
| 28 | 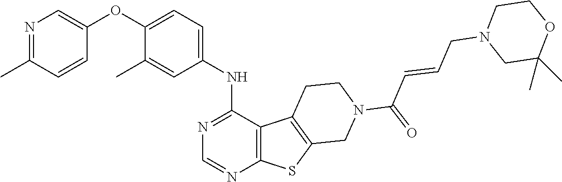 | Intermediate K | 585.3 |
| 29 | 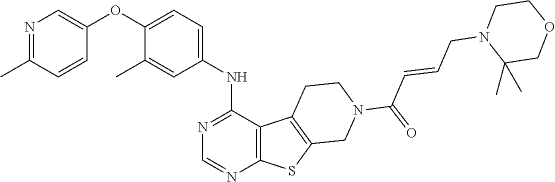 [SMILES: Cc1ccc(Oc2ccc(Nc3ncnc4sc5c(c34)CCN(C(=O)/C=C/CN3CCOCC3(C)C)C5)cc2C)cn1] | Intermediate K | 585.3 |
| 30 | 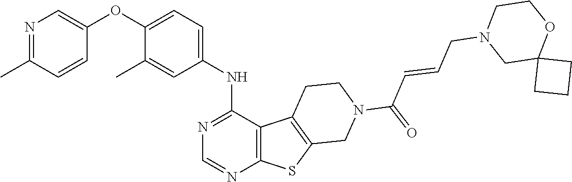 [SMILES: Cc1ccc(Oc2ccc(Nc3ncnc4sc5c(c34)CCN(C(=O)/C=C/CN3CCOC4(CCC4)C3)C5)cc2C)cn1] | Intermediate K | 597.3 |
| 31 | 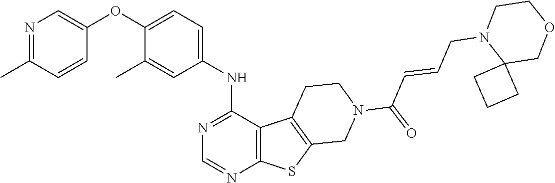 [SMILES: Cc1ccc(Oc2ccc(Nc3ncnc4sc5c(c34)CCN(C(=O)/C=C/CN3CCOCC34CCC4)C5)cc2C)cn1] | Intermediate K | 597.3 |
| 32 | 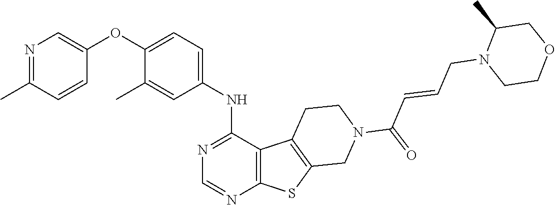 [SMILES: Cc1ccc(Oc2ccc(Nc3ncnc4sc5c(c34)CCN(C(=O)/C=C/CN3CCOC[C@@H]3C)C5)cc2C)cn1] | Intermediate K | 571.3 |
| 33 | 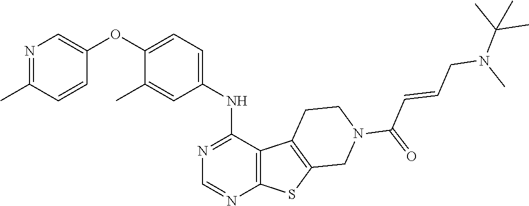 [SMILES: Cc1ccc(Oc2ccc(Nc3ncnc4sc5c(c34)CCN(C(=O)/C=C/CN(C)C(C)(C)C)C5)cc2C)cn1] | Intermediate K | 557.2 |
| 34 | 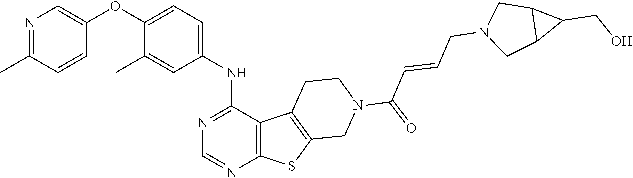 [SMILES: Cc1ccc(Oc2ccc(Nc3ncnc4sc5c(c34)CCN(C(=O)/C=C/CN3CC4C(CO)C4C3)C5)cc2C)cn1] | Intermediate K | 583.3 |
| 36 | 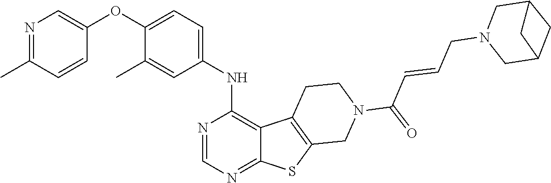 [SMILES: Cc1ccc(Oc2ccc(Nc3ncnc4sc5c(c34)CCN(C(=O)/C=C/CN3CC4CC(C4)C3)C5)cc2C)cn1] | Intermediate K | 567.2 |
| 37 | 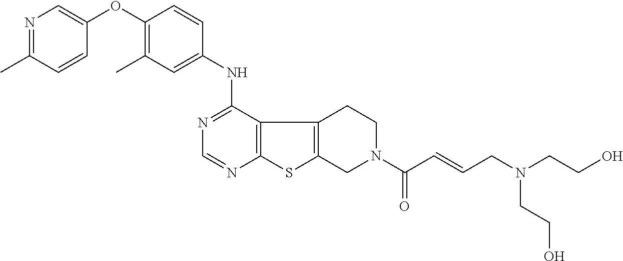 [SMILES: Cc1ccc(Oc2ccc(Nc3ncnc4sc5c(c34)CCN(C(=O)/C=C/CN(CCO)CCO)C5)cc2C)cn1] | Intermediate K | 575.4 |
| 38 | 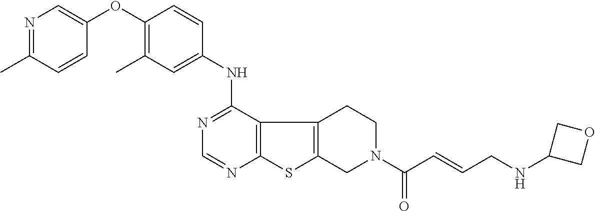 [SMILES: Cc1ccc(Oc2ccc(Nc3ncnc4sc5c(c34)CCN(C(=O)/C=C/CNC3COC3)C5)cc2C)cn1] | Intermediate K | 543.3 |
| 39 | 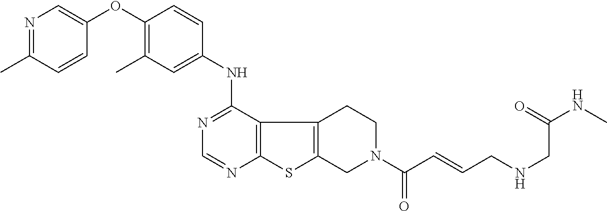 | Intermediate K | 558.3 |
| 40 | 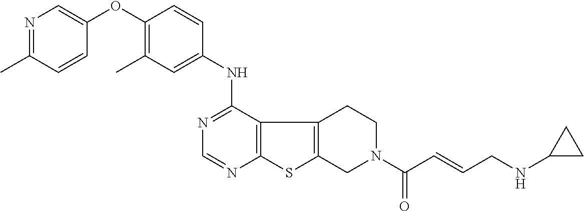 [SMILES: Cc1ccc(Oc2ccc(Nc3ncnc4sc5c(c34)CCN(C(=O)/C=C/CNC3CC3)C5)cc2C)cn1] | Intermediate K | 527.3 |
| 41 | 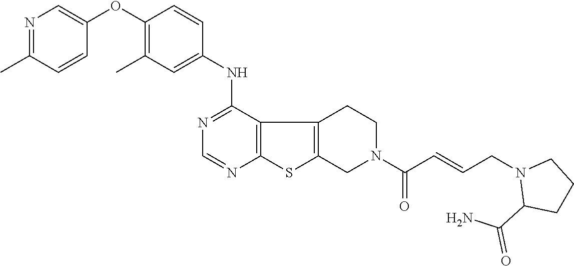 [SMILES: Cc1ccc(Oc2ccc(Nc3ncnc4sc5c(c34)CCN(C(=O)/C=C/CN3CCCC3C(N)=O)C5)cc2C)cn1] | Intermediate K | 584.4 |
| 42 | 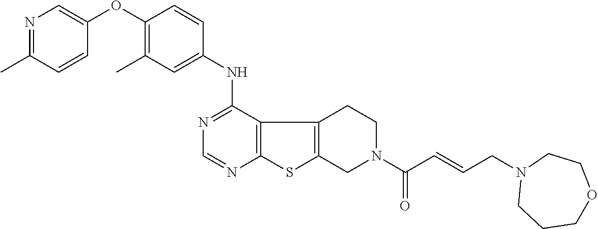 [SMILES: Cc1ccc(Oc2ccc(Nc3ncnc4sc5c(c34)CCN(C(=O)/C=C/CN3CCCOCC3)C5)cc2C)cn1] | Intermediate K | 571.3 |
| 43 | 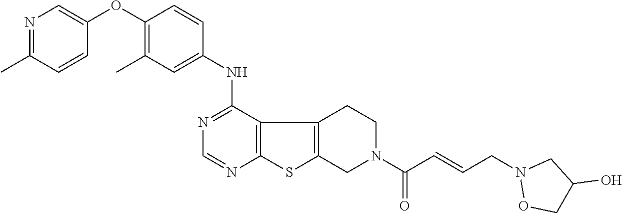 [SMILES: Cc1ccc(Oc2ccc(Nc3ncnc4sc5c(c34)CCN(C(=O)/C=C/CN3CC(O)CO3)C5)cc2C)cn1] | Intermediate K | 559.3 |
| 44 | 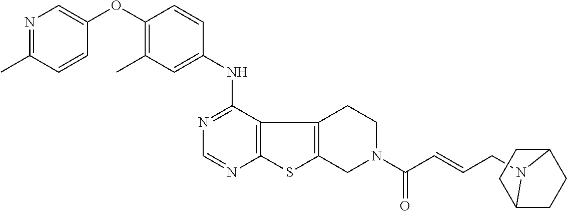 [SMILES: Cc1ccc(Oc2ccc(Nc3ncnc4sc5c(c34)CCN(C(=O)/C=C/CN3C4CCC3CC4)C5)cc2C)cn1] | Intermediate K | 567.3 |
| 45 | 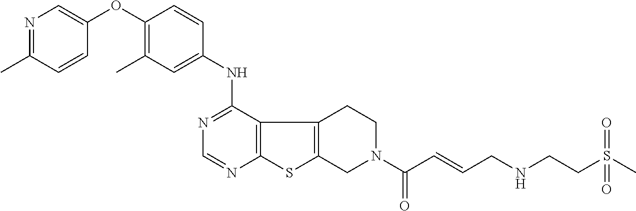 | Intermediate K | 593.3 |
| 46 | 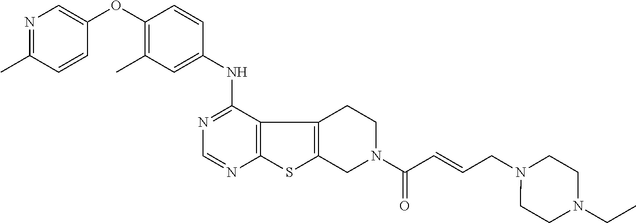 [SMILES: CCN1CCN(C/C=C/C(=O)N2CCc3c(sc4ncnc(Nc5ccc(Oc6ccc(C)nc6)c(C)c5)c34)C2)CC1] | Intermediate K | 584.4 |
| 47 | 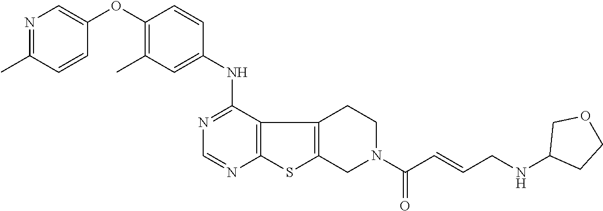 [SMILES: Cc1ccc(Oc2ccc(Nc3ncnc4sc5c(c34)CCN(C(=O)/C=C/CNC3CCOC3)C5)cc2C)cn1] | Intermediate K | 557.3 |
| 48 | 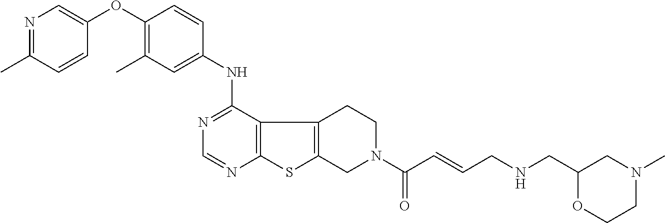 | Intermediate K | 599.2 |
| 49 | 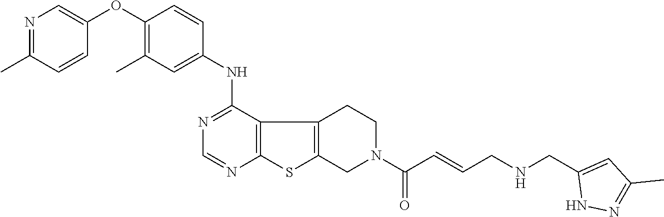 | Intermediate K | 581.3 |
| 51 | 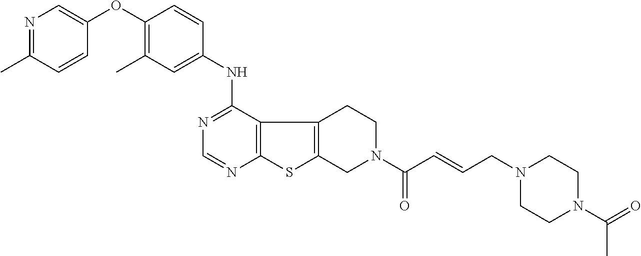 [SMILES: CC(=O)N1CCN(C/C=C/C(=O)N2CCc3c(sc4ncnc(Nc5ccc(Oc6ccc(C)nc6)c(C)c5)c34)C2)CC1] | Intermediate K | 598.4 |
| 52 | 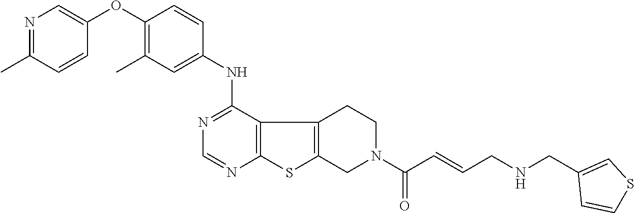 [SMILES: Cc1ccc(Oc2ccc(Nc3ncnc4sc5c(c34)CCN(C(=O)/C=C/CNCc3ccsc3)C5)cc2C)cn1] | Intermediate K | 583.2 |
| 53 | 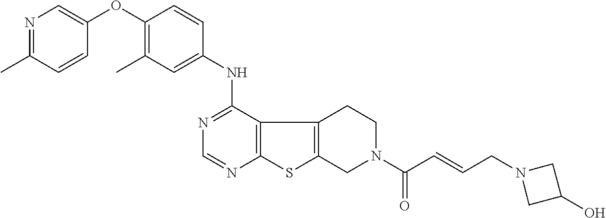 [SMILES: Cc1ccc(Oc2ccc(Nc3ncnc4sc5c(c34)CCN(C(=O)/C=C/CN3CC(O)C3)C5)cc2C)cn1] | Intermediate K | 543.3 |
| 54 | 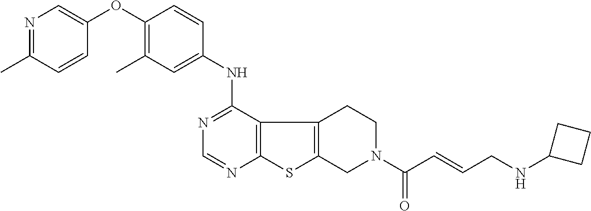 [SMILES: Cc1ccc(Oc2ccc(Nc3ncnc4sc5c(c34)CCN(C(=O)/C=C/CNC3CCC3)C5)cc2C)cn1] | Intermediate K | 541.3 |
| 55 | 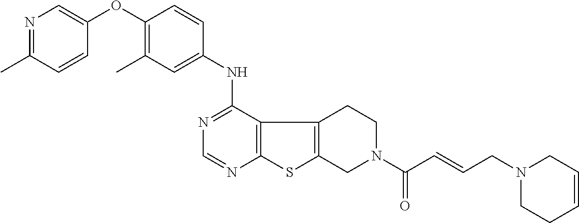 [SMILES: Cc1ccc(Oc2ccc(Nc3ncnc4sc5c(c34)CCN(C(=O)/C=C/CN3CC=CCC3)C5)cc2C)cn1] | Intermediate K | 553.3 |
| 56 | 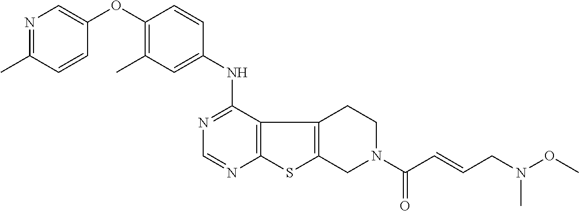 | Intermediate K | 531.3 |
| 57 | 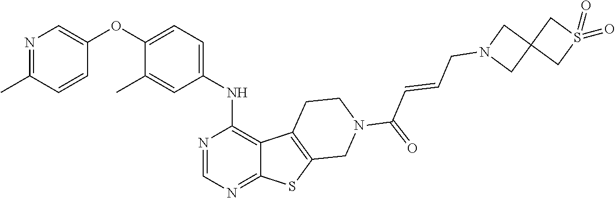 [SMILES: Cc1ccc(Oc2ccc(Nc3ncnc4sc5c(c34)CCN(C(=O)/C=C/CN3CC4(C3)CS(=O)(=O)C4)C5)cc2C)cn1] | Intermediate K | 617.3 |
| 58 | 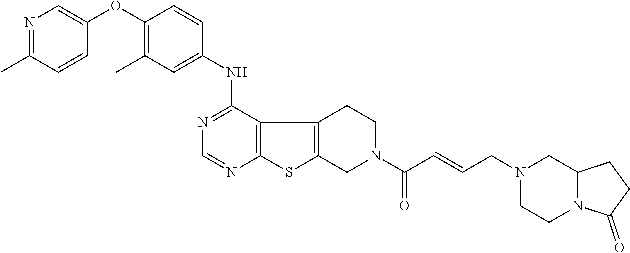 [SMILES: Cc1ccc(Oc2ccc(Nc3ncnc4sc5c(c34)CCN(C(=O)/C=C/CN3CCN4C(=O)CCC4C3)C5)cc2C)cn1] | Intermediate K | 610.3 |
| 59 | 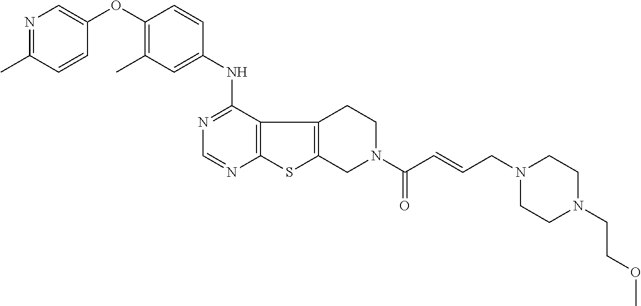 | Intermediate K | 614.4 |
| 60 | 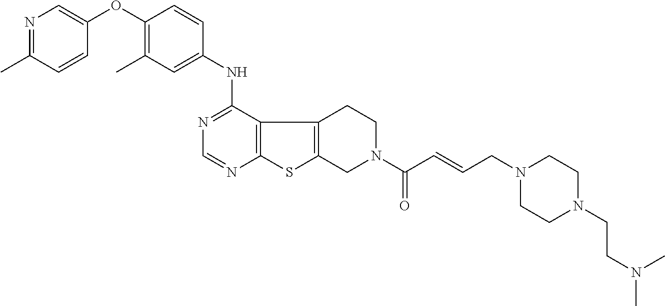 | Intermediate K | 642.4 |
| 61 | 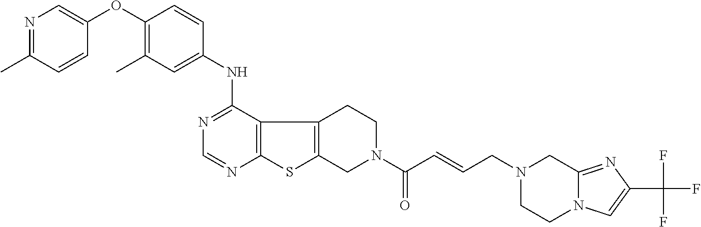 [SMILES: Cc1ccc(Oc2ccc(Nc3ncnc4sc5c(c34)CCN(C(=O)/C=C/CN3CCn4cc(C(F)(F)F)nc4C3)C5)cc2C)cn1] | Intermediate K | 661.2 |
| 62 | 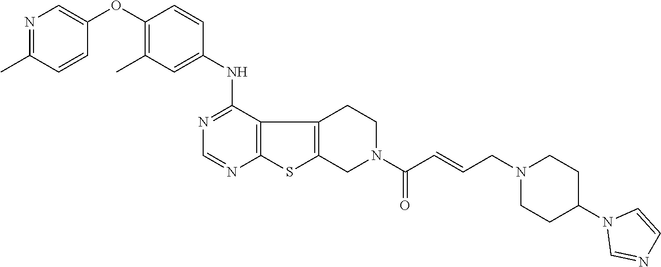 [SMILES: Cc1ccc(Oc2ccc(Nc3ncnc4sc5c(c34)CCN(C(=O)/C=C/CN3CCC(n4ccnc4)CC3)C5)cc2C)cn1] | Intermediate K | 621.3 |
| 65 | 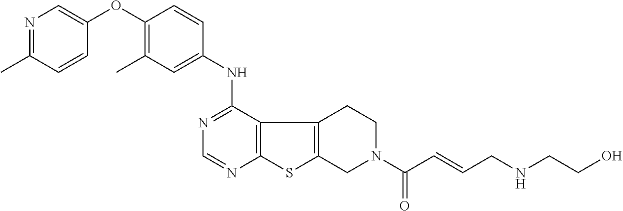 [SMILES: Cc1ccc(Oc2ccc(Nc3ncnc4sc5c(c34)CCN(C(=O)/C=C/CNCCO)C5)cc2C)cn1] | Intermediate K | 531.3 |
| 66 | 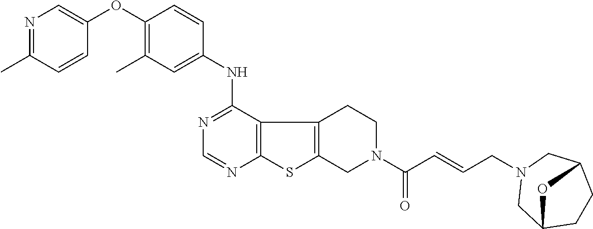 [SMILES: Cc1ccc(Oc2ccc(Nc3ncnc4sc5c(c34)CCN(C(=O)/C=C/CN3C[C@H]4CC[C@@H](C3)O4)C5)cc2C)cn1] | Intermediate K | 583.4 |
| 67 | 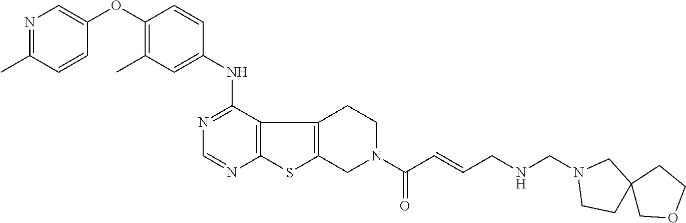 [SMILES: Cc1ccc(Oc2ccc(Nc3ncnc4sc5c(c34)CCN(C(=O)/C=C/CNCN3CCC4(CCOC4)C3)C5)cc2C)cn1] | Intermediate K | 597.4 |
| 68 | 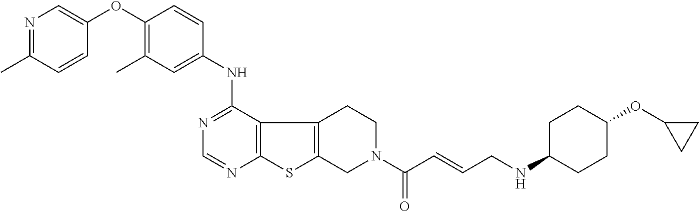 [SMILES: Cc1ccc(Oc2ccc(Nc3ncnc4sc5c(c34)CCN(C(=O)/C=C/CN[C@H]3CC[C@H](OC4CC4)CC3)C5)cc2C)cn1] | Intermediate K | 625.3 |
| 69 | 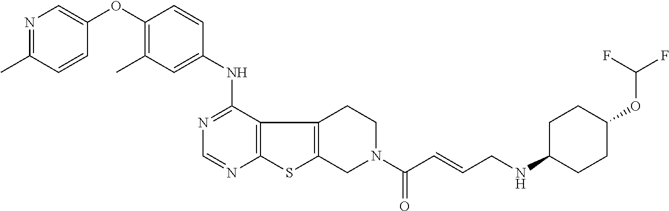 [SMILES: Cc1ccc(Oc2ccc(Nc3ncnc4sc5c(c34)CCN(C(=O)/C=C/CN[C@H]3CC[C@H](OC(F)F)CC3)C5)cc2C)cn1] | Intermediate K | 635.3 |
| 70 | 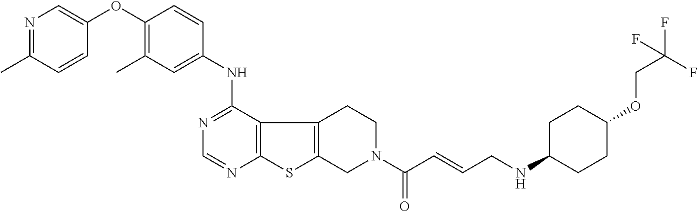 [SMILES: Cc1ccc(Oc2ccc(Nc3ncnc4sc5c(c34)CCN(C(=O)/C=C/CN[C@H]3CC[C@H](OCC(F)(F)F)CC3)C5)cc2C)cn1] | Intermediate K | 667.3 |
| 71 | 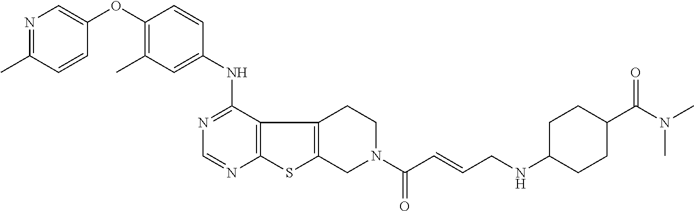 | Intermediate K | 640.4 |
| 72 | 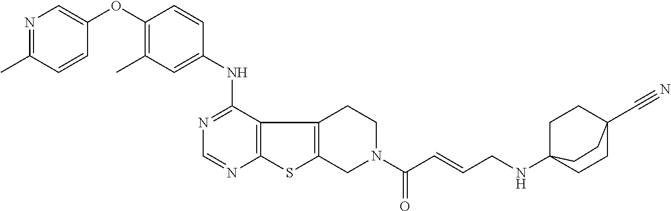 [SMILES: Cc1ccc(Oc2ccc(Nc3ncnc4sc5c(c34)CCN(C(=O)/C=C/CNC34CCC(C#N)(CC3)CC4)C5)cc2C)cn1] | Intermediate K | 620.3 |
| 73 | 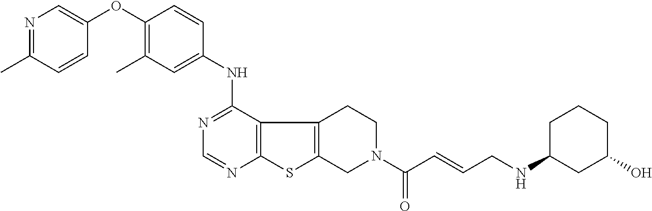 [SMILES: Cc1ccc(Oc2ccc(Nc3ncnc4sc5c(c34)CCN(C(=O)/C=C/CN[C@H]3CCC[C@H](O)C3)C5)cc2C)cn1] | Intermediate K | 585.4 |
| 74 | 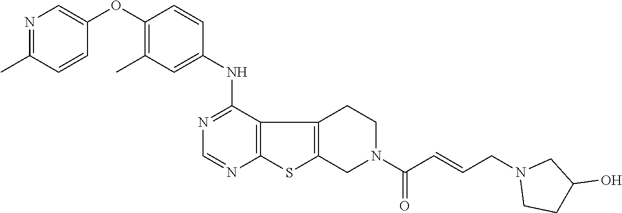 [SMILES: Cc1ccc(Oc2ccc(Nc3ncnc4sc5c(c34)CCN(C(=O)/C=C/CN3CCC(O)C3)C5)cc2C)cn1] | Intermediate K | 557.4 |
| 75 | 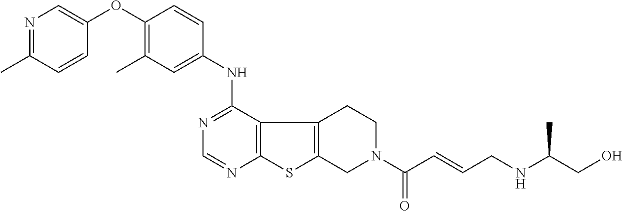 [SMILES: Cc1ccc(Oc2ccc(Nc3ncnc4sc5c(c34)CCN(C(=O)/C=C/CN[C@@H](C)CO)C5)cc2C)cn1] | Intermediate K | 545.4 |
| 76 | 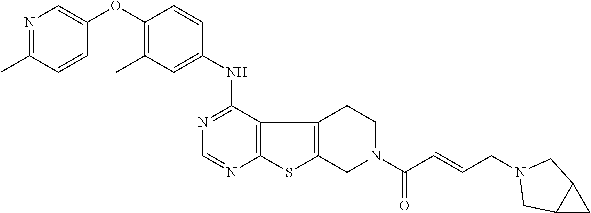 [SMILES: Cc1ccc(Oc2ccc(Nc3ncnc4sc5c(c34)CCN(C(=O)/C=C/CN3CC4CC4C3)C5)cc2C)cn1] | Intermediate K | 553.3 |
| 77 | 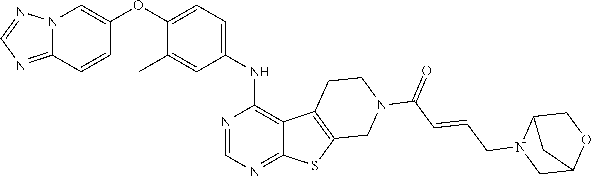 [SMILES: Cc1cc(Nc2ncnc3sc4c(c23)CCN(C(=O)/C=C/CN2CC3CC2CO3)C4)ccc1Oc1ccc2ncnn2c1] | Intermediate M | 595.3 |
| 79 | 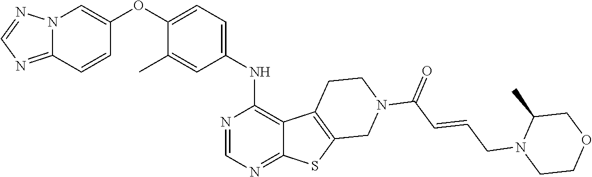 [SMILES: Cc1cc(Nc2ncnc3sc4c(c23)CCN(C(=O)/C=C/CN2CCOC[C@@H]2C)C4)ccc1Oc1ccc2ncnn2c1] | Intermediate M | 597.4 |
| 80 | 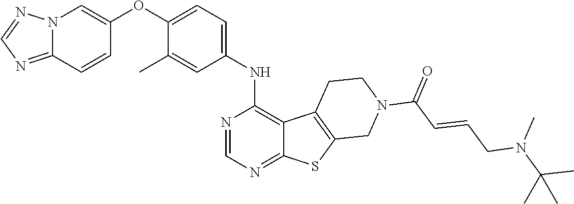 [SMILES: Cc1cc(Nc2ncnc3sc4c(c23)CCN(C(=O)/C=C/CN(C)C(C)(C)C)C4)ccc1Oc1ccc2ncnn2c1] | Intermediate M | 583.4 |
| 81 | 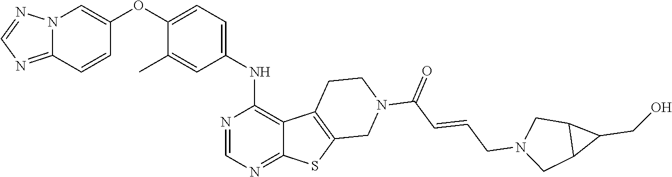 [SMILES: Cc1cc(Nc2ncnc3sc4c(c23)CCN(C(=O)/C=C/CN2CC3C(CO)C3C2)C4)ccc1Oc1ccc2ncnn2c1] | Intermediate M | 609.3 |
| 82 | 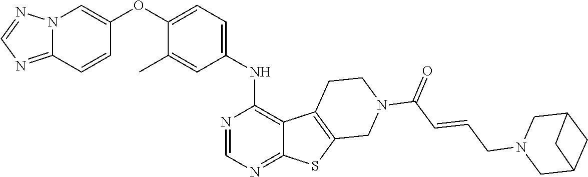 [SMILES: Cc1cc(Nc2ncnc3sc4c(c23)CCN(C(=O)/C=C/CN2CC3CC(C3)C2)C4)ccc1Oc1ccc2ncnn2c1] | Intermediate M | 593.2 |
| 83 | 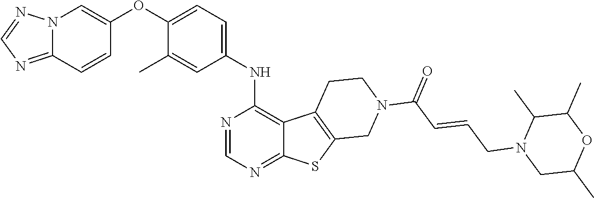 [SMILES: Cc1cc(Nc2ncnc3sc4c(c23)CCN(C(=O)/C=C/CN2CC(C)OC(C)C2C)C4)ccc1Oc1ccc2ncnn2c1] | Intermediate M | 625.5 |
| 84 | 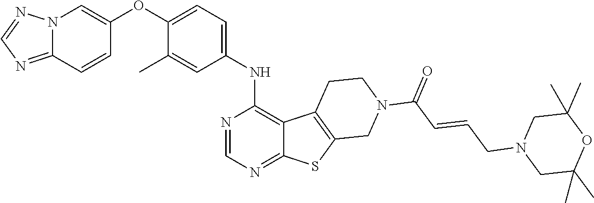 [SMILES: Cc1cc(Nc2ncnc3sc4c(c23)CCN(C(=O)/C=C/CN2CC(C)(C)OC(C)(C)C2)C4)ccc1Oc1ccc2ncnn2c1] | Intermediate M | 639.3 |
| 85 | 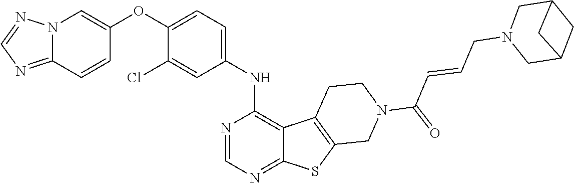 [SMILES: O=C(/C=C/CN1CC2CC(C2)C1)N1CCc2c(sc3ncnc(Nc4ccc(Oc5ccc6ncnn6c5)c(Cl)c4)c23)C1] | Intermediate N | 307.5 (M/2 + 1) |
| 87 | 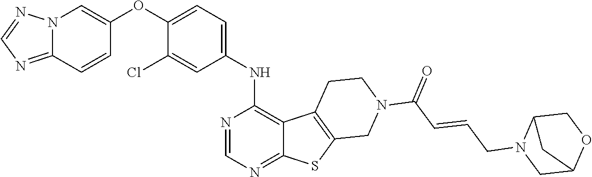 [SMILES: O=C(/C=C/CN1CC2CC1CO2)N1CCc2c(sc3ncnc(Nc4ccc(Oc5ccc6ncnn6c5)c(Cl)c4)c23)C1] | Intermediate N | 615.3 |
| 88 | 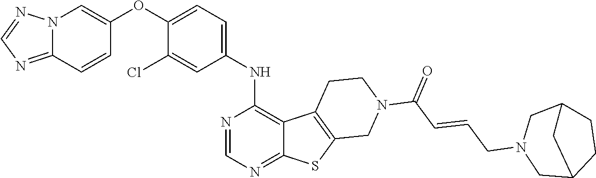 [SMILES: O=C(/C=C/CN1CC2CCC(C2)C1)N1CCc2c(sc3ncnc(Nc4ccc(Oc5ccc6ncnn6c5)c(Cl)c4)c23)C1] | Intermediate N | 627.3 |
| 90 | 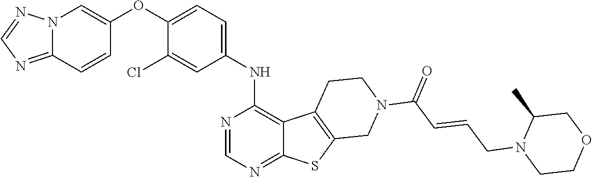 [SMILES: C[C@H]1COCCN1C/C=C/C(=O)N1CCc2c(sc3ncnc(Nc4ccc(Oc5ccc6ncnn6c5)c(Cl)c4)c23)C1] | Intermediate N | 617.3 |
| 91 | 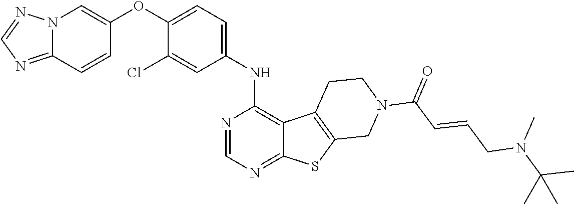 [SMILES: CN(C/C=C/C(=O)N1CCc2c(sc3ncnc(Nc4ccc(Oc5ccc6ncnn6c5)c(Cl)c4)c23)C1)C(C)(C)C] | Intermediate N | 603.3 |
| 92 | 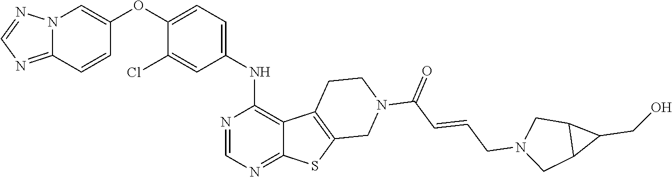 [SMILES: O=C(/C=C/CN1CC2C(CO)C2C1)N1CCc2c(sc3ncnc(Nc4ccc(Oc5ccc6ncnn6c5)c(Cl)c4)c23)C1] | Intermediate N | 629.3 |
| 112 | 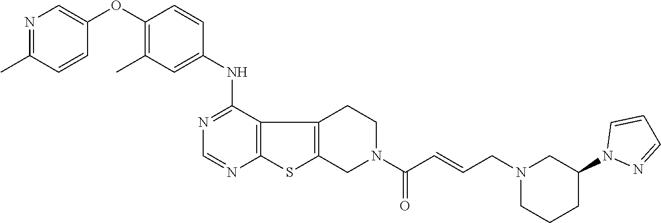 [SMILES: Cc1ccc(Oc2ccc(Nc3ncnc4sc5c(c34)CCN(C(=O)/C=C/CN3CCC[C@H](n4cccn4)C3)C5)cc2C)cn1] | Intermediate K | 621.4 |
| 113 | 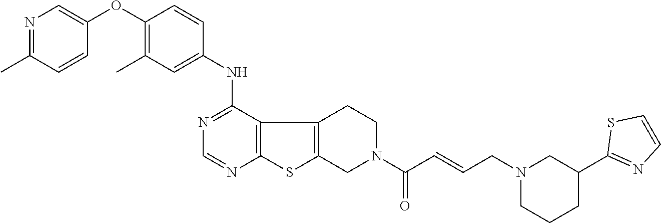 [SMILES: Cc1ccc(Oc2ccc(Nc3ncnc4sc5c(c34)CCN(C(=O)/C=C/CN3CCCC(c4nccs4)C3)C5)cc2C)cn1] | Intermediate K | 638.3 |
| 114 | 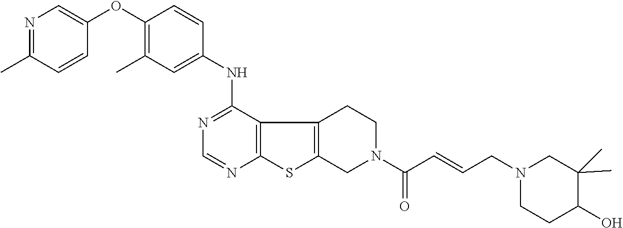 | Intermediate K | 599.3 |
| 115 | 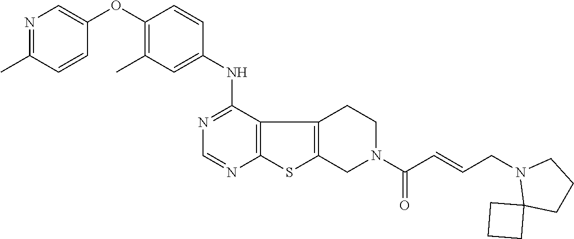 [SMILES: Cc1ccc(Oc2ccc(Nc3ncnc4sc5c(c34)CCN(C(=O)/C=C/CN3CCCC34CCC4)C5)cc2C)cn1] | Intermediate K | 581.4 |
| 116 | 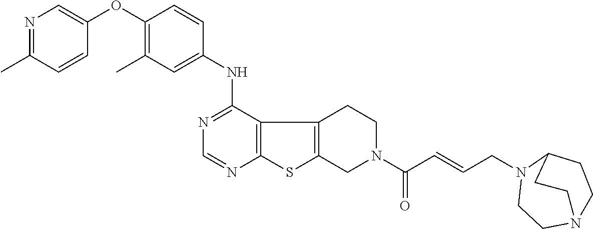 [SMILES: Cc1ccc(Oc2ccc(Nc3ncnc4sc5c(c34)CCN(C(=O)/C=C/CN3CCN4CCC3CC4)C5)cc2C)cn1] | Intermediate K | 596.4 |
| 117 | 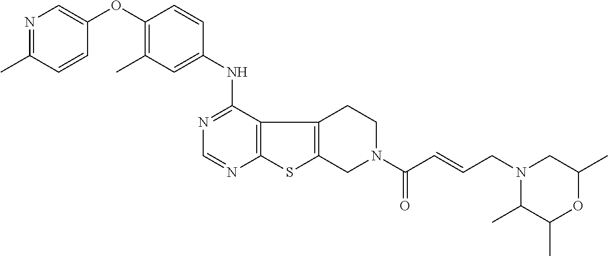 | Intermediate K | 599.4 |
| 118 | 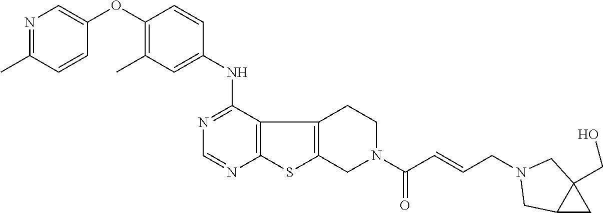 [SMILES: Cc1ccc(Oc2ccc(Nc3ncnc4sc5c(c34)CCN(C(=O)/C=C/CN3CC4CC4(CO)C3)C5)cc2C)cn1] | Intermediate K | 583.3 |
| 119 | 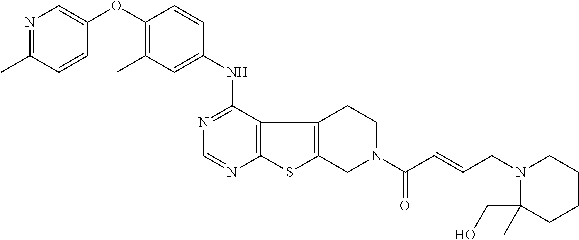 [SMILES: Cc1ccc(Oc2ccc(Nc3ncnc4sc5c(c34)CCN(C(=O)/C=C/CN3CCCCC3(C)CO)C5)cc2C)cn1] | Intermediate K | 599.3 |
| 120 | 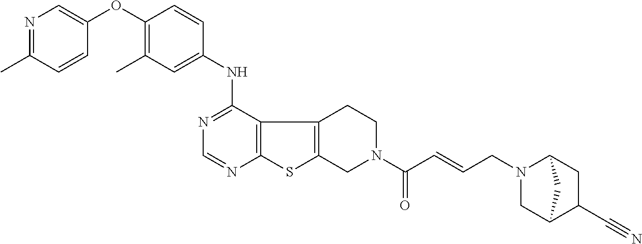 [SMILES: Cc1ccc(Oc2ccc(Nc3ncnc4sc5c(c34)CCN(C(=O)/C=C/CN3C[C@H]4C[C@@H]3CC4C#N)C5)cc2C)cn1] | Intermediate K | 592.3 |
| 121 | 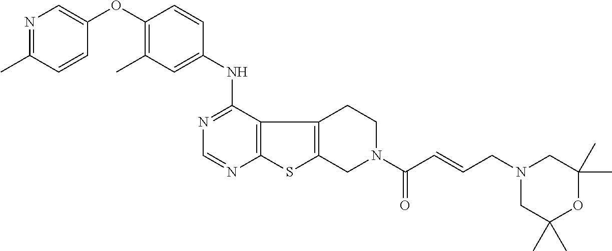 | Intermediate K | 613.4 |
| 122 | 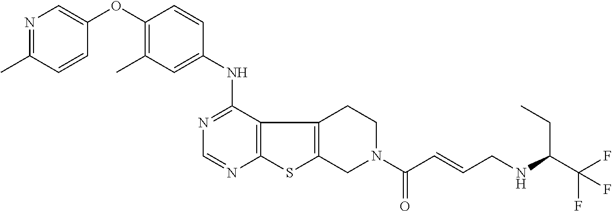 [SMILES: CC[C@H](NC/C=C/C(=O)N1CCc2c(sc3ncnc(Nc4ccc(Oc5ccc(C)nc5)c(C)c4)c23)C1)C(F)(F)F] | Intermediate K | 597.3 |
| 123 | 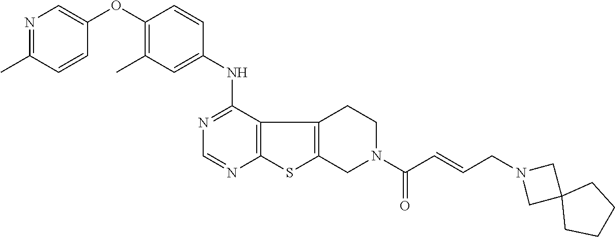 [SMILES: Cc1ccc(Oc2ccc(Nc3ncnc4sc5c(c34)CCN(C(=O)/C=C/CN3CC4(CCCC4)C3)C5)cc2C)cn1] | Intermediate K | 581.3 |
| 124 | 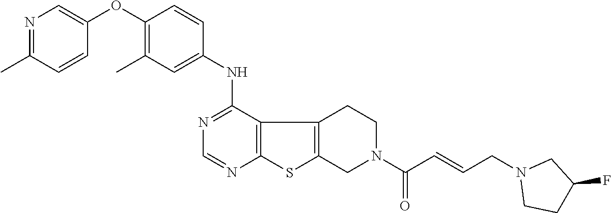 [SMILES: Cc1ccc(Oc2ccc(Nc3ncnc4sc5c(c34)CCN(C(=O)/C=C/CN3CC[C@H](F)C3)C5)cc2C)cn1] | Intermediate K | 559.3 |
| 131 | 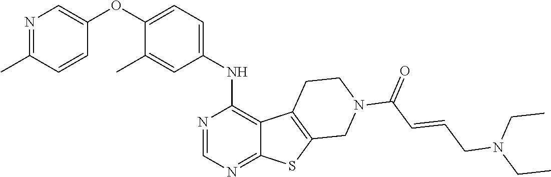 [SMILES: CCN(CC)C/C=C/C(=O)N1CCc2c(sc3ncnc(Nc4ccc(Oc5ccc(C)nc5)c(C)c4)c23)C1] | Intermediate K | 543.2 |
| 132 | 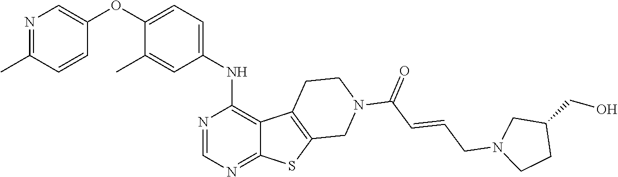 [SMILES: Cc1ccc(Oc2ccc(Nc3ncnc4sc5c(c34)CCN(C(=O)/C=C/CN3CC[C@@H](CO)C3)C5)cc2C)cn1] | Intermediate K | 571.3 |
| 133 | 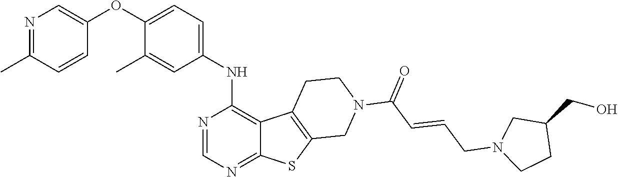 [SMILES: Cc1ccc(Oc2ccc(Nc3ncnc4sc5c(c34)CCN(C(=O)/C=C/CN3CC[C@H](CO)C3)C5)cc2C)cn1] | Intermediate K | 571.3 |
| 134 | 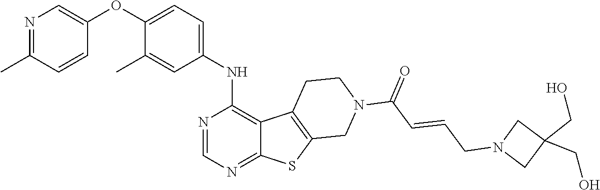 [SMILES: Cc1ccc(Oc2ccc(Nc3ncnc4sc5c(c34)CCN(C(=O)/C=C/CN3CC(CO)(CO)C3)C5)cc2C)cn1] | Intermediate K | 587.2 |
| 135 | 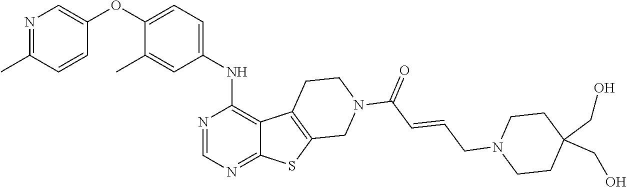 [SMILES: Cc1ccc(Oc2ccc(Nc3ncnc4sc5c(c34)CCN(C(=O)/C=C/CN3CCC(CO)(CO)CC3)C5)cc2C)cn1] | Intermediate K | 615.2 |
| 136 | 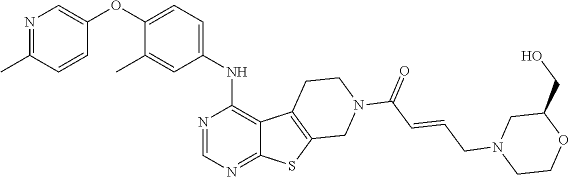 [SMILES: Cc1ccc(Oc2ccc(Nc3ncnc4sc5c(c34)CCN(C(=O)/C=C/CN3CCO[C@H](CO)C3)C5)cc2C)cn1] | Intermediate K | 587.2 |
| 137 | 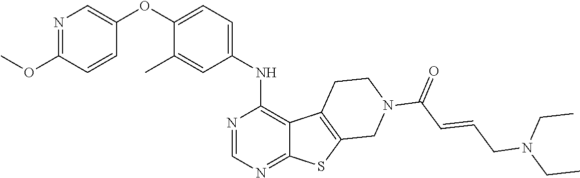 [SMILES: CCN(CC)C/C=C/C(=O)N1CCc2c(sc3ncnc(Nc4ccc(Oc5ccc(OC)nc5)c(C)c4)c23)C1] | Intermediate L | 559.3 |
| 139 | 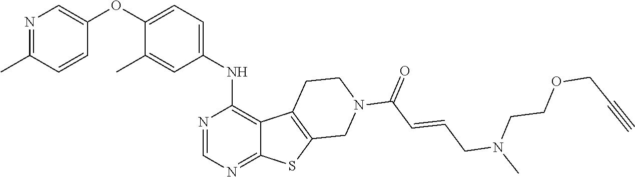 [SMILES: C#CCOCCN(C)C/C=C/C(=O)N1CCc2c(sc3ncnc(Nc4ccc(Oc5ccc(C)nc5)c(C)c4)c23)C1] | Intermediate K | 583.3 |
| 140 | 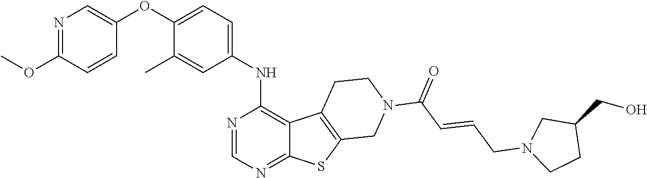 [SMILES: COc1ccc(Oc2ccc(Nc3ncnc4sc5c(c34)CCN(C(=O)/C=C/CN3CC[C@H](CO)C3)C5)cc2C)cn1] | Intermediate L | 587.2 |
| 143 | 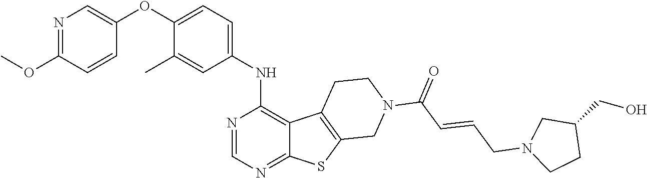 [SMILES: COc1ccc(Oc2ccc(Nc3ncnc4sc5c(c34)CCN(C(=O)/C=C/CN3CC[C@@H](CO)C3)C5)cc2C)cn1] | Intermediate L | 587.2 |
| 144 | 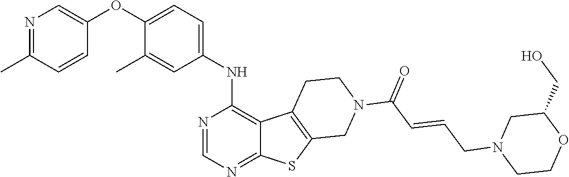 [SMILES: Cc1ccc(Oc2ccc(Nc3ncnc4sc5c(c34)CCN(C(=O)/C=C/CN3CCO[C@@H](CO)C3)C5)cc2C)cn1] | Intermediate K | 587.3 |
| 150 | 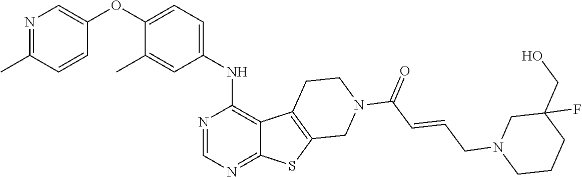 [SMILES: Cc1ccc(Oc2ccc(Nc3ncnc4sc5c(c34)CCN(C(=O)/C=C/CN3CCCC(F)(CO)C3)C5)cc2C)cn1] | Intermediate K | 603.3 |
| 151 | 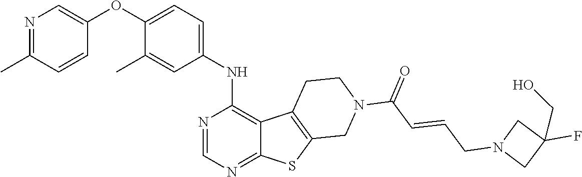 [SMILES: Cc1ccc(Oc2ccc(Nc3ncnc4sc5c(c34)CCN(C(=O)/C=C/CN3CC(F)(CO)C3)C5)cc2C)cn1] | Intermediate K | 575.2 |
| 154 | 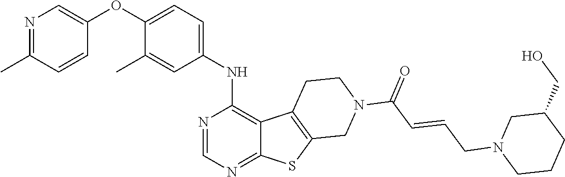 [SMILES: Cc1ccc(Oc2ccc(Nc3ncnc4sc5c(c34)CCN(C(=O)/C=C/CN3CCC[C@@H](CO)C3)C5)cc2C)cn1] | Intermediate K | 585.2 |
| 155 | 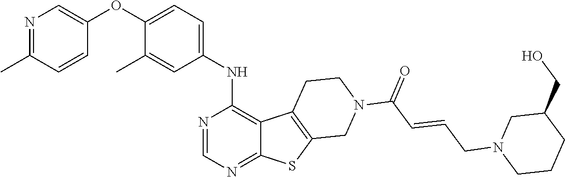 [SMILES: Cc1ccc(Oc2ccc(Nc3ncnc4sc5c(c34)CCN(C(=O)/C=C/CN3CCC[C@H](CO)C3)C5)cc2C)cn1] | Intermediate K | 585.3 |
| 163 | 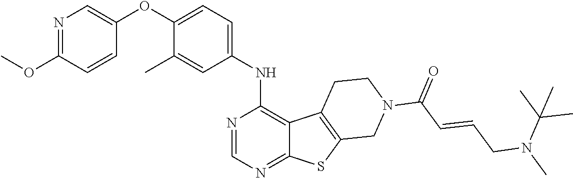 [SMILES: COc1ccc(Oc2ccc(Nc3ncnc4sc5c(c34)CCN(C(=O)/C=C/CN(C)C(C)(C)C)C5)cc2C)cn1] | Intermediate L | 573.2 |
| 164 | 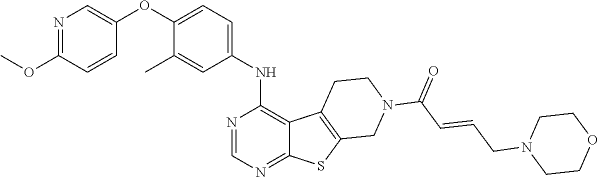 [SMILES: COc1ccc(Oc2ccc(Nc3ncnc4sc5c(c34)CCN(C(=O)/C=C/CN3CCOCC3)C5)cc2C)cn1] | Intermediate L | 573.2 |

### Example 401: Enzymatic Assay Protocols for Potency Assessment Against HER2 WT, HER2YVMA, and EGFR WT

A 2× stock solution of HER2 WT (ERBB2 0108-0000-1; ProQinase, Freiburg, Germany) corresponding to method a, was prepared as described below. Alternatively, a 2× stock solution of HER2YVMA (custom purification; Viva biotech, Shanghai, China) corresponding to method b, was prepared as described below. For method c, a 2× stock of EGFR (ERBB1 PR7295B, Thermo Fisher, Carlsbad, CA) was prepared as described below. All 2× solutions were prepared in buffer containing 50 mM HEPES, pH 7.5, 10 mM MnCl₂, MgCl₂, 0.005% Tween 20, 1 mM TCEP. 25 ul of 2× enzyme solution was added to a Seahorse low volume 384-well polypropylene assay plate (201288-100, Seahorse Bioscience, Chicopee, MA) containing 1 μl of compound serially diluted in 50% DMSO. To initiate the reaction, 25 μl of solution containing 2× final concentration of ATP and ProfilerPro Peptide 22 (760366, Perkin Elmer Hopkinton, MA) was added to each well. The reactions were separated on an LC3000 (Caliper Life Sciences) or a LabChip EZReader (Perkin Elmer) in kinetic mode.

At the conclusion of each assay, the data were exported from the instrument as a .rda file (LC3000) and processed in proprietary software, EZReviewer, which determines the percent of substrate converted to product within a given cycle as (P/P+S)*100 by measuring the amplitude of substrate and product peaks. Cycle time varied with amount of compounds tested between 8 and 12 cycles. Initial velocity (0-3600 s) from each reaction was determined from the slope of a plot of percent conversion at each cycle versus time (seconds). Percent inhibition was determined by dividing the slope of each reaction by the uninhibited control using 100*(1-(x/uninhibited control)). IC₅₀ estimates were obtained by plotting the concentration of inhibitor versus percent inhibition in GraphPad Prism (GraphPad Software, San Diego, CA) using log[inhibitor] versus Response (Variable Slope Model); Y=Min+((Max-Min)/(1+((IC50/x)\hat{ }Hillslope)))

### Methods

a) [HER2 WT]=12.5 nM, [ATP]=25 μM, [Peptide 22]=1 μM (ATP K_{Mapp}˜25 μM)
b) [HER2YVMA]=5 nM, [ATP]=25 μM, [Peptide 22]=1 μM (ATP K_{Mapp}˜15 μM)
c) [EGFR WT]=2 nM, [ATP]=1 mM, [Peptide 22]=1 μM (ATP K_{Mapp}˜25 μM)

The results for some of the test compounds are provided in Table 3.

TABLE 3
Summary of Enzymatic Data for Test Compounds

| | HER2 WT IC₅₀ | HER2YVMA IC₅₀ | EGFR WT IC₅₀ |
|---|---|---|---|
| Compound | Method a | Method b | Method c |
| 2 | A | A | C |
| 3 | A | B | C |
| 4 | A | A | D |
| 5 | A | A | D |
| 6 | A | A | D |
| 7 | A | A | D |
| 8 | A | A | C |
| 11 | A | A | C |
| 12 | A | B | D |
| 13 | A | B | D |
| 14 | A | A | D |
| 15 | A | B | D |
| 16 | A | A | C |
| 17 | A | A | D |
| 18 | A | A | D |
| 20 | A | A | C |
| 21 | B | B | D |
| 22 | A | A | C |
| 23 | A | A | C |
| 26 | A | A | D |
| 27 | A | A | C |
| 28 | A | B | D |
| 29 | A | A | D |
| 30 | A | B | D |
| 31 | A | B | D |
| 32 | A | B | D |
| 33 | A | A | D |
| 34 | A | A | D |
| 36 | A | A | C |
| 37 | A | A | C |
| 38 | A | A | D |
| 39 | A | A | D |
| 40 | A | A | C |
| 41 | A | A | C |
| 42 | A | A | D |
| 43 | B | B | D |
| 44 | A | A | C |
| 45 | A | A | D |
| 46 | B | B | D |
| 47 | A | A | C |
| 48 | A | A | D |
| 49 | A | A | C |
| 51 | A | B | D |
| 52 | A | A | C |
| 53 | A | A | D |
| 54 | A | A | D |
| 55 | A | A | D |
| 56 | B | B | D |
| 57 | A | A | D |
| 58 | B | B | D |
| 59 | A | B | D |
| 60 | A | A | D |
| 61 | C | C | D |
| 62 | A | A | D |
| 64 | A | A | C |
| 65 | A | A | C |
| 66 | A | A | D |
| 67 | A | A | D |
| 68 | A | A | D |
| 69 | A | A | C |
| 70 | A | A | C |
| 71 | A | A | C |
| 72 | A | A | C |
| 73 | A | A | C |
| 74 | A | A | C |
| 75 | A | A | C |
| 76 | A | A | C |
| 77 | B | B | D |
| 79 | B | B | D |
| 80 | A | A | D |
| 81 | A | A | D |
| 82 | A | A | D |
| 83 | B | B | D |
| 84 | B | B | D |
| 85 | A | A | C |
| 86 | B | C | D |
| 87 | A | A | C |
| 88 | A | A | D |
| 90 | B | B | D |
| 91 | A | A | D |
| 92 | A | A | D |
| 94 | A | A | D |
| 95 | A | A | D |
| 96 | A | A | D |
| 97 | A | A | D |
| 98 | A | A | D |
| 99 | B | A | D |
| 100 | B | B | D |
| 101 | A | A | D |
| 102 | A | A | D |
| 103 | B | A | D |
| 104 | A | A | D |
| 105 | A | A | D |
| 106 | A | A | D |
| 107 | A | A | C |
| 108 | A | A | D |
| 109 | B | A | D |
| 110 | B | A | D |
| 111 | A | A | D |
| 112 | A | A | D |
| 113 | A | A | D |
| 114 | A | A | D |
| 115 | A | A | D |
| 116 | C | C | D |
| 117 | A | A | D |
| 118 | A | A | D |
| 119 | A | A | D |
| 120 | A | A | D |
| 121 | C | B | D |
| 122 | A | A | D |
| 123 | A | A | D |
| 124 | A | A | D |
| 126 | A | A | D |
| 127 | B | A | D |
| 128 | B | A | D |
| 129 | A | A | D |
| 130 | A | A | D |
| 131 | A | A | C |
| 132 | A | A | C |
| 133 | A | A | D |
| 134 | A | A | C |
| 135 | A | A | D |
| 136 | A | A | D |
| 137 | A | A | D |
| 138 | A | A | D |
| 139 | A | A | D |
| 140 | A | A | D |
| 141 | A | A | D |
| 142 | A | A | C |
| 143 | A | A | C |
| 144 | A | B | D |
| 145 | A | A | D |
| 146 | A | A | C |
| 147 | A | A | D |
| 148 | C | C | D |
| 149 | A | A | D |
| 150 | A | A | C |
| 151 | A | A | D |
| 152 | A | A | D |
| 153 | B | A | D |
| 154 | A | A | D |
| 155 | A | A | D |
| 158 | A | A | C |
| 159 | B | B | D |
| 160 | A | A | D |
| 161 | A | A | D |
| 162 | C | C | D |
| 163 | A | A | D |
| 164 | B | B | D |
| 165 | B | B | D |
| 166 | B | B | D |
| 167 | B | B | D |
| 168 | A | B | D |
| 169 | A | A | D |
| 170 | C | C | D |
| 171 | A | A | D |
| 172 | A | A | D |
| 173 | B | B | D |
| 174 | B | A | D |
| 175 | A | A | D |
| 176 | B | B | D |
| 177 | A | A | D |
| 178 | B | B | D |
| 179 | A | A | D |

A: <100 nM;
B: ≥100 nM and <1000 nM;
C: ≥1000 nM and <10000 nM;
D: ≥10000 nM

### Example 402: Assay Protocols for Assessment of Inhibition of HER2 WT, HER2YVMA, HER2VC, and EGFR WT cellular signaling and Cellular Proliferation in BT474, N87, NCI-H1781, and Ba/F3 cells

### Abbreviations

| | |
|---|---|
| FBS | fetal bovine serum |
| P/S | Penicillin-Streptomycin |
| HER2 | Human endothelial growth factor receptor type 2, alternatively |
| | ERBB2, NEU, NGL, TKR1, CD340, HER-2, MLN 19, HER- |
| | 2/neu. |
| IL3 | Interleukin 3 |
| EGF | Epidermal growth factor |
| PBS | phosphate buffered saline |
| WT | wildtype |
| YVMA | YVMA insertion |

### Preparation HER2YVMA and HER2 WT Lentiviral Constructs

Cell culture, plasmid cloning, and lentiviral transduction were carried out according to standard procedures. The Human HER2 cDNA construct in pCMV6-Entry for the HER2 wildtype (WT) expression was obtained from Origene. The HER2 cDNA including the 12-nucleotide insertion corresponding to YVMA insertion (HER2YVMA) was codon optimized, synthesized and cloned by GenScript (Piscataway, NJ) in pcDNA3.1. The lentiviral constructs expressing HER2YVMA and HER2 WT were sub-cloned using PCR at System Biosciences (SBI, Mountainview, CA) into lentiviral vectors pCDH-CMV-MCS-EF1-Neo and lentiviral particles were generated.

### Cell Lines

Engineered lines expressing HER2YVMA and HER2 WT were prepared in HEK293 (human embryonic kidney cells), Ba/F3 (Interleukin 3 (IL3)-dependent murine pro B cell line), and BEAS2B (primary immortalized human bronchial epithelial cell line). Ba/F3, BEAS2B, and HEK293 cells were obtained from the American Type Culture Collection (ATCC, Manassas, VA). Parental Ba/F3 cells were grown in suspension in complete RPMI 1640 supplemented with 10% FBS and 1% P/S and 10 ng/ml interleukin 3 (IL3, Sigma-Aldrich). Hek293 and BEAS2B cells were grown as monolayers in DMEM supplemented with 10% FBS and 1% P/S. All cells were maintained and propagated in a humidified 5% CO2 incubator at 37° C.

To generate Ba/F3, BEAS2B, and Hek-293 cells engineered with lentivirus to express HER2 WT or HER2YVMA, the cells were transduced with lentiviruses at multiplicity of infection (MOI) of 1 or 10; transduced cells were selected and maintained with G418 (600 g/mL) (Geneticin, Life Technologies, Carlsbad, CA). Ba/F3 engineered cells were subjected to gradual withdrawal of interleukin 3 (IL-3, Sigma-Aldrich). After selection, IL3 independent Ba/F3 HER2YVMA, and BEAS2B HER2YVMA cells were cloned by limiting dilution. Hek293 HER2YVMA cells were used as pool. HER2 and phospho HER2 levels were confirmed by flow cytometery and immunoassay (MSD, Meso-Scale Discovery, Rockville, Maryland).

In addition to the engineered cell lines, the following cell lines were used: BT474 (human breast carcinoma), N87 (human gastric carcinoma), NCI-H1781 (human lung adenocarcinoma), and A431 (human epidermoid carcinoma); all cell lines were obtained from the American Type Culture Collection (ATCC, Manassas, VA). NCI-H1781 were grown in complete RPMI 1640 (Life Technologies, Carlsbad, CA) supplemented with 15% fetal bovine serum (FBS, Life Technologies, Carlsbad, CA) and 1% Penicillin-Streptomycin (P/S, Life Technologies, Carlsbad, CA). N87 cells were grown in complete RPMI 1640 supplemented with 10% FBS and 1% P/S. BT474 and A431 cells were grown in DMEM (Life Technologies, Carlsbad, CA) supplemented with 10% FBS and 1% P/S. All cells were maintained and propagated in a humidified 5% CO2 incubator at 37° C.

### HER2 Signaling

HER2 signaling was assessed in BT474 and N87 cell lines which endogenously overexpress the HER2 wildtype (WT) protein, in NCI-H1781 cells which endogenously expresses a HER2 mutant protein containing a VC insertion in the kinase domain (HER2VC or G776delinsVC), and in engineered cell lines transduced to express the Her2 mutant protein containing the YVMA insertion (Hek293-HER2YVMA and BEAS2B-HER2YVMA).

BT474, N87, and NCI-H1781 were plated in 96-well tissue-culture treated plates (Corning Costar, Sigma-Aldrich) in full media and incubated overnight. Hek293-HER2YVMA and BEAS2B-HER2YVMA cells were plated in 96-well poly-D-lysine treated plates (BD Bioscience, San Jose, CA). For treatment with test compounds, the media was replaced with corresponding low serum media (1% FBS) containing test compound at 1.1, 0.37, 0.12, 0.041, 0.014, 0.005, 0.0014, 0.0005 μM or DMSO control (0.1%). Cells were incubated with compound for 1.5 hr at 37° C. After compound treatment, the media was removed and cells were lysed for 30 min on ice in complete MSD lysis buffer (prepared using MSD base lysis buffer (Meso-Scale Discovery) supplemented with cOmplete, EDTA-free Protease inhibitor cocktail (Roche), Phosphatase Inhibitor Cocktails 2 and 3 (Sigma-Aldrich), and 1 mM PMSF (Sigma-Aldrich)). Lysates were stored at −70° C. until further analysis.

Compound treatment of Ba/F3-HER2YVMA suspension cells was done as described with minor modifications. Cells were plated at 300000 cell/well in 96-well round bottom plates in RPMI supplemented with 2% FBS and immediately dosed with compounds serially diluted in media without serum for a final concentration of 1% FBS, 0.1% DMSO. Cells were incubate with compounds for 1.5 hr, and plates were spun at 1500 rpm for 5 mins to remove media without disturbing pellet. Cells were lysed as described above.

HER2 signaling was evaluated using phospho (Tyr1248)/total ErbB2 whole cell lysate MSD plates (Meso-Scale Discovery, Gaithersburg, MD). The phospho HER2 signal was normalized to total HER2 for each sample; results are reported as % DMSO control. The normalized data was fitted using a sigmoidal curve analysis program (Graph Pad Prism version 6) with variable Hill slope to determine EC50 values.

The HER2 signaling results are provided in Table 4, under columns titled “BT474-pHER2 EC50”, “N87-pHER2 EC50”, and “NCI-H1781-pHER2 EC50”. Hek293-HER2YVMA ins fall under the heading “HEKYVMA-pHER2 EC50”, Ba/F3 HER2YVMA fall under the heading “Ba/F3 YVMA-pHER2 EC50” and BEAS2B-HER2YVMA fall under the heading “BEAS2B YVMA-pHER2 EC50”. HER2 EC50 is the concentration of test compound that produces 50% inhibition of HER2 phosphorylation relative to vehicle control.

### EGFR Signaling

A431 Cells were plated in 96-well tissue-culture treated plates in full media for 6 h, then starved with media containing 0.1% FBS for overnight. Media was replaced with low serum media (1% FBS) containing compound at 10, 3.3, 1.1, 0.37, 0.12, 0.041, 0.014, 0.005 μM or DMSO control (0.1%). Cells were incubated for 1.5 hr, then stimulated with 100 ng/mL human EGF (PeproTech, Rocky Hill, NJ) for 10 minutes. Cells were washed with cold PBS, and lysed for 30 min on ice in complete MSD lysis buffer (prepared as described in HER2 Signaling Method); lysates were stored at −70° C. until further analysis. EGFR signaling was evaluated using phospho (Tyr 1068)/total EGFR whole cell lysate kit (Meso-Scale Discovery, Gaithersburg, MD). The phospho EGFR signal was normalized to total EGFR for each sample; results are reported as % DMSO control. The normalized data was fitted using a sigmoidal curve analysis program (Graph Pad Prism version 6) with variable Hill slope to determine EC50 values. The results are provided in Table 4, under column titled “A431-pEGFR EC50”. EGFR EC50 is the concentration of test compound that produces 50% inhibition of EGFR phosphorylation relative to vehicle control.

### Cellular Proliferation in Adherent Cells and Non-Adherent Cells

Cell proliferation in adherent cells was investigated in two HER2 amplified cell lines (BT474, N87), and a HER2VC expressing cell line (NCI-H1781). BT474, NCI-H1781, and N87 were plated in their appropriate growth media supplemented with either 10% FBS (BT474, N87) or 15% FBS (NCI-H1781) and 1% P/S in 96-well white clear-bottom tissue culture plates (Costar, Sigma-Aldrich). The starting cell densities were 3500, 5500, and 5000 cells for BT474, NCI-H1781, and N87 cells, respectively. An 8-point standard curve with varying cell densities (starting at 50000 cells and serially diluting down 2-fold) was prepared for each cell line and read 6 h after plating. Cells were allowed to attach overnight prior to compound treatment. Compound solutions were prepared by serially diluting test compounds in DMSO (Sigma-Aldrich), then diluting 500× in their corresponding serum free media. Cells were treated by adding a 1:1 volume of test compound to a final concentration of 3, 1, 0.33, 0.11, 0.04, 0.001, 0.004, 0.0014, or 0.0005 μM, in 0.1% DMSO and 5% FBS (for BT474 and N87) or 7.5% FBS (NCI-H1781). In BT474 and NCI-H1781, cell media was replaced and cells were redosed with fresh test compound solutions after 72 h. Viability was determined by CellTiter Glo (Promega, Madison, WI) after 72 h for N87 cells, and after 120 h for BT474 and NCI-H1787 cells. The results were converted to cell numbers using a standard curve; growth inhibition (GI50 values) were determined by Graph Pad Prism. GI50 is the concentration of test compound that produces 50% inhibition of growth relative to vehicle control. The results are provided in Table 4, under columns titled “BT474-GI50”, “N87-GI50”, and “NCI-H1781-GI50”.

Cell proliferation in non-adherent cells was investigated in Ba/F3 parental (IL3-dependent) and in the two IL3 independent cell clones tranduced to express mutant HER2 protein containing the YVMA insertion (Ba/F3 HER2YVMA). Cells were plated in DMEM supplemented with 10% FBS, 1% P/S, and 10 ng/ml IL3 (for parental Ba/F3 only) in 96-well white clear-bottom tissue culture plates (Costar, Sigma-Aldrich) at a starting cell density of 7500 cells. An 8-point standard curve with varying cell densities (starting at 100,000 cells and serially diluting down 2-fold) was prepared for each cell line and read after plating. Compound solutions were prepared in serum free media, and cells were treated as described above for adherent cells. Cell viability was determined by CellTiter Glo (Promega, Madison, WI) after 72 h. The results were converted to cell numbers using a standard curve; growth inhibition (GI50 values) were determined by Graph Pad Prism.

The results of these experiments show the ability of compounds to inhibit cell growth in HER2 dependent cell lines and a control cell line (Ba/F3 parental) are depicted in Table 4 in the columns titled “G150 (nM)”. Ba/F3 parental results fall under the heading “Ba/F3 parental-G150”, and Ba/F3 HER2YVMA fall under the heading “Ba/F3 YVMA-G150”.

### Example 403: Assay Protocols for Assessment of HER2 WT, HER2YVMA, and EGFR WT Occupancy in Cells

### HER2 Occupancy Assay

In order to assess free HER2 protein, a biotinylated probe was utilized. BT474, N87, and Ba/F3-HER2YVMA cells were treated with test compound as described for HER2 signaling. After compound treatment, cells were washed three times with cold phosphate buffer saline (PBS, Life Technologies, Carlsbad, CA), and lysed in modified occupancy lysis buffer prepared with an irreversible biotinylated tool compound (1 μM) in 25 mM Tris pH 7.5, 150 mM NaCl, 1% Triton, and supplemented with cOmplete, EDTA-free Protease inhibitor cocktail (Roche), Phosphatase Inhibitor Cocktails 2 and 3 (Sigma-Aldrich), and 1 mM PMSF (Sigma-Aldrich).

To determine % occupancy, lysates were loaded into duplicate plates of the phospho (Tyr1248)/total ErbB2 whole cell lysate MSD kit (Meso-Scale Discovery) and incubated overnight. Plates were washed and incubation with either HER2-sulfoTag (1:50, Meso Scale Discovery) or Strep-Sulfo Tag (1:1000, Meso Scale Discovery), for detection of free and total HER2, respectively. A standard curve of untreated cells in serial dilution was included in each plate to independently fit streptavidin (Free HER2) and total HER2 MSD signals; non-specific signal (BSA control spots) was subtracted from each well. Free HER2 was then normalized by total HER2 for each sample. The results are reported as % DMSO control. The normalized data was fitted using a sigmoidal curve analysis program (Graph Pad Prism version 6) with variable Hill slope to determine EC50 values. The results are provided in Table 4, under columns titled “BT474-HER2 Occ”, “N87-HER2 Occ”, and “Ba/F3 YVMA-HER2 Occ”. HER2 Occ is the concentration of test compound at which 50% of HER2 is irreversible bound.

### EGFR Occupancy

In order to assess free EGFR protein, a biotinylated probe was utilized as described for HER2 occupancy. N87 cells were plated, treated, and lysed as described for HER2 occupancy method. After treatment, the cells were washed three times with cold PBS, lysed at room temperature for 1 hr in modified occupancy lysis buffer (prepared as described in section titled “HER2 Occupancy Assay”). Lysates were stored at −70° C. until further analysis. For determination of % occupancy, lysates were loaded into duplicate plates of the phospho (Tyr 1068)/total EGFR whole cell lysate kit (Meso-Scale Discovery) and incubated overnight. Plates were washed and incubated with either EGFR-sulfoTag (1:50, Meso Scale Discovery) or Strep-Sulfo Tag (1:1000, Meso Scale Discovery), for detection of free and total EGFR, respectively. BSA control spots included in MSD plates were used to subtract non-specific signal. A standard curve of untreated cells in serial dilution was included in each plate to convert streptavidin and total EGFR MSD signals into free EGFR and total EGFR. Free EGFR was normalized to total EGFR for each sample. Data, reported as % DM50 control, was fitted using a sigmoidal curve analysis program (Graph Pad Prism version 6) with variable Hill slope to determine EC50 values. The results are provided in Table 4, under column titled “N87-EGFR Occ”. EGFR Occ is the concentration of test compound at which 50% of EGFR is irreversible bound.

TABLE 4
Summary of Cellular Data for Test Compounds

| | BT474 | | | N87 | | | | NCI-H1781 | | HEKYVMA |
|---|---|---|---|---|---|---|---|---|---|---|
| | pHER2 | HER2 | | pHER2 | HER2 | EGFR | | pHER2 | | pHER2 |
| Compound | EC50 | Occ | GI50 | EC50 | Occ | Occ | GI50 | EC50 | GI50 | EC50 |
| 3 | A | | | | | | | A | | B |
| 4 | A | | | | | | | A | | A |
| 5 | A | A | | A | A | | | A | | A |
| 6 | A | | | | | | | A | | A |
| 7 | A | | | | | | | A | | A |
| 8 | A | A | A | A | A | A | A | A | A | A |
| 11 | A | | | | | | | | | A |
| 12 | A | A | | A | A | D | | | | A |
| 13 | A | | | | | | | | | A |
| 14 | A | | | A | A | A | | | | A |
| 15 | A | | | A | A | A | | | | A |
| 16 | A | | | A | A | B | | | | A |
| 17 | A | | A | A | A | B | A | A | A | A |
| 18 | A | | | | | | | | | A |
| 20 | A | | | | | | | | | A |
| 21 | A | | | | | | | | | A |
| 22 | A | | | | | | | | | A |
| 23 | A | | | | | | | | | A |
| 26 | A | | | | | | | | | A |
| 27 | A | | | | | | | | | |
| 28 | A | | | | | | | | | |
| 29 | A | | | | | | | | | |
| 30 | A | | | | | | | | | |
| 31 | A | | | | | | | | | |
| 32 | A | | | | | | | | | |
| 33 | A | | A | A | | | A | A | A | A |
| 34 | A | | A | A | | | A | A | A | A |
| 36 | A | | | | | | | | | |
| 37 | A | | | | | | | | | B |
| 38 | A | | | | | | | | | A |
| 39 | B | | | | | | | | | B |
| 40 | A | | | | | | | | | A |
| 41 | A | | | | | | | | | A |
| 42 | A | | A | | | | A | | A | A |
| 43 | C | | | | | | | | | C |
| 44 | A | | | | | | | | | A |
| 45 | A | | | | | | | | | A |
| 46 | A | | | | | | | | | A |
| 47 | A | | | | | | | | | A |
| 48 | A | | | | | | | | | A |
| 49 | A | | | | | | | | | A |
| 51 | B | | | | | | | | | B |
| 52 | A | | | | | | | | | A |
| 53 | A | | | | | | | | | B |
| 54 | A | | | | | | | | | A |
| 55 | A | | | | | | | | | A |
| 56 | C | | | | | | | | | C |
| 57 | A | | | | | | | | | A |
| 58 | B | | | | | | | | | C |
| 59 | A | | | | | | | | | A |
| 60 | A | | | | | | | | | B |
| 61 | C | | | | | | | | | C |
| 62 | A | | | | | | | | | B |
| 64 | A | | | | | | | | | A |
| 65 | A | | | | | | | | | A |
| 66 | A | | | | | | | | | A |
| 67 | A | | | | | | | | | A |
| 68 | A | | | | | | | | | A |
| 69 | A | | | | | | | | | A |
| 70 | A | | | | | | | | | A |
| 71 | A | | | | | | | | | A |
| 72 | A | | | | | | | | | A |
| 73 | A | | | | | | | | | A |
| 74 | A | | | | | | | | | A |
| 75 | A | | | | | | | | | A |
| 76 | A | | | | | | | | | A |
| 77 | A | | | | | | | | | B |
| 80 | A | | | | | | | | | A |
| 81 | A | | | | | | | | | A |
| 82 | A | | | | | | | | | A |
| 83 | A | | | | | | | | | A |
| 84 | A | | | | | | | | | B |
| 85 | A | | | | | | | | | A |
| 86 | B | | | | | | | | | A |
| 87 | A | | | | | | | | | A |
| 88 | A | | | | | | | | | A |
| 91 | A | | | | | | | | | A |
| 92 | A | | | | | | | | | A |
| 94 | A | | | | | | | | | A |
| 95 | A | | A | A | A | B | A | A | A | A |
| 96 | A | | | | | | | | | A |
| 97 | A | | | | | | | | | B |
| 98 | A | | A | | | | A | A | A | A |
| 99 | A | | A | | | | A | | A | A |
| 100 | A | | A | A | | | A | A | A | A |
| 101 | A | | | | | | | | | A |
| 102 | A | | | | | | | | | B |
| 103 | A | | | | | | | | | A |
| 104 | A | | | | | | | | | A |
| 105 | A | | A | A | | | A | A | A | A |
| 106 | A | | A | A | | | A | A | A | A |
| 107 | A | | | | | | | | | A |
| 108 | A | | A | | | | A | | A | A |
| 109 | A | | | | | | | | | A |
| 110 | A | | | | | | | | | B |
| 111 | A | | | | | | | | | B |
| 112 | A | | | | | | | | | A |
| 113 | A | | | | | | | | | A |
| 114 | A | | A | | | | A | | A | A |
| 115 | A | | | | | | | | | A |
| 117 | A | | | | | | | | | A |
| 118 | A | | A | | | | A | | A | A |
| 119 | A | | | | | | | | | A |
| 120 | A | | | | | | | | | A |
| 121 | A | | | | | | | | | A |
| 122 | A | | | | | | | | | A |
| 123 | A | | | | | | | | | A |
| 124 | A | | A | | | | A | | A | A |
| 126 | A | | A | | | | A | | A | A |
| 127 | A | | A | | | | A | | B | A |
| 128 | A | | A | A | A | D | A | B | A | A |
| 129 | A | | A | A | A | B | A | A | A | A |
| 130 | A | | A | | | | A | | A | A |
| 131 | A | | | | | | | | | A |
| 132 | A | | | | | | | | | A |
| 133 | A | | | | | | | | | A |
| 134 | B | | | | | | | | | B |
| 135 | A | | | | | | | | | B |
| 136 | A | | | | | | | | | A |
| 137 | A | | | | | | | | | A |
| 138 | A | | | | | | | | | A |
| 139 | A | | | | | | | | | A |
| 140 | A | | | | | | | | | A |
| 141 | A | | | | | | | | | A |
| 142 | A | | | | | | | | | A |
| 143 | A | | | | | | | | | A |
| 144 | A | | | | | | | | | B |
| 145 | A | | | | | | | | | A |
| 146 | A | | | | | | | | | A |
| 147 | A | | A | A | | | A | A | A | A |
| 148 | B | | | | | | | | | C |
| 149 | A | | | | | | | | | A |
| 150 | A | | | | | | | | | A |
| 151 | A | | | | | | | | | B |
| 152 | A | | A | | | | A | A | A | A |
| 153 | A | | | | | | | | | A |
| 154 | A | | | | | | | | | A |
| 155 | A | | | | | | | | | A |
| 158 | A | | A | | | | A | A | A | A |
| 159 | A | | A | A | | | A | B | B | A |
| 160 | A | | A | A | | | A | A | A | A |
| 161 | A | | | A | | | | | | A |
| 162 | B | | | C | | | | | | B |
| 163 | A | | | | | | | | | A |
| 164 | A | | A | | | | A | A | B | A |
| 165 | A | | | | | | | | | B |
| 166 | A | | | A | | | | | | A |
| 167 | A | | | | | | | | | B |
| 168 | A | | | | | | | | | B |
| 169 | A | | A | A | | | A | A | A | A |
| 170 | C | | B | | | | B | B | C | C |
| 171 | A | | | | | | | | | A |
| 172 | A | | | | | | | | | B |
| 173 | A | | | | | | | | | B |
| 174 | A | | | | | | A | A | B | A |
| 175 | A | | | | | | | | | A |
| 176 | A | | | | | | | | | A |
| 177 | A | | | A | | | | A | | A |
| 178 | A | | | | | | | | | A |
| 179 | A | | | | | | | | | A |

For BT474, N87, NCI-H1781, and HEKYVMA: A: ≤ 100 nM; B: > 100 nM and < 350 nM; C: ≥ 350 nM and < 1000 nM; D: ≥ 1000 nM

TABLE 4
Summary of Cellular Data for Test Compounds (continued)

| | BEAS2B | | | | | | Selectivity | |
|---|---|---|---|---|---|---|---|---|
| | YVMA | Ba/F3 YVMA | | | Ba/F3 | A431 | A431 | A431 |
| | pHER2 | pHER2 | HER2 | | parental | pEGFR | EC50/BT474 | EC50/HEKYVMA |
| Compound | EC50 | EC50 | Occ | GI50 | GI50 | EC50 | EC50 | EC50 |
| 3 | | | | | | B | B | E |
| 4 | | | | | | B | B | A |
| 5 | | A | | | | B | | B |
| 6 | | | | | | A | E | E |
| 7 | | | | | | A | D | F |
| 8 | A | A | A | A | C | A | C | C |
| 11 | | | | | | A | E | E |
| 12 | | A | | | | C | A | B |
| 13 | | | | | | B | C | C |
| 14 | | | | | | A | D | E |
| 15 | | | | | | B | B | B |
| 16 | | A | A | | | B | D | D |
| 17 | | A | A | | | B | C | A |
| 18 | | | | | | B | B | B |
| 20 | | | | | | A | B | B |
| 21 | | | | | | B | B | B |
| 22 | | | | | | A | D | E |
| 23 | | | | | | A | D | E |
| 26 | | | | | | A | D | D |
| 27 | | | | | | A | B | |
| 28 | | | | | | A | C | |
| 29 | | | | | | A | C | |
| 30 | | | | | | B | A | |
| 31 | | | | | | B | D | |
| 32 | | | | | | B | B | |
| 33 | | A | | A | C | B | B | C |
| 34 | | A | | A | C | B | C | E |
| 36 | | | | | | A | D | |
| 37 | | | | | | D | A | B |
| 38 | | | | | | B | C | D |
| 39 | | | | | | C | C | D |
| 40 | | | | | | A | B | B |
| 41 | | | | | | A | B | C |
| 42 | | | | | | B | B | B |
| 43 | | | | | | D | B | C |
| 44 | | | | | | B | D | E |
| 45 | | | | | | B | C | D |
| 46 | | | | | | C | B | C |
| 47 | | | | | | B | C | C |
| 48 | | | | | | B | B | B |
| 49 | | | | | | B | B | D |
| 51 | | | | | | C | C | E |
| 52 | | | | | | A | D | C |
| 53 | | | | | | C | C | D |
| 54 | | | | | | B | D | D |
| 55 | | | | | | B | B | B |
| 56 | | | | | | C | C | B |
| 57 | | | | | | A | D | D |
| 58 | | | | | | C | C | D |
| 59 | | | | | | B | B | C |
| 60 | | | | | | B | C | E |
| 61 | | | | | | D | B | C |
| 62 | | | | | | B | B | E |
| 64 | | | | | | B | C | E |
| 65 | | | | | | B | D | E |
| 66 | | | | | | B | C | C |
| 67 | | | | | | A | D | D |
| 68 | | | | | | A | D | E |
| 69 | | | | | | A | A | D |
| 70 | | | | | | A | D | E |
| 71 | | | | | | A | A | E |
| 72 | | | | | | A | D | E |
| 73 | | | | | | B | D | E |
| 74 | | | | | | B | D | D |
| 75 | | | | | | A | D | D |
| 76 | | | | | | A | C | C |
| 77 | | | | | | C | B | D |
| 80 | | | | | | B | B | B |
| 81 | | | | | | B | B | C |
| 82 | | | | | | B | C | C |
| 83 | | | | | | C | A | B |
| 84 | | | | | | D | A | B |
| 85 | | | | | | B | C | C |
| 86 | | | | | | D | B | A |
| 87 | | | | | | A | B | D |
| 88 | | | | | | A | C | C |
| 91 | | | | | | A | C | D |
| 92 | | | | | | A | C | C |
| 94 | | | | | | B | C | E |
| 95 | A | | | A | C | B | D | C |
| 96 | | | | | | B | C | D |
| 97 | | | | | | B | B | E |
| 98 | | | | | | B | C | D |
| 99 | | | | A | C | B | B | B |
| 100 | | | | | | B | B | C |
| 101 | | | | | | B | C | C |
| 102 | | | | | | C | C | C |
| 103 | | | | | | B | B | B |
| 104 | | | | | | A | D | D |
| 105 | | | | | | B | C | D |
| 106 | | | | A | C | B | B | C |
| 107 | | | | | | A | C | A |
| 108 | | | | A | C | A | C | C |
| 109 | | | | | | B | D | E |
| 110 | | | | | | C | B | C |
| 111 | | | | | | C | A | B |
| 112 | | | | | | B | B | C |
| 113 | | | | | | B | B | C |
| 114 | | | | | | B | B | B |
| 115 | | | | | | B | C | D |
| 117 | | | | | | B | B | B |
| 118 | | | | | | B | A | B |
| 119 | | | | | | B | B | B |
| 120 | | | | | | B | B | B |
| 121 | | | | | | C | B | C |
| 122 | | | | | | B | B | B |
| 123 | | | | | | B | C | D |
| 124 | | | | A | C | A | B | C |
| 126 | | | | A | C | B | B | B |
| 127 | | | | B | C | C | A | B |
| 128 | | | | A | C | C | B | B |
| 129 | | | | A | | B | B | B |
| 130 | | | | A | C | C | A | A |
| 131 | | | | | | A | C | D |
| 132 | | | | | | B | B | C |
| 133 | | | | | | B | B | C |
| 134 | | | | | | C | B | B |
| 135 | | | | | | C | B | D |
| 136 | | | | | | B | B | C |
| 137 | | | | | | B | D | D |
| 138 | | | | | | B | B | A |
| 139 | | | | | | A | B | B |
| 140 | | | | | | B | B | C |
| 141 | | | | | | B | B | B |
| 142 | | | | | | B | B | B |
| 143 | | | | | | A | C | E |
| 144 | | | | | | B | B | D |
| 145 | | | | | | B | C | E |
| 146 | | | | | | A | C | D |
| 147 | | | | | | A | B | D |
| 148 | | | | | | D | B | C |
| 149 | | | | | | B | B | D |
| 150 | | | | | | B | B | E |
| 151 | | | | | | B | B | E |
| 152 | | | | | | B | C | C |
| 153 | | | | | | B | B | C |
| 154 | | | | | | A | C | E |
| 155 | | | | | | A | B | E |
| 158 | | | | | | B | B | B |
| 159 | | | | | | B | C | E |
| 160 | | | | | | B | D | D |
| 161 | | | | | | A | D | E |
| 162 | | | | | | D | B | B |
| 163 | | | | | | A | D | E |
| 164 | | | | | | B | C | D |
| 165 | | | | | | C | B | D |
| 166 | | | | | | B | B | C |
| 167 | | | | | | B | B | E |
| 168 | | | | | | C | A | B |
| 169 | | | | | | B | C | D |
| 170 | | | | | | A | B | B |
| 171 | | | | | | A | A | E |
| 172 | | | | | | C | B | C |
| 173 | | | | | | C | B | C |
| 174 | | | | | | B | B | C |
| 175 | | | | | | A | C | E |
| 176 | | | | | | C | B | B |
| 177 | | | | | | B | C | D |
| 178 | | | | | | B | D | E |
| 179 | | | | | | A | B | E |

For BEAS2B: A: ≤ 100 nM; B: > 100 nM and < 350 nM; C: ≥ 350 nM and < 1000 nM; D: ≥ 1000 nM
For Ba/F3 and A431: A: ≤ 100 nM; B: > 100 nM and < 1000 nM; C: ≥ 1000 nM and < 10000 nM; D: ≥ 10000 nM
For selectivity: A: ≥100; B: ≥ 20 and < 100; C: ≥ 10 and < 20; D: ≥ 5 and < 10; E: >1 and < 5; F: < 1

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

### ENUMERATED EMBODIMENTS

1. A compound of Formula (I):

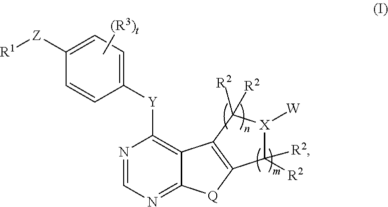

or a pharmaceutically acceptable salt, solvate, hydrate, prodrug, tautomer, stereoisomer, enantiomer, or isotopologue thereof, or a mixture thereof, wherein:
X is CR⁵ or N;
Y is NR⁶, CR⁷R⁸, or O;
Z is NR⁶ or O;
Q is S, NR⁶, or CR⁷R⁸;
n is 1, 2, 3, or 4;
m is 1, 2, 3, or 4;
t is 0, 1, 2, 3, or 4;
R¹ is aryl, heteroaryl, cycloalkyl, or heterocyclyl;
each instance of R² is independently hydrogen, alkyl, alkenyl, alkynyl, haloalkyl, hydroxyalkyl, heteroalkyl, halogen, cyano, nitro, amido, cycloalkyl, heterocyclyl, aryl, heteroaryl, —OR′, or
   —NR′R″, wherein R′ and R¹¹ are independently hydrogen, alkyl, heteroalkyl, aryl, or heteroaryl, or R′ and R¹¹ are taken together with nitrogen to form a cyclic moiety;
or two R² are taken together to form a C₁-C₃ alkylene;
each instance of R³ is independently alkyl, alkenyl, alkynyl, haloalkyl, hydroxyalkyl, heteroalkyl, halogen, hydroxyl, alkoxy, cyano, nitro, amino, amido, cycloalkyl, heterocyclyl, aryl, or heteroaryl;
each instance of R⁵, R⁶, R⁷, and R⁸ is independently hydrogen or alkyl;
W is
   -L-(NR ⁶)ₛ—C(═O)—(CR¹⁰=CR¹⁰)—R¹¹,
   -L-(NR ⁶)ₛ—C(═O)—C(═CR¹⁰R¹⁰)—R¹¹,
   -L-(CR¹⁰=CR¹⁰)—C(═O)—(NR ⁶)ₛ—R¹¹,
   -L-C(═CR¹⁰R¹⁰)—C(═O)—(NR ⁶)ₛ—R¹¹,
   -L-(NR⁶)ₛ—S(═O)₂—(CR¹⁰=CR¹⁰)—R¹¹,
   -L-(NR⁶)ₛ—S(═O)₂—C(═CR¹⁰R¹⁰)—R¹¹,
   -L-(CR¹⁰=CR¹⁰)—S(═O)₂—(NR ⁶)ₛ—R¹¹; or
   -L-C(═CR¹⁰R¹⁰)—S(═O)₂—(NR ⁶)ₛ—R¹¹;
L is a bond or C₁-C₃ alkylene;
s is 0 or 1;
each instance of R¹⁰ is independently hydrogen, alkyl, haloalkyl, hydroxyalkyl, heteroalkyl, alkoxy, cycloalkyl, heterocyclyl, aryl, heteroaryl, or halo;
R¹¹ is hydrogen, —OR¹², —(C₁-C₃ alkylene)-OR¹², —NR²R¹³, —(C₁-C₃ alkylene)-NR¹²R¹³, cycloalkyl, —(C₁-C₃ alkylene)-cycloalkyl, heterocyclyl, —(C₁-C₃ alkylene)-heterocyclyl, aryl, —(C₁-C₃ alkylene)-aryl, heteroaryl, or —(C₁-C₃ alkylene)-heteroaryl; and
R¹² and R¹³ are independently hydrogen, alkyl, haloalkyl, hydroxyalkyl, heteroalkyl, alkoxy, cycloalkyl, heterocyclyl, aryl, heteroaryl, cycloalkylalkyl, heterocyclylalkyl, arylalkyl, or heteroarylalkyl, or R¹² and R¹³ are taken together with nitrogen to form a cyclic moiety.

2. The compound of claim 1, wherein n is 1.

3. The compound of claim 1, wherein n is 1, and m is 1 or 2.

4. The compound of claim 1, wherein m is 1.

5. The compound of claim 1, wherein n is 1 or 2, and m is 1.

6. The compound of claim 1, which is of the Formula (II-1), (II-2), (II-3), (II-4), or (II-5):

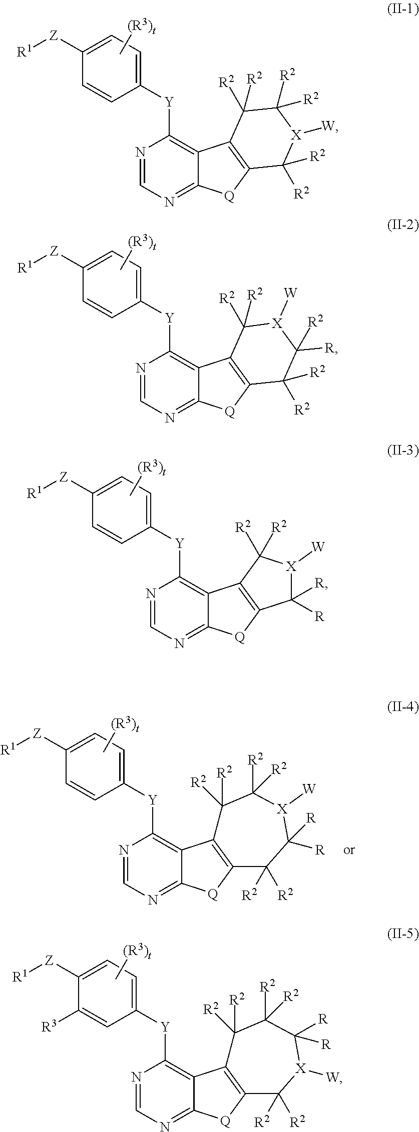

or a pharmaceutically acceptable salt, solvate, hydrate, prodrug, tautomer, stereoisomer, enantiomer, or isotopologue thereof, or a mixture thereof.

7. The compound of any one of claims 1-2, wherein t is 1.

8. The compound of claim 1, which is of the Formula (III-1), (III-2), (III-3), (III-4), or (III-5):

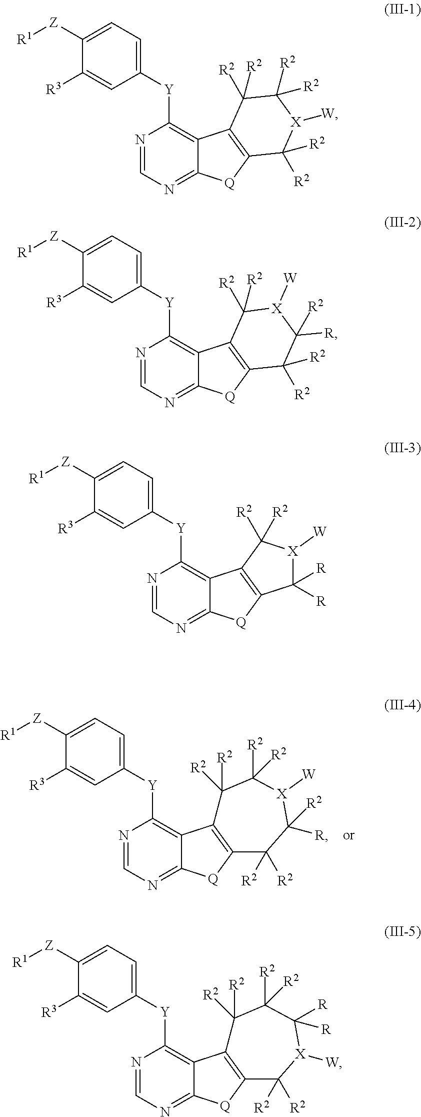

or a pharmaceutically acceptable salt, solvate, hydrate, prodrug, tautomer, stereoisomer, enantiomer, or isotopologue thereof, or a mixture thereof.

9. The compound of any one of claims 1-3, wherein X is CH.

10. The compound of any one of claims 1-3, wherein X is N.

11. The compound of any one of claims 1-10, wherein Y is NH.

12. The compound of any one of claims 1-10, wherein Y is CH₂.

13. The compound of any one of claims 1-10, wherein Y is O.

14. The compound of any one of claims 1-13, wherein Z is NH.

15. The compound of any one of claims 1-13, wherein Z is O.

16. The compound of any one of claims 1-15, wherein Q is S.

17. The compound of any one of claims 1-15, wherein Q is NH.

18. The compound of any one of claims 1-15, wherein Q is CH₂.

19. The compound of claim 1, which is of the Formula (IV-1), (IV-2), (IV-3), (IV-4), (IV-5), IV-6), or (IV-7):

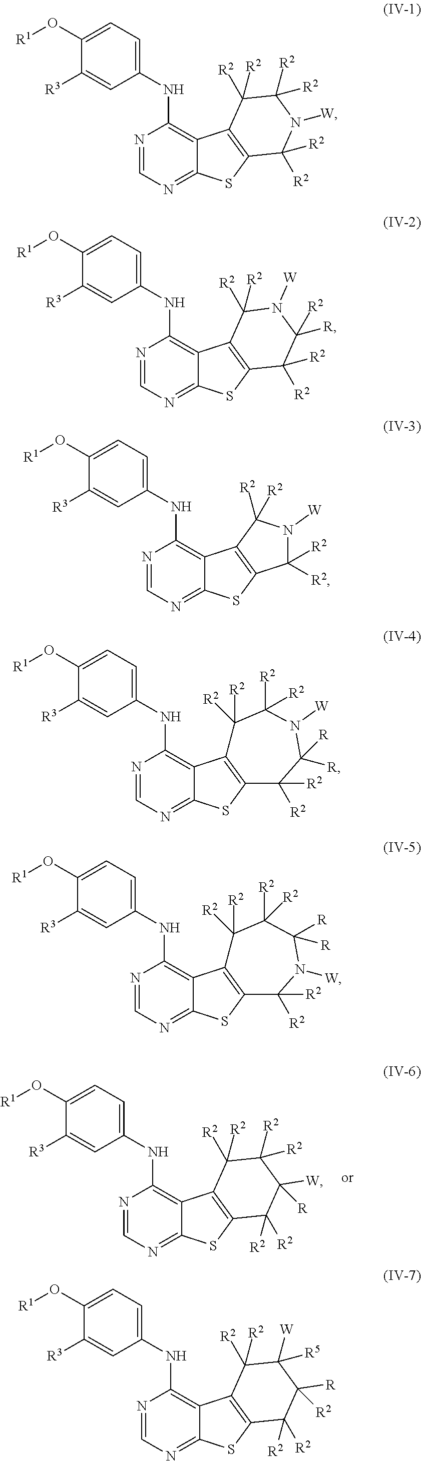

or a pharmaceutically acceptable salt, solvate, hydrate, prodrug, tautomer, stereoisomer, enantiomer, or isotopologue thereof, or a mixture thereof.

20. The compound of any one of claims 1-4, wherein L is a bond.

21. The compound of any one of claims 1-4, wherein L is CH₂.

22. The compound of any one of claims 1-4, wherein s is 0.

23. The compound of any one of claims 1-4, wherein s is 1.

24. The compound of any one of claims 1-4, wherein all of R¹⁰ are hydrogen.

25. The compound of any one of claims 1-4, wherein R¹¹ is —CH₂—NR¹²R¹³.

26. The compound of claim 25, wherein R¹¹ is —CH₂—NMe₂.

27. The compound of claim 25, wherein R¹¹ is —CH₂—NR¹²R¹³, wherein R¹² and R¹³ are taken together with nitrogen to form a cyclic moiety.

28. The compound of claim 27, wherein the cyclic moiety formed by NR¹²R¹³ is a heterocyclyl.

29. The compound of claim 28, wherein the cyclic moiety formed by NR²R¹³ is a monocyclic heterocyclyl.

30. The compound of claim 29, wherein the cyclic

moiety formed by NR¹²R¹³ is aziridinyl, azetidinyl, pyrrolidinyl, dihydropyrrolyl, piperidinyl, dihydropyridinyl, piperazinyl, morpholinyl, azepanyl, oxazepanyl, diazepanyl, or azocanyl.

31. The compound of claim 28, wherein the cyclic moiety formed by NR¹²R¹³ is a bicyclic heterocyclyl.

32. The compound of claim 31, wherein the cyclic

moiety formed by NR¹²R¹³ is a bicyclic heterocyclyl, wherein a first ring selected from the group consisting of aziridinyl, azetidinyl, pyrrolidinyl, dihydropyrrolyl, piperidinyl, dihydropyridinyl, piperazinyl, morpholinyl, azepanyl, oxazepanyl, diazepanyl, and azocanyl is fused, bridged, or spiroed with a second ring.

33. The compound of claim 27, wherein the cyclic

moiety formed by NR¹²R¹³ is a heteroayl.

34. The compound of any one of claims 1-19, wherein W is —C(═O)—(CR¹⁰=CR¹⁰)—R¹¹, —NR⁶—C(═O)—(CR¹⁰=CR¹⁰)—R¹¹, —C(═O)—C(═CR¹R¹⁰)—R¹¹, —NR⁶—C(═O)—C(═CR¹OR¹⁰)—R¹¹, —CH₂—(CR¹⁰=CR¹⁰)—C(═O)—R¹¹, —CH₂—C(═CR¹OR¹O)—C(═O)—R¹¹, —S(═O)₂—(CR¹⁰=CR¹⁰)—R¹¹, —NR⁶—S(═O)₂—(CR¹⁰=CR¹⁰)—R¹¹, —S(═O)₂—C(═CR¹OR¹O)—R¹¹, —NR⁶—S(═O)₂—C(═CR¹OR¹O)—R¹¹, —CH₂—(CR¹⁰=CR¹⁰)—S(═O)₂—R¹¹, or —CH₂—C(═CR¹OR¹O)—S(═O)₂—R¹¹.

35. The compound of claim 34, wherein W is

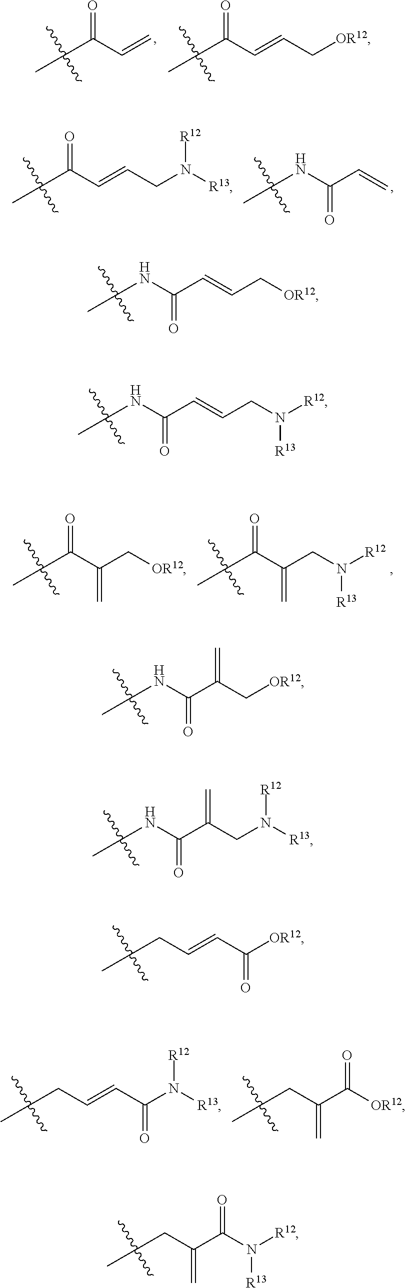

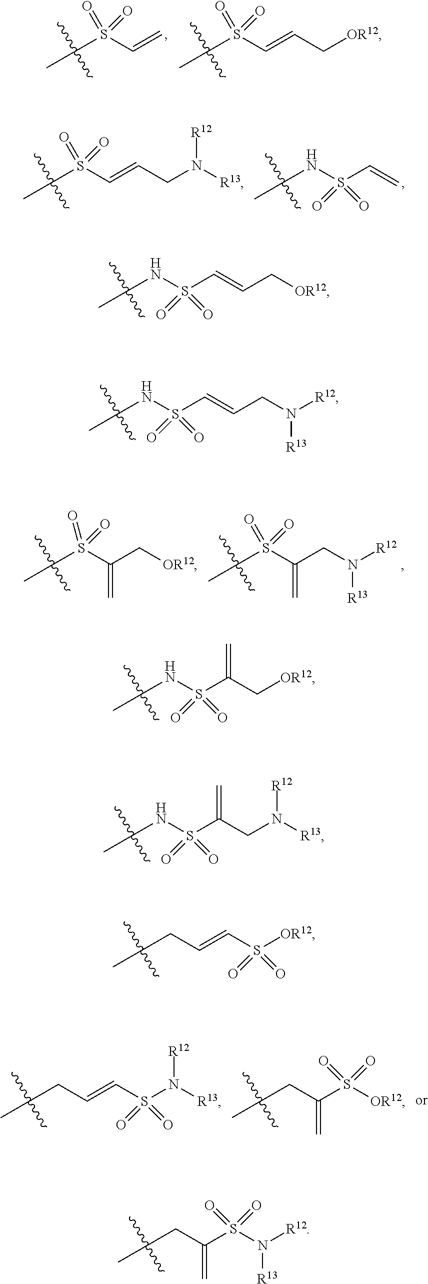

36. The compound of any one of claims 1-35, wherein the carbon-carbon double bond in W has a E-configuration.

37. The compound of claim 1, which is of the Formula (V-1), (V-2), (V-3), (V-4), (V-5), (V-6), (V-7), (V-8), (V-9), (V-10), (V-11), (V-12), (V-13), or (V-14):

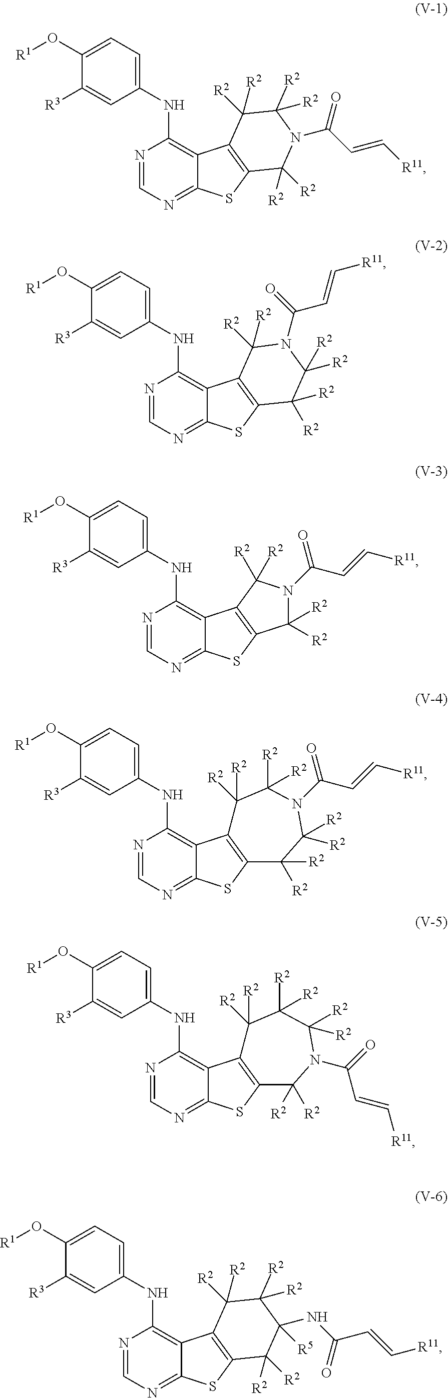

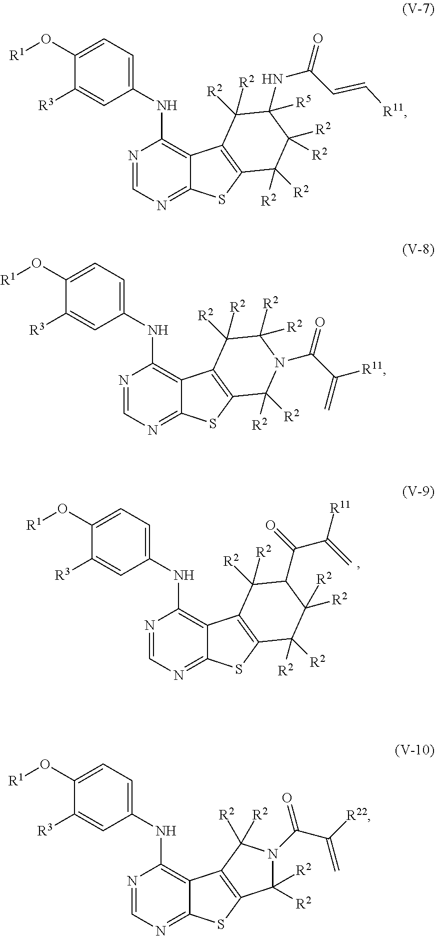

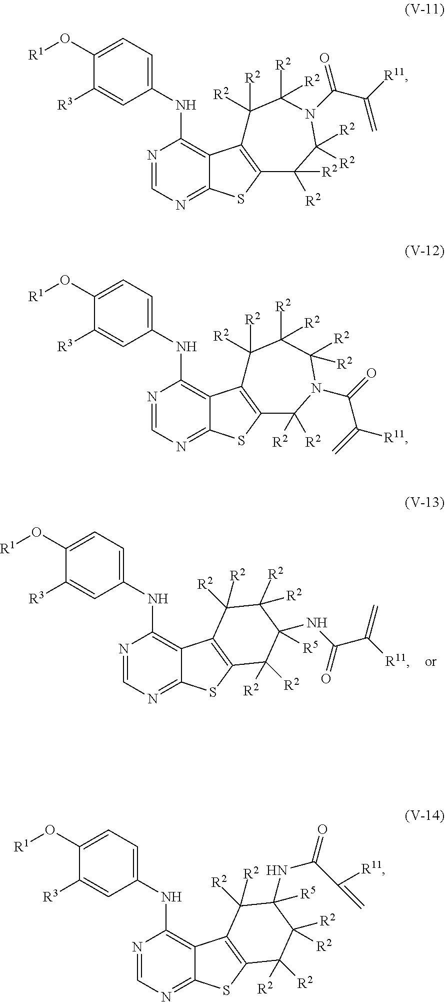

or a pharmaceutically acceptable salt, solvate, hydrate, prodrug, tautomer, stereoisomer, enantiomer, or isotopologue thereof, or a mixture thereof.

38. The compound of any one of claims 1-37, wherein the cycloalkyl, heterocyclyl, aryl, or heteroaryl group in R¹¹, R¹², and R¹³ or the cyclic moiety formed by NR²R¹³ is optionally substituted with 1, 2, 3, 4, 5, or 6 of R¹⁴ independently selected from the group consisting of alkyl, alkenyl, alkynyl, haloalkyl, heteroalkyl, halogen, hydroxyl, alkoxy, hydroxyalkyl, cyano, nitro, amino, amido, cycloalkyl, heterocyclyl, aryl, heteroaryl, acyl, N-acyl, carbonyl, oxo, sulfonyl, sulfonamide, and boronic acid, wherein each instance of R¹⁴ independently is optionally substituted with one or more groups selected from alkyl, alkenyl, alkynyl, haloalkyl, heteroalkyl, halogen, hydroxyl, alkoxy, hydroxyalkyl, cyano, nitro, amino, amido, cycloalkyl, heterocyclyl, aryl, heteroaryl, acyl, N-acyl, carbonyl, oxo, sulfonyl, sulfonamide, and boronic acid.

39. The compound of claim 38, wherein each instance of R¹⁴ is independently selected from the group consisting of methyl, ethyl, hydroxyl, and hydroxylmethyl.

40. The compound of any one of claims 1-39, wherein

R¹ is C₆-C₁₀ aryl.

41. The compound of claim 40, wherein R¹ is phenyl.

42. The compound of any one of claims 1-39, wherein R¹ is 5- to 18-membered heteroaryl.

43. The compound of claim 42, wherein the heteroaryl comprises 1, 2, or 3 of heteroatoms independently selected from the group consisting of N, S, and O.

44. The compound of claim 42, wherein R¹ is monocyclic heteroaryl.

45. The compound of claim 44, wherein R¹ is pyridyl, pyridazinyl, oxazolyl, thiazolyl, oxadizolyl, piperidinyl, pyrazolo, or pyrrolo.

46. The compound of claim 45, wherein R¹ is pyridyl.

47. The compound of claim 46, wherein R¹ is 3-pyridyl.

48. The compound of claim 42, wherein R¹ is bicyclic heteroaryl.

49. The compound of claim 48, wherein R¹ is

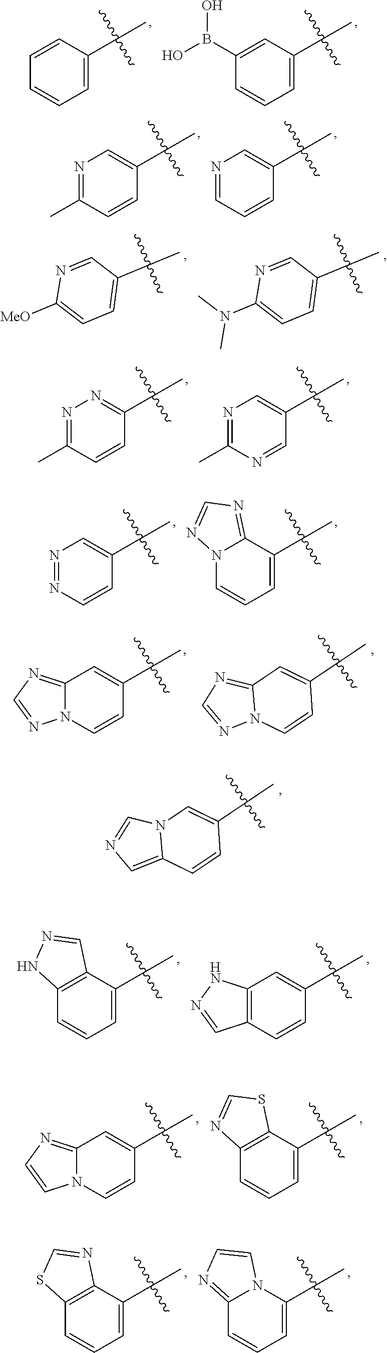

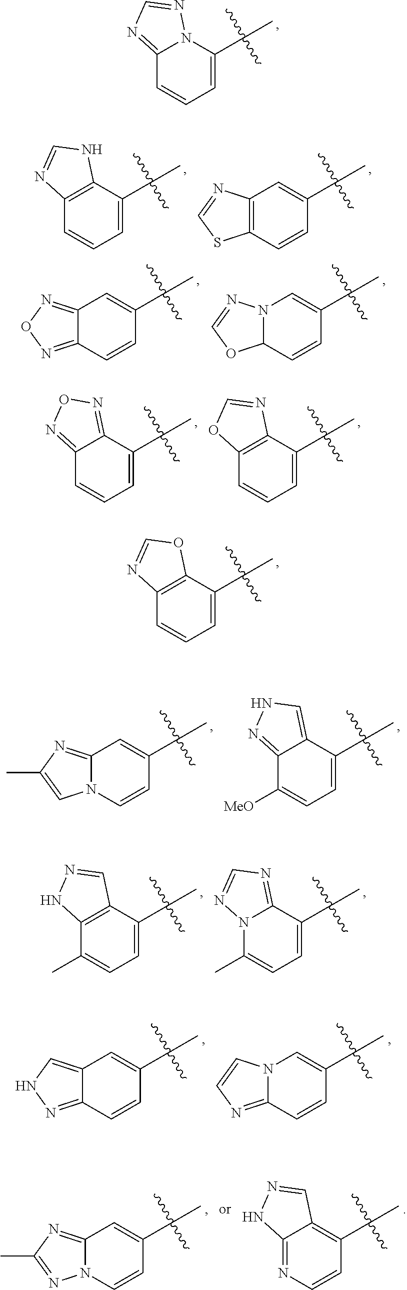

50. The compound of any one of claims 1-49, wherein R¹ is optionally substituted with 1, 2, 3, 4, 5, or 6 of R⁹ independently selected from the group consisting of alkyl, alkenyl, alkynyl, haloalkyl, heteroalkyl, halogen, hydroxyl, alkoxy, hydroxyalkyl, cyano, nitro, amino, amido, cycloalkyl, heterocyclyl, aryl, heteroaryl, acyl, N-acyl, carbonyl, oxo, sulfonyl, sulfonamide, and boronic acid.

51. The compound of claim 50, wherein R¹ is substituted with 1 of R⁹.

52. The compound of claim 1, which is of the Formula (VI-1):

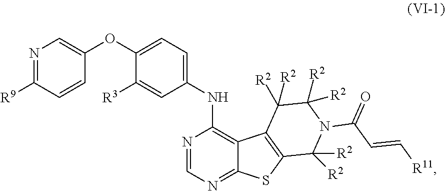

or a pharmaceutically acceptable salt, solvate, hydrate, prodrug, tautomer, stereoisomer, enantiomer, or isotopologue thereof, or a mixture thereof.

53. The compound of any one of claims 1-52, wherein each instance of R⁹ is independently hydrogen, alkyl, alkenyl, alkynyl, haloalkyl, heteroalkyl, halogen, hydroxyl, alkoxy, cyano, nitro, amino, amido, cycloalkyl, heterocyclyl, aryl, heteroaryl, N-acyl, carbonyl, sulfonyl, sulfonamide, or boronic acid.

54. The compound of claim 53, wherein each instance of R⁹ is independently hydrogen, alkyl, alkoxy, boronic acid.

55. The compound of claim 54, wherein R⁹ is methyl or methoxy.

56. The compound of any one of claims 1-55, wherein each instance of R² is independently hydrogen, alkyl, or alkoxy.

57. The compound of any one of claims 1-56, wherein all of R² are hydrogen.

58. The compound of any one of claims 1-56, wherein at least one of R² is not hydrogen.

59. The compound of any one of claims 1-56, wherein the

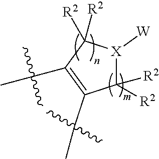

moiety in a compound of Formula (I) is

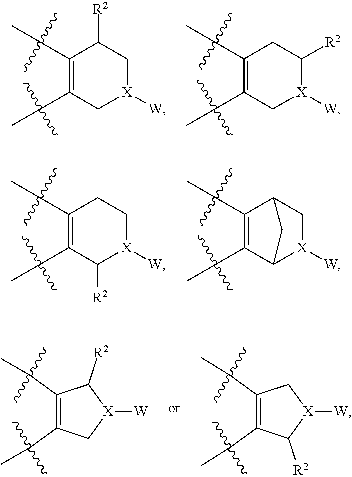

or an enantiomer thereof.

60. The compound of any one of claims 1-59, wherein each instance of R³ is independently alkyl, haloalkyl, alkoxy, or halogen.

61. The compound of claim 60, wherein R³ is methyl. —CF₃, methoxy, fluoro, or chloro.

62. The compound of claim 1, wherein the compound is Table 1, or a pharmaceutically acceptable salt, solvate, hydrate, prodrug, tautomer, stereoisomer, enantiomer, or isotopologue thereof, or a mixture thereof.

63. A pharmaceutical composition comprising a compound of any one of claims 1-62 and one or more pharmaceutically acceptable excipients.

64. A method of treating, preventing, or managing a disorder mediated by HER2 or a HER2 mutant in a subject, comprising administering a therapeutically or prophylactically effective amount of a compound of any one of claims 1-62 or a pharmaceutical composition of claim 7 to the subject.

65. The method of claim 64, wherein the disorder is mediated by expression, overexpression, amplification, or activation of HER2.

66. The method of claim 64, wherein the disorder is mediated by expression, overexpression, amplification, or activation of one or more HER2 mutants.

67. The method of claim 66, wherein the one or more HER2 mutants are selected from HER2YVMA, HER2VC, HER2 L755S, HER2 G776C, and HER2 V777_G778insCG.

68. The method of claim 64, wherein the disorder is cancer.

69. The method of claim 68, wherein the cancer is a solid tumor.

70. The method of claim 68, wherein the cancer is breast cancer, gastric cancer, esophageal cancer, ovarian cancer, endometrial cancer, endometrial serous carcinoma, cervix cancer, bladder cancer, lung cancer, colorectal cancer, head and neck cancer, cholangial cancer, germ cell cancer, glioblastoma, liver cancer, melanoma, osteosarcoma, pancreatic cancer, renal cell cancinoma, salivary duct carcinoma, or soft tissue cancer.

71. The method of claim 70, wherein the cancer is breast cancer, gastric cancer, esophageal cancer, ovarian cancer, or endometrial cancer.

72. The method of claim 71, wherein the cancer is breast cancer.

73. The method of claim 72, wherein the breast cancer is a metastatic breast cancer that spreads to brain.

74. The method of claim 72, wherein the breast cancer is a HER2 amplified metastatic breast cancer.

75. The method of claim 72, wherein the breast cancer is characterized by the presence of one or more HER2 mutants.

76. The method of claim 75, wherein the HER2 mutant is HER2 L755_T759del, HER2 L755S, HER2 V777L, HER2 R896C, HER2 D769H, HER2 D769Y, HER2 G309A, HER2 V842I, or HER2 P780_Y781insGSP.

77. The method of claim 70, wherein the cancer is lung cancer.

78. The method of claim 77, wherein the lung cancer is non-small cell lung carcinoma.

79. The method of claim 77 or 78, wherein the lung cancer or non-small cell lung carcinoma is characterized by the presence of one or more HER2 mutants.

80. The method of claim 79, wherein the HER2 mutant is HER2YVMA, HER2VC, HER2 L755S, HER2 G776C, or HER2 V777_G778insCG.

81. The method of claim 70, wherein the cancer is colorectal cancer.

82. The method of claim 81, wherein the colorectal cancer is characterized by the presence of one or more HER2 mutants.

83. The method of claim 82, wherein the HER2 mutant is HER2 L755S, HER2 V777L, HER2 V777M, HER2 V842I, HER2 S310F, or HER2 L866M.

1. A compound of Formula (I): 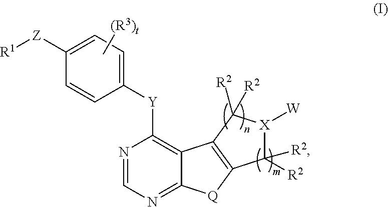
or a pharmaceutically acceptable salt thereof, wherein:
X is CR⁵ or N;
Y is NR⁶, CR⁷R, or O;
Z is NR⁶ or O;
Q is S, NR⁶, or CR⁷R;
n is 1, 2, 3, or 4;
m is 1, 2, 3, or 4;
t is 0, 1, 2, 3, or 4;
R¹ is aryl, heteroaryl, cycloalkyl, or heterocyclyl;
each instance of R² is independently hydrogen, alkyl, alkenyl, alkynyl, haloalkyl, hydroxyalkyl, heteroalkyl, halogen, cyano, nitro, amido, cycloalkyl, heterocyclyl, aryl, heteroaryl,
—OR′, or
—NR′R″, wherein R′ and R¹¹ are independently hydrogen, alkyl, heteroalkyl, aryl, or heteroaryl, or
R′ and R¹¹ are taken together with nitrogen to form a cyclic moiety;
or two R² are taken together to form a C₁-C₃ alkylene;
each instance of R³ is independently alkyl, alkenyl, alkynyl, haloalkyl, hydroxyalkyl, heteroalkyl, halogen, hydroxyl, alkoxy, cyano, nitro, amino, amido, cycloalkyl, heterocyclyl, aryl, or heteroaryl;
each instance of R⁵, R⁶, R⁷, and R¹ is independently hydrogen or alkyl;
W is
-L-(NR⁶)ₛ—C(═O)—(CR¹⁰)=CR¹⁰)—R¹¹,
-L-(NR⁶)ₛ—C(═O)—C(═CR¹⁰R¹⁰)—R¹¹,
-L-(CR¹⁰=CR¹⁰)—C(═O)—(NR⁶)ₛ—R¹¹,
-L-C(═CR¹⁰R¹⁰)—(═O)—(NR⁶)ₛ—R¹¹,
-L-(NR⁶)ₛ—S(═O)₂—(CR¹⁰=CR¹⁰)—R¹¹,
-L-(NR⁶)ₛ—S(═O)₂—C(═CR¹⁰R¹⁰)—R¹¹,
-L-(CR¹⁰=CR¹⁰)—S(═O)₂—(N⁶)ₛ—R¹¹; or
-L-C(═CR¹⁰=R¹⁰)—S(═O)₂—NR⁶)ₛ—R¹¹;
L is a bond or C₁-C₃ alkylene;
s is 0 or 1;
each instance of R¹⁰ is independently hydrogen, alkyl, haloalkyl, hydroxyalkyl, heteroalkyl, alkoxy, cycloalkyl, heterocyclyl, aryl, heteroaryl, or halo;
R¹¹ is hydrogen, —OR¹², —(C₁-C₃ alkylene) —OR¹²NR¹²R¹³, —(C₁-C₃ alkylene) —NR¹²R¹³, cycloalkyl, —(C₁-C₃ alkylene)-aryl, heteroaryl, or —(C₁-C₃ alkylene)-heteroaryl; and
R¹² and R¹³ are independently hydrogen, alkyl, haloalkyl, hydroxyalkyl, heteroalkyl, alkoxy, cycloalkyl, heterocyclic, aryl, heteroaryl, cycloalkylalkyl, heterocyclylalkyl, arylalkyl, or heteroarylalkyl, or R¹² and R¹³ are taken together with nitrogen to form a cyclic moiety.

2. The compound of claim 1, which is of the Formula (II-1), (II-2), (II-3), (II-4), (II-5): 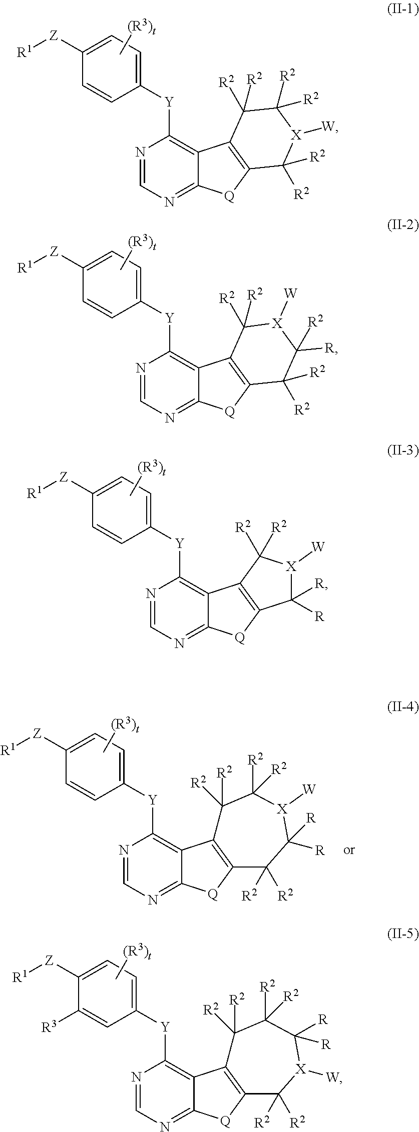
or a pharmaceutically acceptable salt thereof.

3. The compound of claim 1, which is of the Formula (III-1), (III-2), (III-3), (III-4), (III-5): 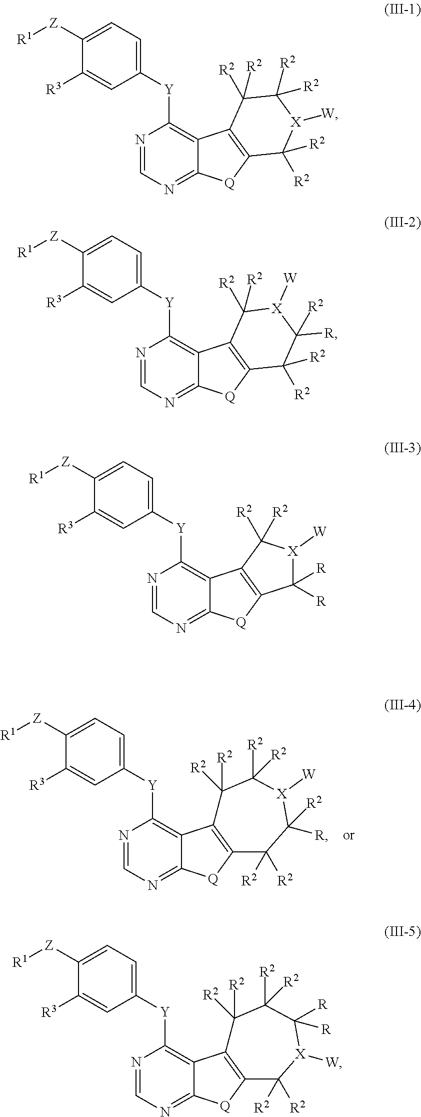
or a pharmaceutically acceptable salt thereof.

4. The compound of claim 1, which is of the Formula (IV-1), (IV-2), (IV-3), (IV-4), (IV-5), (IV-6), OR (IV-7): 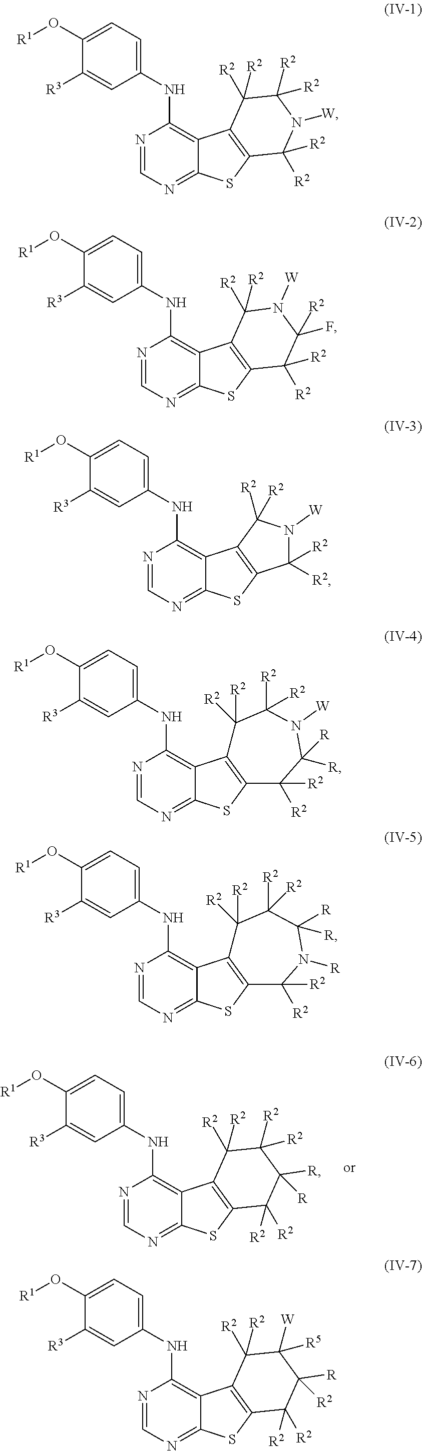
or a pharmaceutically acceptable salt thereof.

5. The compound of claim 1, which is of the Formula (V-1), (V-2), (V-3), (V-4), (V-5), (V-6), (V-7), (V-s), (V-9), (V-10), (V-11), (V-12), (V-13), or (V-14): 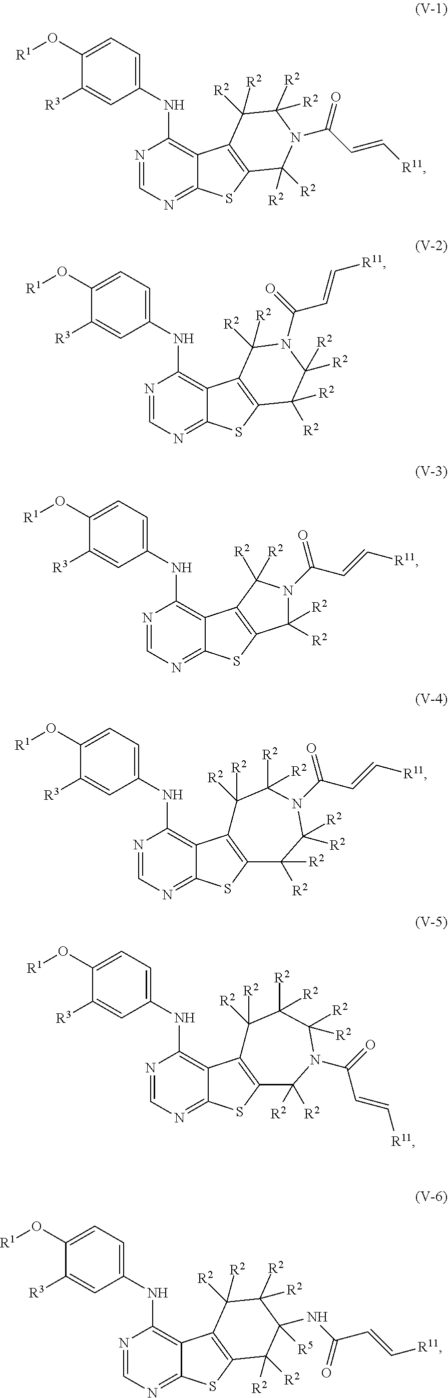 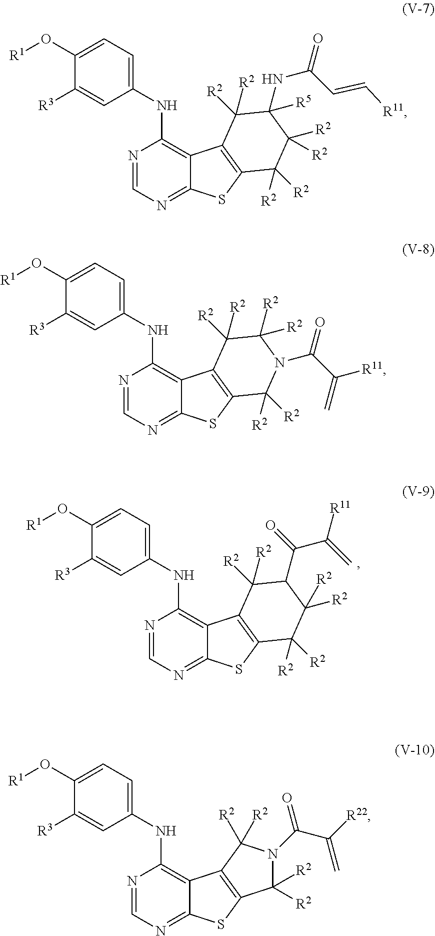 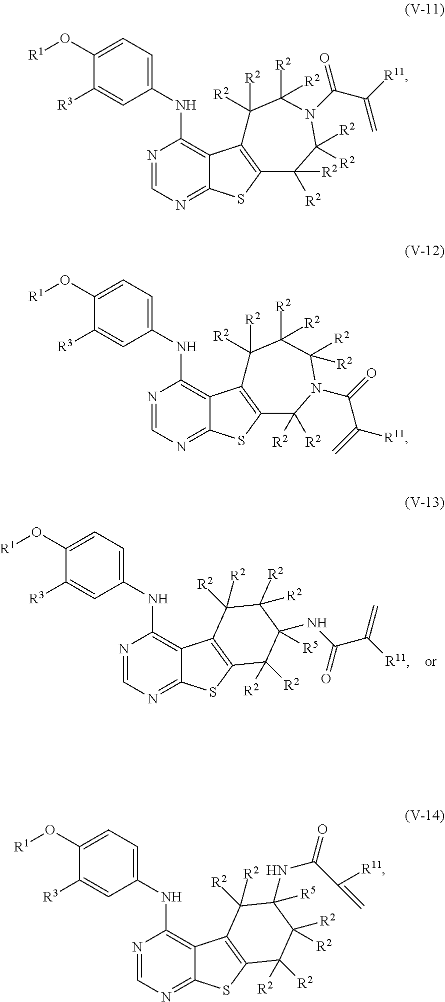
or a pharmaceutically acceptable salt thereof.

6. The compound of claim 1 which is of the Formula (VI-1) 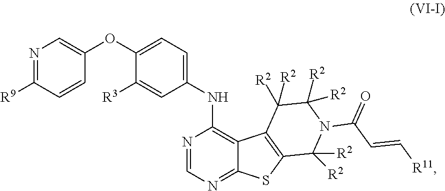
or a pharmaceutically acceptable salt thereof.

7. A compound, or salt thereof according to any one of the preceding claims wherein the compound is selected from:

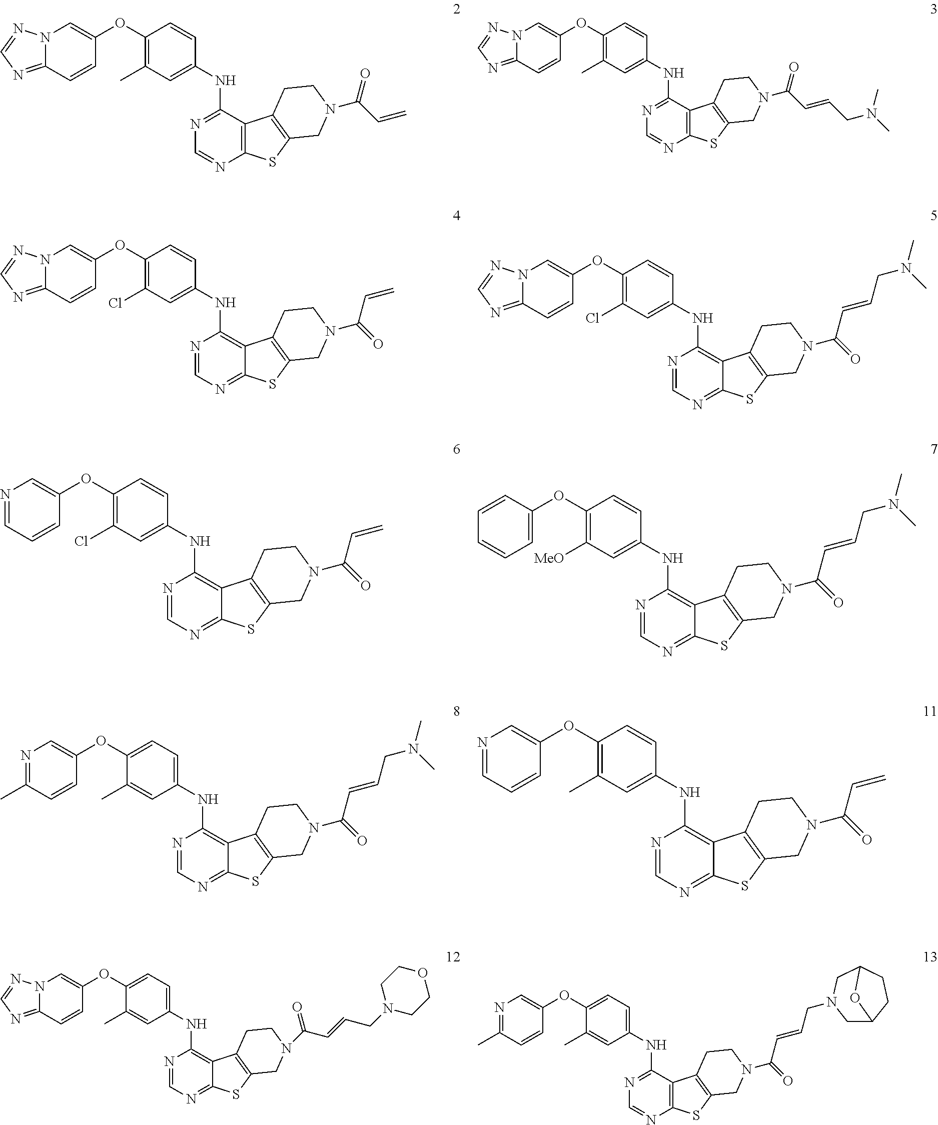

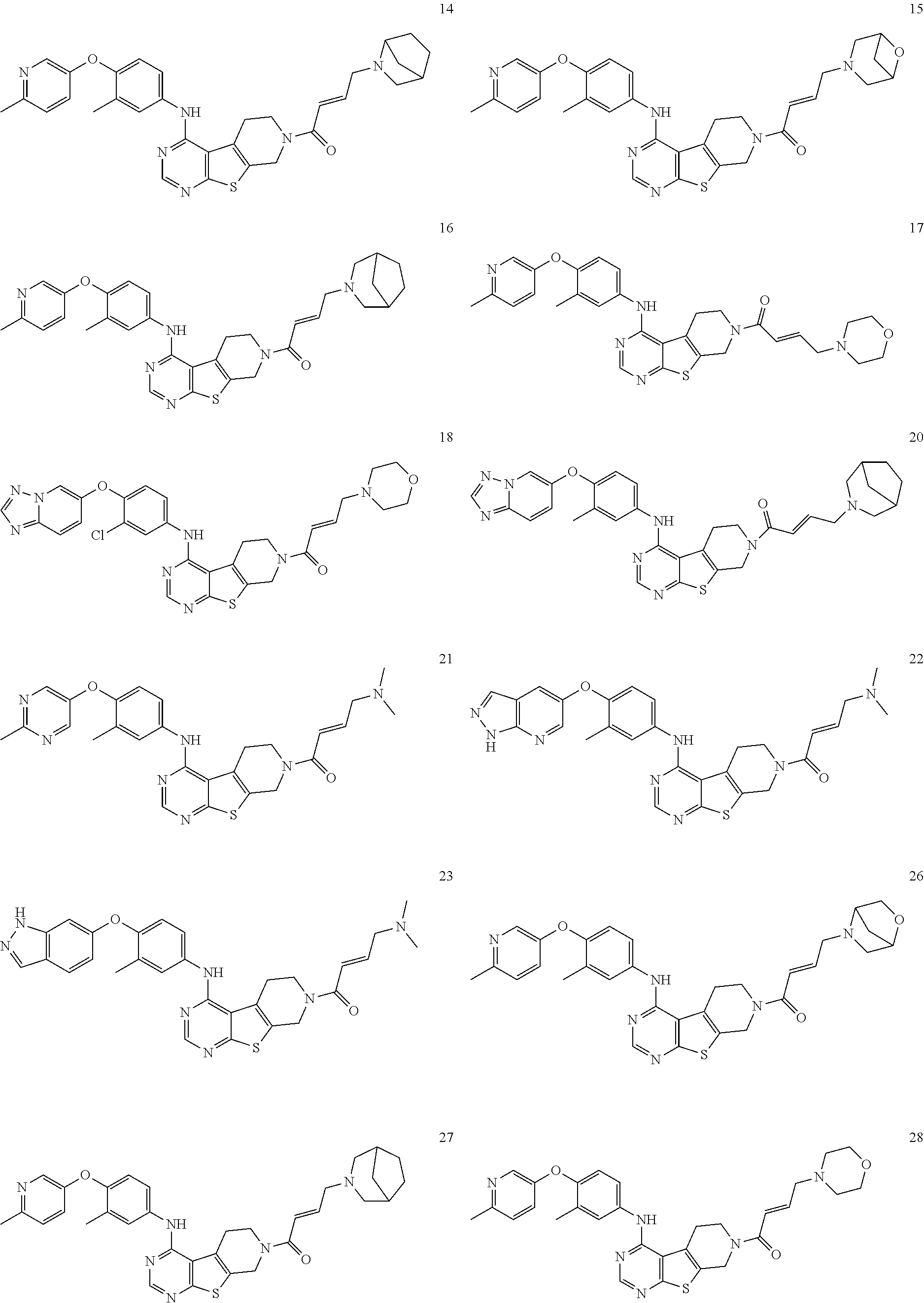

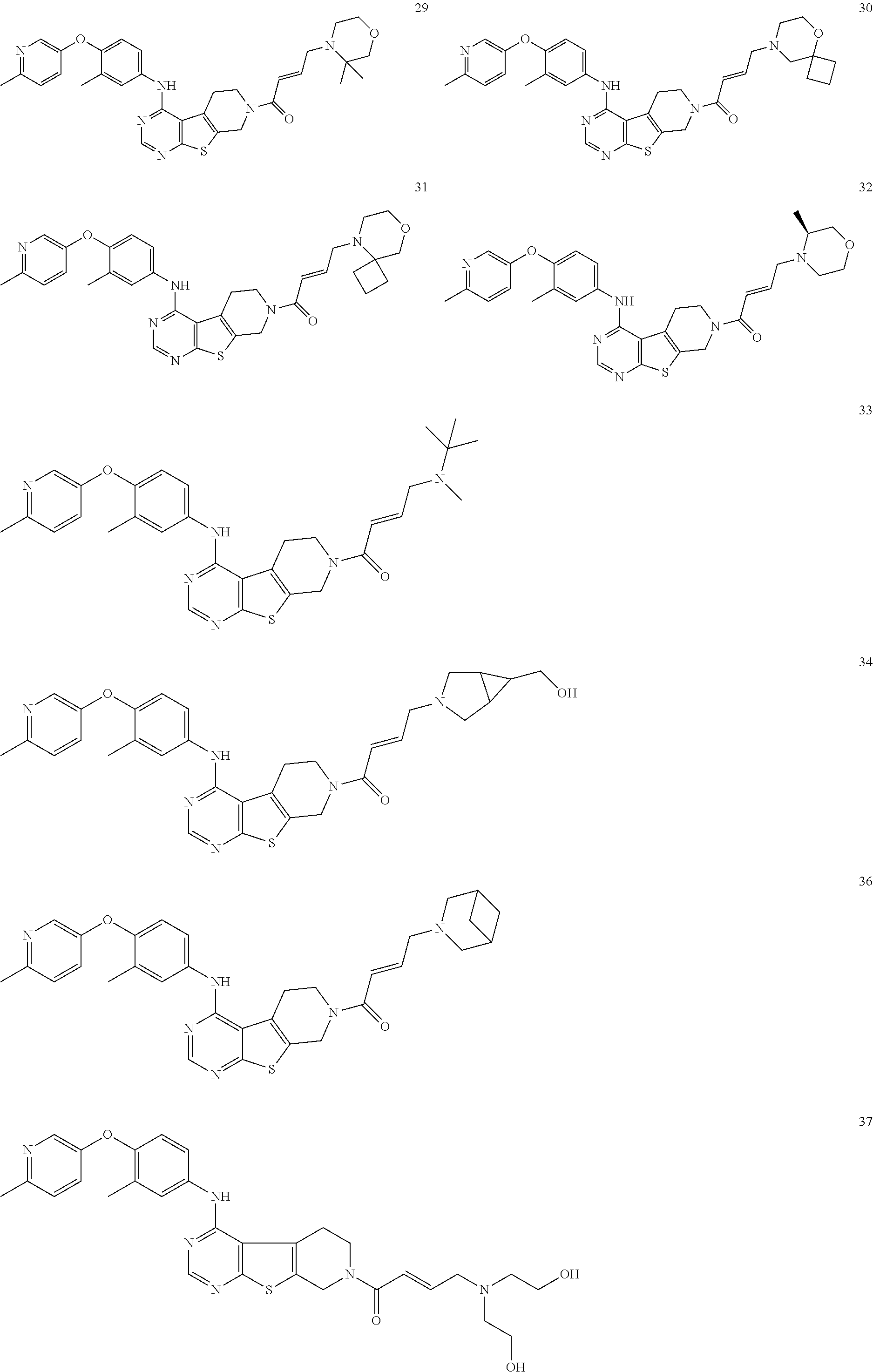

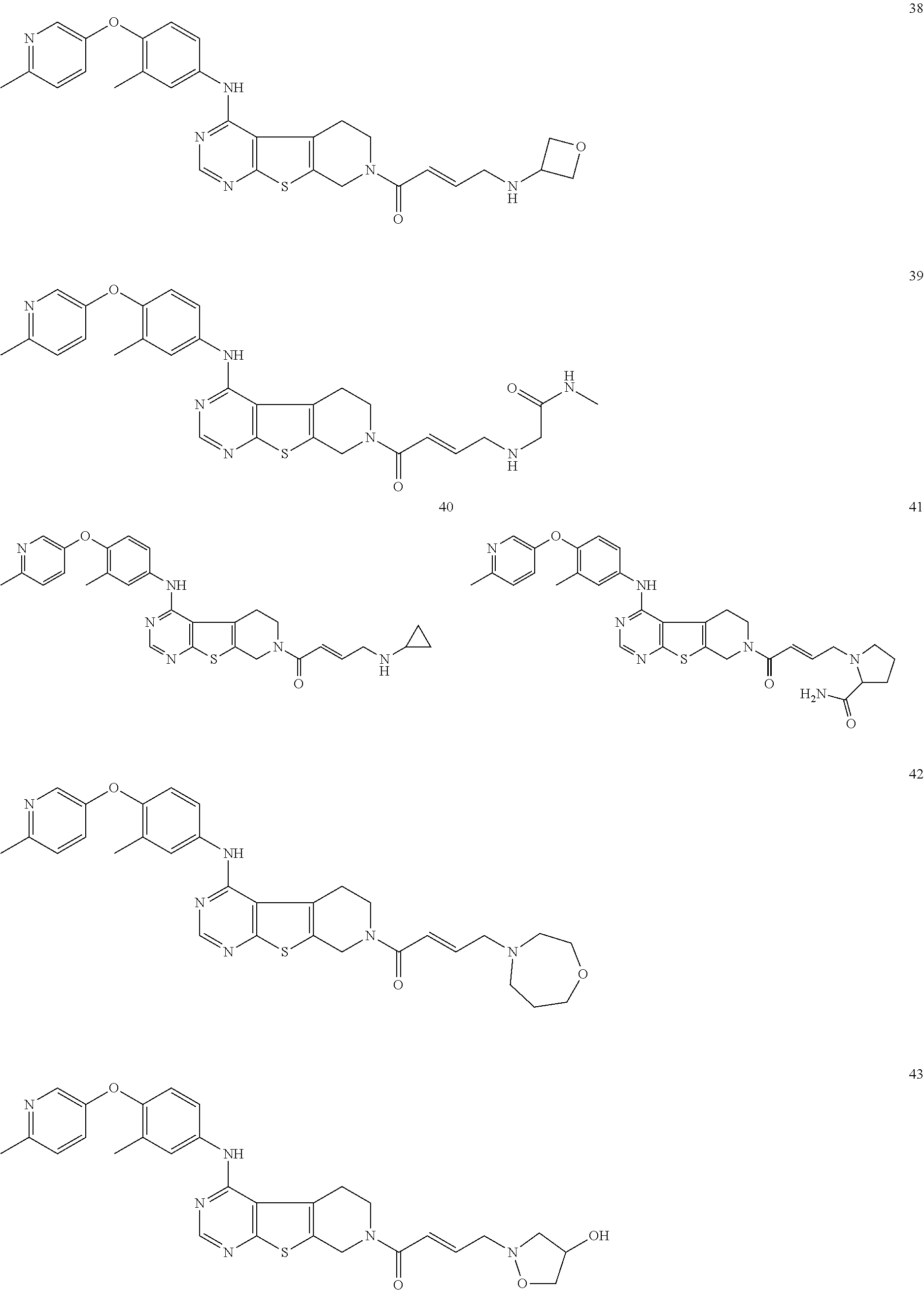

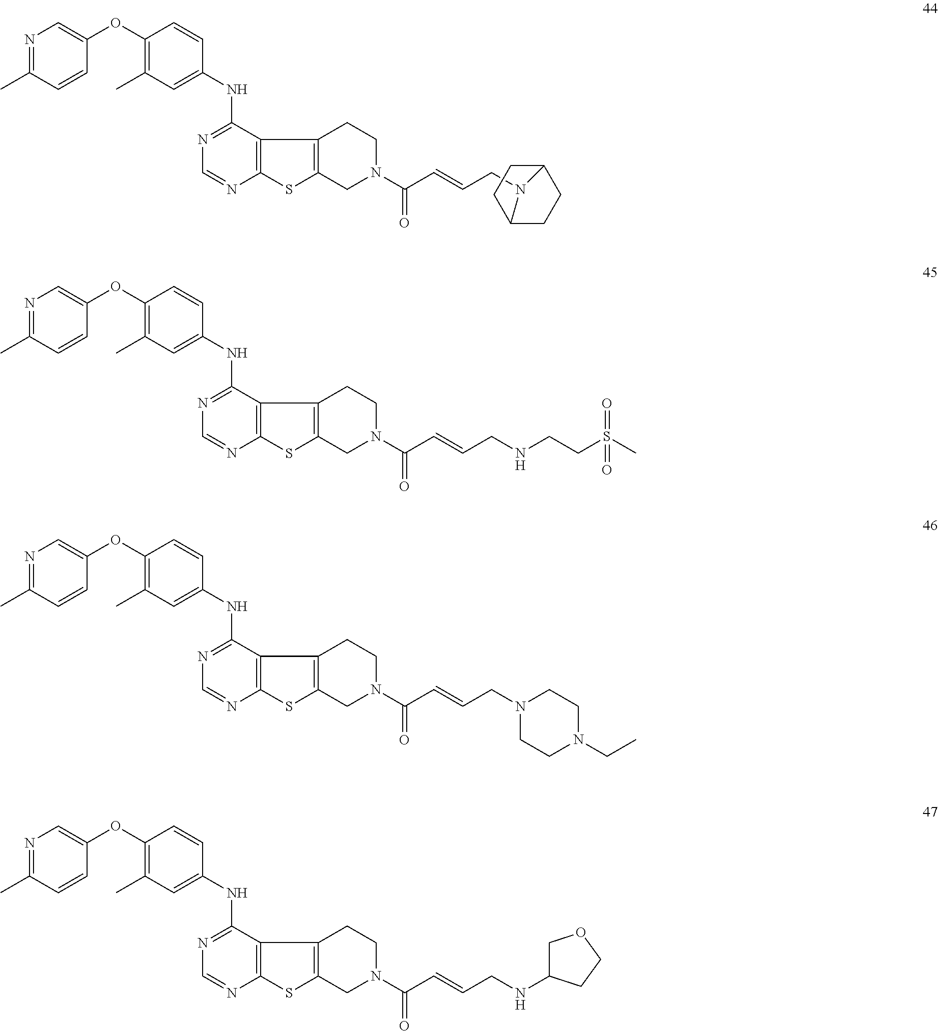

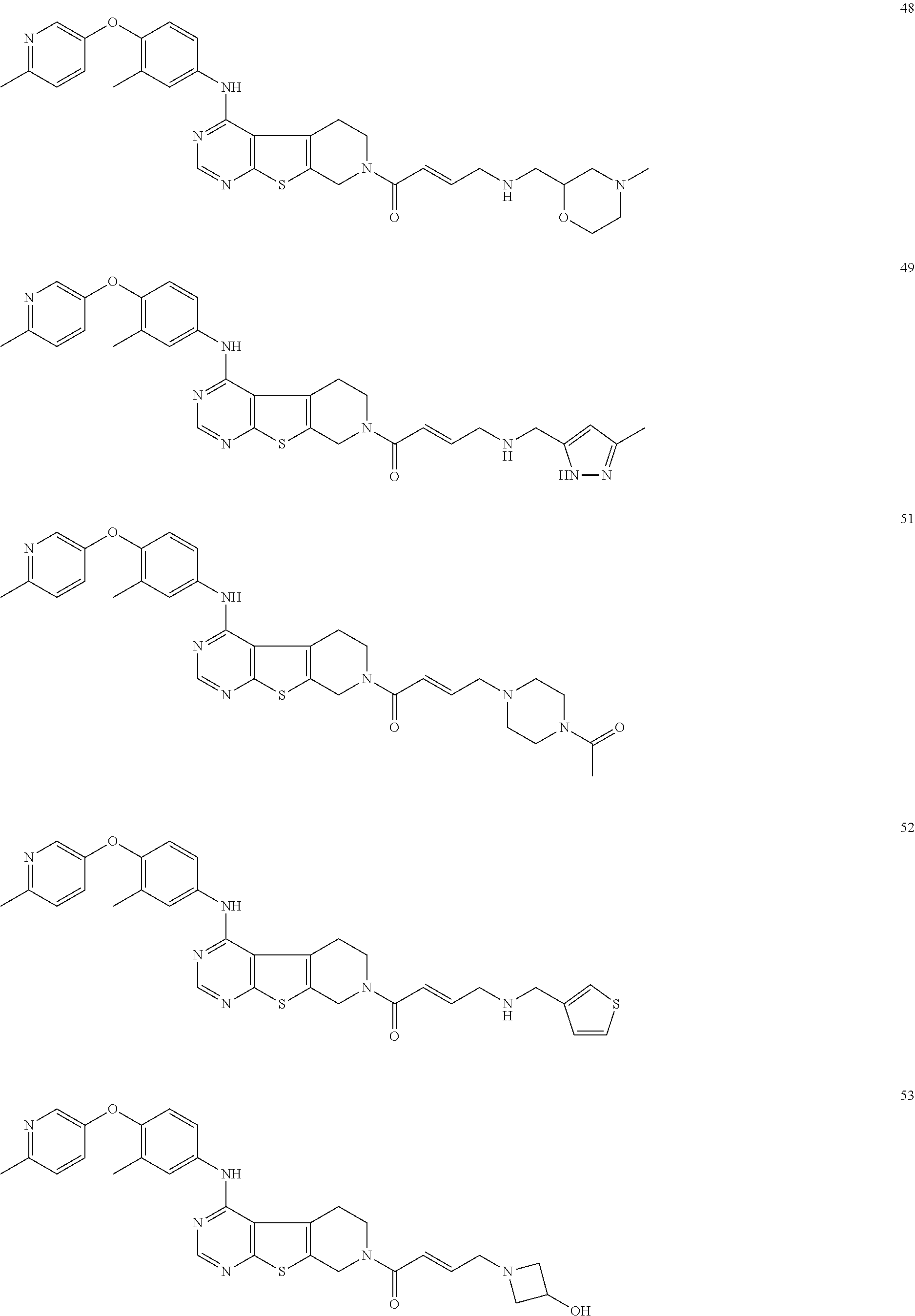

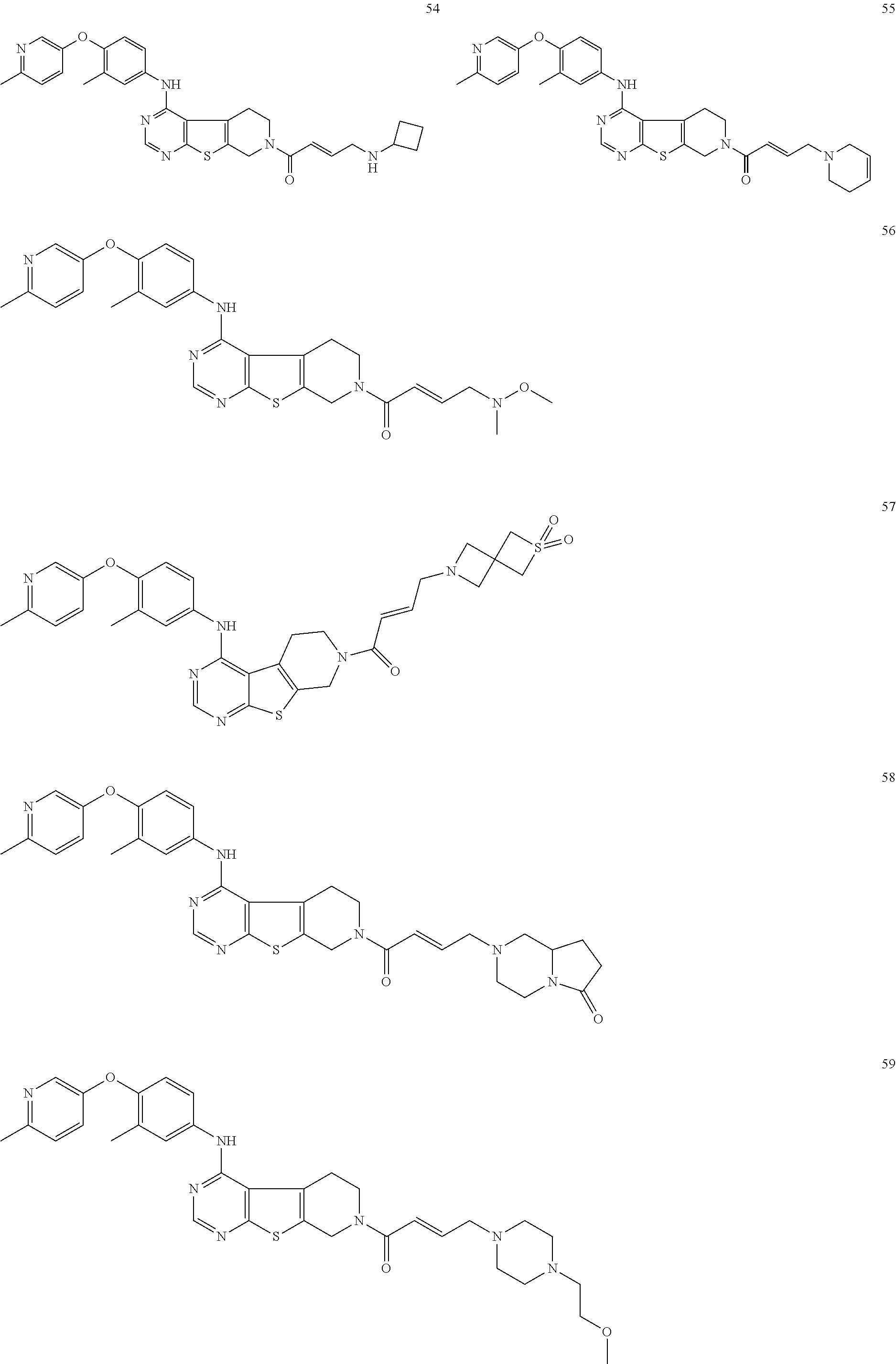

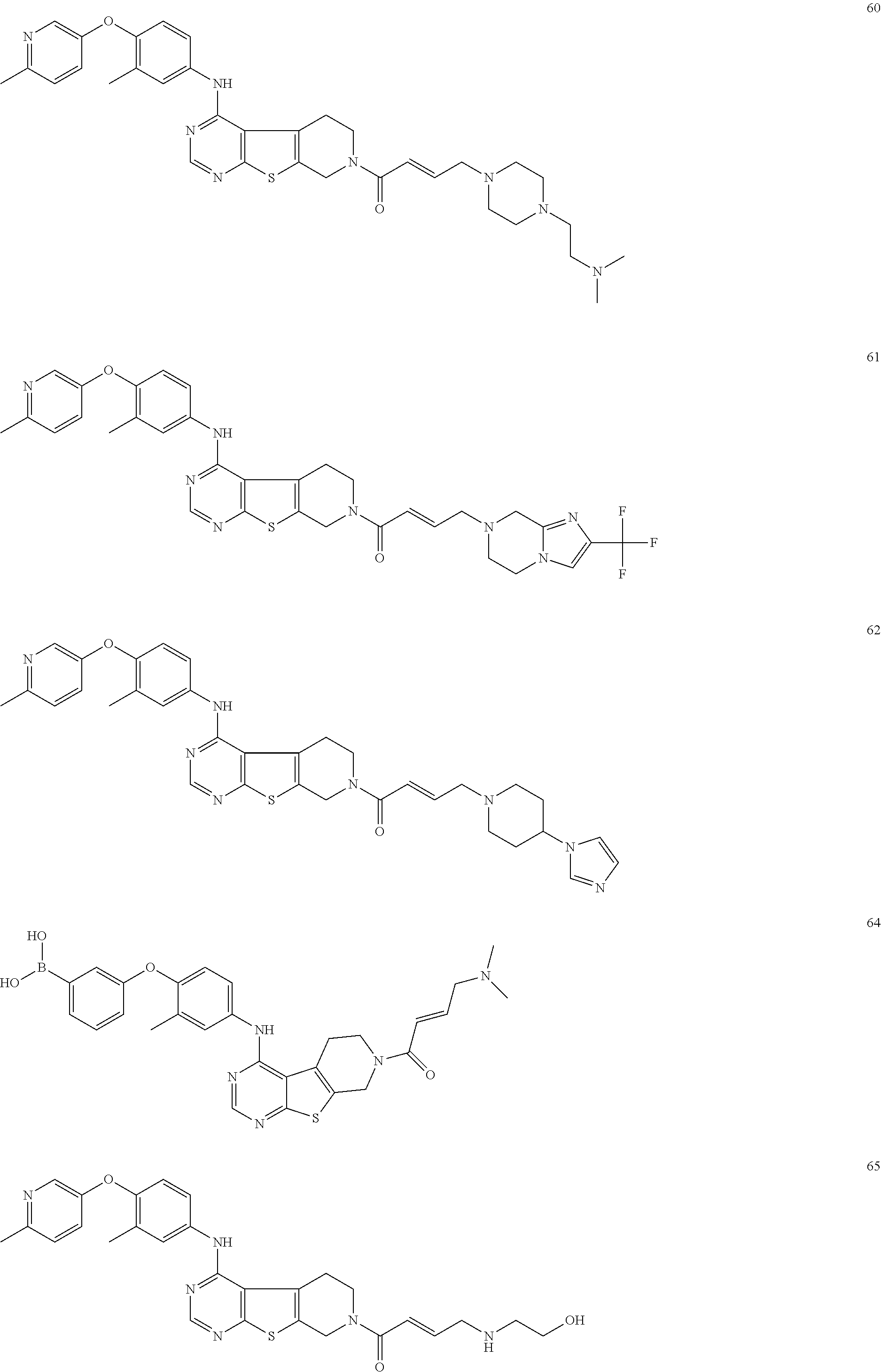

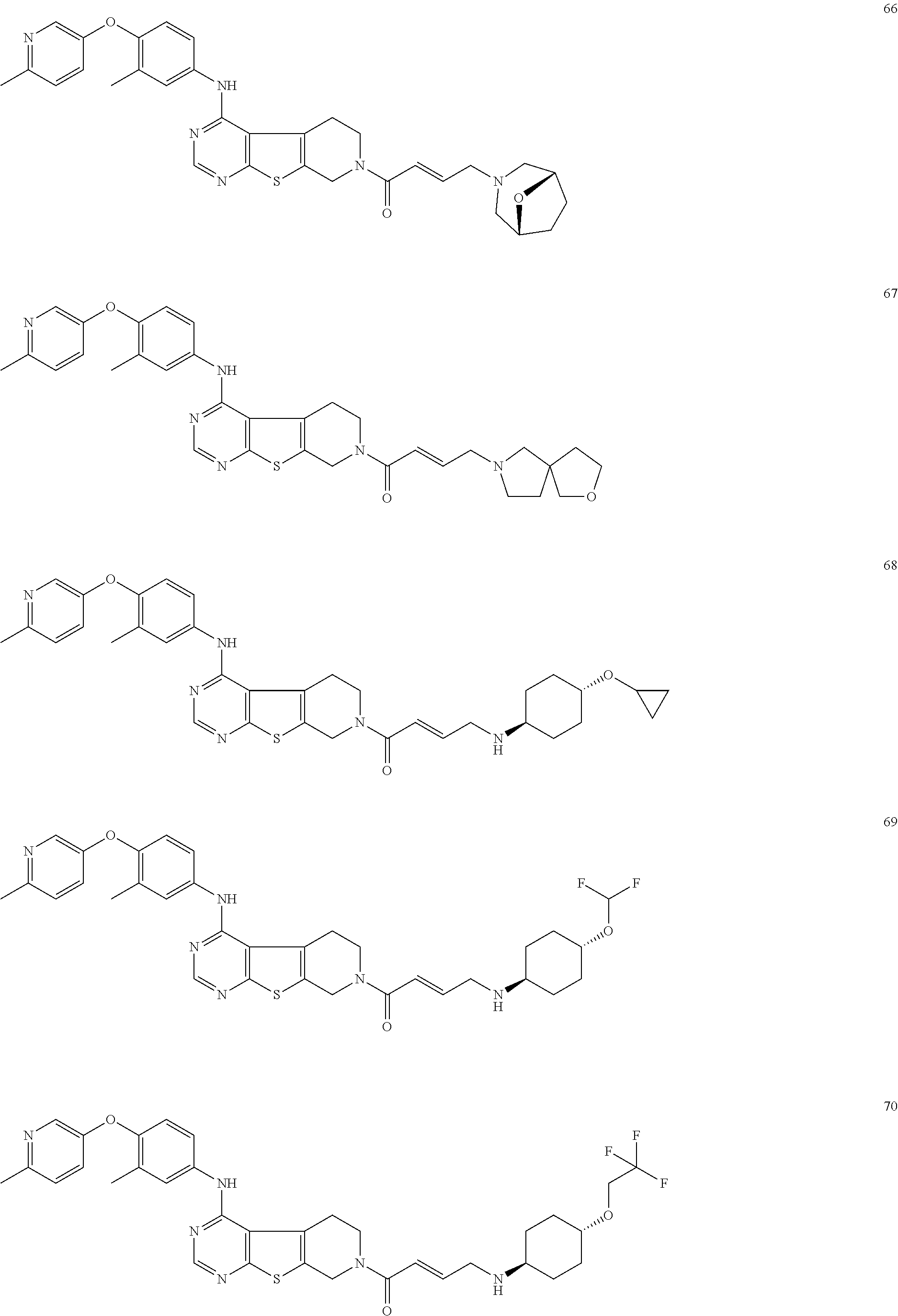 [SMILES: Cc1ccc(Oc2ccc(Nc3ncnc4sc5c(c34)CCN(C(=O)/C=C/CN3C[C@H]4CC[C@@H](C3)O4)C5)cc2C)cn1 ; Cc1ccc(Oc2ccc(Nc3ncnc4sc5c(c34)CCN(C(=O)/C=C/CN3CCC4(CCOC4)C3)C5)cc2C)cn1 ; Cc1ccc(Oc2ccc(Nc3ncnc4sc5c(c34)CCN(C(=O)/C=C/CN[C@H]3CC[C@H](OC4CC4)CC3)C5)cc2C)cn1 ; Cc1ccc(Oc2ccc(Nc3ncnc4sc5c(c34)CCN(C(=O)/C=C/CN[C@H]3CC[C@H](OC(F)F)CC3)C5)cc2C)cn1 ; Cc1ccc(Oc2ccc(Nc3ncnc4sc5c(c34)CCN(C(=O)/C=C/CN[C@H]3CC[C@H](OCC(F)(F)F)CC3)C5)cc2C)cn1]

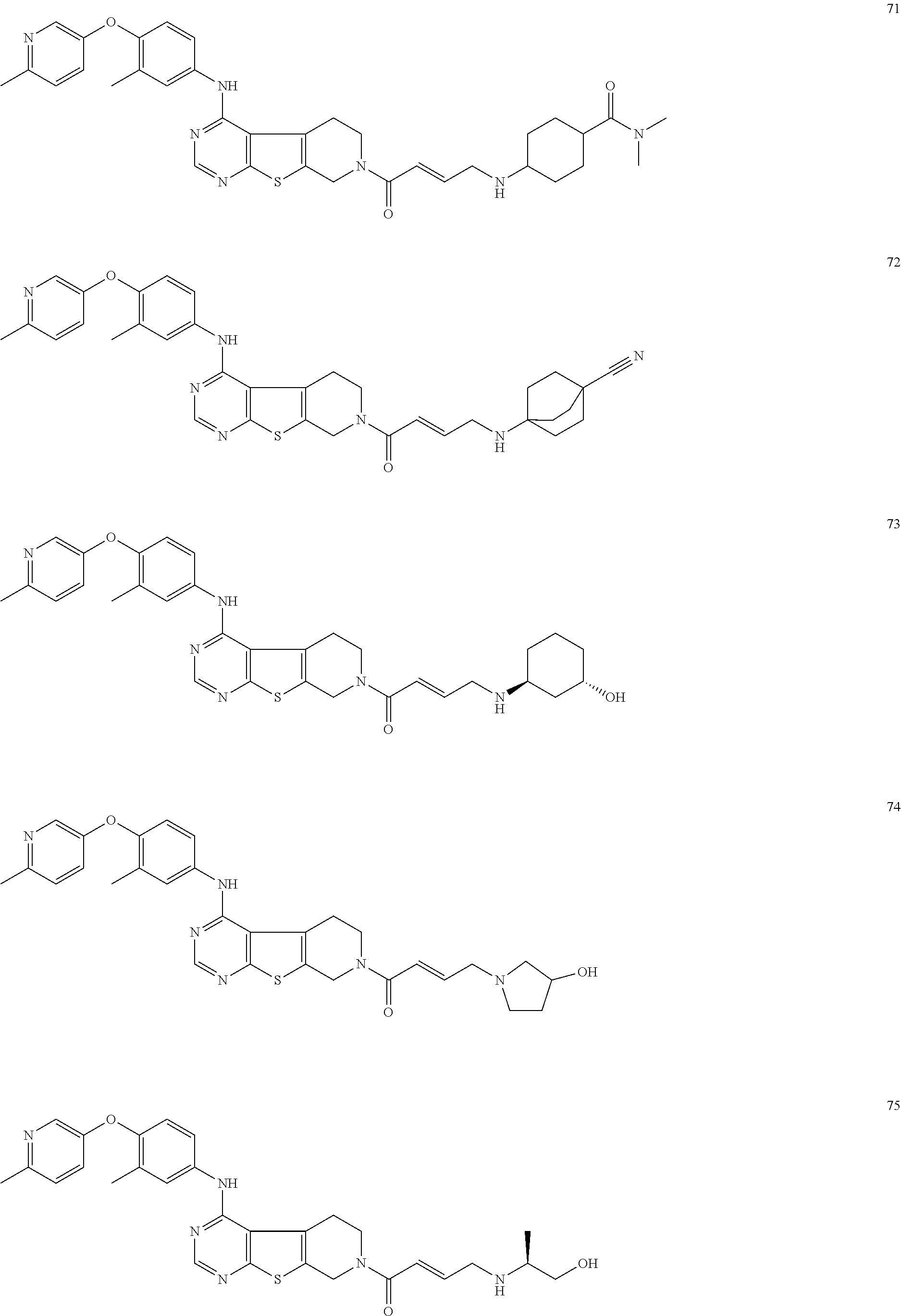

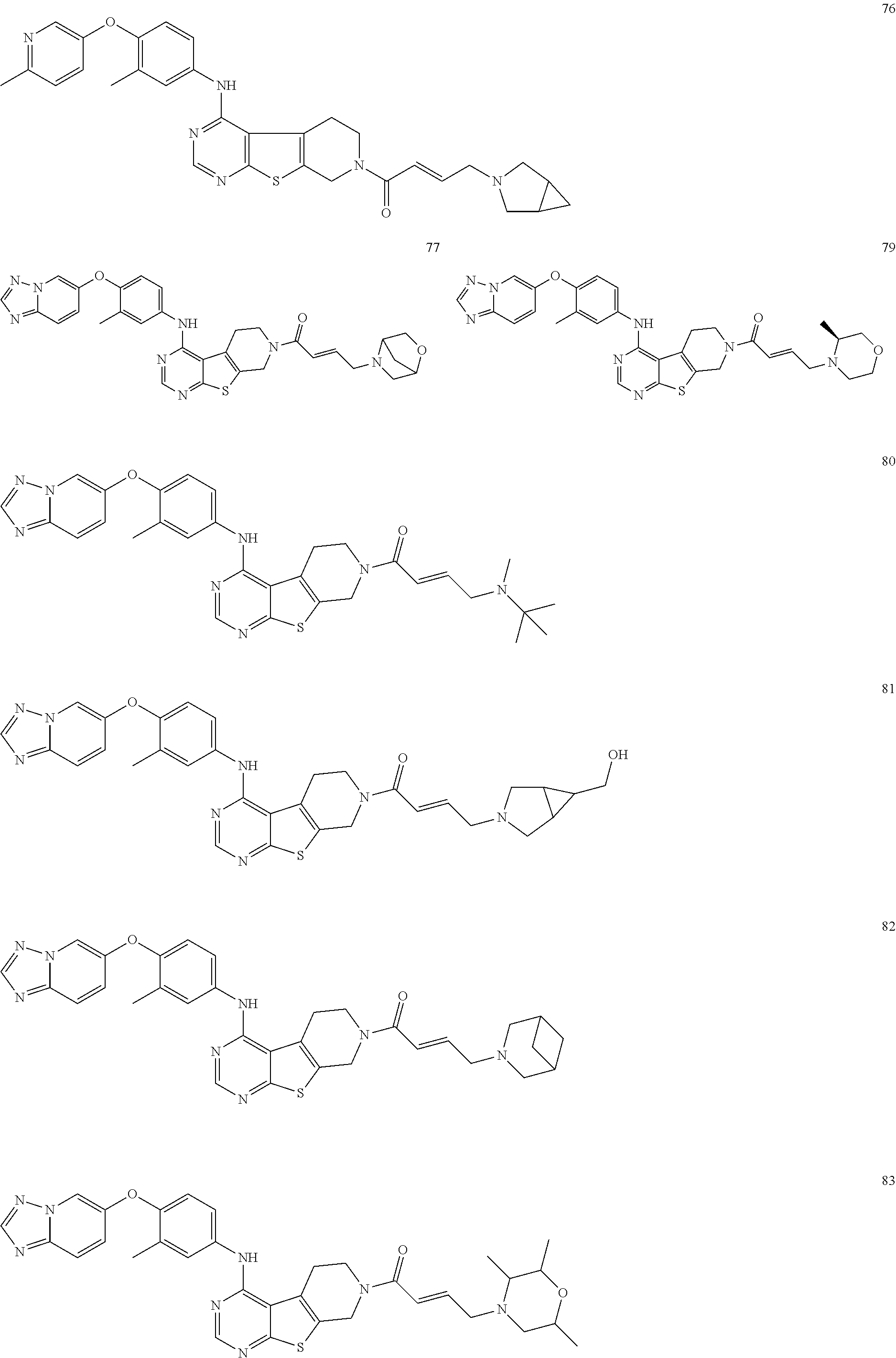

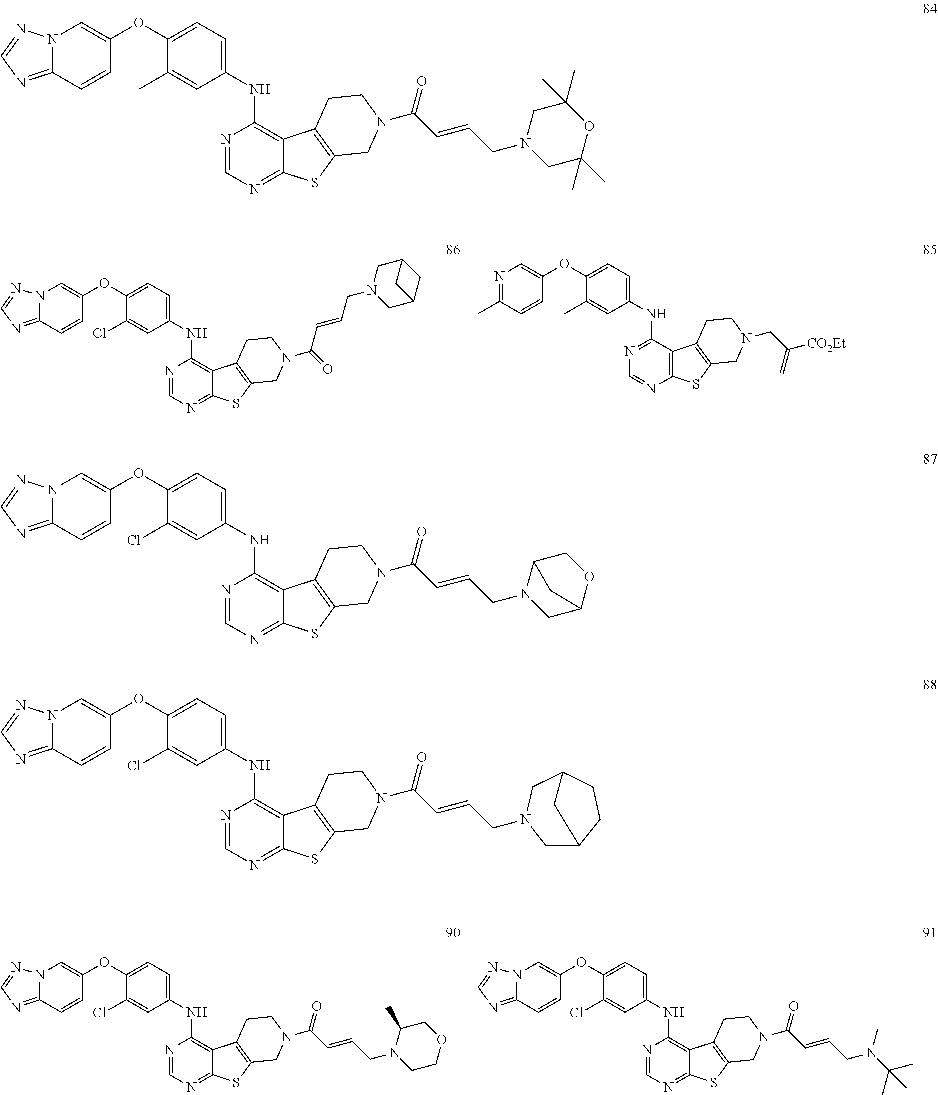

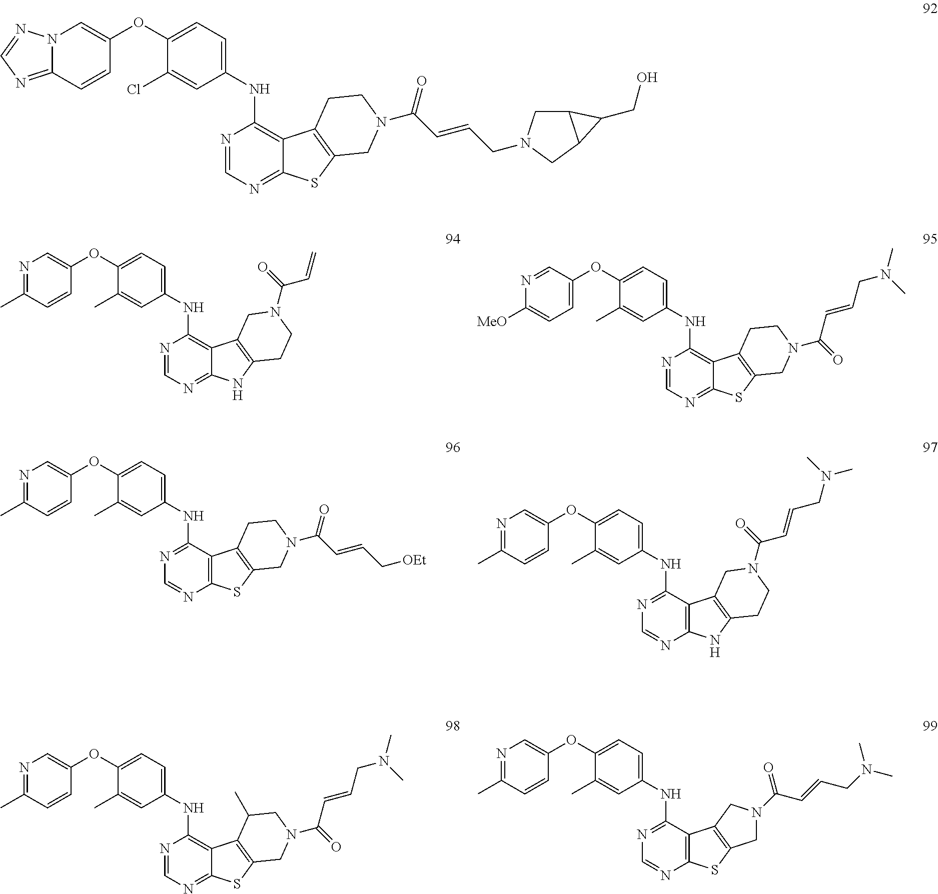

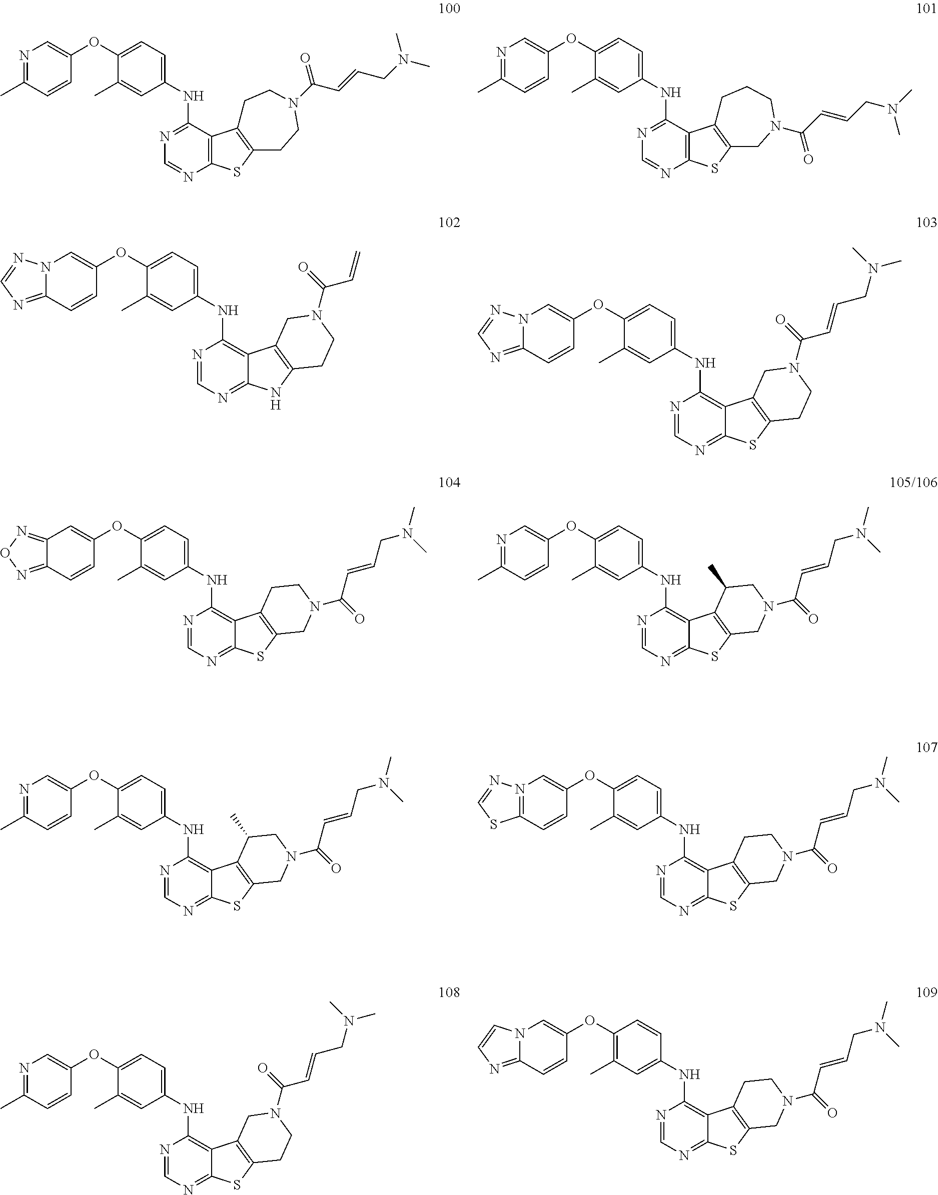

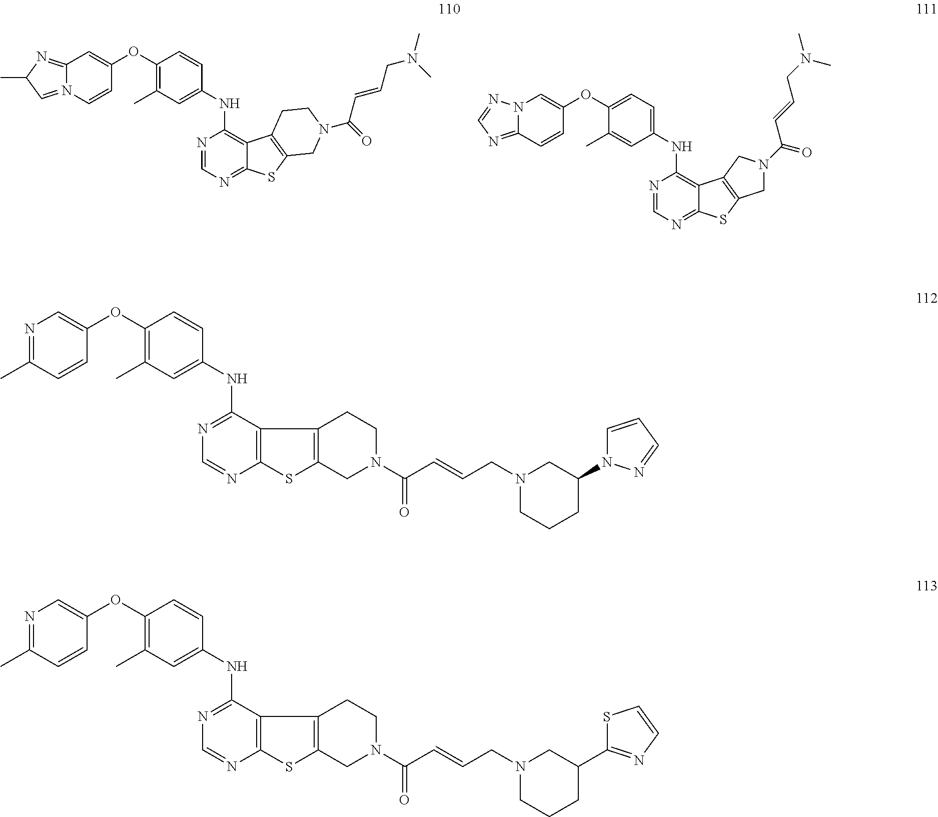

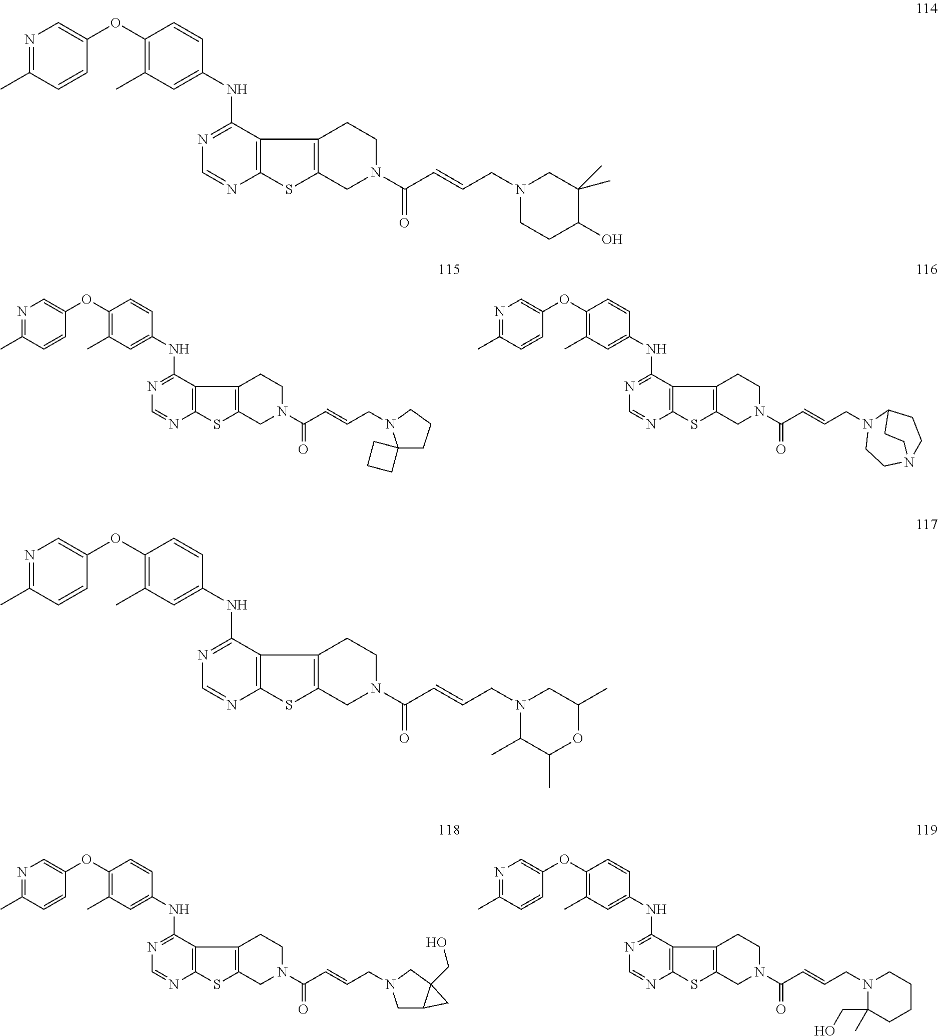

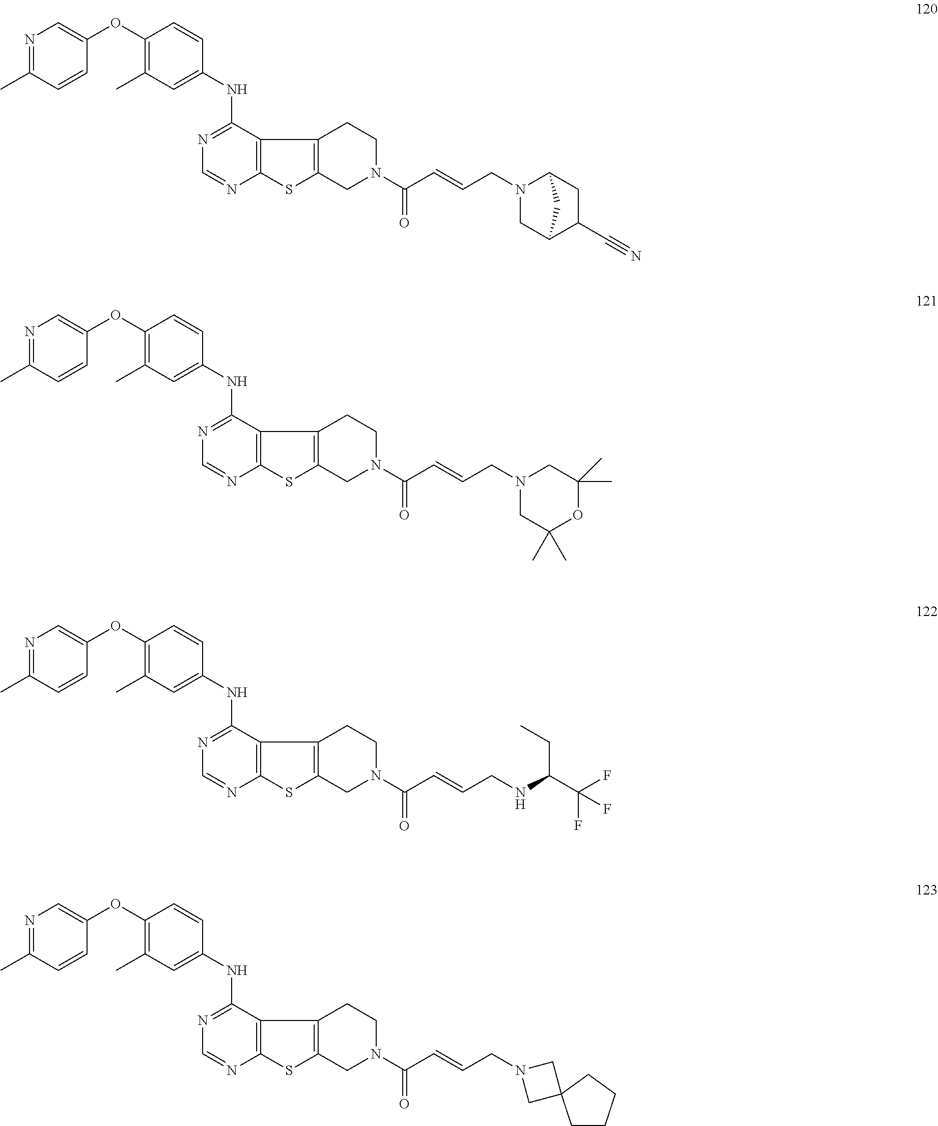

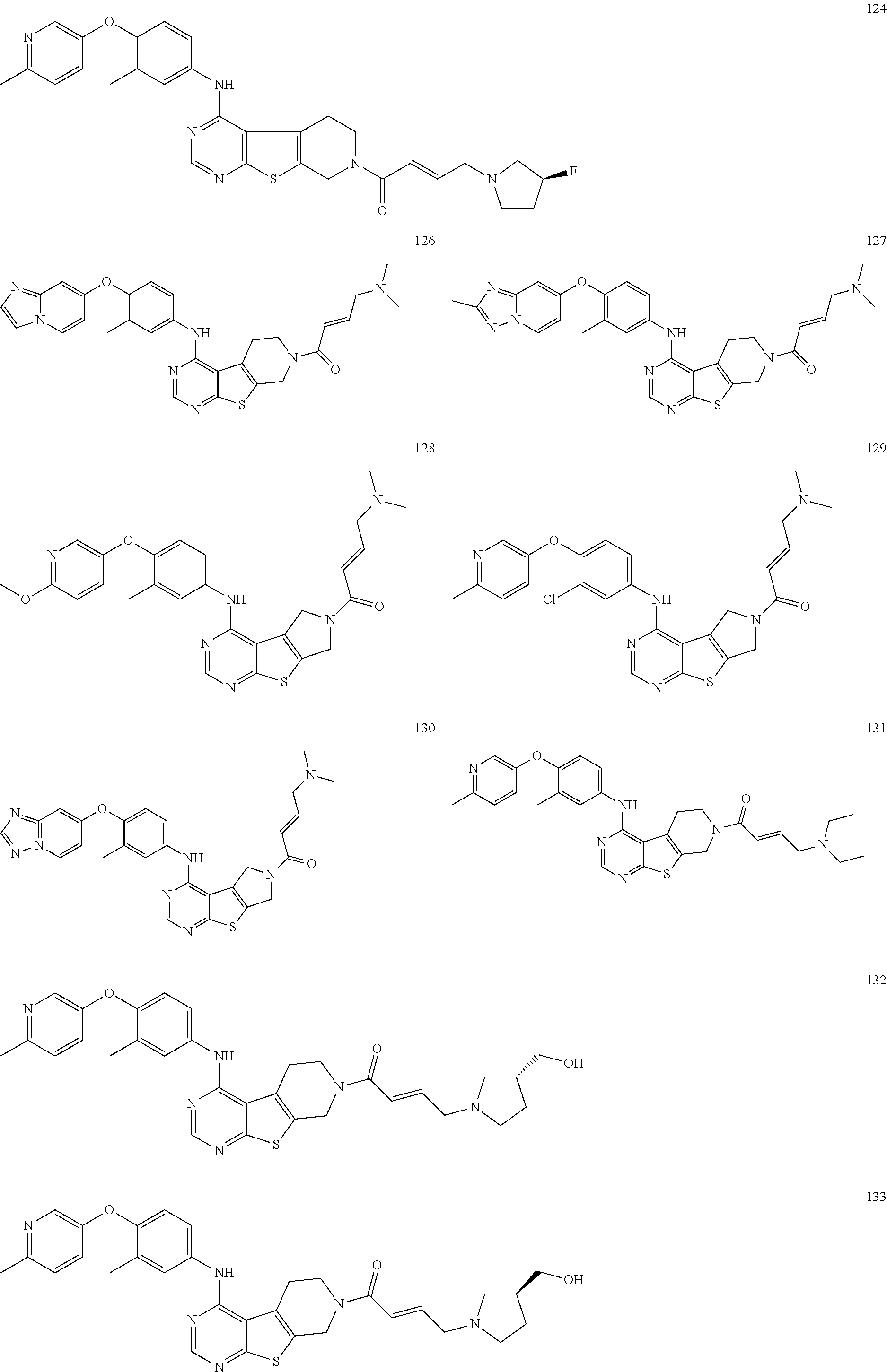

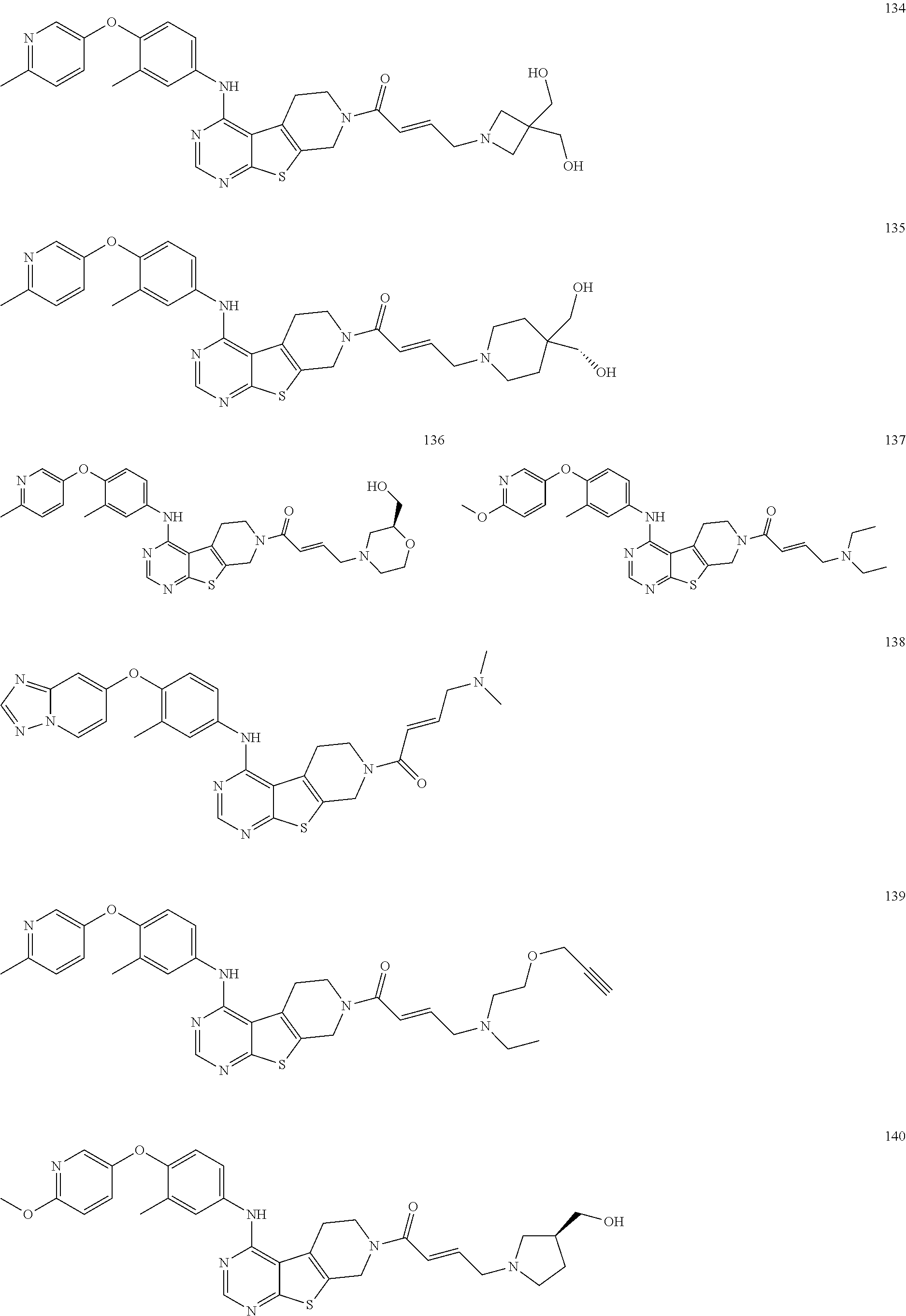

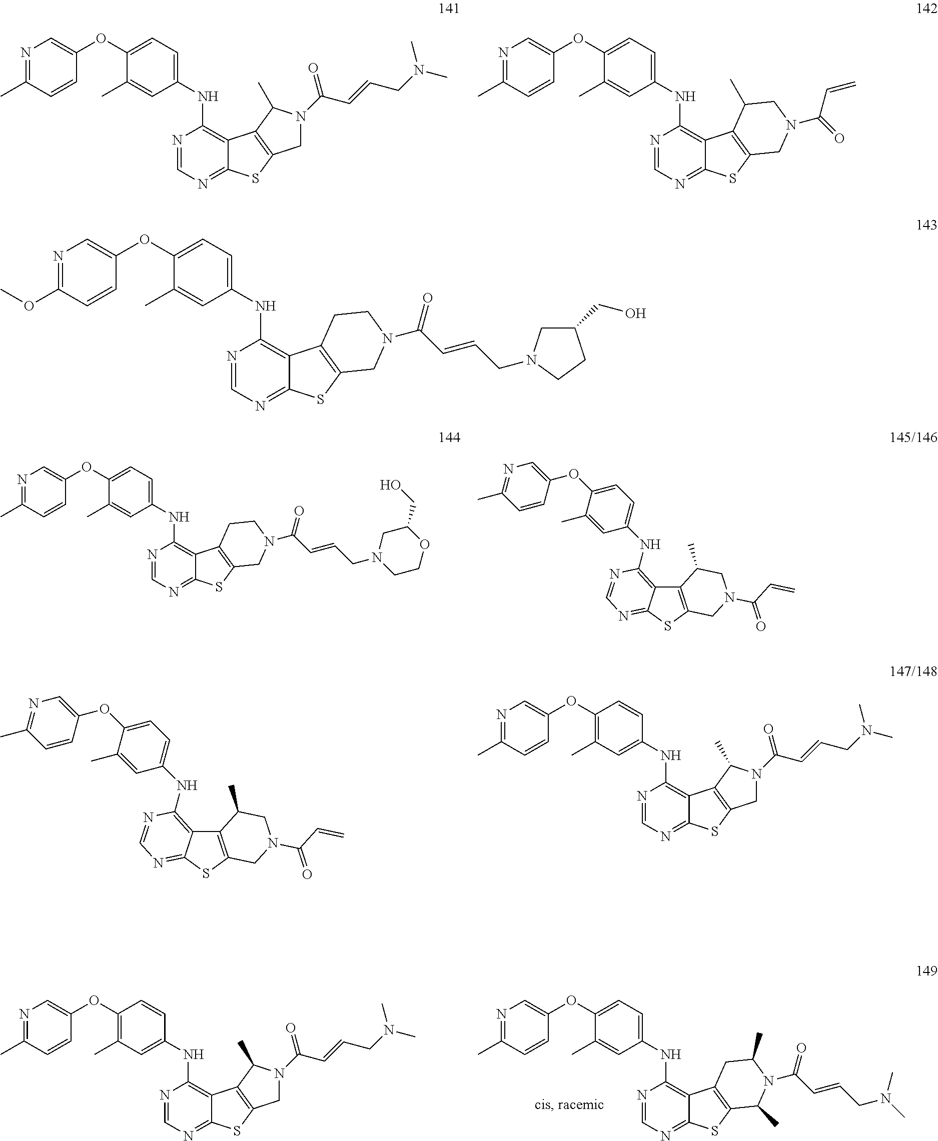

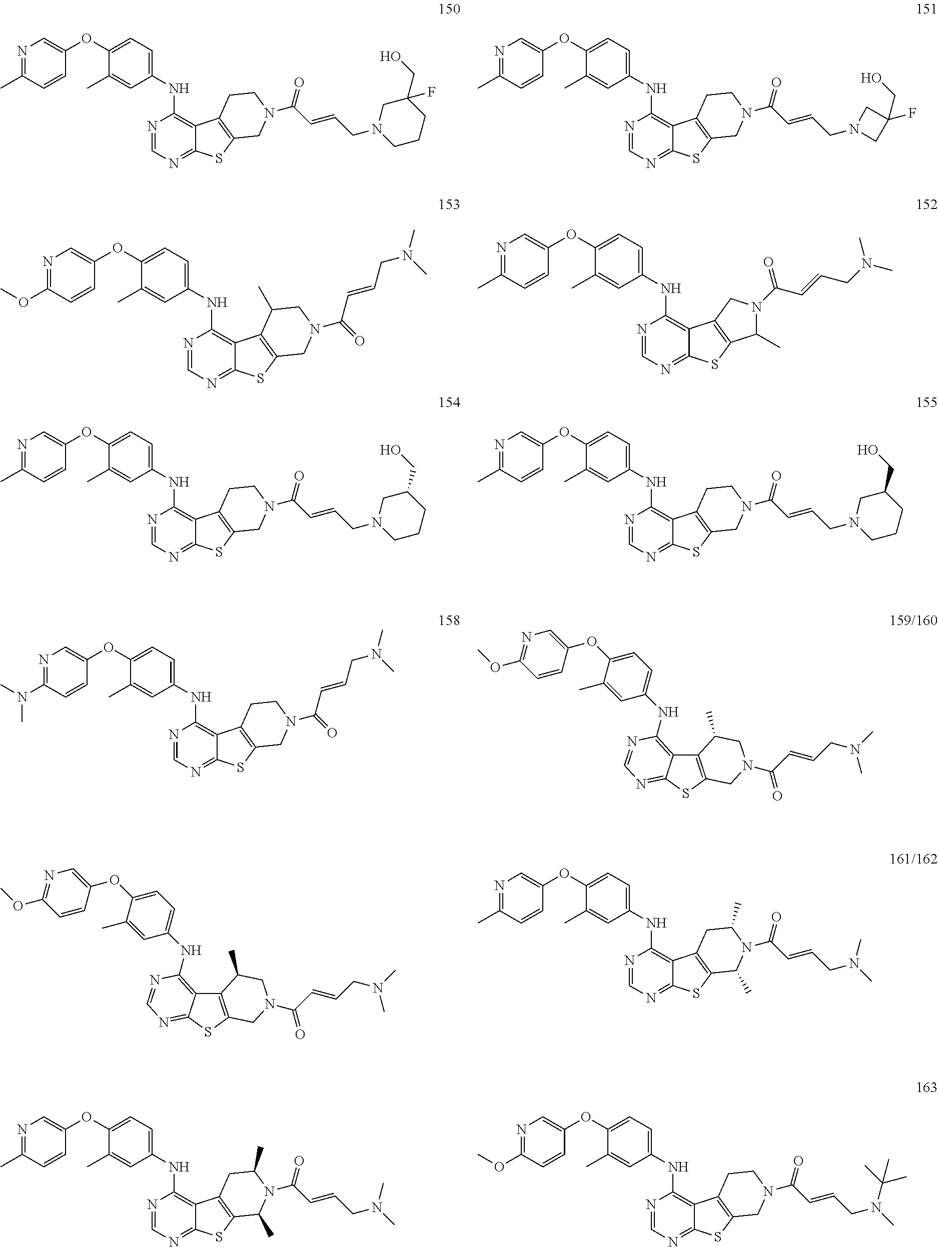

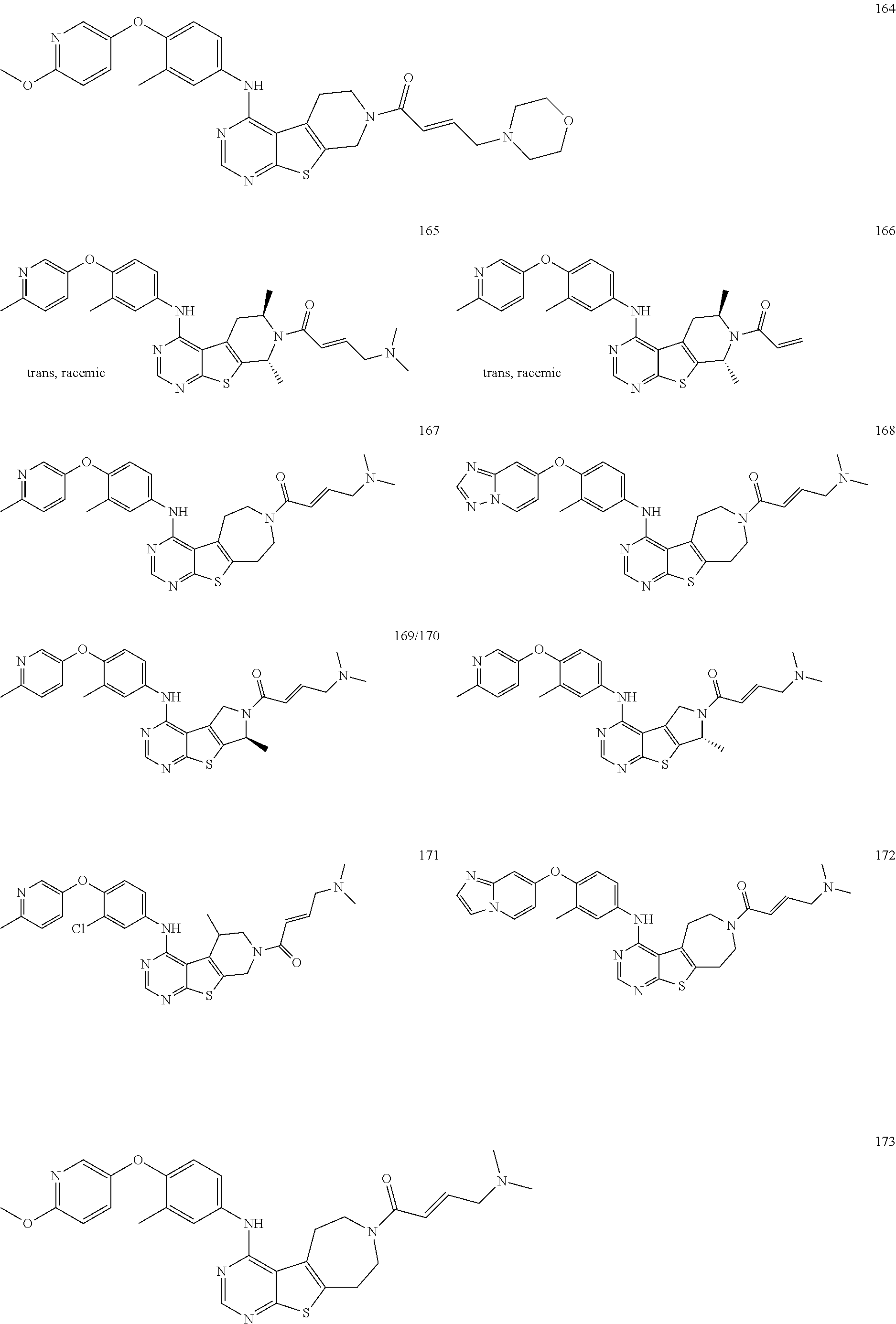

8. A pharmaceutical composition comprising a compound of claim 1 and one or more pharmaceutically acceptable excipients.

9. A method of treating, preventing, or managing a disorder mediated by HER2 or a HER2 mutant in a subject, comprising administering a therapeutically or prophylactically effective amount of claim 1, a composition, or a pharmaceutical composition thereof, to the subject.

10. The method of claim 9, wherein the disorder is cancer.

11. The method of claim 10, wherein the cancer is a solid tumor.

12. The method of claim 11, wherein the cancer is breast cancer, gastric cancer, esophageal cancer, ovarian cancer, endometrial cancer, endometrial serous carcinoma, cervix cancer, bladder cancer, lung cancer, colorectal cancer, head and neck cancer, cholangial cancer, germ cell cancer, glioblastoma, liver cancer, melanoma, osteosarcoma, pancreatic cancer, renal cell carcinoma, salivary duct carcinoma, or soft tissue cancer.

13. The method of claim 12, wherein the cancer is breast cancer, gastric cancer, esophageal cancer, ovarian cancer, or endometrial cancer.

14. The method of claim 13, wherein the cancer is breast cancer.

15. The method of claim 14, wherein the breast cancer is a metastatic breast cancer that spreads to the brain.

16. The method of claim 14, wherein the breast cancer is a HER2 amplified metastatic breast cancer.

17. The method of claim 14, wherein the breast cancer is characterized by the presence of one or more HER2 mutants.

18. The compound of claim 1, wherein:
X is N;
Y is NR⁶;
Z is 0;
Q is S;
n is 1 or 2;
m is 1 or 2;
t is 0, 1, or 2;
R¹ is aryl or heteroaryl;
each instance of R² is independently hydrogen, alkyl, alkenyl, alkynyl, haloalkyl, hydroxyalkyl, heteroalkyl, halogen, cyano, nitro, amido, cycloalkyl, heterocyclyl, aryl, or heteroaryl;
each instance of R³ is independently alkyl, alkenyl, alkynyl, haloalkyl, hydroxyalkyl, heteroalkyl, halogen, hydroxyl or alkoxy;
W is
-L-(NR⁶)ₛ—C(═O)—(CR¹⁰)=CR¹⁰)—R¹¹, or
-L-(NR⁶)ₛ—C(═O)—C(═CR¹⁰R¹⁰)—R¹¹;
L is a bond or C₁-C₃ alkylene;
s is 0 or 1;
each instance of R¹⁰ is independently hydrogen, alkyl, haloalkyl, hydroxyalkyl, or heteroalkyl;
R¹¹ is hydrogen, —OR¹², —(C₁-C₃ alkylene) —OR¹², —NR¹²R¹³, —(C₁-C₃ alkylene) —NR¹²R¹³, or cycloalkyl; and
R¹² and R¹³ are independently hydrogen, alkyl, haloalkyl, hydroxyalkyl, heteroalkyl, alkoxy, cycloalkyl, heterocyclic, aryl, heteroaryl, cycloalkylalkyl, heterocyclylalkyl, arylalkyl, or heteroarylalkyl, or R¹² and R¹³ are taken together with nitrogen to form a cyclic moiety.

19. The compound of claim 1, wherein:
X is N;
Y is NR⁶;
Z is O;
Q is S;
n is 1 or 2;
m is 1;
t is 0 or 1;
R¹ is heteroaryl;
each instance of R² is independently hydrogen, alkyl, alkenyl, alkynyl, haloalkyl, hydroxyalkyl, or heteroalkyl;
each instance of R³ is independently alkyl, haloalkyl, hydroxyalkyl, heteroalkyl, or halogen;
W is
-L-(NR⁶)ₛ—C(═O)—(CR¹⁰)=CR¹⁰)—R¹¹;
L is a bond or C₁-C₃ alkylene;
s is 0;
each instance of R¹⁰ is independently hydrogen, alkyl, or haloalkyl;
R¹¹ is, —(C₁-C₃ alkylene) —NR¹²R¹³; and
R¹² and R¹³ are independently hydrogen, alkyl, haloalkyl, hydroxyalkyl, heteroalkyl, alkoxy, cycloalkyl, heterocyclic, or R¹² and R¹³ are taken together with nitrogen to form a cyclic moiety.

20. A compound, or a salt thereof, according to claim 1, wherein the compound is selected from: [chemical structure]
